(19) United States
(12) Patent Application Publication
(10) Pub. No.: US 2025/0000098 A1
(43) Pub. Date: Jan. 2, 2025

(54) INCREASED BIOLOGICAL AGENT PERFORMANCE AND REDUCED VARIATION ACROSS AREAS OF APPLICATION

(71) Applicant: BioControl, LLC, St. Paul, MN (US)

(72) Inventor: Linda L. KINKEL, Forest Lake, MN (US)

(21) Appl. No.: 18/710,338

(22) PCT Filed: Nov. 16, 2022
(86) PCT No.: PCT/US2022/079992

§ 371 Date: May 15, 2024

Related U.S. Application Data
(60) Provisional application No. 63/279,975, filed on Nov. 16, 2021.

(51) Int. Cl. A01N 63/28; A01N 63/22; A01N 63/30; A01P 1/00; A01P 3/00; A01P 21/00

(52) U.S. Cl. CPC A01N 63/28; A01N 63/22; A01N 63/30; A01P 1/00; A01P 3/00; A01P 21/00

(57) ABSTRACT

The disclosure provides microbial signalers that increase the plant growth-promoting function of target microbes present in commercial biological agents. The plant-growth promoting functions may include plant pathogen inhibiting functions, zinc solubilizing functions, or phosphate solubilizing functions. The disclosure further relates to compositions, comprising: the microbial signalers, and to methods of using the compositions to produce improved soil for plant growth.

### CROSS REFERENCE TO RELATED APPLICATIONS

The present application claims the benefit of priority to U.S. Provisional Application No. 63/279,975 filed Nov. 16, 2021, the contents of which are hereby incorporated by reference in their entirety for all purposes.

### REFERENCE TO AN ELECTRONIC SEQUENCE LISTING

The contents of the electronic sequence listing (BICL_002_02WO_SeqList_ST26.xml; Size: 86,879 bytes; and Date of Creation: Nov. 16, 2022) are herein incorporated by reference in its entirety.

### BACKGROUND

Commercially available biological agents (e.g. biological control agents, biofertilizers) may be used to improve soil quality and promote plant growth, based on their ability to suppress the growth of plant pathogens, and/or increase soil nutrient availability.

However, the use of commercial biological agents has been limited by, for example, a limited base of microbes upon which development is focused and/or a focus on single-strain inoculants. In particular, single strain inoculants can fail to provide a level of plant growth promotion and/or disease suppression that is sufficient to satisfy market demands. The capacity for a single microbial strain to provide protection against any possible soil borne pathogen on diverse crops in a wide range of physical and environmental conditions, and in the presence of complex and highly-variable naturally occurring soil microbial communities, is low. For instance, many commercial biological plant pathogen control agents, such as biological pesticides, perform poorly under low nutrient conditions.

Therefore, there is a need for compositions and methods for improving the quality of soil and promoting plant growth, particularly, compositions and methods that increase the activity of commercial biological agents to suppress plant pathogens, increase soil nutrient availability, and/or enable the commercial agents to function in low nutrient environments.

[symbol]

The disclosure provides compositions, comprising at least one microbial signaler, wherein the at least one microbial signaler is capable of enhancing a plant growth-promoting function of a target microbe. The disclosure further provides compositions, comprising: (a) at least one microbial signaler, and (b) at least one target microbe, wherein the at least one microbial signaler is capable of enhancing a plant growth-promoting function of the at least one target microbe. In some embodiments, the the plant growth-promoting function comprises: (a) plant pathogen-inhibiting function, (b) zinc solubilizing function, (c) phosphate solubilizing function, (d) production of an antibiotic, or (e) any combination thereof.

The disclosure also provides methods of method of producing a composition, the method comprising: bringing at least one target microbe in the physical proximity of at least one microbial signaler, wherein the at least one microbial signaler is capable of enhancing a plant growth-promoting function of the at least one target microbe, and methods of enhancing a plant growth-promoting function of a target microbe, the method comprising: bringing the target microbe in the physical proximity of at least one microbial signaler, wherein the at least one microbial signaler is capable of enhancing a plant growth-promoting function of the at least one target microbe.

The disclosure provides methods of producing an improved soil for plant growth, comprising: applying any one of the compositions disclosed herein to soil, thereby producing the improved soil for plant growth. In some embodiments, the methods comprise allowing a plant to grow in the improved soil. In some embodiments, the growth of the plant is more enhanced in the improved soil, as compared to the growth of the plant in a control soil, wherein the composition is not applied to the control soil. In some embodiments,

### BRIEF DESCRIPTION OF THE FIGURES

FIG. 1A (upper panel) shows a culture plate showing colonies of the active microbe in Streptomyces spp. in commercial product 1 and the microbial signaler MS2 inoculated >3 cm apart or 1 cm apart in the presence of the indicator microbe 22-D-2. The bottom panels of FIGS. 1A-1C are schematic legends showing the identity of the colonies on the plate. FIG. 1B (upper panel) shows a culture plate showing colonies of the active microbe in Streptomyces spp. in commercial product 2 and the microbial signaler MS8 inoculated >3 cm apart or 1 cm apart in the presence of the indicator microbe B3. FIG. 1C (upper panel) shows a culture plate showing colonies of the active microbe in

[symbol]

signaler MS5 inoculated >3 cm apart or 1 cm apart in the presence of the indicator microbe 22-D-2.

FIG. 2 shows a culture plate showing colonies of the active microbe in Streptomyces spp. in commercial product 1 and the microbial signaler MS5 inoculated >3 cm apart or 1 cm apart in the presence of the indicator microbe 33-U-4 either under low or high nutrient conditions.

FIG. 3 shows a graph depicting the inhibition zone size for Streptomyces spp. in commercial product 1 against the indicator microbes 33-U-4 or B3 under high or low nutrient conditions.

FIGS. 4A-4B show a graph depicting an increase in inhibition zone for Streptomyces spp. in commercial product 1 against the indicator microbes 33-U-4 (FIG. 4A) or B3 (FIG. 4B) under high nutrient conditions or low nutrient conditions in the presence of the each of the microbial signalers as indicated.

FIG. 5A depicts the percentage increase in the inhibition of five indicator microbes (Bacillus spp.) in the presence of a combination of: (a) each of the microbial signalers listed on the X axis, and (b) Streptomyces spp. in commercial product 1, as compared to the inhibition of the indicator microbe (Bacillus spp.) in the presence of Streptomyces spp. in commercial product 1 alone. FIG. 5B depicts the percentage increase in the inhibition of a indicator microbe (Bacillus spp. (22-D2)) in the presence of a combination of: (a) each of the microbial signalers listed on the X axis, and (b) Streptomyces spp. in commercial product 1, as compared to the inhibition of the indicator microbe (Bacillus spp. (22-D2)) in the presence of Streptomyces spp. in commercial product 1 alone. FIG. 5C depicts the percentage increase in the inhibition of a indicator microbe (Bacillus spp. (33-U-4)) in the presence of a combination of: (a) each of the microbial signalers listed on the X axis, and (b) Streptomyces spp. in commercial product 1, as compared to the inhibition of the indicator microbe (Bacillus spp. (33-U-4)) in the presence of Streptomyces spp. in commercial product 1 alone. FIG. 5D depicts the percentage increase in the inhibition of a indicator microbe (Bacillus spp. (B3)) in the presence of a combination of: (a) each of the microbial signalers listed on the X axis, and (b) Streptomyces spp. in commercial product 1, as compared to the inhibition of the indicator microbe (Bacillus spp. (B3)) in the presence of Streptomyces spp. in commercial product 1 alone. FIG. 5E depicts the percentage increase in the inhibition of a indicator microbe (Bacillus spp. (62-D-2)) in the presence of a

[symbol]

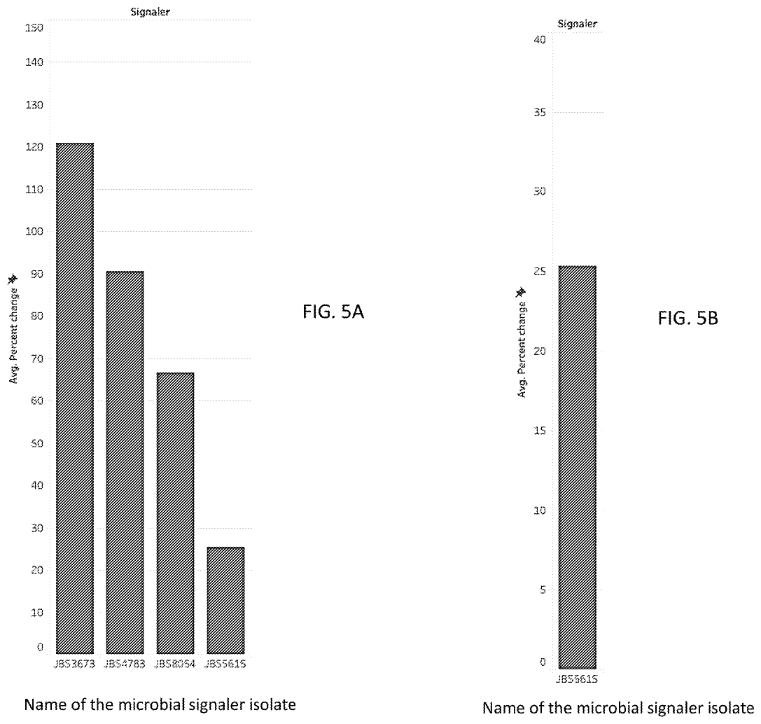

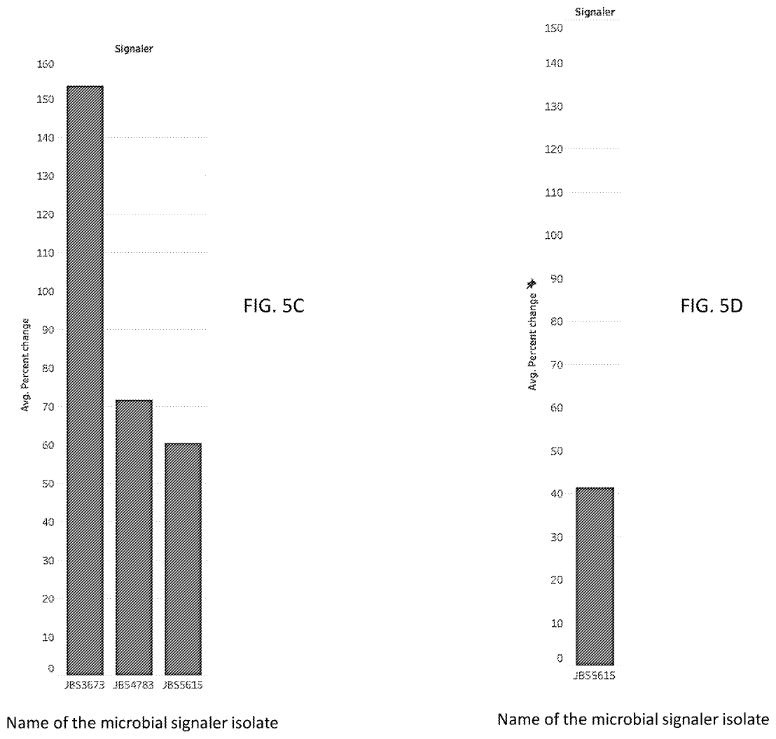

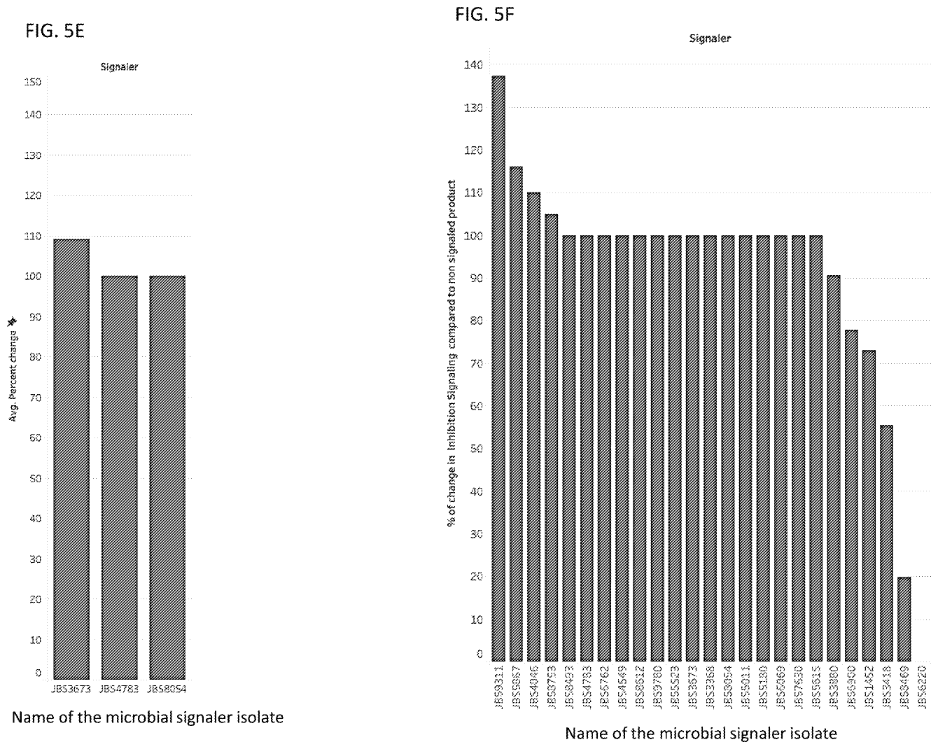

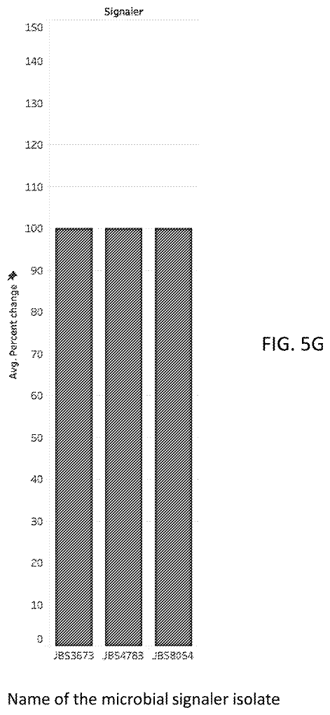

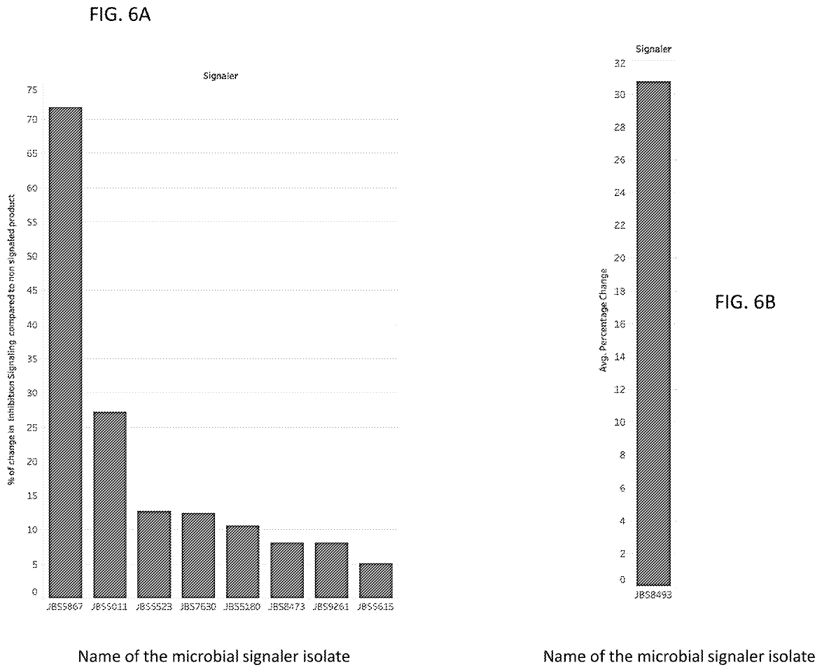

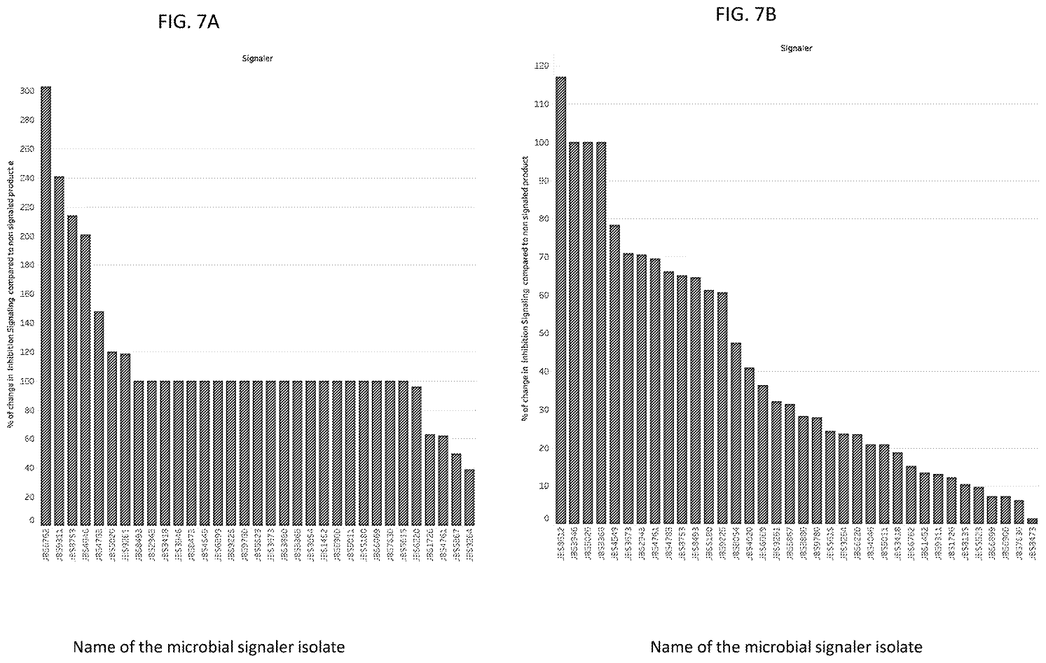

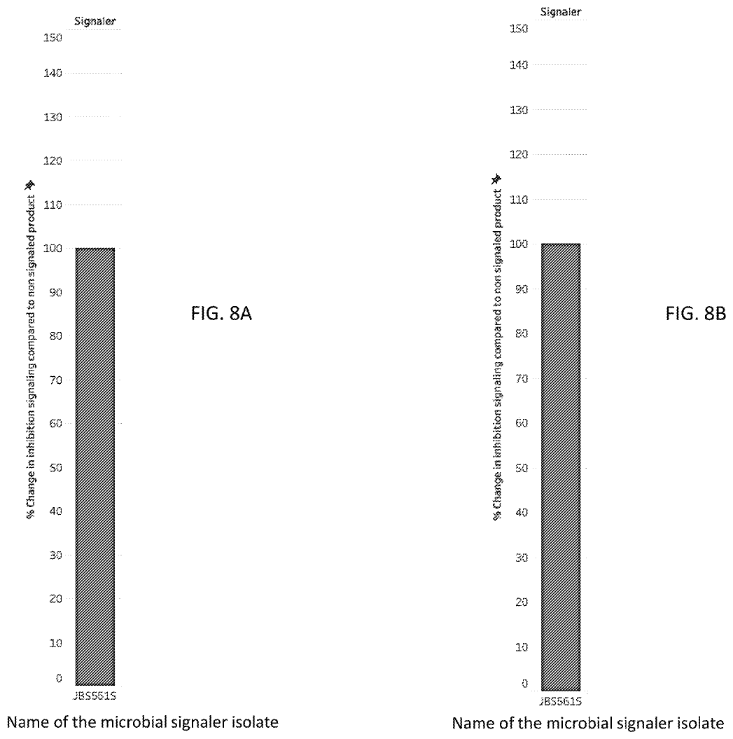

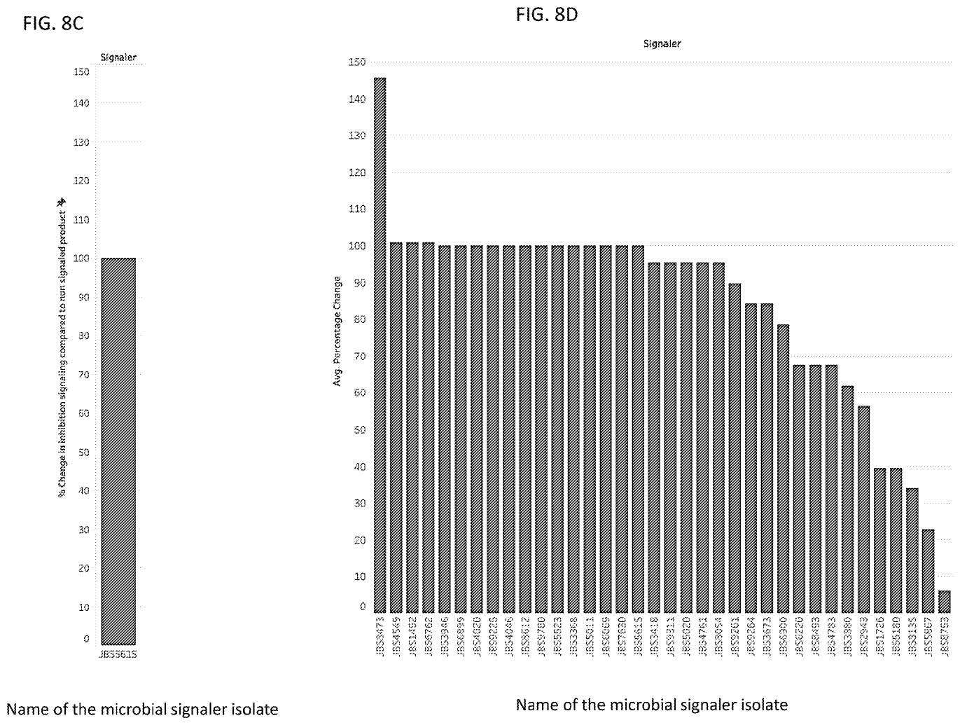

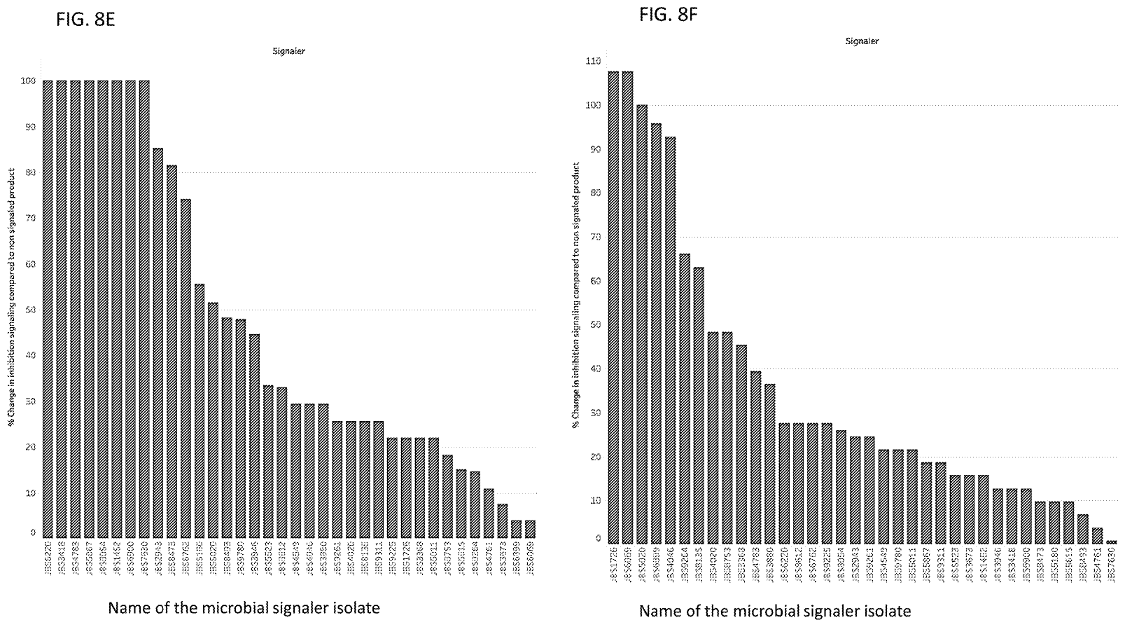

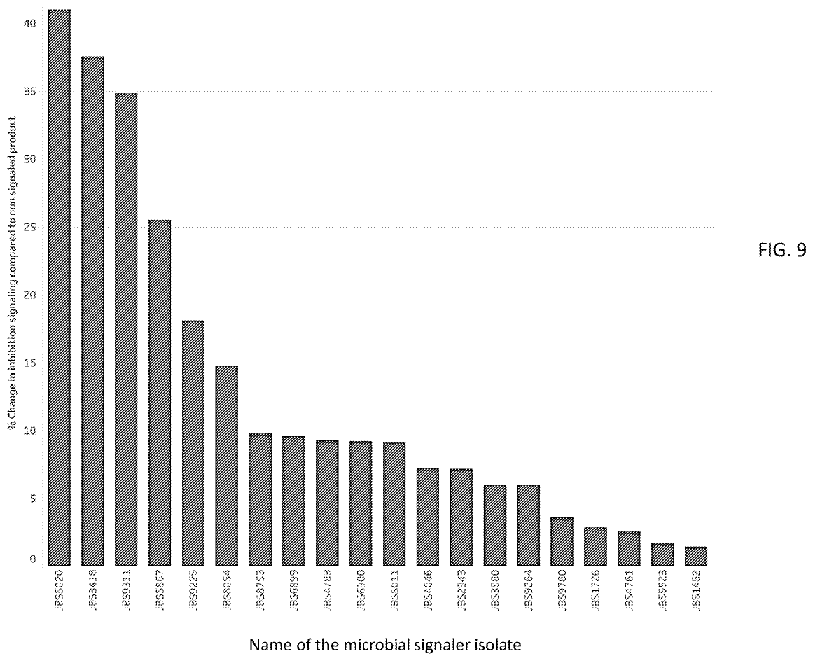

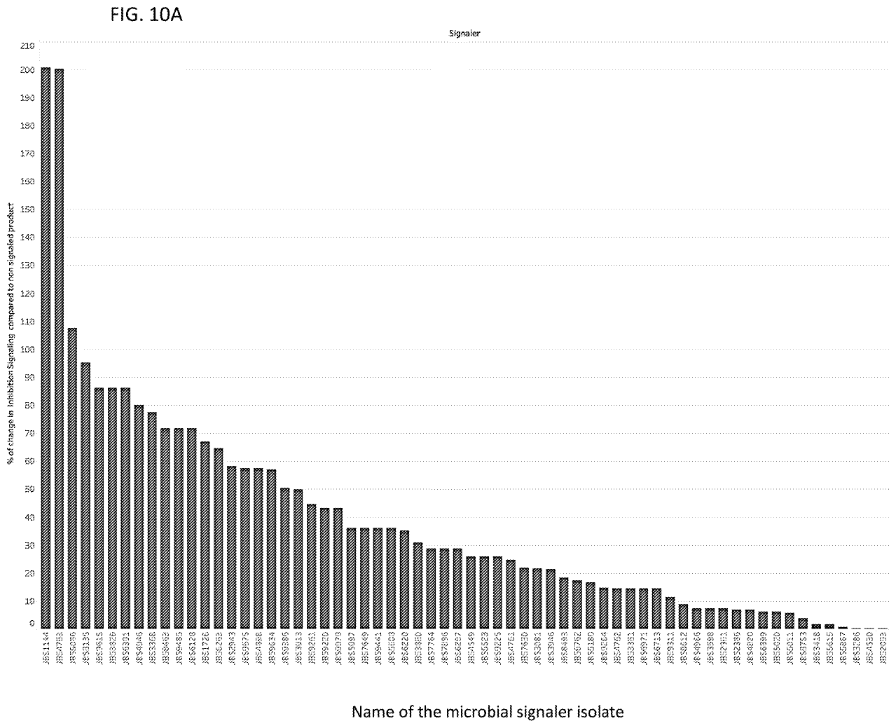

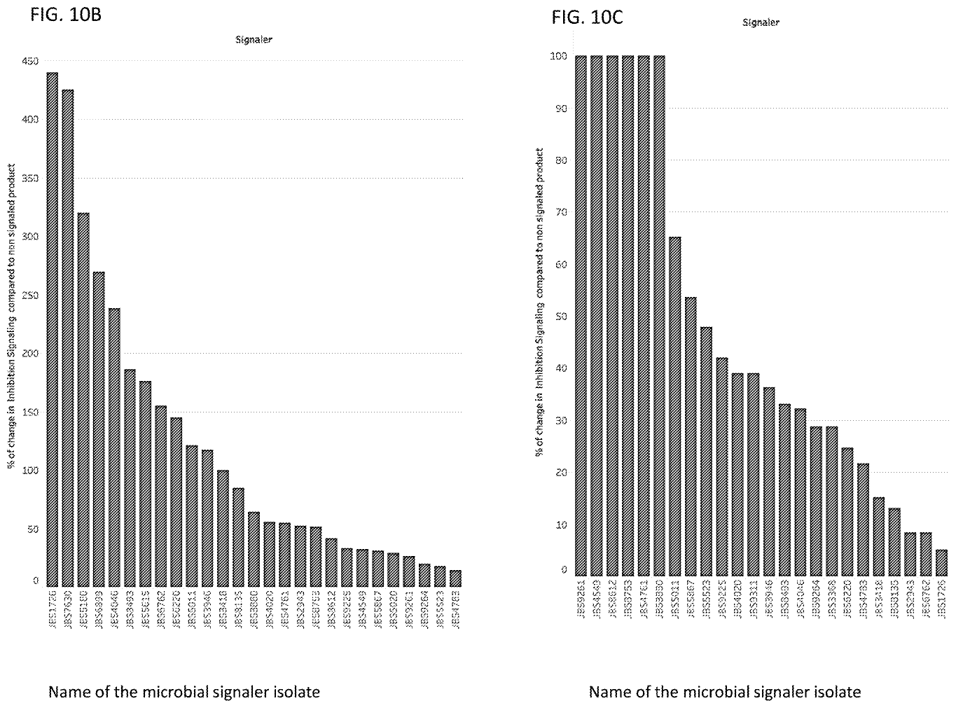

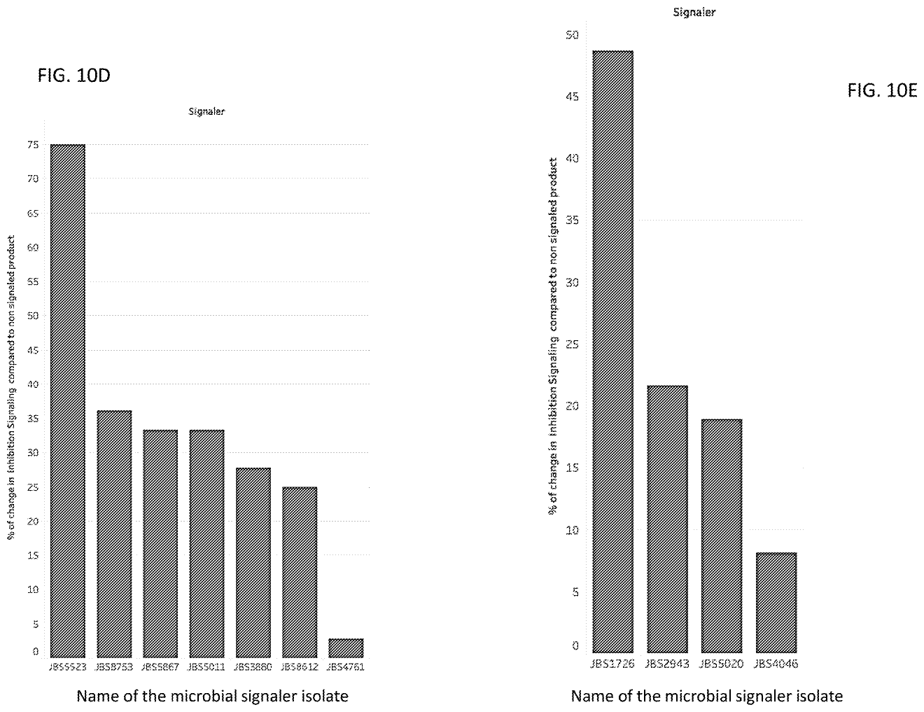

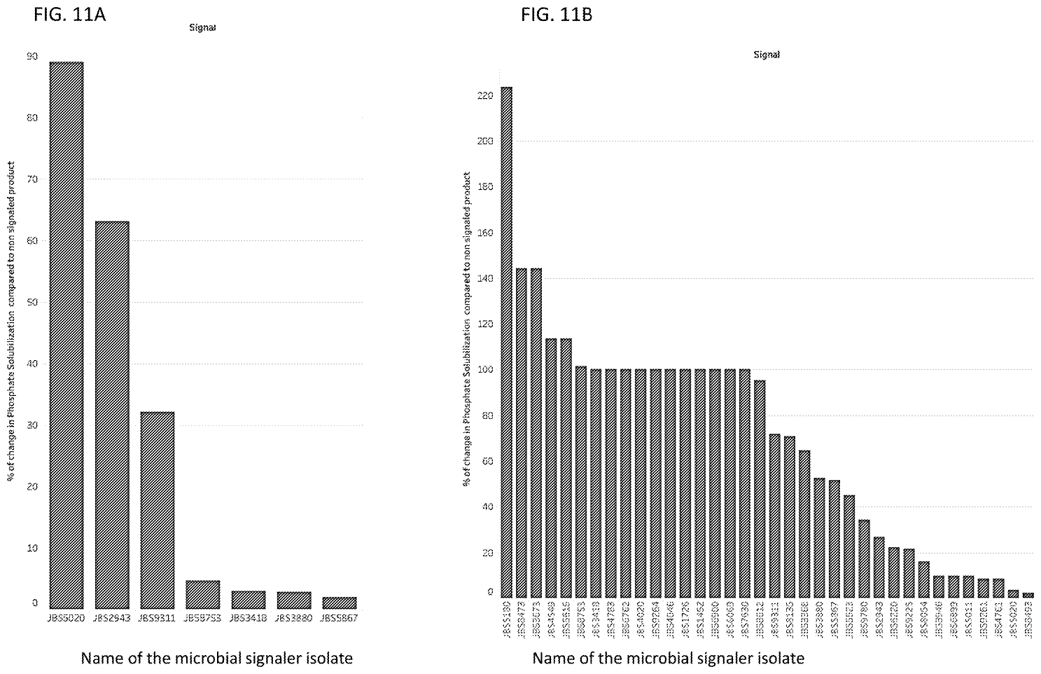

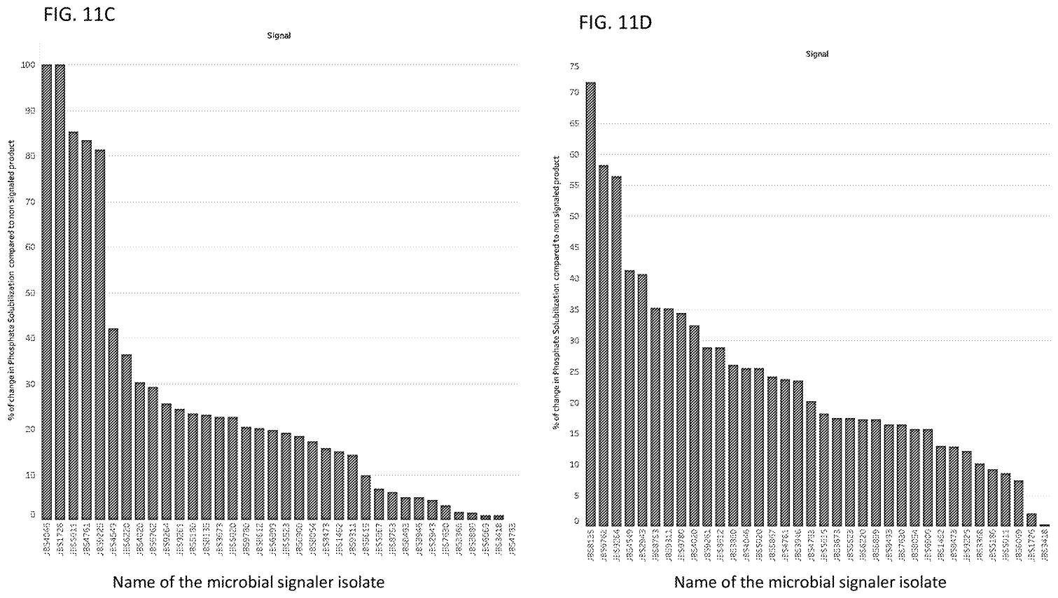

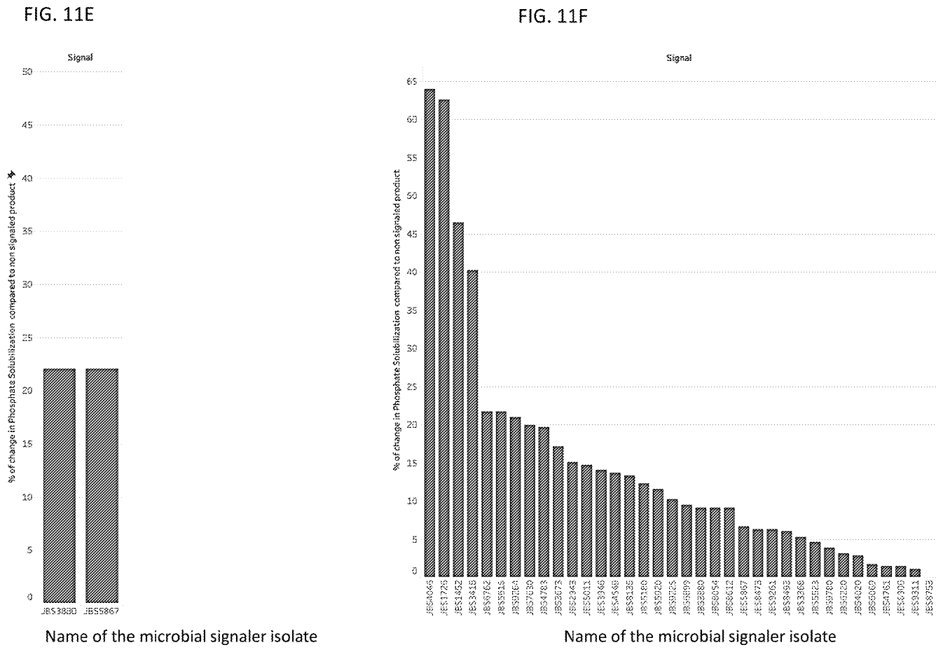

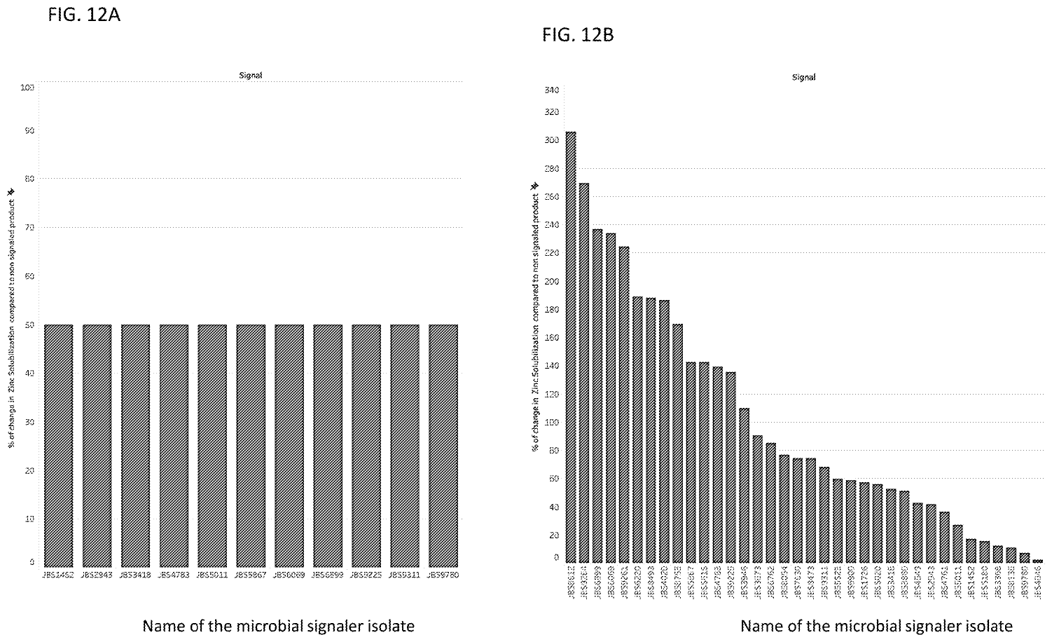

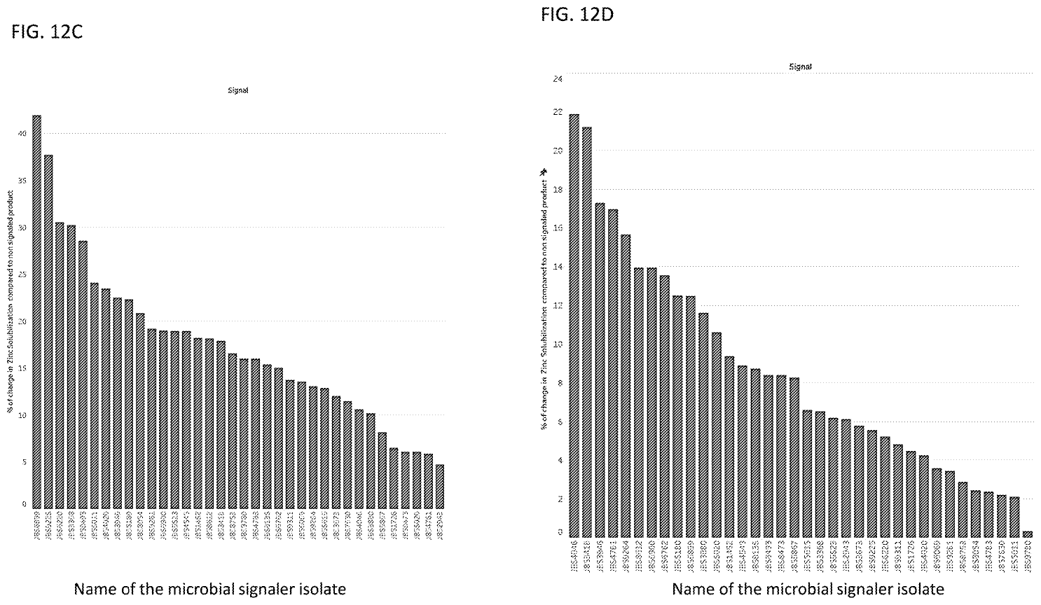

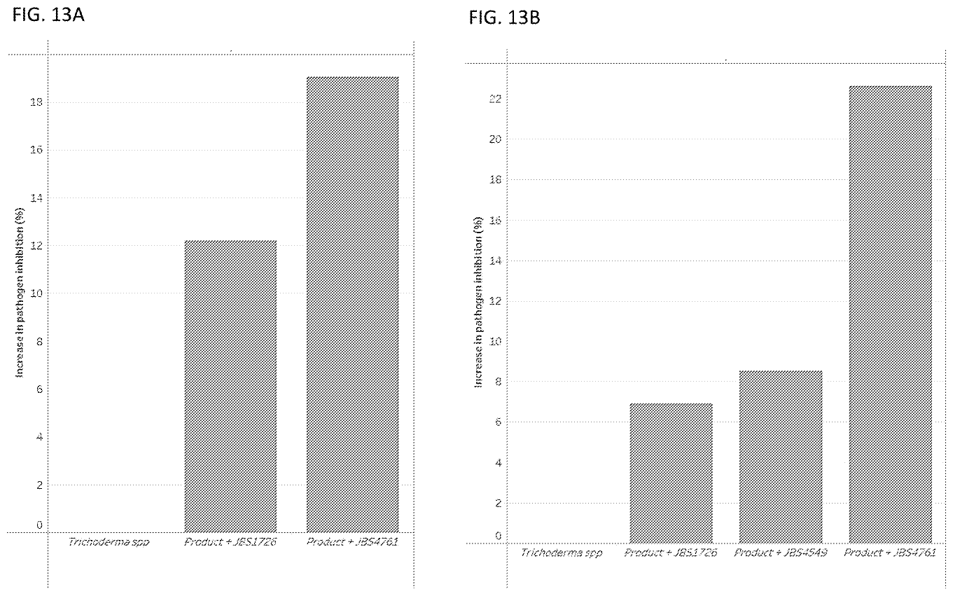

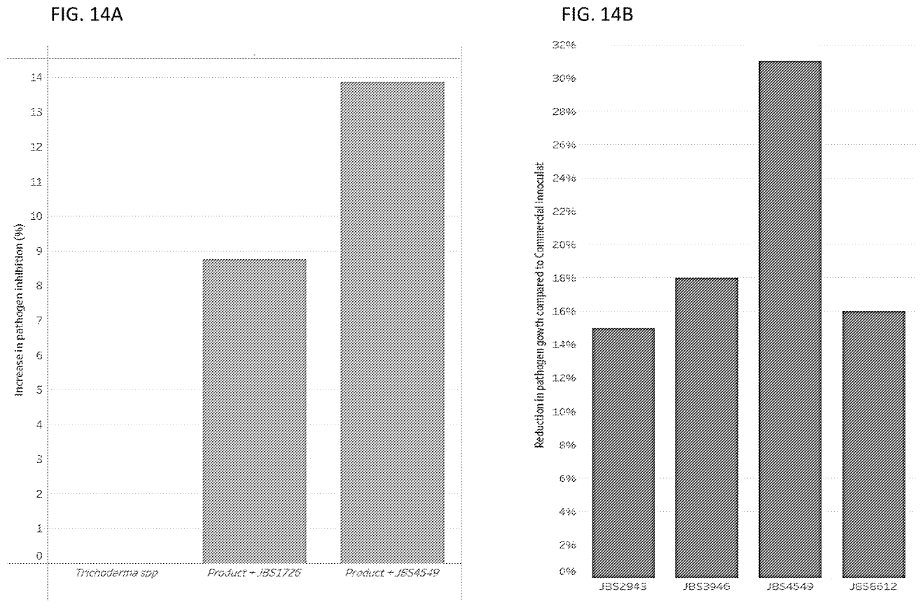

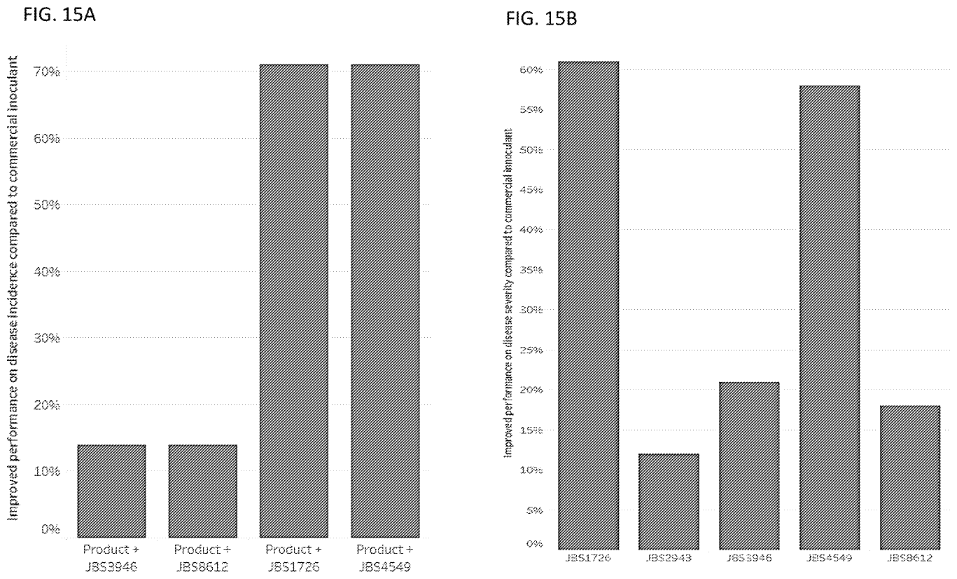

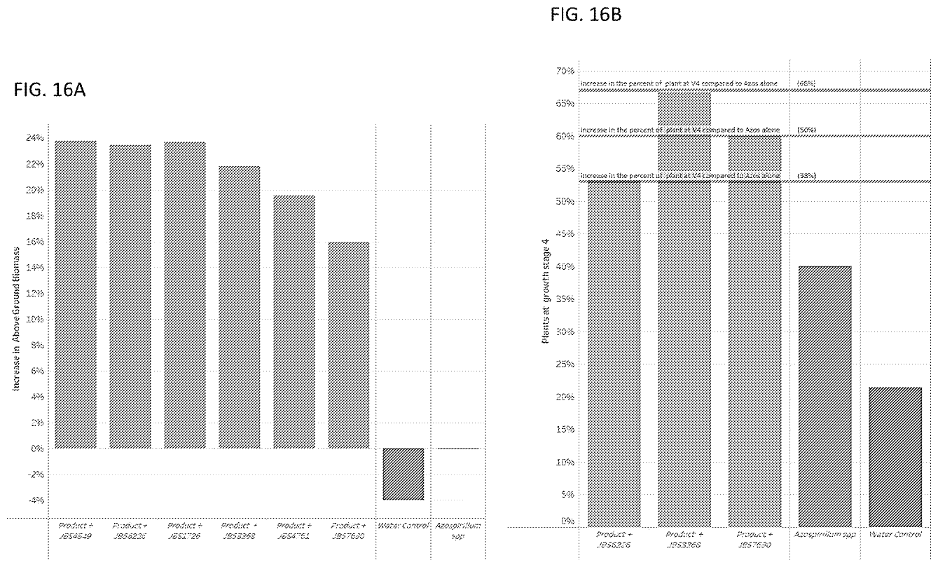

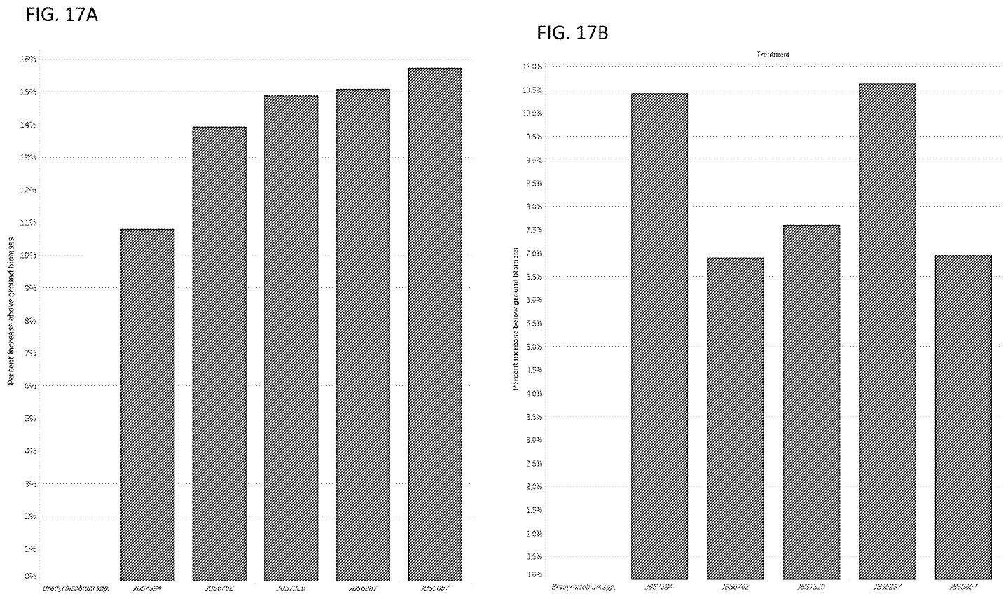

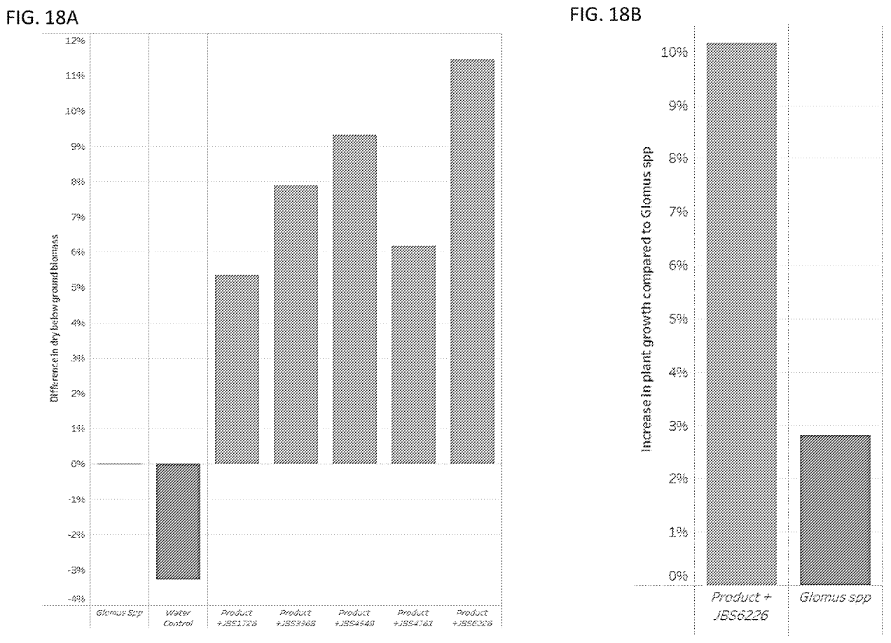

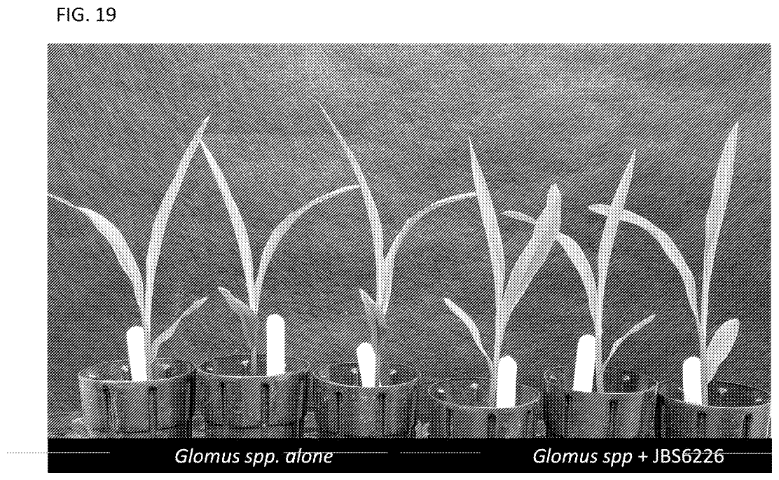

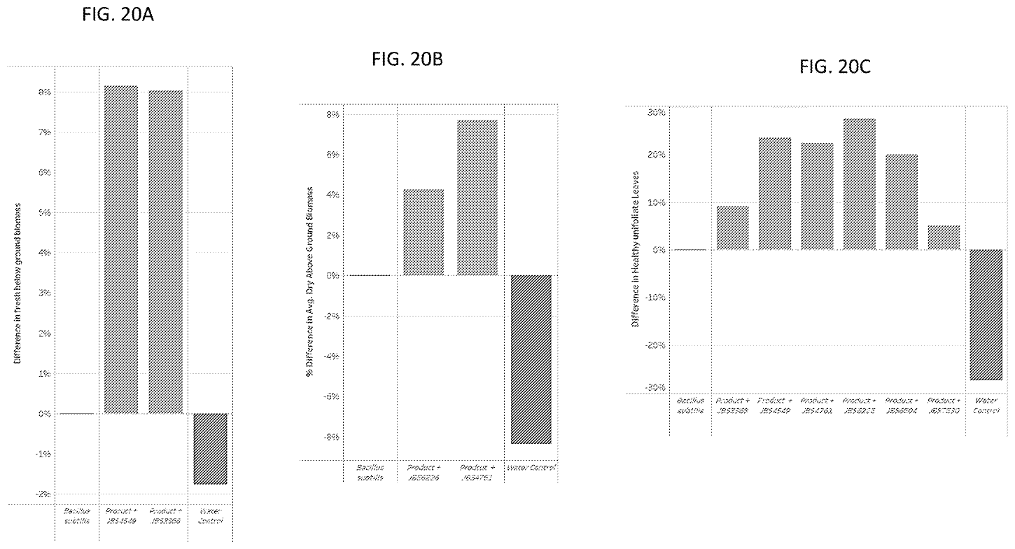

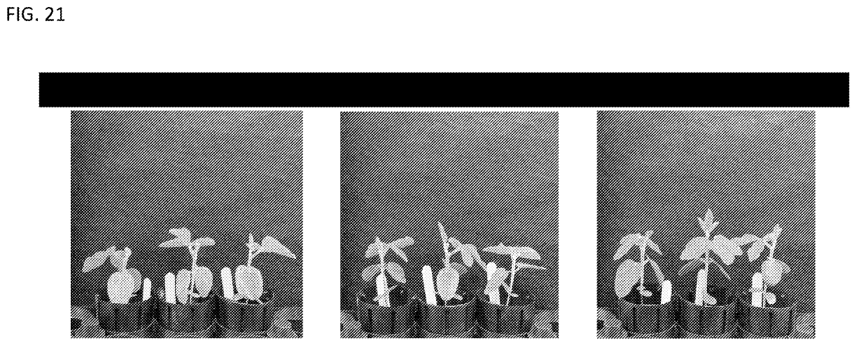

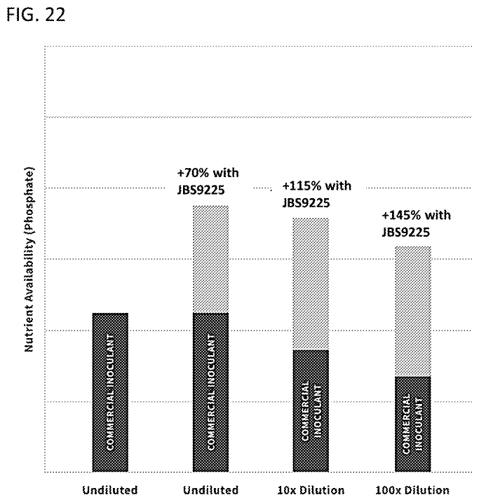

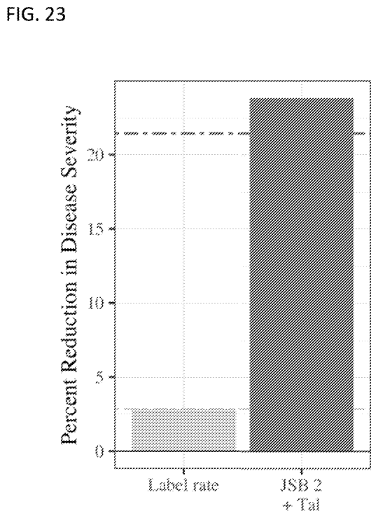

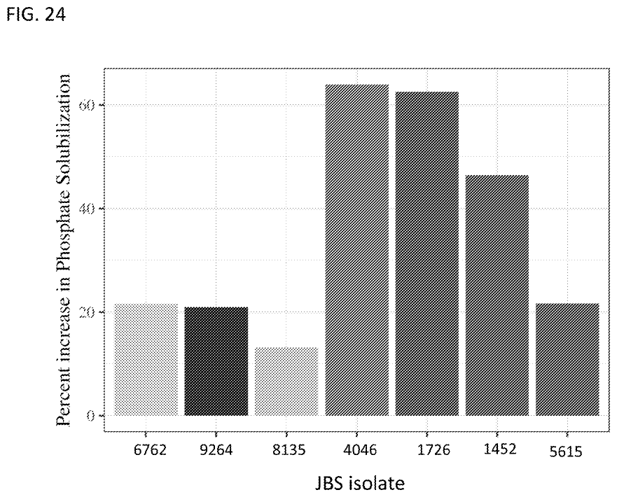

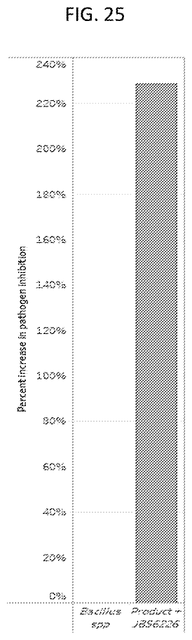

the X axis, and (b) Streptomyces spp. in commercial product 1, as compared to the inhibition of the indicator microbe (Bacillus spp. (62-D-2)) in the presence of Streptomyces spp. in commercial product 1 alone. FIG. 5F depicts the percentage increase in the inhibition of a plant pathogen (Streptomyces scabies) in the presence of a combination of: (a) each of the microbial signalers listed on the X axis, and (b) Streptomyces spp. in commercial product 1, as compared to the inhibition of the plant pathogen (Streptomyces scabies) in the presence of Streptomyces spp. in commercial product 1 alone. FIG. 5G depicts the percentage increase in the inhibition of a indicator microbe (Bacillus spp. (52-U-1)) in the presence of a combination of: (a) each of the microbial signalers listed on the X axis, and (b) Streptomyces spp. in commercial product 1, as compared to the inhibition of the indicator microbe (Bacillus spp. (52-U-1)) in the presence of Streptomyces spp. in commercial product 1 alone.

FIG. 6A depicts the percentage increase in the inhibition of a plant pathogen (Colletotrichum graminicola) in the presence of a combination of: (a) each of the microbial signalers listed on the X axis, and (b) target microbe Bacillus spp., as compared to the inhibition of the plant pathogen (Colletotrichum graminicola) in the presence of Bacillus spp. alone. FIG. 6B depicts the percentage increase in the inhibition of a plant pathogen (Pectobacterium caratovorum) in the presence of a combination of: (a) each of the microbial signalers listed on the X axis, and (b) target microbe Bacillus spp., as compared to the inhibition of the plant pathogen (Pectobacterium caratovorum) in the presence of target microbe Bacillus spp. alone.

FIG. 7A depicts the percentage increase in the inhibition of a plant pathogen (Rhizoctonia solani) in the presence of a combination of: (a) each of the microbial signalers listed on the X axis, and (b) target microbe Bacillus spp., as compared to the inhibition of the plant pathogen (Rhizoctonia solani) in the presence of target microbe Bacillus spp. alone. FIG. 7B depicts the percentage increase in the inhibition of a plant pathogen (Sclerotinia sclerotiorum) in the presence of a combination of: (a) each of the microbial signalers listed on the X axis, and (b) target microbe Bacillus spp., as compared to the inhibition of the plant pathogen (Sclerotinia sclerotiorum) in the presence of target microbe Bacillus spp. alone.

FIG. 8A depicts the percentage increase in the inhibition of a indicator microbe (Bacillus spp. (22-D2)) in the presence of a combination of: (a) each of the microbial signalers listed on the X axis, and (b) Streptomyces spp. in commercial product 2, as compared to the inhibition of the indicator microbe (Bacillus spp. (22-D2)) in the presence of Streptomyces spp. in commercial product 2 alone. FIG. 8B depicts the percentage increase in the inhibition of a indicator microbe (Bacillus spp.

[symbol]

the microbial signalers listed on the X axis, and (b) Streptomyces spp. in commercial product 2, as compared to the inhibition of the indicator microbe (Bacillus spp. (B3, 22-D2)) in the presence of Streptomyces spp. in commercial product 2 alone. FIG. 8C depicts the percentage increase in the inhibition of a indicator microbe (Bacillus spp. (B3)) in the presence of a combination of: (a) each of the microbial signalers listed on the X axis, and (b) Streptomyces spp. in commercial product 2, as compared to the inhibition of the indicator microbe (Bacillus spp. (B3)) in the presence of Streptomyces spp. in commercial product 2 alone. FIG. 8D depicts the percentage increase in the inhibition of a plant pathogen (Pythium irregulare) in the presence of a combination of: (a) each of the microbial signalers listed on the X axis, and (b) Streptomyces spp. in commercial product 2, as compared to the inhibition of the plant pathogen (Pythium irregulare) in the presence of Streptomyces spp. in commercial product 2 alone. FIG. 8E depicts the percentage increase in the inhibition of a plant pathogen (Rhizoctonia solani) in the presence of a combination of: (a) each of the microbial signalers listed on the X axis, and (b) Streptomyces spp. in commercial product 2, as compared to the inhibition of the plant pathogen (Rhizoctonia solani) in the presence of Streptomyces spp. in commercial product 2 alone. FIG. 8F depicts the percentage increase in the inhibition of a plant pathogen (Streptomyces scabies) in the presence of a combination of: (a) each of the microbial signalers listed on the X axis, and (b) Streptomyces spp. in commercial product 2, as compared to the inhibition of the plant pathogen (Streptomyces scabies) in the presence of Streptomyces spp. in commercial product 2 alone.

FIG. 9 depicts the percentage increase in the inhibition of a plant pathogen (Streptomyces scabies) in the presence of a combination of: (a) each of the microbial signalers listed on the X axis, and (b) Bacillus spp. in a commercial product, as compared to the inhibition of the plant pathogen (Streptomyces scabies) in the presence of Bacillus spp. in a commercial product alone.

FIG. 10A depicts the percentage increase in the inhibition of a plant pathogen (Fusarium culmorum) in the presence of a combination of: (a) each of the microbial signalers listed on the X axis, and (b) Talaromyces spp. in a commercial product, as compared to the inhibition of the plant pathogen (Fusarium culmorum) in the presence of Talaromyces spp. in a commercial product alone. FIG. 10B depicts the percentage increase in the inhibition of a plant pathogen (Fusarium graminearum) in the presence of a combination of: (a) each of the microbial signalers listed on the X axis, and (b) Talaromyces spp. in a commercial product, as compared to the inhibition of the

[symbol]

Talaromyces spp. in a commercial product alone. FIG. 10C depicts the percentage increase in the inhibition of a plant pathogen (Fusarium oxysporum) in the presence of a combination of: (a) each of the microbial signalers listed on the X axis, and (b) Talaromyces spp. in a commercial product, as compared to the inhibition of the plant pathogen (Fusarium oxysporum) in the presence of Talaromyces spp. in a commercial product alone. FIG. 10D depicts the percentage increase in the inhibition of a plant pathogen (Pythium irregulare) in the presence of a combination of: (a) each of the microbial signalers listed on the X axis, and (b) Talaromyces spp. in a commercial product, as compared to the inhibition of the plant pathogen (Pythium irregulare) in the presence of Talaromyces spp. in a commercial product alone. FIG. 10E depicts the percentage increase in the inhibition of a plant pathogen (Rhizoctonia solani) in the presence of a combination of: (a) each of the microbial signalers listed on the X axis, and (b) Talaromyces spp. in a commercial product, as compared to the inhibition of the plant pathogen (Rhizoctonia solani) in the presence of Talaromyces spp. in a commercial product alone.

FIG. 11A depicts the percentage increase in the phosphate solubilization in the presence of a combination of: (a) each of the microbial signalers listed on the X axis, and (b) Streptomyces spp. in commercial product 1, as compared to the phosphate solubilization in the presence of Streptomyces spp. in commercial product 1 alone. FIG. 11B depicts the percentage increase in the phosphate solubilization in the presence of a combination of: (a) each of the microbial signalers listed on the X axis, and (b) Pseudomonas spp.; Comamonas spp.; Citrobacter spp.; and Enterobacter spp. in a commercial product, as compared to the phosphate solubilization in the presence of Pseudomonas spp.; Comamonas spp.; Citrobacter spp.; and Enterobacter spp. in a commercial product alone. FIG. 11C depicts the percentage increase in the phosphate solubilization in the presence of a combination of: (a) each of the microbial signalers listed on the X axis, and (b) Streptomyces spp. in commercial product 2, as compared to the phosphate solubilization in the presence of Streptomyces spp. in commercial product 2 alone. FIG. 11D depicts the percentage increase in the phosphate solubilization in the presence of a combination of: (a) each of the microbial signalers listed on the X axis, and (b) Trichoderma spp. in a commercial product, as compared to the phosphate solubilization in the presence of Trichoderma spp. in a commercial product alone. FIG. 11E depicts the percentage increase in the phosphate solubilization in the presence of a combination of: (a) each of the microbial signalers listed on the X axis, and (b) Bacillus spp. in a commercial product, as compared to the phosphate

[symbol]

product alone. FIG. 11F depicts the percentage increase in the phosphate solubilization in the presence of a combination of: (a) each of the microbial signalers listed on the X axis, and (b) Talaromyces spp. in a commercial product, as compared to the phosphate solubilization in the presence of Talaromyces spp. in a commercial product alone.

FIG. 12A depicts the percentage increase in the zinc solubilization in the presence of a combination of: (a) each of the microbial signalers listed on the X axis, and (b) Streptomyces spp. in commercial product 1, as compared to the zinc solubilization in the presence of Streptomyces spp. in commercial product 1 alone. FIG. 12B depicts the percentage increase in the zinc solubilization in the presence of a combination of: (a) each of the microbial signalers listed on the X axis, and (b) Streptomyces spp. in commercial product 2, as compared to the zinc solubilization in the presence of Streptomyces spp. in commercial product 2 alone. FIG. 12C depicts the percentage increase in the zinc solubilization in the presence of a combination of: (a) each of the microbial signalers listed on the X axis, and (b) Trichoderma spp. in a commercial product, as compared to the zinc solubilization in the presence of Trichoderma spp. in a commercial product alone. FIG. 12D depicts the percentage increase in the zinc solubilization in the presence of a combination of: (a) each of the microbial signalers listed on the X axis, and (b) Talaromyces spp. in a commercial product, as compared to the zinc solubilization in the presence of Talaromyces spp. in a commercial product alone.

FIG. 13A depicts the percentage increase in the inhibition of Fusarium culmorum as measured by Assay 1 described in Example 3 in the presence of a combination of: (a) the microbial signaler listed on the X axis, and (b) a Trichoderma spp. microbe in a commercial product, as compared to the inhibition of the plant pathogen in the presence of the Trichoderma spp. microbe in the commercial product alone. FIG. 13B depicts the percentage increase in the inhibition of Fusarium culmorum as measured by Assay 2 described in Example 3 in the presence of a combination of: (a) the microbial signaler listed on the X axis, and (b) a Trichoderma spp. microbe in a commercial product, as compared to the inhibition of the plant pathogen in the presence of the Trichoderma spp. microbe in the commercial product alone.

FIG. 14A depicts the percentage increase in the inhibition of a plant pathogen (Phytophthora sojae) as measured by Assay 1 in the presence of a combination of: (a) each of the microbial signalers listed on the X axis, and (b) a Trichoderma spp. microbe in a commercial product, as compared to the inhibition of the plant pathogen (Phytophthora sojae) in the presence of the Trichoderma spp. microbe in the commercial product on its own. FIG. 14B depicts the percentage increase

[symbol]

as measured by Assay 2 in the presence of a combination of: (a) each of the microbial signalers listed on the X axis, and (b) a Trichoderma spp. microbe in a commercial product, as compared to the inhibition of the plant pathogen (Phytophthora sojae) in the presence of the Trichoderma spp. microbe in the commercial product on its own.

FIG. 15A depicts the percentage increase in Phytophthora sojae disease suppression (wherein a reduction in disease incidence is measured based on a reduction in the proportion of infected plants) on soybean plants inoculated with: (a) each of the microbial signalers listed on the X axis, and (b) a Trichoderma spp. microbe in a commercial product, as compared to the Trichoderma spp. microbe in the commercial product on its own. FIG. 15B depicts the percentage increase in Phytophthora sojae disease suppression (wherein a reduction in disease severity is assessed on a scale of 1 through 5) on soybean plants inoculated with: (a) each of the microbial signalers listed on the X axis, and (b) a Trichoderma spp. microbe in a commercial product, as compared to the Trichoderma spp. microbe in the commercial product on its own.

FIG. 16A depicts the percentage increase in aboveground biomass of corn inoculated with: (a) each of the microbial signalers listed on the X axis, and (b) a Azospirillum spp. microbe in a commercial product, as compared to the Azospirillum spp. microbe in the commercial product on its own, or just water. FIG. 16B depicts the percentage increase in corn plants reaching vegetative growth stage 4 (V4), which were inoculated with: (a) each of the microbial signalers listed on the X axis, and (b) a Azospirillum spp. microbe in a commercial product, as compared to the Azospirillum spp. microbe in the commercial product on its own or just water.

FIG. 17A depicts the percentage increase in aboveground biomass of soybean inoculated with: (a) each of the microbial signalers listed on the X axis, and (b) a Bradyrhizobium spp. microbe in a commercial product, as compared to the Bradyrhizobium spp. microbe in the commercial product on its own. FIG. 17B depicts the percentage increase in aboveground biomass of soybean inoculated with: (a) each of the microbial signalers listed on the X axis, and (b) a Bradyrhizobium spp. microbe in a commercial product, as compared to the Bradyrhizobium spp. microbe in the commercial product on its own.

FIG. 18A depicts the percentage increase in belowground biomass of corn plants inoculated with: (a) each of the microbial signalers listed on the X axis, and (b) a Glomus spp. in a mycorrhizal commercial

[symbol]

commercial product on its own or just water. FIG. 18B depicts the percentage increase in average growth stage among soybean plants inoculated with: (a) each of the microbial signalers listed on the X axis, and (b) a Glomus spp. in a mycorrhizal commercial product, as compared to the Glomus spp. in a mycorrhizal commercial product on its own.

FIG. 19 is an image of corn plants inoculated with with: (a) the indicated microbial signaling isolate, and (b) a Glomus spp. in a mycorrhizal commercial product, as compared to the Glomus spp. in a mycorrhizal commercial product on its own. The addition of the microbial signaling isolate enhances plant vigor and reduces purple coloration associated with nutrient stress.

FIG. 20A depicts the percentage increase in fresh belowground biomass of corn plants inoculated with: (a) each of the microbial signalers listed on the X axis, and (b) a Bacillus spp. in a commercial product, as compared to the Bacillus spp. in a commercial product on its own or just water. FIG. 20B depicts the percentage increase in dry aboveground biomass of soybean plants inoculated with: (a) each of the microbial signalers listed on the X axis, and (b) a Bacillus spp. in a commercial product, as compared to the Bacillus spp. in a commercial product on its own or just water. FIG. 20C depicts the percentage increase in frequency of healthy unifoliate leaves of soybean inoculated with: (a) each of the microbial signalers listed on the X axis, and (b) a Bacillus spp. in a commercial product, as compared to the Bacillus spp. in a commercial product on its own or just water.

FIG. 21 shows images of soybean plants inoculated with: (a) the indicated microbial signaler listed, and (b) a Bacillus spp. in a commercial product, as compared to the Bacillus spp. in a commercial product on its own or just water.

FIG. 22 depicts the percentage increase in in vitro phosphate solubilization by Pseudomonas spp.; Comamonas spp.; Citrobacter spp.; and Enterobacter spp. in a commercial product at three different inoculum densities (1× undiluted, 10× diluted, 100× diluted) in presence of a microbial signaler disclosed herein, JBS9225.

FIG. 23 depicts the percentage reduction in Pythium disease severity in plants inoculated with: (a) a combination of microbial signalers JBS4783, JBS8135, JBS3880, and (b) a Talaromyces spp. target microbe in a commercial product, as compared to the Talaromyces spp. target microbe in a commercial product on its own.

FIG. 24 depicts the

[symbol]

of a combination of: (a) each of the microbial signalers listed on the X axis, and (b)) a Talaromyces spp. target microbe in a commercial product, as compared to the phosphate solubilization in the presence of) a Talaromyces spp. target microbe in a commercial product alone.

FIG. 25 depicts the percentage increase in the inhibition of Fusarium culmorum in the presence of a combination of: (a) JBS6226, and (b) a Bacillus spp. microbe in a commercial product, as compared to the inhibition of the plant pathogen in the presence of the Bacillus spp. microbe in the commercial product alone.

### DETAILED DESCRIPTION

### Definitions

While the following terms are believed to be well understood by one of ordinary skill in the art, the following definitions are set forth to facilitate explanation of the presently disclosed subject matter.

The term “a” or “an” may refer to one or more of that entity, i.e. can refer to plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

Reference throughout this specification to “one embodiment”, “an embodiment”, “one aspect”, or “an aspect” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

As used herein, in particular embodiments, the terms “about” or “approximately” when preceding a numerical value indicates the value plus or minus a range of 10% unless otherwise stated or otherwise evident by the context, and except where such a range would exceed 100% of a possible value, or fall below 0% of a possible value, such as less than 0 CFU/ml of a bacteria, or more than 100% of a inhibition of growth.

As used herein the terms

[symbol]

terms are used interchangeably and include, but are not limited to, the two prokaryotic domains, Bacteria and Archaea, eukaryotic fungi and protozoa, as well as viruses.

The term “microbial community” means a group of microbes comprising two or more species or strains. Unlike microbial consortia, a microbial community does not have to be carrying out a common function, or does not have to be participating in, or leading to, or correlating with, a recognizable parameter, such as a phenotypic trait of interest (e.g. antimicrobial activity or production of compounds beneficial to plant growth).

As used herein, “isolate,” “isolated,” “isolated microbe,” and like terms, are intended to mean that the one or more microorganisms has been separated from at least one of the materials with which it is associated in a particular environment (for example soil, water, plant tissue).

As used herein, “soil” refers to any plant growth medium including any agriculturally acceptable growing media. Growing media may include, for example, soil, sand, compost, peat, soilless growing media containing organic and/or inorganic ingredients, artificial plant-growth substrates, polymer-based growth matrices, hydroponic nutrient and growth solutions, and combinations or mixtures thereof.

Microbes of the present disclosure may include spores and/or vegetative cells. In some embodiments, microbes of the present disclosure include microbes in a viable but non-culturable (VBNC) state, or a quiescent state. See Liao and Zhao (US Publication US2015267163A1). In some embodiments, microbes of the present disclosure include microbes in a biofilm. See Merritt et al. (U.S. Pat. No. 7,427,408).

Thus, an “isolated microbe” does not exist in its naturally occurring environment; rather, it is through the various techniques described herein that the microbe has been removed from its natural setting and placed into a non-naturally occurring state of existence. Thus, the isolated strain or isolated microbe may exist as, for example, a biologically pure culture, or as spores (or other forms of the strain) in association with an acceptable carrier.

As used herein, “spore” or “spores” refer to structures produced by bacteria and fungi that are adapted for survival and dispersal. Spores are generally characterized as dormant structures; however, spores are capable of differentiation through the process of germination. Germination is the differentiation of spores into vegetative cells that are capable of metabolic activity, growth, and reproduction. The germination of a single spore results in a single fungal or bacterial vegetative cell. Fungal

[symbol]

are necessary structures in fungal life cycles. Bacterial spores are structures for surviving conditions that may ordinarily be nonconductive to the survival or growth of vegetative cells.

As used herein, “microbial composition” refers to a composition comprising one or more microbes or one or more microbial signalers of the present disclosure, wherein a microbial composition, in some embodiments, is administered to the soil, field, or plants described herein.

As used herein, “carrier”, “acceptable carrier”, or “agricultural carrier” refers to a diluent, adjuvant, excipient, or vehicle with which the compound is administered.

In some embodiments, carriers may be granular in structure, such as soil, sand, soil particles, or sand particles. In further embodiments, the carriers may be dry, as opposed to a moist or wet carrier. In some embodiments, carriers can be in solid or liquid form.

The terms “multi strain inoculate composition”, “consortium”, “bioconsortia,” “microbial consortia,” and “synthetic consortia” interchangeably refer to a composition comprising two or more microbes. In some embodiments, the microbes in the consortium do not exist together in a naturally occurring environment. In some embodiments, the microbes are present in the consortium at ratios or amounts that are not naturally occurring. In some embodiments, the consortium comprises two or more species, or two or more strains of a species, of microbes.

In certain embodiments of the disclosure, the isolated microbes exist as isolated and biologically pure cultures (e.g., microbial isolate(s)). It will be appreciated by one of skill in the art, that an isolated and biologically pure culture of a particular microbe, denotes that said culture is substantially free (within scientific reason) of other living organisms and contains only the individual microbe in question. The culture can contain varying concentrations of said microbe. The present disclosure notes that isolated and biologically pure microbes often “necessarily differ from less pure or impure materials.” See, e.g. In re Bergstrom, 427 F.2d 1394, (CCPA 1970)(discussing purified prostaglandins), see also, In re Bergy, 596 F.2d 952 (CCPA 1979)(discussing purified microbes), see also, Parke-Davis & Co. v. H. K. Mulford & Co., 189 F. 95 (S.D.N.Y. 1911) (Learned Hand discussing purified adrenaline), aff'd in part, rev'd in part, 196 F. 496 (2d Cir. 1912), each of which are incorporated herein by reference. Furthermore, in some embodiments, the disclosure provides for certain quantitative measures of the concentration, or purity limitations, that must be found within an isolated and biologically pure microbial

[symbol]

embodiments, is a further attribute that distinguishes the presently disclosed microbes from those microbes existing in a natural state. See, e.g., Merck & Co. v. Olin Mathieson Chemical Corp., 253 F.2d 156 (4th Cir. 1958) (discussing purity limitations for vitamin B12 produced by microbes), incorporated herein by reference.

As used herein, “individual isolates” should be taken to mean a composition, or culture, comprising a predominance of a single genera, species, or strain, of microorganism, following separation from one or more other microorganisms. The phrase should not be taken to indicate the extent to which the microorganism has been isolated or purified. However, “individual isolates” can comprise substantially only one genus, species, or strain, of microorganism.

The term “growth medium” as used herein, is any medium which is suitable to support growth of a microbe. By way of example, the media may be natural or artificial. It should be appreciated that the media may be used alone or in combination with one or more other media. It may also be used with or without the addition of exogenous nutrients.

The medium may be amended or enriched with additional compounds or components, for example, a component which may assist in the interaction and/or selection of specific groups of microorganisms. For example, antibiotics (such as penicillin) or sterilants (for example, quaternary ammonium salts and oxidizing agents) could be present and/or the physical conditions (such as salinity, nutrients (for example organic and inorganic minerals (such as phosphorus, nitrogenous salts, ammonia, potassium and micronutrients such as cobalt and magnesium), pH, and/or temperature), methionine, prebiotics, ionophores, and beta glucans could be amended.

As used herein, “improved” or “enhanced” should be taken broadly to encompass improvement of a characteristic of interest, as compared to a control group, or as compared to a known average quantity associated with the characteristic in question. In the present disclosure, “improved” does not necessarily demand that the data be statistically significant (i.e. p<0.05); rather, any quantifiable difference demonstrating that one value (e.g. the average treatment value) is different from another (e.g. the average control value) can rise to the level of “improved.”

As used herein, “inhibiting” and “suppressing” are used interchangeably and these and other like terms should not be construed to require complete inhibition or suppression, although this may be desired in some embodiments.

The term “marker” or

[symbol]

microorganism type, microorganism strain or activity of a microorganism strain. A marker can be measured in biological samples and includes without limitation, a nucleic acid-based marker such as a ribosomal RNA gene, a peptide- or protein-based marker, and/or a metabolite or other small molecule marker.

The term “metabolite” as used herein is an intermediate or product of metabolism. A metabolite in one embodiment is a small molecule. Metabolites have various functions, including in fuel, structural, signaling, stimulatory and inhibitory effects on enzymes, as a cofactor to an enzyme, in defense, and in interactions with other organisms (such as pigments, odorants and pheromones). A primary metabolite is directly involved in normal growth, development and reproduction. A secondary metabolite is not directly involved in these processes but usually has an important ecological function. Examples of metabolites include but are not limited to antibiotics and pigments such as resins and terpenes, etc. Some antibiotics use primary metabolites as precursors, such as actinomycin which is created from the primary metabolite, tryptophan. Metabolites, as used herein, include small, hydrophilic carbohydrates; large, hydrophobic lipids and complex natural compounds.

As used herein, the term “genotype” refers to the genetic makeup of an individual cell, cell culture, tissue, organism, or group of organisms.

As used herein, the term “allele(s)” means any of one or more alternative forms of a gene, all of which alleles relate to at least one trait or characteristic. In a diploid cell, the two alleles of a given gene occupy corresponding loci on a pair of homologous chromosomes. Since the present disclosure, in embodiments, relates to QTLs, i.e. genomic regions that may comprise one or more genes or regulatory sequences, it is in some instances more accurate to refer to “haplotype” (i.e. an allele of a chromosomal segment) instead of “allele”, however, in those instances, the term “allele” should be understood to comprise the term “haplotype”. Alleles are considered identical when they express a similar phenotype. Differences in sequence are possible but not important as long as they do not influence phenotype.

As used herein, the term “locus” (loci plural) means a specific place or places or a site on a chromosome where for example a gene or genetic marker is found.

As used herein, the term “genetically linked” refers to two or more traits that are co-inherited at a high rate during breeding such that they are difficult to separate through crossing.

A “recombination” or

[symbol]

crossing over or independent assortment. The term “recombinant” refers to an organism having a new genetic makeup arising as a result of a recombination event.

As used herein, the term “molecular marker” or “genetic marker” refers to an indicator that is used in methods for visualizing differences in characteristics of nucleic acid sequences. Examples of such indicators are restriction fragment length polymorphism (RFLP) markers, amplified fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Markers further include polynucleotide sequences encoding 16S or 18S rRNA, and internal transcribed spacer (ITS) sequences, which are sequences found between small-subunit and large-subunit rRNA genes that have proven to be especially useful in elucidating relationships or distinctions among when compared against one another. Mapping of molecular markers in the vicinity of an allele is a procedure which can be performed by the average person skilled in molecular-biological techniques.

The primary structure of major rRNA subunit 16S comprise a particular combination of conserved, variable, and hypervariable regions that evolve at different rates and enable the resolution of both very ancient lineages such as domains, and more modern lineages such as genera. The secondary structure of the 16S subunit include approximately 50 helices which result in base pairing of about 67% of the residues. These highly conserved secondary structural features are of great functional importance and can be used to ensure positional homology in multiple sequence alignments and phylogenetic analysis. Over the previous few decades, the 16S rRNA gene has become the most sequenced taxonomic marker and is the cornerstone for the current systematic classification of bacteria and archaea (Yarza et al. 2014. Nature Rev. Micro. 12:635-45).

A sequence identity of 94.5% or lower for two 16S rRNA genes is strong evidence for distinct genera, 86.5% or lower is strong evidence for distinct families, 82% or lower is strong evidence for distinct orders, 78.5% is strong evidence for distinct classes, and 75% or lower is strong evidence for distinct phyla. The comparative analysis of 16S rRNA gene sequences enables the establishment of taxonomic thresholds that are useful not only for the classification of cultured microorganisms but also for the classification of the many environmental sequences. Yarza et al. 2014. Nature Rev. Micro. 12:635-45).

As used herein, the term

[symbol]

inherited in a dominant or recessive manner, or in a partial or incomplete-dominant manner. A trait may be monogenic (i.e. determined by a single locus) or polygenic (i.e. determined by more than one locus) or may also result from the interaction of one or more genes with the environment.

As used herein, the term “phenotype” refers to the observable characteristics of an individual cell, cell culture, organism (e.g., a bacterium), or group of organisms which results from the interaction between that individual's genetic makeup (i.e., genotype) and the environment.

As used herein, the term “chimeric” or “recombinant” when describing a nucleic acid sequence or a protein sequence refers to a nucleic acid, or a protein sequence, that links at least two heterologous polynucleotides, or two heterologous polypeptides, into a single macromolecule, or that re-arranges one or more elements of at least one natural nucleic acid or protein sequence. For example, the term “recombinant” can refer to an artificial combination of two otherwise separated segments of sequence, e.g., by chemical synthesis or by the manipulation of isolated segments of nucleic acids by genetic engineering techniques.

As used herein, a “synthetic nucleotide sequence” or “synthetic polynucleotide sequence” is a nucleotide sequence that is not known to occur in nature or that is not naturally occurring. Generally, such a synthetic nucleotide sequence will comprise at least one nucleotide difference when compared to any other naturally occurring nucleotide sequence.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence

[symbol]

desired parameters.

As used herein, the term “homologous” or “homologue” or “ortholog” is known in the art and refers to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, CA). Another alignment program is Sequencher (Gene Codes, Ann Arbor, Michigan), using default parameters.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension

[symbol]

exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

In some embodiments, the cell or organism has at least one heterologous trait. As used herein, the term “heterologous trait” refers to a phenotype imparted to a transformed host cell or transgenic organism by an exogenous DNA segment, heterologous polynucleotide or heterologous nucleic acid. These results can be achieved by providing expression of heterologous products or increased expression of endogenous products in organisms using the methods and compositions of the present disclosure.

As used herein “shelf-stable” refers to a functional attribute and new utility acquired by the microbes formulated according to the disclosure, which enable said microbes to exist in a useful/active state outside of their natural environment in a plant or soil (i.e. a markedly different characteristic). Thus, shelf-stable is a functional attribute created by the formulations/compositions of the disclosure and denoting that the microbe formulated into a shelf-stable composition can exist under ambient conditions for a period of time that can be determined depending upon the particular formulation utilized, but in general means that the microbes can be formulated to exist in a composition that is stable under ambient conditions for at least a few days and generally at least one week. Accordingly, a “shelf-stable soil treatment” is a composition comprising one or more microbes of the disclosure, said microbes formulated in a composition, such that the composition is stable under ambient conditions for at least one week.

As used herein, a “microbial signaler” or a “signaling microbe” refers to any microbe that has the capability to influence or alter a characteristic or function of a second microbe (referred to herein, as a “target microbe” or a “signaled microbe”) which is in its physical proximity. As used herein, “physical proximity” refers to a distance at which a “microbial signaler” is able to influence or alter a characteristic or function of the second microbe (or the target microbe). In some embodiments, the distance is less than around 3 cm. In some embodiments, the microbial signaler is adjacent (at a distance of less than or equal to around 1 cm) to the target microbe.

In some embodiments, the

[symbol]

promoting function of the target microbe. As used herein, the “plant growth promoting function” refers to the ability of a microbe (e.g. a microbial signaler, a target microbe, or a combination thereof) to enhance the growth of a plant. The growth of the plant may be reflected by the height of the plant, the yield of the plant, disease resistance, or any combination thereof.

In some embodiments, the plant growth-promoting function comprises one or more of the following functions: (a) plant pathogen-inhibiting function, (b) zinc solubilizing function, (c) phosphate solubilizing function, (d) production of an antibiotic, (e) nitrogen fixation, (f) nutrient acquisition, (g) production of plant growth hormones or (e) any combination thereof. In some embodiments, the microbial signaler influences or alters the characteristic or function of the target microbe through the use of chemical, physical and/or biological signaling moieties. In some embodiments, the microbial signaler and the target microbe are in contact with each other. In some embodiments, the microbial signaler and the target microbe are part of the same composition, such as, a composition disclosed herein.

As used herein, “bioavailability” refers to a form of an element or a compound that is accessible to an organism (e.g. a plant) for uptake, adsorption, and/or absorption. In some embodiments, bioavailable forms of an element or a compound are soluble forms of an element or a compound (e.g. zinc, or phosphate).

As used herein, “zinc solubilization” refers to the process by which non-bioavailable form of zinc is converted into a bioavailable form of zinc. In some embodiments, the non-bioavailable form of zinc is an insoluble form of zinc. In some embodiments, the bioavailable form of zinc is the soluble form of zinc. In some embodiments, zinc solubilization occurs in the soil, converting an insoluble form of zinc in the soil to a soluble form that is bioavailable for organisms (e.g. plants). Microbes that are capable of promoting or causing zinc solubilization are referred to herein as “zinc-solubilizing microbes”. Zinc solubilization may depend on a variety of factors, such as, pH of the soil, soil moisture, soil temperature, the presence of zinc solubilizing microbes, or any combination thereof.

As used herein, “phosphate solubilization” refers to the process by which non-bioavailable form of phosphate is converted into a bioavailable form of phosphate. In some embodiments, the non-bioavailable form of phosphate is an insoluble form of phosphate. In some embodiments, the bioavailable form of phosphate is the soluble form of phosphate. In some embodiments, phosphate solubilization occurs in the soil, converting an insoluble form of phosphate in the soil to a

[symbol]

Microbes that are capable of promoting or causing phosphate solubilization are referred to herein as “phosphate-solubilizing microbes”. Phosphate solubilization may depend on a variety of factors, such as, pH of the soil, soil moisture, soil temperature, the presence of phosphate solubilizing microbes, or any combination thereof.

As used herein, “nutrient acquisition” refers to the ability and/or process by which a plant acquires or uptakes growth-promoting nutrients, such as phosphate, zinc and other nutrients. In some embodiments, the disclosed microbial signalers, the disclosed target microbes, or any combinations thereof enhance the nutrient acquisition of a plant, thereby promoting plant growth. Enhancing nutrient acquisition may be affected by a variety of ways, for example, through improved phosphate solubilization and/or improved zinc solubilization, as described herein.

As used herein, a “high nutrient” condition, environment, medium, or soil refers to the condition, environment, medium, or soil comprising nutrients at a concentration that is in the standard range for a particular microbe. The standard nutrient concentration for a particular microbe can be ascertained by a person of ordinary skill in the art. As used herein, a “low nutrient” condition, environment, medium, or soil refers to a condition, environment, medium, or soil which comprises a concentration of nutrients that is lower than what is considered standard for that particular microbe. For example, the concentration of nutrients in the “low nutrient” condition may be half, ⅓^{rd}, ¼^{th}, ⅕^{th}, ⅙^{th}, 1/7^{th}, ⅛^{th}, 1/9^{th}, 1/10^{th}, 1/15^{th}, 1/20^{th}, or 1/50^{th} of the standard concentration of nutrients for that particular microbe. In some embodiments, the low nutrient condition is unable to support the pathogen-suppressing and/or antibiotic producing activities of a microbe that is known to possess pathogen-suppressing and/or antibiotic production activities. In some embodiments, the high nutrient condition is a condition under which a microbe that is known to possess pathogen-suppressing and/or antibiotic production activities exhibits the pathogen-suppressing and/or antibiotic production activities.

### Compositions Comprising Microbial Signalers Disclosed Herein

The disclosure provides microbial signalers that increase the performance of commercial biological agents, such as target microbes present in target commercial products. Additionally, the microbial signalers disclosed herein reduce variation in the performance of these target commercial products

[symbol]

nutrient availability.

The microbial signalers disclosed herein are capable of enhancing the plant growth-promoting function of one or more target microbes. For instance, the microbial signalers disclosed herein are capable of enhancing the plant pathogen-inhibiting function, zinc solubilizing function, phosphate solubilizing function, antibody-producing function, nitrogen fixing function, function of improving a plant's nutrient acquisition, production of plant growth hormones, or any combination thereof, of one or more target microbes. Furthermore, the microbial signalers disclosed herein are able to rescue the reduction in the plant growth-promoting function (e.g., reduction in pathogen suppression and/or reduction in antibody production) exhibited by the target microbes in low nutrient soils. Therefore, the compositions disclosed herein (comprising at least one of the microbial signalers disclosed herein; and one or more target microbes disclosed herein) have unexpectedly superior plant growth-promotion functions (e.g. remarkably enhanced plant pathogen-inhibiting function, remarkably enhanced zinc solubilizing function, remarkably enhanced phosphate solubilizing function, remarkably enhanced antibody-producing function, remarkably enhanced nitrogen fixing function, remarkably enhanced function of improving a plant's nutrient acquisition, remarkably enhanced production of plant growth hormones, or any combination thereof), as compared to the one or more target microbes disclosed herein alone, in standard soils as well as in low nutrient soils.

The disclosure provides compositions, comprising at least one microbial signaler, wherein the at least one microbial signaler is capable of enhancing a plant growth-promoting function of a target microbe. The disclosure further provides compositions, comprising: (a) at least one microbial signaler, and (b) at least one target microbe, wherein the at least one microbial signaler is capable of enhancing a plant growth-promoting function of the at least one target microbe.

In some embodiments, the compositions disclosed herein are microbial consortia.

In some embodiments, the compositions disclosed herein comprise one or more microbial signalers disclosed herein, and one or more target microbes disclosed herein. For instance, in some embodiments, the compositions disclosed herein comprise one or more microbial signalers listed in Table 1, and one or more target microbes listed in Table A.

In some embodiments,

[symbol]

comprising one or more microbial signalers disclosed herein, and one or more target microbes disclosed herein) have an enhanced ability to inhibit one or more plant pathogens, as compared to the one or more target microbes present in the compositions. In some embodiments, compositions comprising one or more microbial signalers listed in Table 1 and one or more target microbes listed in Table A have an enhanced ability to inhibit one or more plant pathogens listed in Table B, as compared to the one or more target microbes alone. In some embodiments, compositions comprising one or more microbial signalers listed in Table 1 and one or more target microbes listed in Table C have an enhanced ability to inhibit one or more plant pathogens listed in Table B, as compared to the one or more target microbes alone.

### Target Microbes

In some embodiments, the at least one target microbe belongs to the genus Talaromyces, Streptomyces, Bacillus, Trichoderma, Pseudomonas, Comamonas, or Enterobacter. In some embodiments, the at least one target microbe is Talaromyces flavus, Streptomyces griseoviridis, Streptomyces lydicus, Bacillus subtilis, Bacillus amyloliquefaciens, Bacillus amyloliquefaciens, Trichoderma harzianum, Pseudomonas putida, Pseudomonas chlororaphis, Comamonas testosterone, Citrobacter freundii, Enterobacter cloacae, or any combination thereof. In some embodiments, the at least one target microbe is Talaromyces flavus SAY-Y-94-01. In some embodiments, the at least one target microbe is Streptomyces lydicus WYEC 108.

In some embodiments, the target microbe is one or more of Talaromyces flavus, Trichoderma harzianum, Bacillus amyloliquefaciens, Streptomyces sp., Bacillus subtilis, Bacillus amyloliquefaciens, Streptomyces lydicus, Pseudomonas chlororaphis, Bacillus subtilis, Azospirillum brasilense, Trichoderma asperellum, Trichoderma gamsii, Pseudomonas putida, Comamonas testosterone, Citrobacter freundii, Enterobacter cloacae, Streptomyces spp., Trichoderma viride, Bacillus megaterium, Azospirillum spp., Bradyrhizobium japonicum, Rhizobium leguminosarum biovar viciae, Bradyrhizobium spp., Rhizobium leguminosarum, Azospirillum amazonense, Azospirillum lipoferum, Glomus intraradices, Rhizophagus intraradices, Glomus mosseaem, or any combination thereof.

In some embodiments, the target microbe belongs to a genus listed in Table A.

Table A: Exemplary Genera of Target Microbes Present in the Compositions and Used in the Methods Disclosed Herein.

[symbol]

In some embodiments, the target microbe is present in a commercial product. Non-limiting examples of target microbes and the corresponding commercial products that may be present in the compositions disclosed herein, and/or used in the methods disclosed herein are listed in Table C below. Table C also lists the plant-growth promoting function of these exemplary target microbes. As described herein, the disclosed microbial signalers are capable of enhancing any one or more of the plant growth promoting functions of one or more of the target microbes listed in Table C.

Table C: Exemplary Target Microbes Contained in Exemplary Commercial Products and Their Plant-growth Promoting Functions

[symbol]

[symbol]

### Plant-Growth Promoting Functions

In some embodiments, the plant growth-promoting function comprises: (a) plant pathogen-inhibiting function, (b) zinc solubilizing function, (c) phosphate solubilizing function, (d) production of an antibiotic, (e) nitrogen fixing function, (f) a function of improving a plant's nutrient acquisition, (f) production of plant growth hormones, or (g) any combination thereof.

In some embodiments, the at least one microbial signaler is capable of enhancing the plant pathogen-inhibiting function of the target microbe by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween). In some embodiments, the at least one microbial signaler is capable of enhancing the plant pathogen-inhibiting function of the target microbe by at least about 5%.

In some embodiments, the at least one microbial signaler is capable of enhancing the plant pathogenic disease suppression function of the target microbe by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least

[symbol]

about 300%, at least about 400%, at least about 500%, at least about 600%, at least about 700%, at least about 800%, at least about 900% or at least about 1000%, including all values and subranges that lie therebetween). In some embodiments, the at least one microbial signaler is capable of enhancing the plant pathogen-inhibiting function of the target microbe by at least about 5%.

The enhancement of plant pathogenic disease suppression function as disclosed herein may be seen in any plant. Non-limiting examples of plants that may used with the microbial signalers and/or compositions disclosed herein, or used in the methods disclosed herein are corn, soybean, wheat, alfalfa, cotton, rice, oats, barley, oil crops (e.g. sunflower, canola), vegetable crops (e.g. potato, sweet potato, corn, cassava, yams, plantains, tomato, beans, sugar beets), sugar cane, fruit crops, grain crops, pulses, legumes, maize, millet, sorghum, tuber crops, feed crops, ornamental crops, industrial crops, food crops, fiber crops, beverage plants (e.g. tea, coffee), seed trees, nut trees, herb plants, bioenergy crops (e.g. switch grass, aspen), forest trees, and spices plants (e.g. saffron).

The plant pathogen is not limited, and may be a soil-borne plant pathogen, a seed-borne plant pathogen, a pathogen of leaves and/or foliage, a pathogen of fruit, a pathogen of stem, a pathogen of root, or any combination thereof. Non-limiting examples of the plant pathogen include plant pathogens listed below in Table B.

Table B: Exemplary Plant Pathogens Inhibited by the Disclosed Signaling Microbes and/or Disclosed Compositions, Comprising the Disclosed Signaling Microbes and the Disclosed Target Microbes.

[symbol]

In some embodiments, the at least one microbial signaler is capable of enhancing the zinc solubilizing function of the target microbe by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween). In some embodiments, the at least one microbial signaler is capable of enhancing the zinc solubilizing function of the target microbe by at least about 5%.

In some embodiments, the at least one microbial signaler is capable of enhancing the phosphate solubilizing function of the target microbe by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween). In some embodiments, the at least one microbial signaler is capable of enhancing the phosphate solubilizing function of the target microbe by at least about 5%. In some embodiments, the at least one microbial signaler is capable of enhancing the phosphate solubilizing function of the at least one target microbe under low nutrient conditions.

In some embodiments,

[symbol]

enhancing the nitrogen fixing function of the target microbe by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween). In some embodiments, the at least one microbial signaler is capable of enhancing the nitrogen fixing function of the target microbe by at least about 5%. In some embodiments, the at least one microbial signaler is capable of enhancing the nitrogen fixing of the at least one target microbe under low nutrient conditions.

### Microbial Signalers

In some embodiments, the microbial signalers comprise polynucleotide sequences that share at least 70%, 75%, 80%, 81%, 82%, 83%, 84%, 85%, 86%, 87%, 88%, 89%, 90%, 91%, 92%, 93%, 94%, 95%, 95.1%, 95.2%, 95.3%, 95.4%, 95.5%, 95.6%, 95.7%, 95.8%, 95.9%, 96%, 96.1%, 96.2%, 96.3%, 96.4%, 96.5%, 96.6%, 96.7%, 96.8%, 96.9%, 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, or 100% sequence identity with the 16S rRNA sequence, 18S rRNA sequence, 23S rRNA sequence, the internal transcribed spacer (ITS1) sequence and/or ITS2 sequence of any one of the microbial signalers listed in this specification.

The microbes disclosed herein may be matched to their nearest taxonomic groups by utilizing classification tools of the Ribosomal Database Project (RDP) for 16s rRNA sequences and the User-friendly Nordic ITS Ectomycorrhiza (UNITE) database for ITS rRNA sequences. Examples of matching microbes to their nearest taxa may be found in Lan et al. (2012. PLOS one. 7(3):e32491), Schloss and Westcott (2011. Appl. Environ. Microbiol. 77(10):3219-3226), and Koljalg et al. (2005. New Phytologist. 166(3):1063-1068).

In some embodiments, the at least one microbial signaler is any microbe listed in Table 1 or Table D.

TABLE 1
Exemplary Microbial Signalers

| | 16S | | |
|---|---|---|---|
| Name of | rRNA | | |
| microbial | SEQ | | Possible Taxonomic |
| signaler | ID | | Assignments for the microbial |
| isolate | NO: | 16s rRNA sequence | signaler isolate |
| JBS6220 | 6 | AGGCGCGTCATAACACATGCAGCCGAACGATCAAACACTGCAAGGTGAAGA | Streptomyces avidinii |
| | | AGACCCCAACGGAGGAGTAACAAACGGGCAACCTGACATTCACTCAGGGAC | Streptomyces colombiensis |
| | | AAGCCCTGCAAAACGAGTCTAATACACGATACCACTCCTCCACGCATGGCC | Streptomyces lavendulae |
| | | AGGGGTTGAAAAAGCCCGCGGTGAAGGATGAGCCCCCGCCCCATCAGCTTG | Streptomyces roseochromogenus |
| | | TTGGGGGGGGAATGACCCAACAAGGCGACGACGGGTAGCCGGCCTGAGAG | Streptomyces spororaveus |
| | | GGCCAACCGCCACACTGGGACTGAGACACGGCCCAGACTCCTACGGGAGGC | Streptomyces sporoverrucosus |
| | | AGCAGTGGGGAATATTGCACAATGGGCGAAAGCCTGATGCAGCGACGCCGC | Streptomyces venezuelae |
| | | GTGAGGGATGACGGCCTTCGGGTTGTAAACCTCTTTCAGCAGGGAAGAAGC | Streptomyces xanthophaeus |
| | | GAAAGTGACGGTACCTGCAGAAGAAGCGCCGGCTAACTACGTGCCAGCAGC | |
| | | CGCGGTAATACGTAGGGCGCAAGCGTTGTCCGGGAATTATTGGGCGTAAAG | |
| | | AGCTCGTAGGCGGCTTGTCACGTCGGATGTGAAAGCCCGAGGCTTAACCCC | |
| | | GGGTCTGCATTCGATACGGGCAAGCTAGAGTGCGGTAGGGGAGATCGGAAT | |
| | | TCCTGGTGTAGCGGTGAAATGCGCAGATATCAGGAGGAACACCGGTGGCGA | |
| | | AGGCGGATCTCTGGGCCAATACTGACGCTGAGGAGCGAAAGCGTGGGGAGC | |
| | | GAACAGGATTAGATACCCTGGTAGTCCACGCCGTAAACGTTGGGAACTAGG | |
| | | TGTGGGCGACATTCCACGTCGTCCGTGCCGCAGCTAACGCATTAAGTTCCCC | |
| | | GCCTGGGGAGTACGGCCGCAAGGCTAAAACTCAAAGGAATTGACGGGGGCC | |
| | | CGCACAAGCAGCGGAGCATGTGGCTTAATTCGACGCAACGCGAAGAACCTT | |
| | | ACCAAGGCTTGACATACACCGGAAAACCATGAAGACAGGGCCCCCCTTGTG | |
| | | GTCGGTATACAGGTGGTGCATGGCTGTCGTCAGCTCGTGTCGTGAGATGTTG | |
| | | GGTTAAGTCCCGCAACGAGCGCAACCCTTGTCCTGTGTTGCCAGCATGCCCT | |
| | | TCGGGGTGATGGGGACTCACAGGAAACCGCCGGGGTCAACTCGGAGAAAG | |
| | | GGGACGACGACAGCAAGTCATCATGCCCCAAAGGTCCTGGCCGCCACACGT | |
| | | GCTACAAGAGCCGGTACAATGACCGCAGATACCGCGACGTGAAGCGAAGAT | |
| | | CAAAAACCCGATCTCAGTACGAATTGAGGACTGCAACTCAACACCAGAAAC | |
| | | GCGAAACGGCCAGTAATCGCAGATCAGCATGCTGCGTATTCCGG | |
| JBS8493 | 20 | AGGGCGGGTGCTTACCAAGCAACTCGAAACATGAAACTCCCAAGGGAGAAC | Streptomyces angustmyceticus |
| | | AGTAAAGGCGAAAGGGGAAATAACACGTAGGAAATCGGCAATTCACCATC | Streptomyces hygroscopicus |
| | | GGACAAGCCCTGGAAACCGGGTCCAATACACGATACGACCTCCAACACCAT | Streptomyces libani |
| | | AGCATCGGGGGGAAAAGCTCCGCCGGAGAAGGATGAGCACCCGCCCTATCA | Streptomyces lydicus |
| | | GCTGGGTGAAGGAGTGATGGCCCACAAAAGCGACGACGGGTAGCAGCCCC | Streptomyces nigrescens |
| | | GAAAAGGCCAACGGCCACACTGGGACTGAGACACGGCCCAGACTCCTACGG | Streptomyces platensis |
| | | GAGGCAGCAGTGGGGAATATGCAAAAAGGGGCCAAAACCTGATGCAGCGA | Streptomyces rimosus |
| | | CGCCGCGTGAGGGATGACGGCCTTCGGGTTGTAAACCTCTTTCAGCAGGGA | Streptomyces tubercidicus |
| | | AGAAGCGAAAGTGACGGTACCTGCAGAAGAAGCGCCGGCTAACTACGTGCC | |
| | | AGCAGCCGCGGTAATACGTAGGGCGCAAGCGTTGTCCGGAATTATTGGGCG | |
| | | TAAAGAGCTCGTAGGCGGCTTGTCACGTCGGATGTGAAAGCCCGGGGCTTA | |
| | | ACCCCGGGTCTGCATTCGATACGGGCAAGCTAGAGTGCGGTAGGGGAGATC | |
| | | GGAATTCCTGGTGTAGCGGTGAAATGCGCAGATATCAGGAGGAACACCGGT | |
| | | GGCGAAGGCGGATCTCTGGGCCAATACTGACGCTGAGGAGCGAAAGCGTGG | |
| | | GGAGCGAACAGGATTAGATACCCTGGTAGTCCACGCCGTAAACGGTGGGAA | |
| | | CTAGGTGTGGGCGACATTCCACGTCGTCCGTGCCGCAGCTAACGCATTAAGT | |
| | | TCCCCGCCTGGGGAGTACGGCCGCAAGGCTAAAACTCAAAGGAATTGACGG | |
| | | GGGCCCGCACAAGCAGCGGAGCATGTGGCTTAATTCGACGCAACGCGAAGA | |
| | | ACCTTACCAAGGCTTGACATACACCGGAAAACCCCAGAGAACAGCGCCCCC | |
| | | TTGTGGTCGGTGTACAGGTGGTGCATGGCTGTCGTCAGCTCGTGTCGTGAGA | |
| | | TGTTGGGTTAAGTCCCGCAACGAGCGCAACCCTTGTTCTGGTGTTGCCAGCA | |
| | | TGCCCTTCGGGGTGATGGGGACTCAAAAGAAAACGCCCGGGTCAACTCGGA | |
| | | GAAAGGGGACGACGAAGTCAACACATCATGCCCCTTATGTCTTGGGCTGCA | |
| | | AACGGCCAAAAAGGGCACGTACAATGACCGGCAAACCCGAGAGGCGGAAC | |
| | | GAATCTCAAAAAGCCAGTCGAAGGGCGCACTGCACTCTGCAACGCAACCCA | |
| | | ATGAAATCGGAGGAGCTAGTAATCGCAGATCAGCATGCTGCGTATGCAG | |
| JBS2943 | 3 | CGGGGCGCGTGCTTACACATGCACTAAAACAATCAAACAAGTCGAAACGAA | Streptomyces bungoensis |
| | | ATACCAGCCAACGGGAGAATAACACGGGGACAACCTGCCATTGCACACTCG | Streptomyces cyslabdanicus |
| | | GAAAAGCCAGCGAAACGGGGTCTAATACCGGATACCAATCCAGCAGCAACA | Streptomyces galbus |
| | | TCCCGGAGTCGAAAGCTCCGCCGGAGAAGGATGAACCAGCCCCCGATAAGC | Streptomyces kagawaensis |
| | | AGCTGGGTGAGGTAGTGGCTCACCAAGGCGACGACGGGTAGCCGCCCAGAG | Streptomyces lasaliensis |
| | | AGAGCGACACCCCACACTGGGACTGAGACACGGCCCAGACTCCTACGGGAG | Streptomyces lasalocidi |
| | | GCAGCAGTGGGGAATATTGCACAATGGGCGAAAGCCTGATGCAGCGACGCC | Streptomyces longwoodensis |
| | | GCGTGAGGGATGACGGCCTTCGGGTTGTAAACCTCTTTCAGCAGGGAAGAA | Streptomyces spinichromogenes |
| | | GCGAAAGTGACGGTACCTGCAGAAGAAGCGCCGGCTAACTACGTGCCAGCA | |
| | | GCCGCGGTAATACGTAGGGCGCAAGCGTTGTCCGGGAATTATTGGGCGTAA | |
| | | AGAGCTCGTAGGCGGCTTGTCACGTCGGGTGTGAAAGCCCGGGGCTTAACC | |
| | | CCGGGTCTGCATTCGATACGGGCAGGCTAGAGTGTGGTAGGGGAGATCGGA | |
| | | ATTCCTGGTGTAGCGGTGAAATGCGCAGATATCAGGAGGAACACCGGTGGC | |
| | | GAAGGCGGATCTCTGGGCCATTACTGACGCTGAGGAGCGAAAGCGTGGGGA | |
| | | GCGAACAGGATTAGATACCCTGGTAGTCCACGCCGTAAACGGTGGGAACTA | |
| | | GGTGTTGGCGACATTCCACGTCGTCGGTGCCGCAGCTAACGCATTAAGTTCC | |
| | | CCGCCTGGGGAGTACGGCCGCAAGGCTAAAACTCAAAGGAATTGACGGGGG | |
| | | CCCGCACAAGCAGCGGAGCATGTGGCTTAATTCGACGCAACGCGAAGAAAC | |
| | | CTTACAAAGGCTTGACATACACCGGAAACGTCTGGAGACAGGCGCCCCCTT | |
| | | GTGGTCGGTGTACAAGGGGGGCAAGGCTGTCGTCAGCTCGTGTCGTGAGAT | |
| | | GTTGGGTTAAGTCCCGCAACGAGCGCAACCCTGGCCCTGTGTTGCCAGCATG | |
| | | CCCTTCGGGGTGATGGGAACCCACAGGAAACCGCCGGGGCCAACTCGGAGG | |
| | | AAGGTGGGGACGACGTCAAGTCATCATGCCCCTTATGTCTTGGGCTGCACAC | |
| | | GTGCTACAATGGCCGGTACAATGAGCTGCGATACCGTGAGGTGGAGCGAAT | |
| | | CTCAAAAAGCCGGTCTCAGTTCGGATTGGGGTCTGCAACTCGACCCCATGAA | |
| | | GTCGGAGTTGCTAGTAATCGCAGATCAGCATGCTGCGTAATGCTC | |
| JBS3418 | 36 | AAGGGCGGCGAGATCACACACGAAAGTCGAACGACGAAACACTGCGGGGT | Streptomyces cirratus |
| | | GGATTAGTGGCGAACGGGACAGTAACACCAGGGCAATATGCCAACGTATAA | Streptomyces nojiriensis |
| | | CACGGGCATTCGCACAACCACAGCAAAAACGGTCTAATACCAGATAATAAT | Streptomyces sporoverrucosus |
| | | ACCGCCTGCATGGCCGGGGGGTGAAAGCCCCGGCGGTGAAAGATGAGCCCG | Streptomyces venezuelae |
| | | CGGCCTATCAGCTTGTTGGGGGGGGAAAGGCCCACCAAGGCGACGACGGGT | Streptomyces verne |
| | | AGCCCGCCTGAAAAGGCCAACGGCCACACTGGGACTGAGACACGGCCCAGA | Streptomyces vinaceus |
| | | CTCCTACGGGAGGCAGCAGTGGGGAATATTGCACAATGGGCGAAAACCTGA | Streptomyces virginiae |
| | | TGCAGCGACGCCGCGTGAGGGATGACGGCCTTCGGGTTGTAAACCTCTTTCA | Streptomyces xanthophaeus |
| | | GCAGGGAAGAAGCGAAAGTGACGGTACCTGCAGAAGAAGCGCCGGCTAAC | |
| | | TACGTGCCAGCAGCCGCGGTAATACGTAGGGCGCAAGCGTTGTCCGGAATT | |
| | | ATTGGGCGTAAAGAGCTCGTAGGCGGCTTGTCACGTCGGATGTGAAAGCCC | |
| | | GAGGCTTAACCTCGGGTCTGCATTCGATACGGGCTAGCTAGAGTGTGGTAG | |
| | | GGGAGATCGGAATTCCTGGTGTAGCGGTGAAATGCGCAGATATCAGGAGGA | |
| | | ACACCGGTGGCGAAGGCGGATCTCTGGGCCATTACTGACGCTGAGGAGCGA | |
| | | AAGCGTGGGGAGCGAACAGGATTAGATACCCTGGTAGTCCACGCCGTAAAC | |
| | | GTTGGGAACTAGGTGTTGGCGACATTCCACGTCGTCGGTGCCGCAGCTAACG | |
| | | CATTAAGTTCCCCGCCTGGGGAGTACGGCCGCAAGGCTAAAACCAAAAGAA | |
| | | ATGAACGGGGGCCCGCACAAGCGGCGGAGCATGTGGCTTAATTCGACGCAA | |
| | | CGCGAAGAACCTTACCAAGGCTTGACATATACCGGAAAGCATTAGAGATAG | |
| | | GGCCCCCCTTGTGGTCGGTATACAGGGGGTGCATGGCTGTCGTCAGCTCGTG | |
| | | TCCGGAGAGATGTTGGGTTAAGTCCCGCAACGAGCGCAACCCCTGGCCTGT | |
| | | GTTGCCAGCATGCCCTTCGGGGGGATGGGGACTCACAGGAAACCGCCGGGG | |
| | | CAAACCCGGAGGAAGGTGGGGACGACGTCAAGTCATCATGCCCCTTATGTC | |
| | | TTGGGCTGCACACGTGCTACAATGGCCGGTACAATGAGCTGCGATACCGTG | |
| | | AGGTGGAGCGAATCTCAAAAAGCCGGTCTCAGTTCGGATTGGGGTCTGCAA | |
| | | CTCGACCCCATGAAGTCGGAGTCGCTAGTAATCGCAGATCAGCATGCTGCG | |
| | | GTATCCGC | |
| JBS4783 | 21 | CGGGGCGACTCATGACAAATGCAAAAAGAAAGATGAACCCCCTTCGCGAGG | Streptomyces angustmyceticus |
| | | GGATTAGTGGCGAAACGGTGAGTAACACGTGCACAACCTGCACTTCACTCT | Streptomyces catenulae |
| | | GGGACAAAACCCGGAAACCGAGTCTAATACCGGATACGAACACACACCGCA | Streptomyces cinereus |
| | | GCATCGCGGGGTGGAAAGCTCCGGCGGTGAAGAAGAAGCCCGCGGCCTATA | Streptomyces griseocarneus |
| | | ACAGCGGTGGTGGGGTAATGGCCCACCAAGGCGGACGACGGGTAGCCGGCC | Streptomyces hygroscopicus |
| | | CGAAAAGGCCAACGGCCACACTGGGACTGAGAAAACGCCCAAAACCCTAC | Streptomyces libani |
| | | GGGAGGCAGCAGTGGGGAATATTGCACAATGGGCGAAAACCTGATGCAGC | Streptomyces nigrescens |
| | | GACGCCGCGTGAGGGATGACGGCCTTCGGGTTGTAAACCTCTTTCAGCAGG | Streptomyces sioyaensis |
| | | GAAGAAGCGAAAGTGACGGTACCTGCAGAAGAAGCGCCGGCTAACTACGT | |
| | | GCCAGCAGCCGCGGTAATACGTAGGGCGCAAGCGTTGTCCGGAATTATTGG | |
| | | GCGTAAAGAGCTCGTAGGCGGCTTGTCACGTCGGATGTGAAAGCCCGAGGC | |
| | | TTAACCCCGGGTCTGCATTCGATACGGGCAAGCTAGAGTGCGGTAGGGGAG | |
| | | ATCGGAATTCCTGGTGTAGCGGTGAAATGCGCAGATATCAGGAGGAACACC | |
| | | GGTGGCGAAGGCGGATCTCTGGGCCAATACTGACGCTGAGGAGCGAAAGCG | |
| | | TGGGGAGCGAACAGGATTAGATACCCTGGTAGTCCACGCCGTAAACGTTGG | |
| | | GAACTAGGTGTGGGCGACATTCCACGTCGTCCGTGCCGCAGCTAACGCATTA | |
| | | AGTTCCCCGCCTGGGGAGTACGGCCGCAAGGCTAAAACTCAAAGGAATTGA | |
| | | CGGGGGCCCGCACAAGCAGCGGAGCATGTGGCTTAATTCGACGCAACGCGA | |
| | | AGAACCTTACCAAAGCCTTGACATACACCGGAAAACACTAGAGACAGGGCC | |
| | | CCCCTTGTGGGCCGTATACAGGTGGTGCATGGCTGTCGTCAGCTCGTGTCGT | |
| | | GAGAATGTTGGGTTAAGTCCCGCAACGAGCGCAACCCTTGTCCTGTGTTGCC | |
| | | AGCATGCCCTTCGGGGTGATGGGGACTCACAGGAGACCGCCGGGGCAAACT | |
| | | CCGAAGAAGGTGGGGACAACGTCAAGTCATCATGCCCCTTAAGTCCTGGGC | |
| | | TGCACAAGGCCAAAAAGCGCCACAACAATGACATGCGATACCGCGAGGTGG | |
| | | AGCGAATCTCAAAAAGCCGCTCTCAGTCCGAATGGAAGACAGCAACTCGAC | |
| | | CCCATGAAGTCGGAGTCGCTAGTAATCGCAGATCAGCATGCTGCGTTAATGC | |
| | | TC | |
| JBS3946 | 7 | AGGGGAGGTGCTTACACACGCACTCGAACCATGAAGACATCAGGGGTGAAA | Streptomyces avidinii |
| | | GAGTAGCCAACGGGGAAATAACACACGGGCAAACTGCACTGCACTCGGAG | Streptomyces cirratus |
| | | ACAAACCCTGGAAACCGGGTCAAACAACAGACACCACGCATGCCTGCATGG | Streptomyces lavendulae |
| | | GCGGGGGTGGAAAGCAACGCCGCGGAAAGATGAGCCCGCGCCATACAACC | Streptomyces nojiriensis |
| | | GTGTGGGTGGGGTAATGCCCCACAAAAGCGACGACGGCGAGACGGCCGCA | Streptomyces spororaveus |
| | | GAGAGCGACACGCAAAACGGGAACGAAAACACGGCCCAGACTCCTACGGG | Streptomyces subrutilus |
| | | AGGCAGCAGTGGGGAATATTGCACAATGGGCGAAAACCTGATGCAGCGACG | Streptomyces venezuelae |
| | | CCGCGTGAGGGATGACGGCCTTCGGGTTGTAAACCTCTTTCAGCAGGGAAG | Streptomyces xanthophaeus |
| | | AAGCGAAAGTGACGGTACCTGCAGAAGAAGCGCCGGCTAACTACGTGCCAG | |
| | | CAGCCGCGGTAATACGTAGGGCGCAAGCGTTGTCCGGAATTATTGGGCGTA | |
| | | AAGAGCTCGTAGGCGGCTTGTCACGTCGGATGTGAAAGCCCGAGGCTTAAC | |
| | | CCCGGGTCTGCATTCGATACGGGCAAGCTAGAGTGCGGTAGGGGAGATCGG | |
| | | AATTCCTGGTGTAGCGGTGAAATGCGCAGATATCAGGAGGAACACCGGTGG | |
| | | CGAAGGCGGATCTCTGGGCCAATACTGACGCTGAGGAGCGAAAGCGTGGGG | |
| | | AGCGAACAGGATTAGATACCCTGGTAGTCCACGCCGTAAACGTTGGGAACT | |
| | | AGGTGTGGGCGACATTCCACGTCGTCCGTGCCGCAGCTAACGCATTAAGTTC | |
| | | CCCGCCTGGGGAGTACGGCCGCAAGGCTAAAACTCAAAGGAATTGACGGGG | |
| | | GCCCGCACAAGCAGCGGAGCATGTGGCTTAATTCGACGCAACGCGAAGAAC | |
| | | CTTACCAAGGCTTGACATACACCGGAAAACACTAGAGACAGGGCCCCCCTT | |
| | | GGGGTCGGTATACAGGTGGTGCATGGCTGTCGTCAGCTCGTGTCGTGAGATG | |
| | | TTGGGTTAAGTCCCGCAACGAGCGCAACCCTTGTCCTGTGTGCCAACAAGCC | |
| | | CTTCCGGGGGAAGGGACACACACAAAAGACGGCACGCGAAAACGCGGAGG | |
| | | AAGGTGGGGACGACGGCAAGTCATCATGCCCCTTATGTCTGGCGCTGCACA | |
| | | CGTGCTACAAGCGCACATACAATGAACTGCGATACCGGCGAGGTGGAGACA | |
| | | ATCTCAAAAAGCCGGTCTCAGTTCGGATTGGGGTCTGCAACTCGACCCCATG | |
| | | AAGTCGGAGTTGCTAGTAATCGCAGATCAGCATGCTGCGTAATGCCCT | |
| JBS8473 | 22 | TCGGGCCCCTAAACACACAGGCAAGTCCAACGATGAAGCCTTTCGGGACCG | Streptomyces atrolaccus |
| | | ATGAGCCGCGAAAGGGAGAGGAAAACGTGAGCAACCTGCCCATCACTCTGG | Streptomyces auratus |
| | | CACAAGACCAGCAAACGACGGCTAAAACACGATACGACAAACGACCGCAT | Streptomyces griseocarneus |
| | | GGTCTGGGTGTGGAAAGCTCCGGCGGTGAAAGAAGACCCCGCGACCTATCA | Streptomyces hygroscopicus |
| | | GCTTGGTGGTGGGGTGATGGCATACCAAAGCGACGACGGGGAACCCGCCTG | Streptomyces libani |
| | | AGAGGGCGACCGCCAACACTGGGACTGAGACACGGCCCAGACTCCTACGGG | Streptomyces lydicus |
| | | AGGCAGCAGTGGGGAATATTGCACAATGGGCGAAAGCCTGATGCAGCGACG | Streptomyces sioyaensis |
| | | CCGCGTGAGGGATGACGGCCTTCGGGTTGTAAACCTCTTTCAGCAGGGAAG | Streptomyces tubercidicus |
| | | AAGCGAAAGTGACGGTACCTGCAGAAGAAGCGCCGGCTAACTACGTGCCAG | |
| | | CAGCCGCGGTAATACGTAGGGCGCAAGCGTTGTCCGGAATTATTGGGCGTA | |
| | | AAGAGCTCGTAGGCGGCTTGTCACGTCGGATGTGAAAGCCCGGGGCTTAAC | |
| | | CCCGGGTCTGCATTCGATACGGGCAGGCTAGAGTTCGGTAGGGGAGATCGG | |
| | | AATTCCTGGTGTAGCGGTGAAATGCGCAGATATCAGGAGGAACACCGGTGG | |
| | | CGAAGGCGGATCTCTGGGCCGATACTGACGCTGAGGAGCGAAAGCGTGGGG | |
| | | AGCGAACAGGATTAGATACCCTGGTAGTCCACGCCGTAAACGTTGGGAACT | |
| | | AGGTGTGGGCGACATTCCACGTCGTCCGTGCCGCAGCTAACGCATTAAGTTC | |
| | | CCCGCCTGGGGAGTACGGCCGCAAGGCTAAAACTCAAAGGAATTGACGGGG | |
| | | GCCCGCACAAGCAGCGGAGCATGTGGCTTAATTCGACGCAACGCGAAGAAC | |
| | | CTTACCAAGGCTTGACATACACCGGAAAACCCTGGAGACAGGGCCCCCCTG | |
| | | GGGGTCGGTGTACAGGTGGGGCAAGGCCTGTCGTCAGCTCGTGTCGTGAGA | |
| | | TGTTGGGTTAAGTCCCGCAACGAGCGCAACCCTTGTTCTGTGTTGCCAGCAA | |
| | | GCACTTCCGGGGGAGAGGGACTCACAGGAGACTGCCGGGGTCAACTCGGAG | |
| | | GAAGGTGGGGACGACGTCAAGTCATCATGCCCCTTATGTCTTGGGCTGCACA | |
| | | CGTGCTACAATGGCCGGTACAATGAGCTGCGATACCGCGAGGTGGAGCGAA | |
| | | TCTCAAAAAGCCGGTCTCAGTTCGGATTGGGGTCTGCAACTCGACCCCATGA | |
| | | AGTCGGAGTTGCTAGTAATCGCAGATCAGCATGCTGCGGTAATGCTTC | |
| JBS6762 | 23 | CGGGGCGGCCTGCCTAAACACGCAAGTCGAACGATGAAGCCCTTCACGGAG | Streptomyces atrolaccus |
| | | GACTAGCGCCGAACGAATGAGGAAAACGTGAGCAACCTGCACTGCACTCCG | Streptomyces auratus |
| | | GGACAAGACCTGGAAACGGGGTCTAAAACCGGATAAGACAAACCAACGCA | Streptomyces griseocarneus |
| | | ACGCATGTGCGTGGAAAGCGAAGCCGCGGAAGGATGAGCCCGCGGCCCAAT | Streptomyces hygroscopicus |
| | | CAGCTTGTTGGTGGGGTGATGACCCAACAAGGCGACGACGGGTAGCCGGCC | Streptomyces libani |
| | | TGAGAGGGCGAACGGCCACACTGGGACTGAGACACGGCCCAGACTCCTACG | Streptomyces lydicus |
| | | GGAGGCAGCAGTGGGGAATATTGCACAATGGGCGAAAACCTGATGCAGCG | Streptomyces sioyaensis |
| | | ACGCCGCGTGAGGGATGACGGCCTTCCGGGTGTAAACCTCTTTCAGCAGGG | Streptomyces tubercidicus |
| | | AAGAAGCGAAAGTGACGGTACCTGCAGAAGAAGCGCCGGCTAACTACGTGC | |
| | | CAGCAGCCGCGGTAATACGTAGGGCGCAAGCGTTGTCCGGAATTATTGGGC | |
| | | GTAAAGAGCTCGTAGGCGGCTTGTCACGTCGGATGTGAAAGCCCGGGGCTT | |
| | | AACCCCGGGTCTGCATTCGATACGGGCAGGCTAGAGTTCGGTAGGGGAGAT | |
| | | CGGAATTCCTGGTGTAGCGGTGAAATGCGCAGATATCAGGAGGAACACCGG | |
| | | TGGCGAAGGCGGATCTCTGGGCCGATACTGACGCTGAGGAGCGAAAGCGTG | |
| | | GGGAGCGAACAGGATTAGATACCCTGGTAGTCCACGCCGTAAACGTTGGGA | |
| | | ACTAGGTGTGGGCGACATTCCACGTCGTCCGTGCCGCAGCTAACGCATTAAG | |
| | | TTCCCCGCCTGGGGAGTACGGCCGCAAGGCTAAAACTCAAAGGAATTGACG | |
| | | GGGGCCCGCACAAGCAGCGGAGCATGTGGCTTAATTCGACGCAACCCAAAG | |
| | | AACCTTACCAAGGCTTGACATACACCGGAAAAACCCTGGAGACAAGGTCCC | |
| | | CCTTGTGGTCGGTGTACAGGTGGTGCATGGCTGTCGTCAGCTCGTGTCGTGA | |
| | | GATGTTGGGTTAAGTCCCGCAACGAGCGCAACCCTTGTTCTGTGTTGCCAGC | |
| | | ATGCCCTTCGGGGTGATGGGGACTCACAGGAGACTGCCGGGGTCAACTCGG | |
| | | AGGAAGGTGGGGACGACGTCAAGTCATCATGCCCCTTATGTCTTGGGCTGC | |
| | | ACACGTGCTACAATGGCCGGTACAATGAGCTGCGATACCGCGAGGTGGAGC | |
| | | GAATCTCAAAAAGCCGGTCTCAGTTCGGATTGGGGTCTGCAACTCGACCCCA | |
| | | TGAAGTCGGAGTTGCTAGTAATCGCAGATCAGCATGCTGCGTAATGCTA | |
| JBS9261 | 24 | CGGGAGGTGCCTACAAAACCACTCGAAAGACAAACCTACGACTCCGCAGAG | Streptomyces angustmyceticus |
| | | GAATAGGGGCGAACGGGTGAGTAACACCGGGGAAAACTGCACTGCACTCA | Streptomyces atrolaccus |
| | | GGGACAAGCCCTGGAAACGGGCTCTAAAACCAGAAACGACATACGACCGC | Streptomyces griseocarneus |
| | | AACGCATGGTCGTGGAGGAAGCTCCGCCGGTGAAGGAAGAGCCCCCGCCCT | Streptomyces hygroscopicus |
| | | ATCAGCTGGTTGGTGGGGTGATGACCCAACAAGGCGACGACGGGTAGCCGG | Streptomyces libani |
| | | CCGAAAAGGGCGACCGGCCACACTGGGACTGAGAAAACGCCCAAAACCCT | Streptomyces nigrescens |
| | | ACGGGAGGCAGCAGTGGGGAATATGCAAAAATGGGCCAAAACCTGATGCA | Streptomyces sioyaensis |
| | | GCGACGCCGCGTGAGGGATGACGGCCTCCGGGTTGGAAAACCCCTTCAGCA | Streptomyces tubercidicus |
| | | GGGAAGAAGCGAGAGTGACGGTACCTGCAGAAGAAGCGCCGGCTAACTAC | |
| | | GTGCCAGCAGCCGCGGTAATACGTAGGGCGCAAGCGTTGTCCGGAATTATT | |
| | | GGGCGTAAAGAGCTCGTAGGCGGCTTGTCACGTCGGATGTGAAAGCCCGGG | |
| | | GCTTAACCCCGGGTCTGCATTCGATACGGGCAGGCTAGAGTTCGGTAGGGG | |
| | | AGATCGGAATTCCTGGTGTAGCGGTGAAATGCGCAGATATCAGGAGGAACA | |
| | | CCGGTGGCGAAGGCGGATCTCTGGGCCGATACTGACGCTGAGGAGCGAAAG | |
| | | CGTGGGGAGCGAACAGGATTAGATACCCTGGTAGTCCACGCCGTAAACGTT | |
| | | GGGAACTAGGTGTGGGCGACATTCCACGTCGTCCGTGCCGCAGCTAACGCA | |
| | | TTAAGTTCCCCGCCTGGGGAGTACGGCCGCAAGGCTAAAACTCAAAGGAAT | |
| | | TGACGGGGGCCCGCACAAGCAGCGGAGCATGTGGCTTAATTCGACGCAACG | |
| | | CGAAGAACCTTACCAAGGCTTGACATACACCGGAAAACCCTGGAGACAGGG | |
| | | TCCCCCTTGTGGTCGGTGTACAGGTGGTGCATGGCTGTCGTCAGCTCGTGTC | |
| | | GTGAGATGTTGGGTAAAGTCCCGCAACGAGCGCAACCCTTGTTCTGTGTGCC | |
| | | AAGCATGCCCTTCGGGGTGATGGACACTCACAGGAGACTGCCGGGGCAAAC | |
| | | GAGAAGGAAGGACGGGACGAACGCAAGTCATCATGCCCCTAATCTCGTGGC | |
| | | CTGCACACCGGCTACAATGACCCGTACAATGAACTGCGAAAACCCCAAGTG | |
| | | GAGCAAATCTCAAAAAGCCAGACCCAATTCGGACTGCACTCGGCAACTCGA | |
| | | ACCCATGAACCCGGAATTGCTAATAAACACACAACACAATGCTGCGTAATG | |
| | | CCGTC | |
| JBS4549 | 25 | CGGGCGGGTGCTTAAACATGCACGCAAACGATGAACCTCCCTCGTCGGAGG | Streptomyces angustmyceticus |
| | | GGAATAGTGGCAAACGGGTGAGTAACACGTGGACAACCTGCCATTCACTAC | Streptomyces hygroscopicus |
| | | GCGACAAGCCCTGGAAACGGACACCAAAACCGGATAAGACCACCGACCCG | Streptomyces libani |
| | | AAGCATCTCGTGGTGGAAAGCTCCGCCGGTGAAAGATGAGCCCGCCGCCAA | Streptomyces lydicus |
| | | TCAGCTTGTGGGGGGGGTGATGGCCGACAAAGGCGACGAACGGGAACCCGC | Streptomyces nigrescens |
| | | CTGAGAGGGCGACCGGCCACACTGGGACTGAGACACGGCCCAGACTCCTAC | Streptomyces platensis |
| | | GGGAGGCAGCAGTGGGGAATATTGCACAATGGGCGAAAGCCTGATGCAGC | Streptomyces rimosus |
| | | GACGCCGCGTGAGGGATGACCGCCCTTCGGGTTGTAAACCTCTTTCAGCAGG | Streptomyces tubercidicus |
| | | GAAGAAGCGAGAGTGACGGTACCTGCAGAAGAAGCGCCGGCTAACTACGT | |
| | | GCCAGCAGCCGCGGTAATACGTAGGGCGCAAGCGTTGTCCGGAATTATTGG | |
| | | GCGTAAAGAGCTCGTAGGCGGCTTGTCACGTCGGATGTGAAAGCCCGGGGC | |
| | | TTAACCCCGGGTCTGCATTCGATACGGGCAGGCTAGAGTTCGGTAGGGGAG | |
| | | ATCGGAATTCCTGGTGTAGCGGTGAAATGCGCAGATATCAGGAGGAACACC | |
| | | GGTGGCGAAGGCGGATCTCTGGGCCGATACTGACGCTGAGGAGCGAAAGCG | |
| | | TGGGGAGCGAACAGGATTAGATACCCTGGTAGTCCACGCCGTAAACGTTGG | |
| | | GAACTAGGTGTGGGCGACATTCCACGTCGTCCGTGCCGCAGCTAACGCATTA | |
| | | AGTTCCCCGCCTGGGGAGTACGGCCGCAAGGCTAAAACTCAAAGGAATTGA | |
| | | CGGGGGCCCGCACAAGCAGCGGAGCATGTGGCTTAATTCGACGCAACGCGA | |
| | | AGAACCTTACCAAGGCTTGACATACACCGGAAAACCCTGGAGACAGGGTCC | |
| | | CCCTTGTGGTCGGTGTACAGGTGGTGCATGGCTGTCGTCAGCTCGTGTCGTG | |
| | | AAGATGTTGGGTTAAGTCCCGCAACGAGCGCAACCCTGGTCCGGGGTGGCC | |
| | | AGCATGCCCTTCGGGGTGATGGGGACTCACAGAAAACGGCCGGGGTCAACT | |
| | | CGGAGGAAGGTGGGGACGACGTCAAGTCATCATGCCCCTTATGTCTTGGGC | |
| | | TGCACACGTGCTACAATGGCCGGTACAATGAGCTGCGATACCGCGAGGTGG | |
| | | AGCGAATCTCAAAAAGCCGGTCTCAGTTCGGATTGGGGTCTGCAACTCGAC | |
| | | CCCATGAAGTCGGAGTTGCTAGTAATCGCAGATCAGCATGCTGCGTATGCG | |
| | | GC | |
| JBS6899 | 26 | TGATTCGAAAGGCCGCGGCTAACACATGCAGGCGAACGATGAACCTCCTTC | Streptomyces angustmyceticus |
| | | GGGAGGGGATTAGTGGCGAACGGGTGAGTAACACCTCGACAATATGCCAAT | Streptomyces hygroscopicus |
| | | CACGCAGGAACAAGCCATGGAAACAGGGACAAATAAACGGTATATCAAGA | Streptomyces libani |
| | | CCACCCAACGCATGGTCTGCTGGGGGAAAGCTCCCGCGGCGAAAGATGAGC | Streptomyces lydicus |
| | | CCCCGCCCTATCAGCTGGGTGGGGGGATGATGGCCTACAAAAGCGACGACG | Streptomyces nigrescens |
| | | GGTAGCCGACCGCAGAGAGCGACACGCCACACTGGGACTGAGACACGGCCC | Streptomyces platensis |
| | | AGACTCCTACGGGAGGCAGCAGTGGGGAATATGCAAAAAGGGGCCAAAAC | Streptomyces rimosus |
| | | CTGATGCAGCGACGCCGCGTGAGGGATGACGGCCTTCGGGTTGTAAACCTC | Streptomyces sioyaensis |
| | | TTTCAGCAGGGAAGAAGCGAGAGTGACGGTACCTGCAGAAGAAGCGCCGG | |
| | | CTAACTACGTGCCAGCAGCCGCGGTAATACGTAGGGCGCAAGCGTTGTCCG | |
| | | GAATTATTGGGCGTAAAGAGCTCGTAGGCGGCTTGTCACGTCGGATGTGAA | |
| | | AGCCCGGGGCTTAACCCCGGGTCTGCATTCGATACGGGCAGGCTAGAGTTC | |
| | | GGTAGGGGAGATCGGAATTCCTGGTGTAGCGGTGAAATGCGCAGATATCAG | |
| | | GAGGAACACCGGTGGCGAAGGCGGATCTCTGGGCCGATACTGACGCTGAGG | |
| | | AGCGAAAGCGTGGGGAGCGAACAGGATTAGATACCCTGGTAGTCCACGCCG | |
| | | TAAACGTTGGGAACTAGGTGTGGGCGACATTCCACGTCGTCCGTGCCGCAG | |
| | | CTAACGCATTAAGTTCCCCGCCTGGGGAGTACGGCCGCAAGGCTAAAACTC | |
| | | AAAGGAATTGACGGGGGCCCGCACAAGCAGCGGAGCATGTGGCTTAATTCG | |
| | | ACGCAACGCGAAGAACCTTACCAAGGCTTGACATACACCGGAAAACCCTGG | |
| | | AGAACAGGGTCCCCCTTGTGGTCGGTGTACAGGTGGTGCATGGCTGTCGTCA | |
| | | GCTCGTGTCGTGAAATGTTGGGATAAAGCCCCGCAACGAGCGCAAACCCTT | |
| | | GTTCTGTGTTGCCAGCATGCCCCTCCGGGGGAAGGGGACTCACAGAAAACT | |
| | | GCCGGGGCCAACTCGAAGAAAGGGGACGACGAAGTCAAGTCATCAGGCCC | |
| | | CTAAGGTCTTGGGCTGCAAACGTGCCAAAATGGCCGGTACAATGAGCTGCG | |
| | | ATACCGCGAGGTGGAGCGAATCTCAAAAAGCCGGTCTCAGTTCGGATTGGG | |
| | | GTCTGCAACTCGACCCCATGAAGTCGGAGTTGCTAGTAATCGCAGATCAGC | |
| | | ATGCTGCGTAATGCGC | |
| JBS4020 | 4 | AGGGGCGGGGTGCTTACACATGCAAGTCGAACGATGAACCCCGGCGGGCTG | Streptomyces auratus |
| | | GAACACTGGCAAACGAACGAGGAACACCTCGACAATATGCCACCGTCACAC | Streptomyces cyslabdanicus |
| | | CGGGACAAGCCCTGCAAACACAGCCTAAAACCGGATAACACATCCACGCGA | Streptomyces fagopyri |
| | | AGCCAGGTCGGTGAAAAGCTCCGCCGGCGAAAGATGAGCCCGCGGCCCATC | Streptomyces galbus |
| | | AGCTGGGTGGTGAGGTAGTGACGCAACAAGGCGACGACGGGTAGCCGGCCC | Streptomyces kaempferi |
| | | GAAAAGGCCAACCGCCACACTGGGACTGAGACACGGCCCAGAACCCTACGG | Streptomyces mirabilis |
| | | GAAGCAGCAGTGGGGAATATGCAAAAATGGGCCAAAACCTGATGCAGCGA | Streptomyces olivochromogenes |
| | | CGCCGCGTGAGGGATGACGGCCTTCGGGTTGTAAACCTCTTTCAGCAGGGA | Streptomyces olivochromogenes |
| | | AGAAGCGAAAGTGACGGTACCTGCAGAAGAAGCGCCGGCTAACTACGTGCC | |
| | | AGCAGCCGCGGTAATACGTAGGGCGCAAGCGTTGTCCGGGAATTATTGGGC | |
| | | GTAAAGAGCTCGTAGGCGGCTTGTCACGTCGGATGTGAAAGCCCGGGGCTT | |
| | | AACCCCGGGTCTGCATTCGATACGGGCAAGCTAGAGTGCGGTAGGGGAGAT | |
| | | CGGAATTCCTGGTGTAGCGGTGAAATGCGCAGATATCAGGAGGAACACCGG | |
| | | TGGCGAAGGCGGATCTCTGGGCCAATACTGACGCTGAGGAGCGAAAGCGTG | |
| | | GGGAGCGAACAGGATTAGATACCCTGGTAGTCCACGCCGTAAACGGTGGGA | |
| | | ACTAGGTGTGGGCGACATTCCACGTCGTCCGTGCCGCAGCTAACGCATTAAG | |
| | | TTCCCCGCCTGGGGAGTACGGCCGCAAGGCTAAAACTCAAAGGAATTGACG | |
| | | GGGGCCCGCACAAGCAGCGGAGCATGTGGCTTAATTCGACGCAACGCGAAG | |
| | | AACCTTACCAAGGCTTGACATACACCGGAAAACCCCCAGAGACAGGCGCCC | |
| | | CCCTGGGGGCCGTGTACAGGTGGTGCATGGCTGTCGTCAGCTCGTGTCGTGA | |
| | | GATGTTGGGTTAAGTCCCGCAACGAGCGCAACCCTTGTTCTGTGTTGCCAGC | |
| | | ATGCCCTTCCGGGGGAAGGGGACTCACAGGAAAACGGCCGGGGTCAAACTC | |
| | | GGAAGAAGGTGGGGACGACGTCAAGTCATCATGCCCCCTATATCTTCGGGG | |
| | | CCTGCACACGTGCTAAAAAAGCCAGTAAAATGAACTGCGAAACCCCGAGAG | |
| | | CGAAACAACCTCAAAAAGCCGGTCTCAGTTCGGATTGGGGTCTGCAACTCG | |
| | | ACCCCATGAAGTCGGAGTTGCTAGTAATCGCAGATCAGCATGCTGCGTATG | |
| | | GG | |
| JBS9225 | 27 | CGGGCGGGTGCTTACACATGCAGTCGAACGATGAACCTCCTTCGGGAGGGG | Streptomyces angustmyceticus |
| | | ATTAGTGGCAAACGCGTGAGTAACACGTGAACAACATGCACGTAACTCGAG | Streptomyces atrolaccus |
| | | AACAAGACCTGGAAACGCAGGCTAATGACAGCAGACGACACCGGATCGAA | Streptomyces chattanoogensis |
| | | CGACCACCACGTCGAAAACTCCGGCCGCGAAGGATAAGCCCGCGGCCTATC | Streptomyces libani |
| | | AGCTGGTTGGTGGGGTGAGGGACGACAAAAGCGACGACGGGTAGCAGCCCT | Streptomyces lydicus |
| | | GAGAGGGCGACACGCCACACTGGGACTGAGACACGCCACAGACACCCAAC | Streptomyces nigrescens |
| | | GGAGGCAGCAGTGGGGAAAATGCAAAAATGGGCGAAAGCCTGATGCAGCG | Streptomyces sioyaensis |
| | | ACGCCGCGTGAGGGATGACGGCCGCCCGGGTGTAAACCTCTTTCAGCAGGG | Streptomyces tubercidicus |
| | | AAGAAGCGAGAGTGACGGTACCTGCAGAAGAAGCGCCGGCTAACTACGTGC | |
| | | CAGCAGCCGCGGTAATACGTAGGGCGCAAGCGTTGTCCGGAATTATTGGGC | |
| | | GTAAAGAGCTCGTAGGCGGCTTGTCACGTCGGATGTGAAAGCCCGGGGCTT | |
| | | AACCCCGGGTCTGCATTCGATACGGGCAGGCTAGAGTTCGGTAGGGGAGAT | |
| | | CGGAATTCCTGGTGTAGCGGTGAAATGCGCAGATATCAGGAGGAACACCGG | |
| | | TGGCGAAGGCGGATCTCTGGGCCGATACTGACGCTGAGGAGCGAAAGCGTG | |
| | | GGGAGCGAACAGGATTAGATACCCTGGTAGTCCACGCCGTAAACGTTGGGA | |
| | | ACTAGGTGTGGGCGACATTCCACGTCGTCCGTGCCGCAGCTAACGCATTAAG | |
| | | TTCCCCGCCTGGGGAGTACGGCCGCAAGGCTAAAACTCAAAGGAATTGACG | |
| | | GGGGCCCGCACAAGCAGCGGAGCATGTGGCTTAATTCGACGCAACGCGAAG | |
| | | AACCTTACCAAGGCTTGACATACACCGGAAAACCCTGGAGACAGGGTCCCC | |
| | | CTTGTGGTCGGTGTACAGGTGGTGCATGGCTGTCGTCACCTCGTGTCGTGAG | |
| | | ATGTTGGGTTAAGTCCCGCAACAACCGCAACCCTTGTTCCGGGGTGCCAGCA | |
| | | TGCCCTTCGGGGTGATGGGCACTCACAAGAAACGGCCCGGGTCAACCCCGA | |
| | | GGAAGGTGGGGACGACGTCAAGTCATCATGCCCCTTATGTCTTGGGCTGCAC | |
| | | ACGTGCTACAATGGCCGGTACAATGAGCTGCGATACCGCGAGGTGGAGCGA | |
| | | ATCTCAAAAAGCCGGTCTCAGTTCGGATTGGGGTCTGCAACTCGACCCCATG | |
| | | AAGTCGGAGTTGCTAGTAATCGCAGATCAGCATGCTGCGTATGCAC | |
| JBS9264 | 8 | CCAGCCGGTGCTTAAACACACAGTACAAGTCGACAAAAGATAAAGCCCTTC | Streptomyces avidinii |
| | | GGAGGGGATTAGTGGCAAACGGGTGAGTAACACGTGAGCAACCTGCCATTC | Streptomyces colombiensis |
| | | ACCCTCAGACAAACAAGCGGAAACGGAGTCTAATACACGATACCACTCCTC | Streptomyces flaveus |
| | | CACGCATGGTCCGGGGGTGGAAAGCTCCGGCGGTGAAGAAGGAGCCCGCCG | Streptomyces goshikiensis |
| | | CCTATCAGCTGGTGGGGGGGGTAAGAGCCCACCAAGGCGACGACCGGGAAC | Streptomyces lavendulae |
| | | CCGCCTGAGAGGGCGACCGGCCACACTGGGACTGAGACACGGCCCAGACTC | Streptomyces spororaveus |
| | | CTACGGGAGGCAGCAGTGGGGAATATTGCACAATGGGCGAAAGCCTGATGC | Streptomyces subrutilus |
| | | AGCGACGCCGCGTGAGGGATGACGGCCTTCGGGTTGTAAACCTCTTTCAGC | Streptomyces venezuelae |
| | | AGGGAAGAAGCGAAAGTGACGGTACCTGCAGAAGAAGCGCCGGCTAACTA | |
| | | CGTGCCAGCAGCCGCGGTAATACGTAGGGCGCAAGCGTTGTCCGGAATTAT | |
| | | TGGGCGTAAAGAGCTCGTAGGCGGCTTGTCACGTCGGATGTGAAAGCCCGA | |
| | | GGCTTAACCCCGGGTCTGCATTCGATACGGGCAAGCTAGAGTGCGGTAGGG | |
| | | GAGATCGGAATTCCTGGTGTAGCGGTGAAATGCGCAGATATCAGGAGGAAC | |
| | | ACCGGTGGCGAAGGCGGATCTCTGGGCCAATACTGACGCTGAGGAGCGAAA | |
| | | GCGTGGGGAGCGAACAGGATTAGATACCCTGGTAGTCCACGCCGTAAACGT | |
| | | TGGGAACTAGGTGTGGGCGACATTCCACGTCGTCCGTGCCGCAGCTAACGC | |
| | | ATTAAGTTCCCCGCCTGGGGAGTACGGCCGCAAGGCTAAAACTCAAAGGAA | |
| | | TTGACGGGGGCCCGCACAAGCAGCGGAGCATGTGGCTTAATTCGACGCAAC | |
| | | GCGAAGAACCTTACCAAGGCTTGACATACACCGGAAAACACTAGAGACAGG | |
| | | GCCCCCCTTGTGGTCGGTATACAGGTGGTGCATGGCTGTCGTCAGCTCGTGT | |
| | | CGTGAGATGTTGGGTTAAGTCCCCAAACGAGCGCAACCCTTGTCCTGTGTTG | |
| | | CCAGCATGCCCTTCGGGGTGATGGGAACCCACAAGAAACCGCCGGGGTCAA | |
| | | CTCGAAGAAAGGGGGGGAACAACTCAAGTCATCATGCCCCTAATCTCTTGG | |
| | | GCTGCACACGTGCTACAATGGCCGGTACAATGAGCTGCGATACCGCGAGGT | |
| | | GGAGCGAATCTCAAAAAGCCGGTCTCAGTTCGGATTGGGGTCTGCAACTCG | |
| | | ACCCCATGAAGTCGGAGTTGCTAGTAATCGCAGATCAGCATGCTGCGGTAA | |
| | | TGCTC | |
| JBS8135 | 28 | CGGTGCCGACGAACACAGGCAAGGCGAAAGAACAACCAACTGCGAGAAGG | Streptomyces angustmyceticus |
| | | GATAAGCCGCGAACGAATAAGGAAAACGTGAGCAACCTGCCCATCACTCCA | Streptomyces hygroscopicus |
| | | GCACAAACCCTGGAAACGGGGTCTAATACACGATACGACCTCCGACCGCAA | Streptomyces libani |
| | | GGTCTGGGGGTGGAAAGCTCCGCCGGAGAAGGAAGAACCCGCGGCCTATCA | Streptomyces lydicus |
| | | GCTGGGTGGGGGGGTGATGCCCTACAAAGGCGACGACGGGTAGCCGGCCTG | Streptomyces nigrescens |
| | | AGAGGGCGACCACCCAAAATGGGACTGAGACACGGCCCAGACTCCTACGGG | Streptomyces platensis |
| | | AGGCAGCAGTGGGGAATATGGCAAAAAGGGCCAAAACCTGATGCAGCGAC | Streptomyces rimosus |
| | | GCCGCGTGAGGGATGACGGCCTTCGGGTTGGAAACCTCTTTCAGCAGGGAA | Streptomyces tubercidicus |
| | | GAAGCGAGAGTGACGGTACCTGCAGAAGAAGCGCCGGCTAACTACGTGCCA | |
| | | GCAGCCGCGGTAATACGTAGGGCGCAAGCGTTGTCCGGAATTATTGGGCGT | |
| | | AAAGAGCTCGTAGGCGGCTTGTCACGTCGGATGTGAAAGCCCGGGGCTTAA | |
| | | CCCCGGGTCTGCATTCGATACGGGCAGGCTAGAGTTCGGTAGGGGAGATCG | |
| | | GAATTCCTGGTGTAGCGGTGAAATGCGCAGATATCAGGAGGAACACCGGTG | |
| | | GCGAAGGCGGATCTCTGGGCCGATACTGACGCTGAGGAGCGAAAGCGTGGG | |
| | | GAGCGAACAGGATTAGATACCCTGGTAGTCCACGCCGTAAACGTTGGGAAC | |
| | | TAGGTGTGGGCGACATTCCACGTCGTCCGTGCCGCAGCTAACGCATTAAGTT | |
| | | CCCCGCCTGGGGAGTACGGCCGCAAGGCTAAAACTCAAAGGAATTGACGGG | |
| | | GGCCCGCACAAGCAGCGGAGCATGTGGCTTAATTCGACGCAACGCGAAGAA | |
| | | CCTTACCAAGGCTTGACATACACCGGAAAACCCTGGAGACAGGGTCCCCCT | |
| | | TGTGGTCGGTGTACAGGTGGTGCATGGCTGTCGTCAGCTCGTGTCGTGAGAT | |
| | | GTTGGGTTAAGTCCCGCAACGAGCGCAACCCTTGTTCTGTGTTGCCAGCATG | |
| | | CCCTTCGGGGTGATGGGGACCCAAAAGAGACTGCCGGGGCCAACTCGGAGA | |
| | | AAGGGGACCACGACAGCAAGTCATCATGCCCCTAATGTCTGGCGCCAAACA | |
| | | CGTGCTACAAGGGACCGGACAATGAACTGCCATACCGCGAAGGTGGAACGA | |
| | | ATCTCAAAAAGCCGGTCTCAGTTCGGATTGGGGTCTGCAACTCGACCCCATG | |
| | | AAGTCGGAGTTGCTAGTAATCGCAGATCAGCATGCTGCGGTATTGCTC | |
| JBS5867 | 9 | AAGGCGCGCGAACACAAATGCAAGTCCAAAAAGGAAACCCTTCGAGGTGG | Streptomyces cinnamonensis |
| | | AATAGGCGAGAACGGGTGAGTAACACGGGGGCAACCGGCACTGCACTCAC | Streptomyces cirratus |
| | | GGACAAGCCCAAGAAAAACGGTCTAATACACGATAATACTCCTGCCGCAAT | Streptomyces goshikiensis |
| | | GGGCCGGGGTTGAAAGCTCCGGCGCGGAAAGATGAGCCCGCGGCCTATCAG | Streptomyces lavendulae |
| | | CTGGTTGGTGGGGTAATGGCCCACAAAGGCGACGACGGGTAGCCAGCCGCA | Streptomyces senoensis |
| | | GAGAGCGACACGCCACACTGGGACTGAGACACGGCCCAGACTCCTACGGGA | Streptomyces sporoverrucosus |
| | | GGCAGCAGTGGGGAATATTGCACAATGGGCGAAAGCCTGATGCAGCGACGC | Streptomyces vinaceus |
| | | CGCGTGAGGGATGACGGCCTTCGGGTTGTAAACCTCTTTCAGCAGGGAAGA | Streptomyces virginiae |
| | | AGCGAAAGTGACGGTACCTGCAGAAGAAGCGCCGGGCTAACTACGTGCCAG | |
| | | CAGCCGCGGTAATACGTAGGGCGCAAGCGTTGTCCGGAATTATTGGGCGTA | |
| | | AAGAGCTCGTAGGCGGCTTGTCACGTCGGATGTGAAAGCCCGAGGCTTAAC | |
| | | CTCGGGTCTGCATTCGATACGGGCTAGCTAGAGTGTGGTAGGGGAGATCGG | |
| | | AATTCCTGGTGTAGCGGTGAAATGCGCAGATATCAGGAGGAACACCGGTGG | |
| | | CGAAGGCGGATCTCTGGGCCATTACTGACGCTGAGGAGCGAAAGCGTGGGG | |
| | | AGCGAACAGGATTAGATACCCTGGTAGTCCACGCCGTAAACGTTGGGAACT | |
| | | AGGTGTTGGCGACATTCCACGTCGTCGGTGCCGCAGCTAACGCATTAAGTTC | |
| | | CCCGCCTGGGGAGTACGGCCGCAAGGCTAAAACTCAAAGGAATTGACGGGG | |
| | | GCCCGCACAAGCGGCGGAGCATGTGGCTTAATTCGACGCAACGCGAAGAAC | |
| | | CTTACCAAGGCTTGACATATACCGGAAAGCATTAGAGATAGTGCCCCCCTTG | |
| | | TGGTCGGTATACAGGTGGTGCATGGCTGTCGTCAGCTCGTGTCGTGAGATGT | |
| | | TGGGTTAAGTCCCGCAACGAGCGCAACCCTGGCCCGGTGTTGCCAGCATGC | |
| | | CCTTCGGGGGAAGGGACACCAAAAGAAAACCGCCGCGGTCAACTCGAAGA | |
| | | AAGGTGGGGACGACGTCAAGTCATCAAGCCCCTTATGGCTCGGGCTGCACA | |
| | | CGTGCTACAATGCCCGGTACAATGAACTGCCATACCGAGAAGTGAAGCGAA | |
| | | TCTCAAAAAGCCGGCCCCACATCCGATTGGGGGCGGCAACTCGACCCCATG | |
| | | AAGTCGGAGTCGCTAGTAATCGCAGATCAGCATGCTGCGGTTATGCTA | |
| JBS9311 | 10 | AGGGCGGCGTGCTGACACATAAAACTCAAACGATGAAGCAAGTCCGAATCA | Streptomyces cinnamonensis |
| | | ATGAATGCCGAAAGGGAAGAGGAACACGTGAGCAACCGGCCAATCACTCC | Streptomyces flaveus |
| | | GCGACAAGCCCTGGAAACGGGGTCTAATACACGATACCAATCCTGCCTGCA | Streptomyces lavendulae |
| | | TGACCGGGGGGTGAAAGCTAAGGCGGCGAAAAAAGAACCCGCGGCCAATC | Streptomyces nojiriensis |
| | | AGCTTGGCGGTGGGGGAAAAGCCCACCAAGGCGACGACGGGTAGCCGCCCT | Streptomyces spororaveus |
| | | GAAAAGGCCAACGGCCACACTGGGACTGAGACACGGCCCAGACTCCTACGG | Streptomyces venezuelae |
| | | GAGGCAGCAGTGGGGAATATTGCACAATGGGCGAAAACCTGATGCAGCGAC | Streptomyces virginiae |
| | | GCCGCGTGAGGGATGACGGCCTTCGGGTTGTAAACCTCTTTCAGCAGGGAA | Streptomyces xanthophaeus |
| | | GAAGCGAAAGTGACGGTACCTGCAGAAGAAGCGCCGGCTAACTACGTGCCA | |
| | | GCAGCCGCGGTAATACGTAGGGCGCAAGCGTTGTCCGGAATTATTGGGCGT | |
| | | AAAGAGCTCGTAGGCGGCTTGTCACGTCGGATGTGAAAGCCCGAGGCTTAA | |
| | | CCTCGGGTCTGCATTCGATACGGGCTAGCTAGAGTGTGGTAGGGGAGATCG | |
| | | GAATTCCTGGTGTAGCGGTGAAATGCGCAGATATCAGGAGGAACACCGGTG | |
| | | GCGAAGGCGGATCTCTGGGCCATTACTGACGCTGAGGAGCGAAAGCGTGGG | |
| | | GAGCGAACAGGATTAGATACCCTGGTAGTCCACGCCGTAAACGTTGGGAAC | |
| | | TAGGTGTTGGCGACATTCCACGTCGTCGGTGCCGCAGCTAACGCATTAAGTT | |
| | | CCCCGCCTGGGGAGTACGGCCGCAAGGCTAAAACTCAAAGGAATTGACGGG | |
| | | GGCCCGCACAAGCGGCGGAGCATGTGGCTTAATTCGACGCAACGCGAAGAA | |
| | | CCTAACCAAGGCTTGACATATACCGGAAAGCATTAGAGATAGTGCCCCCCT | |
| | | GGGGGTCGCGAAAAAAGTGGTGCATGGCTGTCGTCAGCTCGTGTCGTGAGA | |
| | | TGTTGGGTAAAGCCCCCAAAAGAGCACAACCCTTGTCCTCTGTGGCCAGCAT | |
| | | GCCCTTCGGGGGAAGGAGGACTCACAGGAAACCCCCGGGGTCAACTCGGAG | |
| | | AAAGGTGGGGACGACCTCAAGTCATCATGCCCCAGACGTCTTGGGCTGCAC | |
| | | ACGTGCTACAATGGCCGGTACAATGAGCTGCGATACCGTGAGGTGGAGCGA | |
| | | ATCTCAAAAAGCCGGTCTCAGTTCGGATTGGGGTCTGCAACTCGACCCCATG | |
| | | AAGTCGGAGTCGCTAGTAATCGCAGATCAGCATGCTGCGTAATGGCC | |
| JBS4046 | 1 | AATCATGGCGCCGGGCTAACACATGCAGTCCAACGAAGAAGCCCGGCGGGG | Streptomyces echinatus |
| | | TGGATTAGAGGCGAAACGGTGAGTAACACGTGAGCAACCTGCACCGCACTC | Streptomyces filipinensis |
| | | TGGGACAAGCCCTGGAAAAGGGGTCCAAGACCGGATATAAGACTGAGAGG | Streptomyces gulbargensis |
| | | CAGCATCCCCGGGTGTAAAGCTCCGCCGGTGCAAGATGAGCCCCCCGCCTA | Streptomyces longwoodensis |
| | | TCAGCTAGCTGGTGAGAGAAAGGCTCACCAAGGCGACGACGGGTAGCCCGC | Streptomyces myxogenes |
| | | CTGAGAGGGCGACCGGCCACACTGGGACTGAGACACGGCCCAGACTCCTAC | Streptomyces novaecaesareae |
| | | GGGAGGCAGCAGTGGGGAATATTGCACAATGGGCGAAAGCCTGATGCAGC | Streptomyces spectabilis |
| | | GACGCCGCGTGAGGGATGACGGCCTTCGGGTTGTAAACCTCTTTCAGCAGG | Streptomyces tanashiensis |
| | | GAAGAAGCGAGAGTGACGGTACCTGCAGAAGAAGCGCCGGCTAACTACGT | |
| | | GCCAGCAGCCGCGGTAATACGTAGGGCGCAAGCGTTGTCCGGAATTATTGG | |
| | | GCGTAAAGAGCTCGTAGGCGGCTTGTCACGTCGATTGTGAAAGCCCGAGGC | |
| | | TTAACCTCGGGTCTGCAGTCGATACGGGCTAGCTAGAGTGTGGTAGGGGAG | |
| | | ATCGGAATTCCTGGTGTAGCGGTGAAATGCGCAGATATCAGGAGGAACACC | |
| | | GGTGGCGAAGGCGGATCTCTGGGCCATTACTGACGCTGAGGAGCGAAAGCG | |
| | | TGGGGAGCGAACAGGATTAGATACCCTGGTAGTCCACGCCGTAAACGGTGG | |
| | | GAACTAGGTGTTGGCGACATTCCACGTCGTCGGTGCCGCAGCTAACGCATTA | |
| | | AGTTCCCCGCCTGGGGAGTACGGCCGCAAGGCTAAAACTCAAAGGAATTGA | |
| | | CGGGGGCCCGCACAAGCGGCGGAGCATGTGGCTTAATTCGACGCAACGCGA | |
| | | AGAACCTTACCAAGGCTTGACATACACCGGAAAGCATTAGAGATAGTGCCC | |
| | | CCCTTGTGGTCGGTGTAACAGGTGGTGCATGGCTGTCGTCAGCTCGTGTCGG | |
| | | GAGATGTTGGGTTAAGTCCCGCAACGAGCGCAACCCTTGTTCTGTGTTGCCA | |
| | | GCATGCCCTTCGGGGGAATGGGAACCCACAGAAAACCCCCGGGCAAAACTC | |
| | | GAAGAAAGGGACGGACGACCTCAAGTCATCATGCCCCTCATATCTCGGGCT | |
| | | GCAAACGTGCTACAATGGCCGGTACAATGAACTGCAAAACCGAGAGGTAGA | |
| | | GCGAATCTCAAAAAGCCGGTCTCAGTTCGGATTGGGGTCTGCAACTCGACCC | |
| | | CATGAAGTCGGAGTCGCTAGTAATCGCAGATCAGCATGCTGCGTATGCGAC | |
| JBS1726 | 2 | AGCGCCGCGTGCGTACACAAACAGTCCAACGACGAACACCATCGGGAAGCA | Streptomyces echinatus |
| | | TCAGTGGAGAACGGGTGAGGAAAACGTGAGCAACCGGCACTGCACCATGG | Streptomyces ginsengisoli |
| | | GACAAGCCCTGGAAACGCGGACGAATACAGAAGATGAACCTGGGAGGCAT | Streptomyces graminisoli |
| | | CTCCCGGCGGGTAAAACTCCGGCGGTGCAGCAGAAGACCCCCGCCTATCAA | Streptomyces gulbargensis |
| | | CCTGTGGGGGAGGTAATGGCTCACCAAGGCGACGACGGGTAGCCGGCCGAA | Streptomyces longwoodensis |
| | | AAGGGCCAACGGCCACACTGGGACTGAGACACGGCCCAGACTCCTACGGGA | Streptomyces lucensis |
| | | GGCAGCAGTGGGGAATATTGCACAATGGGCGAAAACCTGATGCAGCGACGC | Streptomyces tanashiensis |
| | | CGCGTGAGGGATGACGGCCTTCGGGTTGTAAACCTCTTTCAGCAGGGAAGA | Streptomyces yaanensis |
| | | AGCGAGAGTGACGGTACCTGCAGAAGAAGCGCCGGCTAACTACGTGCCAGC | |
| | | AGCCGCGGTAATACGTAGGGCGCAAGCGTTGTCCGGAATTATTGGGCGTAA | |
| | | AGAGCTCGTAGGCGGCTTGTCACGTCGATTGTGAAAGCCCGAGGCTTAACCT | |
| | | CGGGTCTGCAGTCGATACGGGCTAGCTAGAGTGTGGTAGGGGAGATCGGAA | |
| | | TTCCTGGTGTAGCGGTGAAATGCGCAGATATCAGGAGGAACACCGGTGGCG | |
| | | AAGGCGGATCTCTGGGCCATTACTGACGCTGAGGAGCGAAAGCGTGGGGAG | |
| | | CGAACAGGATTAGATACCCTGGTAGTCCACGCCGTAAACGGTGGGAACTAG | |
| | | GTGTTGGCGACATTCCACGTCGTCGGTGCCGCAGCTAACGCATTAAGTTCCC | |
| | | CGCCTGGGGAGTACGGCCGCAAGGCTAAAACTCAAAGGAATTGACGGGGGC | |
| | | CCGCACAAGCGGCGGAGCATGTGGCTTAATTCGACGCAACGCGAAGAACCT | |
| | | TACCAAGGCTTGACATACACCGGAAAGCATTAGAGATAGTGCCCCCCTTGT | |
| | | GGTCGGTGTACAGGTGGTGCATGGCTGTCGTCAGCTCGTGTCGTGAGATGTT | |
| | | GGGTTAAGTCCCGCAACGAGCGCAACCCTTGTCCGGGGTTGCCAGCATGCC | |
| | | CTCCGGGGTGATGGGGACTCAAAAGAAACCGCCGGGGTCAACTCGGAGAAA | |
| | | GGTGGGAACAACGTCAAGTCATCATGCCCCTTATGTCTTGGGCCGGCAACAC | |
| | | GTGCTACAATGGCCGGTACAATGAGCTGCGATACCGTGAGGTGGAGCGAAT | |
| | | CTCAAAAAGCCGGTCTCAGTTCGGATTGGGGTCTGCAACTCGACCCCATGAA | |
| | | GTCGGAGTCGCTAGTAATCGCAGATCAGCATGCTGCGGTAATGCGC | |
| JBS8612 | 11 | AGGGGGGGCGAGCATACACACGAAGTCCAACCACGAACATCCTTCGCGAAA | Streptomyces auratus |
| | | CGATGAGCGCCGAAAAGATGAGCAACACGTGGGCAAGATAACCTGCACACT | Streptomyces cinnamonensis |
| | | GCCACAAGCACAGCAAAAACGGACTAATACCGGATAACAAATCAAACCGC | Streptomyces lavendulae |
| | | ATGCACGGCGGGGGGAAAGCTCCGGCGGTGAAAGATGAGCCCGCGGCCTAT | Streptomyces sioyaensis |
| | | CAGCTGGGTGGGGAGGCCACGGCCTACCAAGGCGAAAACGACGACCCGAC | Streptomyces spororaveus |
| | | CGCAGAGAGCGACACGCCACACTGGGACTGAGACACGGCCCAGACTCCTAC | Streptomyces verne |
| | | GGGAGGCAGCAGTGGGGAATATTGCACAATGGGCGAAAGCCTGATGCAGC | Streptomyces virginiae |
| | | GACGCCGCGTGAGGGATGACGGCCTTCGGGTTGTAAACCTCTTTCAGCAGG | Streptomyces xanthophaeus |
| | | GAAGAAGCGAAAGTGACGGTACCTGCAGAAGAAGCGCCGGCTAACTACGT | |
| | | GCCAGCAGCCGCGGTAATACGTAGGGCGCAAGCGTTGTCCGGGAATTATTG | |
| | | GGCGTAAAGAGCTCGTAGGCGGCTTGTCACGTCGGATGTGAAAGCCCGAGG | |
| | | CTTAACCCCGGGTCTGCATTCGATACGGGCAAGCTAGAGTGCGGTAGGGGA | |
| | | GATCGGAATTCCTGGTGTAGCGGTGAAATGCGCAGATATCAGGAGGAACAC | |
| | | CGGTGGCGAAGGCGGATCTCTGGGCCAATACTGACGCTGAGGAGCGAAAGC | |
| | | GTGGGGAGCGAACAGGATTAGATACCCTGGTAGTCCACGCCGTAAACGTTG | |
| | | GGAACTAGGTGTGGGCGACATTCCACGTCGTCCGTGCCGCAGCTAACGCATT | |
| | | AAGTTCCCCGCCTGGGGAGTACGGCCGCAAGGCTAAAACTCAAAGAAATGA | |
| | | ACGGGGGCCCGCACAAGCAGCGGAGCATGTGGCTTAATTCGACGCAAACCC | |
| | | AAAAAACCTAACAAAGCTTGACATACACCGGAAAACACTAAAAACAGGGC | |
| | | CCCCCTTGTGGTCGGTATACAGGGGGGCAAGGCCTGTCGTCAGCTCGTGTCG | |
| | | TGAGATGTTGGGTTAAGTCCCGCAACGAGCGCAACCCTTGTCCTGTGTTGCC | |
| | | AGCATGCCCTTCGGGGTGATGGGGACTCACAGGAGACCGCCGGGGTCAACT | |
| | | CGGAGGAAGGTGGGGACGACGTCAAGTCATCATGCCCCTTATGTCTTGGGC | |
| | | TGCACACGTGCTACAATGGCCGGTACAATGAGCTGCGATACCGTGAGGTGG | |
| | | AGCGAATCTCAAAAAGCCGGTCTCAGTTCGGATTGGGGTCTGCAACTCGAC | |
| | | CCCATGAAGTCGGAGTCGCTAGTAATCGCAGATCAGCATGCTGCGGTAATG | |
| | | GGC | |
| JBS5020 | 29 | CGGGCGCGTGCTTACACATGCAAGTCGAACCAAAAACCACCGTCAAGAGGC | Streptomyces angustmyceticus |
| | | GATAAGTGGCGAACGGGTGACAAACACGGAGGCAATATGCCCCACACTCAG | Streptomyces atrolaccus |
| | | GAACAAGCCATGGAAAACGGGACAAATAACGGGATACAACATACGACCGC | Streptomyces hygroscopicus |
| | | AACGCATGGTCGTGGAAAACTCCGGCCGCGAAAGATGAGCCCGCGCCATAT | Streptomyces libani |
| | | CAGCTTGCAGCTGGGGTGAGGGCATACCAAAGCGACGACGGGTAGCCGGCC | Streptomyces lydicus |
| | | GCAGAGAGCGACACCCCACACTGGGACTGAGACACGGCCCAGACTCCTACG | Streptomyces nigrescens |
| | | GGAGGCAGCAGTGGGGAATATTGCACAATGGGCGAAAGCCTGATGCAGCG | Streptomyces sioyaensis |
| | | ACGCCGCGTGAGGGATGACGGCCTTCCGGGTGGAAACCTCTTTCAGCAGGG | Streptomyces tubercidicus |
| | | AAGAAGCGAGAGTGACGGTACCTGCAGAAGAAGCGCCGGCTAACTACGTGC | |
| | | CAGCAGCCGCGGTAATACGTAGGGCGCAAGCGTTGTCCGGAATTATTGGGC | |
| | | GTAAAGAGCTCGTAGGCGGCTTGTCACGTCGGATGTGAAAGCCCGGGGCTT | |
| | | AACCCCGGGTCTGCATTCGATACGGGCAGGCTAGAGTTCGGTAGGGGAGAT | |
| | | CGGAATTCCTGGTGTAGCGGTGAAATGCGCAGATATCAGGAGGAACACCGG | |
| | | TGGCGAAGGCGGATCTCTGGGCCGATACTGACGCTGAGGAGCGAAAGCGTG | |
| | | GGGAGCGAACAGGATTAGATACCCTGGTAGTCCACGCCGTAAACGTTGGGA | |
| | | ACTAGGTGTGGGCGACATTCCACGTCGTCCGTGCCGCAGCTAACGCATTAAG | |
| | | TTCCCCGCCTGGGGAGTACGGCCGCAAGGCTAAAACTCAAAGGAATTGACG | |
| | | GGGGCCCGCACAAGCAGCGGAGCATGTGGCTTAATTCGACGCAACGCGAAG | |
| | | AACCTTACCAAGGCTTGACATACACCGGAAAACCCTGGAGACAGGGTCCCC | |
| | | CTTGTGGTCGGTGTACAGGTGGTGCATGGCTGTCGTCAGCTCGTGTCGTGAG | |
| | | AAGGTGGGTTAAGTCCCGCAACGAGCGCAACCCTTGTTCTGTGTTGCAACCA | |
| | | AGCCCTTCGGGGTGATGGGGACTCACAGGAGACTGCCGGGGTCAACCCGGA | |
| | | AGAAAGTGGGGACGACGTCAAGTCATCATGCCCCTTATGTCTTGGGCTGCAC | |
| | | ACGTGCTACAATGGCCGGTACAATGAGCTGCGATACCGCGAGGTGGAGCGA | |
| | | ATCTCAAAAAGCCGGTCTCAGTTCGGATTGGGGTCTGCAACTCGACCCCATG | |
| | | AAGTCGGAGTTGCTAGTAATCGCAGATCAGCATGCTGCGTAATGCAC | |
| JBS9780 | 12 | ACGCGGCGGTGCTTAAACACGCAAGCGAACGATGAAGCCCTTCGGGGTGGA | Streptomyces cinnamonensis |
| | | TTAGAGAAGAACGGGTGAGTAACACACGGGCAACCTGCCATTCACTCCGGC | Streptomyces flaveus |
| | | ACAAACCCTGGAAACAGGGTCTAATAACCGATACCACTACAACCTGCAGCA | Streptomyces lavendulae |
| | | GCCGGGGTTGAAAGCTCCCGCGGTGAAGAAAGAACCCGCGGCCTATCAGCT | Streptomyces nojiriensis |
| | | GGTTGGTGGGGGAAAGGCCCAACAAAGCGACGACGGGTAGCCGCCCAGAG | Streptomyces spororaveus |
| | | AAGGCCAACGGCAAAACGGGAACGGAGACACGGCCCAGACTCCTACGGGA | Streptomyces venezuelae |
| | | GGCAGCAGTGGGGAATATTGCACAATGGGCGAAAGCCTGATGCAGCGACGC | Streptomyces virginiae |
| | | CGCGTGAGGGATGACGGCCTTCGGGTTGTAAACCTCTTTCAGCAGGGAAGA | Streptomyces xanthophaeus |
| | | AGCGAAAGTGACGGTACCTGCAGAAGAAGCGCCGGCTAACTACGTGCCAGC | |
| | | AGCCGCGGTAATACGTAGGGCGCAAGCGTTGTCCGGAATTATTGGGCGTAA | |
| | | AGAGCTCGTAGGCGGCTTGTCACGTCGGATGTGAAAGCCCGAGGCTTAACC | |
| | | TCGGGTCTGCATTCGATACGGGCTAGCTAGAGTGTGGTAGGGGAGATCGGA | |
| | | ATTCCTGGTGTAGCGGTGAAATGCGCAGATATCAGGAGGAACACCGGTGGC | |
| | | GAAGGCGGATCTCTGGGCCATTACTGACGCTGAGGAGCGAAAGCGTGGGGA | |
| | | GCGAACAGGATTAGATACCCTGGTAGTCCACGCCGTAAACGTTGGGAACTA | |
| | | GGTGTTGGCGACATTCCACGTCGTCGGTGCCGCAGCTAACGCATTAAGTTCC | |
| | | CCGCCTGGGGAGTACGGCCGCAAGGCTAAAACTCAAAGGAATTGACGGGGG | |
| | | CCCGCACAAGCGGCGGAGCATGTGGCTTAATTCGACGCAACGCGAAGAACC | |
| | | TTACCAAGGCTTGACATATACCGGAAAGCATTAGAGATAGTGCCCCCCTTGT | |
| | | GGTCGGTATACAGGTGGTGCATGGCTGTCGTCAGCTCGTGTCGTGAAAGGTG | |
| | | GGGTAAAGTCCCGCAACAAGCGCAACCCTTGTCCTGTGTTGCCAGCATGCCC | |
| | | TTCGGGGTGATGGGGACTCACAGGAGACCGCCGGGGTCAACTCGGAGGAAG | |
| | | GTGGGGACGACGTCAAGTCATCATGCCCCTTATGTCTTGGGCTGCACACGTG | |
| | | CTACAATGGCCGGTACAATGAGCTGCGATACCGTGAGGTGGAGCGAATCTC | |
| | | AAAAAGCCGGTCTCAGTTCGGATTGGGGTCTGCAACTCGACCCCATGAAGT | |
| | | CGGAGTCGCTAGTAATCGCAGATCAGCATGCTGCGGTAATGCGTC | |
| JBS5523 | 30 | CGGGCGCGCGAAAAAACATGCAAGTCGAACAATGAACCCCCTTCGGAAGGA | Streptomyces caniferus |
| | | GATTAGTGGCGAACGGGTGAGGAAAACGTGGGCAATCTGCCCTTCACTAAC | Streptomyces decoyicus |
| | | GGACAAGCCCCGGAAACCGGACCGAATAACCGATACGACCACACAACGCAT | Streptomyces glebosus |
| | | GGTCTGGTGGGGGAAAGCTCCGCCGGTGAAAGATGAGCCCGCGCCCTATCA | Streptomyces hygroscopicus |
| | | GCTGGTGGGTGAGGTGATGGCCTACCAAAGCGACGACGGGTAGCAGCCCGC | Streptomyces libani |
| | | AGAGAGCGACACGCCACACTGGGACTGAGACACGGCCCAGACCCCTACGGG | Streptomyces lydicus |
| | | AGGCAGCAGTGGGGAATATTGCACAATGGGCGAAAGCCTGATGCAGCGACG | Streptomyces ossamyceticus |
| | | CCGCGTGAGGGATGACGGCCTTCGGGTTGTAAACCTCTTTCAGCAGGGAAG | Streptomyces platensis |
| | | AAGCGAGAGTGACGGTACCTGCAGAAGAAGCGCCGGCTAACTACGTGCCAG | |
| | | CAGCCGCGGTAATACGTAGGGCGCAAGCGTTGTCCGGGAATTATTGGGCGT | |
| | | AAAGAGCTCGTAGGCGGCTTGTCACGTCGGATGTGAAAGCCCGGGGCTTAA | |
| | | CCCCGGGTCTGCATTCGATACGGGCAGGCTAGAGTTCGGTAGGGGAGATCG | |
| | | GAATTCCTGGTGTAGCGGTGAAATGCGCAGATATCAGGAGGAACACCGGTG | |
| | | GCGAAGGCGGATCTCTGGGCCGATACTGACGCTGAGGAGCGAAAGCGTGGG | |
| | | GAGCGAACAGGATTAGATACCCTGGTAGTCCACGCCGTAAACGTTGGGAAC | |
| | | TAGGTGTGGGCGACATTCCACGTCGTCCGTGCCGCAGCTAACGCATTAAGTT | |
| | | CCCCGCCTGGGGAGTACGGCCGCAAGGCTAAAACTCAAAGGAATTGACGGG | |
| | | GGCCCGCACAAGCAGCGGAGCATGTGGCTTAATTCGACGCAACGCGAAGAA | |
| | | CCTTACCAAGGCTTGACATACACCGGAAACGTCTGGAGACAGGCGCCCCCT | |
| | | TGTGGTCGGTGTACAGGTGGTGCATGGCTGTCGTCAGCTCGTGTCGTGAGAT | |
| | | GTTGGGTTAAGTCCCGCAACGAGCGCAACCCTTGTTCTGTGTTGCCAGCATG | |
| | | CCCTTCGGGGGAAGGGGAACCCACAGGAGACTGCCGGGCACAACTCGGAG | |
| | | AAAGGGGGGGAACAACGCAAGTCATCATGCCCCTTATGTCTGGCGCCCAAC | |
| | | ACGTGCTACAATGGCCGGTACAAGGAACTGCAACACAGCGAAATGGAGCCA | |
| | | ATCTCAAAAAGCCGGTCTCAATTCGAATTGGAGGCACCAACTCGACCCCAA | |
| | | GAAGTCGGAGTGGCTAATAATCCAATCACAGATCAGCACGCTGCGTATGCG | |
| | | C | |
| JBS8753 | 13 | CGGGCGGGTGCTTACACATGCAGTCGAACAATGAACCACATCGGGGTAGAT | Streptomyces badius |
| | | TAGTAACCAACGGATGAGATATGAGGAACACGTGAGCAAACTGCCATTCAC | Streptomyces cyaneofuscatus |
| | | ACTGGGACAAGCCCTGGAAACGGAACCGAATACACGATAACACTCTGTCCC | Streptomyces flavogriseus |
| | | GCATCGCACGACCGGTAAAAGCTCCGGCGGTGAAAGATGAGACCCCGCCCT | Streptomyces griseus |
| | | ATCAGCTTGTGGGTGGGGTAATGGCCTACAAAAGCGACGACGGGTAGCCCG | Streptomyces lavendulae |
| | | CCCGAAGAGGGCGACCGGCCACACTGGGACTGAGACACGGCCCAGACTCCT | Streptomyces mediolani |
| | | ACGGGAGGCAGCAGTGGGGAATATTGCACAATGGGCGAAAGCCTGATGCA | Streptomyces praecox |
| | | GCGACGCCGCGTGAGGGATGACGGCCTTCGGGTTGTAAACCTCTTTCAGCA | Streptomyces pratensis |
| | | GGGAAGAAGCGAAAGTGACGGTACCTGCAGAAGAAGCGCCGGCTAACTAC | |
| | | GTGCCAGCAGCCGCGGTAATACGTAGGGCGCAAGCGTTGTCCGGAATTATT | |
| | | GGGCGTAAAGAGCTCGTAGGCGGCTTGTCACGTCGGATGTGAAAGCCCGGG | |
| | | GCTTAACCCCGGGTCTGCATTCGATACGGGCTAGCTAGAGTGTGGTAGGGG | |
| | | AGATCGGAATTCCTGGTGTAGCGGTGAAATGCGCAGATATCAGGAGGAACA | |
| | | CCGGTGGCGAAGGCGGATCTCTGGGCCATTACTGACGCTGAGGAGCGAAAG | |
| | | CGTGGGGAGCGAACAGGATTAGATACCCTGGTAGTCCACGCCGTAAACGTT | |
| | | GGGAACTAGGTGTTGGCGACATTCCACGTCGTCGGTGCCGCAGCTAACGCA | |
| | | TTAAGTTCCCCGCCTGGGGAGTACGGCCGCAAGGCTAAAACTCAAAGGAAT | |
| | | TGACGGGGGCCCGCACAAGCAGCGGAGCATGTGGCTTAATTCGACGCAACG | |
| | | CGAAGAACCTTACCAAGGCTTGACATATACCGGAAAGCATCAGAGATGGTG | |
| | | CCCCCCTTGTGGTCGGTATACAGGTGGTGCATGGCTGTCGTCAGCTCGTGTC | |
| | | GTGAGATGTTGGGTTAAGTCCCGCAACGAGCGCAACCCTTGTTCTGTGTTGC | |
| | | CAGCATGCCCTCCGGGGGAAGGGACACCAAAAGAAGACTGCCGGGGTCAA | |
| | | CCCCGAAAAAAATGGGGACGACGTCAAGTCATCATGCCCCTTATGTCTTGG | |
| | | GCTGCACACGTGCTACAATGGCCGGTACAATGAGCTGCGATGCCGCGAGGC | |
| | | GGAGCGAATCTCAAAAAGCCGGTCTCAGTTCGGATTGGGGTCTGCAACTCG | |
| | | ACCCCATGAAGTCGGAGTTGCTAGTAATCGCAGATCAGCATGCTGCGTAAT | |
| | | GCCTC | |
| JBS4761 | 14 | CGGGGGCACGGTCTAAACTTGTAACTCCAAAAATGAACCCCAATCGAGAGA | Streptomyces avidinii |
| | | GGATCAGTGGAATAATGAAACGGTGAATAAACGGGCATATGTTCTTGCTCA | Streptomyces cirratus |
| | | TTGGACGACAAGCTAAAAACGGTCTAATACAAGAAAACCACTTGCCGCATG | Streptomyces lavendulae |
| | | TGCAGGGCGGGGAAAAAACTCCGCGGAAAAAAAAAACCCCCGCCCCCATC | Streptomyces nojiriensis |
| | | AGCTGTGGTGGGGTAAAGGCCCACCAAGGCAAAACAACAAGAAGCCCGCT | Streptomyces omiyaensis |
| | | GAGAGGGACACCCCCACACTGGGACAGAGACACGCCCCACACTCTAACGGA | Streptomyces spororaveus |
| | | GGCAGCAGGGGGAAAAATGCACAATGGGCGAAAGCCCAAGCAACCACCCC | Streptomyces subrutilus |
| | | CCGTGAAGAAGGACGGCCTTCGGGTTGTAAACCTCCTTCAGCAGGGAAGAA | Streptomyces vinaceus |
| | | GCGAAAGTGACGGTACTGCAGAAGAAGCGCCGGCTAACTACGTGCCAGCAG | |
| | | CCGCGGTAATACGTAGGGCGCAAGCGTTGTCCGGAATTATTGGGCGTAAAG | |
| | | AGCTCGTAGGCGGCTTGTCACGTCGGATGTGAAAGCCCGAGGCTTAACCTC | |
| | | GGGTCTGCATTCGATACGGGCTAGCTAGAGTGTGGTAGGGGAGATCGGAAT | |
| | | TCCTGGTGTAGCGGTGAAATGCGCAGATATCAGGAGGAACACCGGTGGCGA | |
| | | AGGCGGATCTCTGGGCCATTACTGACGCTGAGGAGCGAAAGCGTGGGGAGC | |
| | | GAACAGGATTAGATACCCTGGTAGTCCACGCCGTAAACGTTGGGAACTAGG | |
| | | TGTTGGCGACATTCCACGTCGTCGGTGCCGCAGCTAACGCATTAAGTTCCCC | |
| | | GCCTGGGGAGTACGGCCGCAAGGCTAAAACTCAAAGGAATTGACGGGGGCC | |
| | | CGCACAAGCAGCGGAGCATGTGGCTTAATTCGACGCAACGCGAAGAACCTT | |
| | | ACCAAGGCTTGACATATACCGGAAAGCATTAGAGATAGTGCCCCCCTTGTG | |
| | | GTCGGTATACAGGTGGTGCATGGCTGTCGTCAGCTCGTGTCGTGAGATGTTG | |
| | | GGTTAAGTCCCGCAACGAGCGCAACCCTTGTCCTGTGTTGCCAGCATGCCCT | |
| | | TCGGGGTGATGGGGACTCACAGGAGACCGCCGGGGTCAACTCGGAGGAAG | |
| | | GTGGGGACGACGTCAAGTCATCATGCCCCTTATGTCTTGGGCTGCACACGTG | |
| | | CTACAATGGCCGGTACAATGAGCTGCGATACCGTGAGGTGGAGCGAATCTC | |
| | | AAAAAGCCGGTCTCAGTTCGGATTGGGGTCTGCAACTCGACCCCATGAAGTT | |
| | | GGAGTTGCTAGTAATCGCAGATCAGCATGCTGCGGAATCCGGC | |
| JBS3673 | 15 | CGGGCGCGTGCTTACACATGCAGTCGAACGACAAAAACCGTCGGCGTCGAT | Streptomyces cinnamonensis |
| | | TACTGCAAAACACGTGAGCAACACGGGGACAAGCCAACCTGCAACCTGGGA | Streptomyces flaveus |
| | | AAACCCCTGGAAACCGGAACGAACAACCGATACAACTCCTGCATGCATGGG | Streptomyces lavendulae |
| | | CGGGGGTTGAAAGCGAAGCCCGCGAAAGATGAGCCCGCGGCATATCAGCA | Streptomyces nojiriensis |
| | | GCTGGGTGGGGTAATGCCACACCAAGGCGACGACGGGTAGCCGCCCTCAGA | Streptomyces sporoverrucosus |
| | | GAGCGACACGCCACACTGGGACTGAGACACGGCCCAGACCCCTACGGGAGG | Streptomyces venezuelae |
| | | CAGCAGTGGGGAATATTGCACAATGGGCGAAAGCCTGATGCAGCGACGCCG | Streptomyces vinaceus |
| | | CGTGAGGGATGACGGCCTTCGGGTTGTAAACCTCTTTCAGCAGGGAAGAAG | Streptomyces virginiae |
| | | CGAAAGTGACGGTACCTGCAGAAGAAGCGCCGGCTAACTACGTGCCAGCAG | Streptomyces xanthophaeus |
| | | CCGCGGTAATACGTAGGGCGCAAGCGTTGTCCGGGAATTATTGGGCGTAAA | |
| | | GAGCTCGTAGGCGGCTTGTCACGTCGGATGTGAAAGCCCGAGGCTTAACCT | |
| | | CGGGTCTGCATTCGATACGGGCTAGCTAGAGTGTGGTAGGGGAGATCGGAA | |
| | | TTCCTGGTGTAGCGGTGAAATGCGCAGATATCAGGAGGAACACCGGTGGCG | |
| | | AAGGCGGATCTCTGGGCCATTACTGACGCTGAGGAGCGAAAGCGTGGGGAG | |
| | | CGAACAGGATTAGATACCCTGGTAGTCCACGCCGTAAACGTTGGGAACTAG | |
| | | GTGTTGGCGACATTCCACGTCGTCGGTGCCGCAGCTAACGCATTAAGTTCCC | |
| | | CGCCTGGGGAGTACGGCCGCAAGGCTAAAACTCAAAGGAATTGACGGGGGC | |
| | | CCGCACAAGCGGCGGAGCATGTGGCTTAATTCGACGCAACGCGAAGAACCT | |
| | | TACCAAGGCTTGACATATACCGGAAAGCATTAGAGATAGTGCCCCCCTTGG | |
| | | GGTCGGTATACAGGGGGTGCATGGCTGTCGTCAGCTCGTGTCGTGAGAAGG | |
| | | TGGGGTAAAGCCCCCAAACAACCCAACCCTTGTCCTGTGTTGCCAGCATGCC | |
| | | CTTCGGGGTGATGGGGACTCACAGGAAACCGCCGGGGCAAACCCGAAAGA | |
| | | AGGGGACCACCACATCAACTCATCAGGCCCCAAATGTCTTGGGCTGCACAC | |
| | | GTGCTACAATGGCCGGTACAATGAGCTGCGATACCGTGAGGTGGAGCGAAT | |
| | | CTCAAAAAGCCGGTCTCAGTTCGGATTGGGGTCTGCAACTCGACCCCATGAA | |
| | | GTCGGAGTCGCTAGTAATCGCAGATCAGCATGCTGCGTAATGGCGTC | |
| JBS3880 | 31 | GGGGCGCGTGCTTACACATGCAGTCGAACGATGAACCTCCTTAGGGAGAAG | Streptomyces angustmyceticus |
| | | AGTAAGGCCGAACGAGTGAGAAACACACGGGCAAACTGCCATTCACTCAGG | Streptomyces catenulae |
| | | GACAAGCCCTGGAAACGACAGCTAAAACCGGACAAGACCACACACCCCAC | Streptomyces cinereus |
| | | GGCATGGCGGAGGAAAACTCCGGCGGTGAAAGATGAGCCCGCGCCCTATCA | Streptomyces libani |
| | | GCAGCTGGGGGGGGTAATGGCCCACCAAGGCGACGACGGGAACCCGCCCTG | Streptomyces lydicus |
| | | AAAAGGCCAACGGCCACACTGGGACTGAGACACGGCCCAGACTCCTACGGG | Streptomyces nigrescens |
| | | AGGCAGCAGTGGGGAATATTGCACAATGGGCGAAAGCCTGATGCAGCGACG | Streptomyces platensis |
| | | CCGCGTGAGGGATGACGGCCTTCGGGTTGTAAACCTCTTTCAGCAGGGAAG | Streptomyces tubercidicus |
| | | AAGCGAAAGTGACGGTACCTGCAGAAGAAGCGCCGGGCTAACTACGTGCCA | |
| | | GCAGCCGCGGTAATACGTAGGGCGCAAGCGTTGTCCGGAATTATTGGGCGT | |
| | | AAAGAGCTCGTAGGCGGCTTGTCACGTCGGATGTGAAAGCCCGAGGCTTAA | |
| | | CCCCGGGTCTGCATTCGATACGGGCAAGCTAGAGTGCGGTAGGGGAGATCG | |
| | | GAATTCCTGGTGTAGCGGTGAAATGCGCAGATATCAGGAGGAACACCGGTG | |
| | | GCGAAGGCGGATCTCTGGGCCAATACTGACGCTGAGGAGCGAAAGCGTGGG | |
| | | GAGCGAACAGGATTAGATACCCTGGTAGTCCACGCCGTAAACGTTGGGAAC | |
| | | TAGGTGTGGGCGACATTCCACGTCGTCCGTGCCGCAGCTAACGCATTAAGTT | |
| | | CCCCGCCTGGGGAGTACGGCCGCAAGGCTAAAACTCAAAGGAATTGACGGG | |
| | | GGCCCGCACAAGCAGCGGAGCATGTGGCTTAATTCGACGCAACGCGAAGAA | |
| | | CCTTACCAAGGCTTGACATACACCGGAAAACACTAGAGACAGGGCCCCCCT | |
| | | TGTGGTCCGTATACAGGTGGTGCATGGCTGTCGTCAGCTCGTGTCGTGAGAT | |
| | | GTTGGGTTAAGTCCCGCAACGAGCGCAACCCTTGTCCTGTGTTGCCAGCATG | |
| | | CCCTTCGGGGTGATGGGGACTCACAGGAGACCGCCGGGGTCAACTCGGAGA | |
| | | AAGGGGAGGACCAACGCAAGTCATCATGCCCCTTATGTCCTGGGCTGCACA | |
| | | CGTGCTACAATGCCCGGAAAAAAAAGCTGCCAAACCGCGAGGTGGAGCGA | |
| | | ATCTCAAAAACCCGGGCCCAGTTCGGATTGAGAGATGCAACTCCAACCCAG | |
| | | GAACTCGGAGTCGCTAGAAATCCCAGATAAACACGCTGCGGCATGCGC | |
| JBS3368 | 33 | CAGGGGGGTGCTTACACATGCAGTCGAACGATCAACAACTGCGCGCTGGAT | Streptomyces aquilus |
| | | TAACAGCGAAAGGGTGAGTAAAACGTGGGCAATATGACCTTCACTCAGAGA | Streptomyces caeruleatus |
| | | CAAGAACGGGAAAACCGGGCCAATACCGCATAACACCTGCAACGGCATCCG | Streptomyces fagopyri |
| | | GATACGATAAAAGCTCCGGCATGCAGGTGAAGCTCCGCGAAAGATGAGCCC | Streptomyces griseochromogenes |
| | | GCGCCATAACAGCTGGTTGGTGAGGGAGGGGATCACAAAGGAGACGACGA | Streptomyces mirabilis |
| | | CGAGCAGCCCTGAGAGGGCGACCGCCAACACTGGGACTGAGACACGGCCCA | Streptomyces nojiriensis |
| | | GACTCCAACGGGAGGCAGCAGTGGGGAATATGCAACAAAGGGCCAAAACC | Streptomyces pseudovenezuelae |
| | | TGATGCAGCGACGCCGCGTGAGGGATGACGGCCTTCGGGTTGTAAACCTCTT | Streptomyces viridochromogenes |
| | | TCAGCAGGGAAGAAGCGAAAGTGACGGTACCTGCAGAAGAAGCGCCGGCT | Streptomyces viridochromogenes |
| | | AACTACGTGCCAGCAGCCGCGGTAATACGTAGGGCGCAAGCGTTGTCCGGG | |
| | | AATTATTGGGCGTAAAGAGCTCGTAGGCGGCTTGTCACGTCGGATGTGAAA | |
| | | GCCCGAGGCTTAACCCCGGGTCTGCATTCGATACGGGCTAGCTAGAGTGTG | |
| | | GTAGGGGAGATCGGAATTCCTGGTGTAGCGGTGAAATGCGCAGATATCAGG | |
| | | AGGAACACCGGTGGCGAAGGCGGATCTCTGGGCCATTACTGACGCTGAGGA | |
| | | GCGAAAGCGTGGGGAGCGAACAGGATTAGATACCCTGGTAGTCCACGCCGT | |
| | | AAACGGTGGGAACTAGGTGTTGGCGACATTCCACGTCGTCGGTGCCGCAGC | |
| | | TAACGCATTAAGTTCCCCGCCTGGGGAGTACGGCCGCAAGGCTAAAACTCA | |
| | | AAGGAATTGACGGGGGCCCGCACAAGCAGCGGAGCATGTGGCTTAATTCGA | |
| | | CGCAACGCGAAGAACCTTACCAAGGCTTGACATACACCGGAAACCACCAGA | |
| | | GATAGTCCCCCCCTGGGGGTCGGTATACAGGTGGTGCAAGGCTGTCGTCAG | |
| | | CTCGTGTCGTGAGATGTTGGGTAAAGCCCCCAAACAACCCCAACCCTTGTTC | |
| | | CGGTGTTGCCAGCATGCCCTTCCGGGTGATGGGGACTCACAGGAGACCGCC | |
| | | GGGGTCAACTCGGAGGAAGGTGGGGACGACGTCAAGTCATCATGCCCCTTA | |
| | | TGTCTTGGGCTGCACACGTGCTACAATGGCCGGTACAATGAGCTGCGATACC | |
| | | GTGAGGTGGAGCGAATCTCAAAAAGCCGGTCTCAGTTCGGATTGGGGTCTG | |
| | | CAACTCGACCCCATGAAGTCGGAGTCGCTAGTAATCGCAGATCAGCATGCT | |
| | | GCGTATCCG | |
| JBS8054 | 16 | CGGGGGGCGTGCTTACACATGCAAGTCGAACGATAAAACACTCCGCAATGG | Streptomyces lavendulae |
| | | AATACAGACGAACCCGTCAGTAACACGTGGAAAATCGCAACGTCACTAACG | Streptomyces nojiriensis |
| | | GACAAGCCCTGCAAACGGACAAGAAGACCGGTTTATACCAACACCGCCTGC | Streptomyces spororaveus |
| | | ATGGCCGGGGGTTGAAAGCTCCGGCGCGGAAAGATGAGCCCGCGCCCTATC | Streptomyces subrutilus |
| | | AGCTGGTTGGTGGGGGAAAAGCCCACCAAGGCGACGACGGGTAGCCGCCCG | Streptomyces venezuelae |
| | | CAGAGAGCGACACCCCACACTGGGACTGAGACACGGCCCAGACTCCTACGG | Streptomyces virginiae |
| | | GAGGCAGCAGTGGGGAATATTGCACAATGGGCGAAAGCCTGATGCAGCGAC | Streptomyces xanthophaeus |
| | | GCCGCGTGAGGGATGACGGCCTTCGGGTTGTAAACCTCTTTCAGCAGGGAA | |
| | | GAAGCGAAAGTGACGGTACCTGCAGAAGAAGCGCCGGCTAACTACGTGCCA | |
| | | GCAGCCGCGGTAATACGTAGGGCGCAAGCGTTGTCCGGGAATTATTGGGCG | |
| | | TAAAGAGCTCGTAGGCGGCTTGTCACGTCGGATGTGAAAGCCCGAGGCTTA | |
| | | ACCTCGGGTCTGCATTCGATACGGGCTAGCTAGAGTGTGGTAGGGGAGATC | |
| | | GGAATTCCTGGTGTAGCGGTGAAATGCGCAGATATCAGGAGGAACACCGGT | |
| | | GGCGAAGGCGGATCTCTGGGCCATTACTGACGCTGAGGAGCGAAAGCGTGG | |
| | | GGAGCGAACAGGATTAGATACCCTGGTAGTCCACGCCGTAAACGTTGGGAA | |
| | | CTAGGTGTTGGCGACATTCCACGTCGTCGGTGCCGCAGCTAACGCATTAAGT | |
| | | TCCCCGCCTGGGGAGTACGGCCGCAAGGCTAAAACTCAAAGGAATTGACGG | |
| | | GGGCCCGCACAAGCAGCGGAGCATGTGGCTTAATTCGACGCAACGCGAAGA | |
| | | ACCTTACCAAGGCTTGACATATACCGGAAAGCATTAGAGATAGTGCCCCCCT | |
| | | TGTGGTCGGTATACAGGGGGGCCATGGCTGTCGTCAGCTCGTGTCGTGAGAT | |
| | | GTTGGGTTAAGTCCCGCAACGAGCGCAACCCTTGTCCTGTGTTGCCAGCATG | |
| | | CCCTTCGGGGTGATGGGGACTCACAGGAGACCGCCGGGGTCAACTCGGAGG | |
| | | AAGGTGGGGACGACGTCAAGTCATCATGCCCCTTATGTCTTGGGCTGCACAC | |
| | | GTGCTACAATGGCCGGTACAATGAGCTGCGATACCGTGAGGTGGAGCGAAT | |
| | | CTCAAAAAGCCGGTCTCAGTTCGGATTGGGGTCTGCAACTCGACCCCATGAA | |
| | | GTCGGAGTCGCTAGTAATCGCAGATCAGCATGCTGCGTATGCTAA | |
| JBS1452 | 17 | GAGTCAGCACCGCGTCGCGCGGGGGCTAAAAAATGCAAGTCGAACAAAGA | Streptomyces cinnamonensis |
| | | AGCCCTTCGGGGTGGATTAGTGGCGAACGGGGAAATAACAACGGGGAAAA | Streptomyces flaveus |
| | | CTGCCCATCACTCTGGGACAAGCCCTGCAAACACAGCCGAACACCCGATAC | Streptomyces goshikiensis |
| | | CACGCAAGCCTGCATGCACGGCGGGTGAAAGCTCCGGCGCGGAAAGATGAG | Streptomyces lavendulae |
| | | CCCGCGGCCTAATCAGCTTGTTGGTGGGGTAAGGCCCCAACAAGGCGACGA | Streptomyces nojiriensis |
| | | CGGGTAGCCGGCCGCAGAGAGCGACACGCCACACTGGGACTGAGACACGG | Streptomyces sporoverrucosus |
| | | CCCAGACTCCTACGGGAGGCAGCAGTGGGGAATATTGCACAATGGGCGAAA | Streptomyces venezuelae |
| | | GCCTGATGCAGCGACGCCGCGTGAGGGATGACGGCCTTCGGGTTGTAAACC | Streptomyces virginiae |
| | | TCTTTCAGCAGGGAAGAAGCGAAAGTGACGGTACCTGCAGAAGAAGCGCCG | Streptomyces xanthophaeus |
| | | GCTAACTACGTGCCAGCAGCCGCGGTAATACGTAGGGCGCAAGCGTTGTCC | |
| | | GGGAATTATTGGGCGTAAAGAGCTCGTAGGCGGCTTGTCACGTCGGATGTG | |
| | | AAAGCCCGAGGCTTAACCTCGGGTCTGCATTCGATACGGGCTAGCTAGAGT | |
| | | GTGGTAGGGGAGATCGGAATTCCTGGTGTAGCGGTGAAATGCGCAGATATC | |
| | | AGGAGGAACACCGGTGGCGAAGGCGGATCTCTGGGCCATTACTGACGCTGA | |
| | | GGAGCGAAAGCGTGGGGAGCGAACAGGATTAGATACCCTGGTAGTCCACGC | |
| | | CGTAAACGTTGGGAACTAGGTGTTGGCGACATTCCACGTCGTCGGTGCCGCA | |
| | | GCTAACGCATTAAGTTCCCCGCCTGGGGAGTACGGCCGCAAGGCTAAAACT | |
| | | CAAAGGAATTGACGGGGGCCCGCACAAGCGGCGGAGCATGTGGCTTAATTC | |
| | | GACGCAACGCGAAGAACCTTACCAAGGCTTGACATATACCGGAAAGCATTA | |
| | | GAGATAGTGCCCCCCTTGTGGTCGGTATACAGGTGGTGCATGGCTGTCGTCA | |
| | | GCTCGTGTCGTGAGATGTTGGGTTAAGTCCCGCAACGAGCGCAACCCTTGTC | |
| | | CTGTGTTGCCAGCATGCCCTCCGGGGTGATGGGGACCCAAAAGAGACACCC | |
| | | CCGGGCAACACCGACGAAGGTGGGGACGACGTCAAGTCATCATGCCCCTTA | |
| | | TGTCTTGGGCTGCACACGTGCTACAATGGCCGGTACAATGAGCTGCGATACC | |
| | | GTGAGGTGGAGCGAATCTCAAAAAGCCGGTCTCAGTTCGGATTGGGGTCTG | |
| | | CAACTCGACCCCATGAAGTCGGAGTCGCTAGTAATCGCAGATCAGCATGCT | |
| | | GCGTAATGCGTG | |
| JBS6900 | 18 | CGGGTGCGTGCTTACACATGCAGTCGAACGATGAACCCACTCGCAGTGGAT | Streptomyces colombiensis |
| | | TAGTGGCAAACGAGAGAGTAACACGTGAACAAGCGCACCTTCACGCTAGGA | Streptomyces flaveus |
| | | CAAGCCATGGAAACGGGACAGAAGACCGGACAACACGACAGCATCCAGCG | Streptomyces lavendulae |
| | | GCAGGGCAGGAAAGAACCGCCGCGGAAAGATGAGCCCGCGGCCTATCAGC | Streptomyces senoensis |
| | | TTGGTGGTGGAGTAAAGGCCCACCAAGGCGACCAACGACAGCAGCCCTGAG | Streptomyces sporoverrucosus |
| | | AGGGCGACCGCCAACACTGGGACTGAGACACGGCCCAGACTCCTACGGGAG | Streptomyces venezuelae |
| | | GCAGCAGTGGGGAATATTGCACAATGGGCGAAAGCCGAATGCAGCGACGCC | Streptomyces vinaceus |
| | | GCGTGAGGGATGAACGCCCTTCGGGTTGTAAACCTCTTTCAGCAGGGAAGA | Streptomyces virginiae |
| | | AGCGAAAGTGACGGTACCTGCAGAAGAAGCGCCGGCTAACTACGTGCCAGC | Streptomyces xanthophaeus |
| | | AGCCGCGGTAATACGTAGGGCGCAAGCGTTGTCCGGGAATTATTGGGCGTA | |
| | | AAGAGCTCGTAGGCGGCTTGTCACGTCGGATGTGAAAGCCCGAGGCTTAAC | |
| | | CTCGGGTCTGCATTCGATACGGGCTAGCTAGAGTGTGGTAGGGGAGATCGG | |
| | | AATTCCTGGTGTAGCGGTGAAATGCGCAGATATCAGGAGGAACACCGGTGG | |
| | | CGAAGGCGGATCTCTGGGCCATTACTGACGCTGAGGAGCGAAAGCGTGGGG | |
| | | AGCGAACAGGATTAGATACCCTGGTAGTCCACGCCGTAAACGTTGGGAACT | |
| | | AGGTGTTGGCGACATTCCACGTCGTCGGTGCCGCAGCTAACGCATTAAGTTC | |
| | | CCCGCCTGGGGAGTACGGCCGCAAGGCTAAAACTCAAAGGAATTGACGGGG | |
| | | GCCCGCACAAGCGGCGGAGCATGTGGCTTAATTCGACGCAACGCGAAGAAC | |
| | | CTTACCAAGGCTTGACATATACCGGAAAGCATTAGAGATAGTGCCCCCCTTG | |
| | | TGGTCGGTATACAGGTGGTGCATGGCTGTCGTCAGCTCGTGTCGTGAGATGT | |
| | | TGGGTTAAGTCCCGCAACGAGCGCAACCCTTGTCCTGTGTTGCCAGCATGCC | |
| | | CTTCGGGGTGATGGGGACTCACAGGAGACCGCCGGGGTCAACTCGGAGAAA | |
| | | GGGGACGACGACGTCAAGTCATCATGCCCCTAAGGTCTTGGGCTGCACACG | |
| | | TGCTACAAGGCCCACAACAACGACCAACCATAACGGGAGATCTCAAGCGAA | |
| | | TCAAAAAAAGCCGATCGCAACTCGAACAGGAGTCGGAAACTCGACCCCATG | |
| | | AAGTCCGAGTCACTAGCAATCACAGATCAGCATGCTGCGTAAAGCGTG | |
| JBS5011 | 32 | CGCGGGCGCTGCCTAAACACGCAAGGCGAACGATGAACATCATCCCGCAGA | Streptomyces argenteolus |
| | | GGAAGAGTGGCGAACGGCAAAGTAACAACTGGCCAATCCGCCCTTCACTCT | Streptomyces atrolaccus |
| | | GGGACAAGCCCTGGAAAACCGGTCTAATACCCGATACAACACGGGGTCGCA | Streptomyces chattanoogensis |
| | | TGACCCCCGTGTGGAAAGCCCCGGCGGTGAAAGATGAGCCCGCGCCATATC | Streptomyces chrestomyceticus |
| | | AGCTTGTGGGTGGGGTGATGGCCGACCAAAGCGACGACGGGTAGCCGGCCC | Streptomyces coelicolor |
| | | GAAAAAGCGACACGCCACACTGGGACTGAGAAAACGCCCAAAACCCTACG | Streptomyces lydicus |
| | | GGAGGCAGCAGTGGGGAATATTGCACAATGGGCGAAAGCCTGATGCAGCG | Streptomyces microsporus |
| | | ACGCCGCGTGAGGGATGACGGCCTTCGGGTTGTAAACCTCTTTCAGCAGGG | Streptomyces nigrescens |
| | | AAGAAGCGAGAGTGACGGTACCTGCAGAAGAAGCGCCGGCTAACTACGTGC | Streptomyces rimosus |
| | | CAGCAGCCGCGGTAATACGTAGGGCGCAAGCGTTGTCCGGGAATTATTGGG | Streptomyces sioyaensis |
| | | CGTAAAGAGCTCGTAGGCGGCTTGTCACGTCGGATGTGAAAGCCCGGGGCT | |
| | | TAACCCCGGGTCTGCATTCGATACGGGCAGGCTAGAGTTCGGTAGGGGAGA | |
| | | TCGGAATTCCTGGTGTAGCGGTGAAATGCGCAGATATCAGGAGGAACACCG | |
| | | GTGGCGAAGGCGGATCTCTGGGCCGATACTGACGCTGAGGAGCGAAAGCGT | |
| | | GGGGAGCGAACAGGATTAGATACCCTGGTAGTCCACGCCGTAAACGTTGGG | |
| | | AACTAGGTGTGGGCGACATTCCACGTCGTCCGTGCCGCAGCTAACGCATTAA | |
| | | GTTCCCCGCCTGGGGAGTACGGCCGCAAGGCTAAAACTCAAAGGAATTGAC | |
| | | GGGGGCCCGCACAAGCAGCGGAGCATGTGGCTTAATTCGACGCAACGCGAA | |
| | | GAACCTTACCAAGGCTTGACATACACCGGAAAACCCTGGAGACAGGGTCCC | |
| | | CCTTGTGGTCGGTGTACAGGTGGTGCATGGCTGTCGTCAGCTCGTGTCGTGA | |
| | | GATGTTGGGTTAAGTCCCCCAAACAACCCAACCCTTGTTCTGTGTTGCCAGC | |
| | | ATGCCCTTCCAGGTGATGGGGACTCACAGGAAAACGCCCGGGGTCAACCCC | |
| | | GAGGAAGGGGACGACGACGTCAAGTCATCATGCCCCTTATGTCCTGGGCTG | |
| | | CACACGGGCTACAAGCGCACATAAAAGCAGCTGCGAAACCGCCAAGGGGA | |
| | | ACCAATCTCAAAAAGCCAGTCTCAGTCCGAATTGGGGTCTGCAACCCGAAC | |
| | | ACAAGAACTCAGAGGCGCTAGGAACCCCAGATCAGCATGCTGCTTAATGCG | |
| | | AC | |
| JBS5180 | 34 | CTGGCGCGTCCAAACACAATGCAAGTCCAACGAAGAAGCCCTTCGGGGTGG | Streptomyces aquilus |
| | | ATTAGTGGCCAACGAGTGAGTAACCAACGGGGACAAACTGCCAATCACTCA | Streptomyces aureus |
| | | CGGACAAGACAAGCAAAACGGGTCTAATACCAGAAAATACACCTGCCAGCA | Streptomyces fagopyri |
| | | TCGGCGGGGGTGGAAAAAGCCGGCGGGCAAGAAGAAGACCCCCGCCTATC | Streptomyces lutosisoli |
| | | AGCTGGTGGGGGAGGTAATGGCCCACCAAAGCGACGACGGGTAGCCCGCCG | Streptomyces minoensis |
| | | AAAAGGGCGACCGCCAACACTGGGACTGAAAAAACGGCCCAGACTCCTACG | Streptomyces mirabilis |
| | | GGAGGCAGCAGTGGGGAATATTGCACAATGGGCGAAAGCCTGATGCAGCG | Streptomyces olivochromogenes |
| | | ACGCCGCGTGAGGGATGACGGCCTCCGGGTTGGAAAACCCCTTCAGCAGGG | Streptomyces |
| | | AAGAAGCGAAAGTGACGGTACCTGCAGAAGAAGCGCCGGCTAACTACGTGC | rhizosphaerihabitans |
| | | CAGCAGCCGCGGTAATACGTAGGGCGCAAGCGTTGTCCGGAATTATTGGGC | |
| | | GTAAAGAGCTCGTAGGCGGCTTGTCACGTCGGATGTGAAAGCCCGAGGCTT | |
| | | AACCCCGGGTCTGCATTCGATACGGGCTAGCTAGAGTGTGGTAGGGGAGAT | |
| | | CGGAATTCCTGGTGTAGCGGTGAAATGCGCAGATATCAGGAGGAACACCGG | |
| | | TGGCGAAGGCGGATCTCTGGGCCATTACTGACGCTGAGGAGCGAAAGCGTG | |
| | | GGGAGCGAACAGGATTAGATACCCTGGTAGTCCACGCCGTAAACGGTGGGA | |
| | | ACTAGGTGTTGGCGACATTCCACGTCGTCGGTGCCGCAGCTAACGCATTAAG | |
| | | TTCCCCGCCTGGGGAGTACGGCCGCAAGGCTAAAACTCAAAGGAATTGACG | |
| | | GGGGCCCGCACAAGCAGCGGAGCATGTGGCTTAATTCGACGCAACGCGAAG | |
| | | AACCTTACCAAGGCTTGACATACACCGGAAACCACCAGAGATAGTCCCCCC | |
| | | CTGGGGGCCGGAGAAAAGGTGGTGCATGGCTGTCGTCAGCTCGTGTCGTGA | |
| | | GATGTTGGGTTAAGTCCCGCAACGAGCGCAACCCTTGTTCTGTGTTGCCAGC | |
| | | ATGCCCTTCGGGGTGATGGGGACTCACAGGAGACTGCCGGGGTCAACTCGG | |
| | | AGGAAGGTGGGGACGACGTCAAGTCATCATGCCCCTTATGTCTTGGGCTGC | |
| | | ACACGTGCTACAATGGCAGGTACAATGAGCTGCGAAGCCGTGAGGCGGAGC | |
| | | GAATCTCAAAAAGCCTGTCTCAGTTCGGATTGGGGTCTGCAACTCGACCCCA | |
| | | TGAAGTCGGAGTTGCTAGTAATCGCAGATCAGCATGCTGCGTATGCCTA | |
| JBS6069 | 19 | CGGGCGGGTGCTTACACATGCAGTCGAACGATGAAGCCCTTCGGGGTGGAT | Streptomyces flaveus |
| | | TAAGAACAAACGGGCGAGTCTAACACCAAGGCAAGCTGCCCTACACTCGAA | Streptomyces lavendulae |
| | | GACAAGCACGGGAAAACGGGCCAAAGACCGCATAACAAGCCTCACGCCTCC | Streptomyces sporoverrucosus |
| | | ATGCGAGCGAGGCGAAACATCCCGCGGTGAAGGATCAGCGGGAGATGACC | Streptomyces venezuelae |
| | | CGCGGCCTATCAGCTGGTTGGTGGGGGAAACGCCCACCAAGGCGACGACGG | Streptomyces vinaceus |
| | | GAACCCGGCCCGAAAAGGCCAACGCCAACACTGGGACTGAGACACGGCCC | Streptomyces virginiae |
| | | AGACTCCTACGGGAGGCAGCAGTGGGGAATATTGCACAATGGCCGAAAACC | Streptomyces xanthophaeus |
| | | TGATGCAGCGACGCCGCGTGAGGGATGACGGCCTTCCGGGTGTAAACCTCT | |
| | | TTCAGCAGGGAAGAAGCGAAAGTGACGGTAACTGCAGAAGAAGCGCCGGC | |
| | | TAACTACGTGCCAGCAGCCGCGGTAATACGTAGGGCGCAAGCGTTGTCCGG | |
| | | GAATTATTGGGCGTAAAGAGCTCGTAGGCGGCTTGTCACGTCGGATGTGAA | |
| | | AGCCCGAGGCTTAACCTCGGGTCTGCATTCGATACGGGCTAGCTAGAGTGTG | |
| | | GTAGGGGAGATCGGAATTCCTGGTGTAGCGGTGAAATGCGCAGATATCAGG | |
| | | AGGAACACCGGTGGCGAAGGCGGATCTCTGGGCCATTACTGACGCTGAGGA | |
| | | GCGAAAGCGTGGGGAGCGAACAGGATTAGATACCCTGGTAGTCCACGCCGT | |
| | | AAACGTTGGGAACTAGGTGTTGGCGACATTCCACGTCGTCGGTGCCGCAGCT | |
| | | AACGCATTAAGTTCCCCGCCTGGGGAAGAACGCCCCAAAGCCAAAAACCAA | |
| | | AGGAATTGACGGGGGCCCGCACAAGCGGCGGAGCATGTGGCTTAATTCGAC | |
| | | GCAACGCGAAGAACCTTACCAAGGCTTGACATATACCGGAAAGCATTAGAG | |
| | | ATAGTGCCCCCCTTGTGGTCGGTAAAAAAGGTGGTGCATGGCTGTCGTCAGC | |
| | | TCGTGTCGTGAGATGTTGGGTTAAGTCCCGCAACGAGCGCAACCCTTGTCCT | |
| | | GTGTTGCCAGCATGCCCTTCGGGGTGATGGGGACTCACAGGAGACCGCCGG | |
| | | GGTCAACTCGGAGGAAGGTGGGGACGACGTCAAGTCATCATGCCCCTTATG | |
| | | TCTTGGGCTGCACACGTGCTACAATGGCCGGTACAATGAGCTGCGATACCGT | |
| | | GAGGTGGAGCGAATCTCAAAAAGCCGGTCTCAGTTCGGATTGGGGTCTGCA | |
| | | ACTCGACCCCATGAAGTCGGAGTCGCTAGTAATCGCAGATCAGCATGCTGC | |
| | | GTATGCGGG | |
| JBS7630 | 35 | CGGGGGGCGTGAATACACATGCAATGTCGAACCATAAAACCCTTCGGGGAG | Streptomyces aquilus |
| | | GATTAGTGGCGAACGGGTAAGGAAAACGTGAGCAACCTGCACGCCACTCGG | Streptomyces fagopyri |
| | | GGACAAGCCCTGGAAACGGACCAGAATAACGGATAACACCAGACACCGCA | Streptomyces griseoruber |
| | | ACCAGCTGAGAGAAAAGCTCCGGCGGTGAAGGATGAGCCCGCGGCCTATCA | Streptomyces lutosisoli |
| | | GCTGGTGGGTGAAGTAGTGGCTCACCAAGGCAACGACGACGAGCAGCCCCG | Streptomyces minoensis |
| | | AGAGAGCCAACGCCAACACTGGGACTGAGACACGGCCCAGACTCCTACGGG | Streptomyces mirabilis |
| | | AGGCAGCAGTGGGGAATATTGCACAATGGGCGAAAGCCTGATGCAGCGACG | Streptomyces olivochromogenes |
| | | CCGCGTGAGGGATGACGGCCTTCGGGTTGTAAACCTCTTTCAGCAGGGAAG | Streptomyces scabiei |
| | | AAGCGAAAGTGACGGTACCTGCAGAAGAAGCGCCGGCTAACTACGTGCCAG | |
| | | CAGCCGCGGTAATACGTAGGGCGCAAGCGTTGTCCGGAATTATTGGGCGTA | |
| | | AAGAGCTCGTAGGCGGCTTGTCACGTCGGGTGTGAAAGCCCGGGGCTTAAC | |
| | | CCCGGGTCTGCATTCGATACGGGCTAGCTAGAGTGTGGTAGGGGAGATCGG | |
| | | AATTCCTGGTGTAGCGGTGAAATGCGCAGATATCAGGAGGAACACCGGTGG | |
| | | CGAAGGCGGATCTCTGGGCCATTACTGACGCTGAGGAGCGAAAGCGTGGGG | |
| | | AGCGAACAGGATTAGATACCCTGGTAGTCCACGCCGTAAACGGTGGGAACT | |
| | | AGGTGTTGGCGACATTCCACGTCGTCGGTGCCGCAGCTAACGCATTAAGTTC | |
| | | CCCGCCTGGGGAGTACGGCCGCAAGGCTAAAACTCAAAGAAATGAACGGG | |
| | | GGCCCGCACAAGCAGCGGAGCATGTGGCTTAATTCCAACCAACGCGAAGAA | |
| | | CCTTACCAAGGCTTGACATACACCGGAAACGGCCAGAGATGGTCGCCCCCT | |
| | | GGGGGGCCGTGTACAGGTGGTGCATGGCTGTCGTCAGCTCGTGTCGGAAGA | |
| | | TGTTGGGTTAAGTCCCGCAACGAGCGCAACCCTTGTTCTGTGTTGCCAGCAT | |
| | | GCCCTTCGGGGTGATGGGGACTCACAGGAGACTGCCGGGGTCAACTCGGAG | |
| | | GAAGGTGGGGACGACGTCAAGTCATCATGCCCCTTATGTCTTGGGCTGCACA | |
| | | CGTGCTACAATGGCAGGTACAATGAGCTGCGAAGCCGTGAGGCGGAGCGAA | |
| | | TCTCAAAAAGCCTGTCTCAGTTCGGATTGGGGTCTGCAACTCGACCCCATGA | |
| | | AGTCGGAGTTGCTAGTAATCGCAGATCAGCATGCTGCGTAATTGCCGTG | |
| JBS5615 | 5 | TCGTGGCGCGCGCGTGCTAACACATGAAAGTCGAACAAGGAACCTCCTTCG | Streptomyces achromogenes |
| | | CGAGAAGACTAGTGGCAAACGGGTGAGTAACACCGGAGAAATCTGCCATTC | Streptomyces bungoensis |
| | | ACTACGGGACAAGCCCTGGAAAACGGGTCTAATACCGGATACCAATACACG | Streptomyces canarius |
| | | AGGCAGCACCTGGAGGTGGAAAGCTCCGGCGGTGAAGAAGAAGCCCCCCG | Streptomyces capoamus |
| | | CCTATCAGCTGGGTGGTGAGGTAACGCCCCAACAAAGCGACGACGGGTAGC | Streptomyces cellostaticus |
| | | AGCCCCGAAAAGGCCAACGGCAACACTGGGACTGAGACACGGCCCAGACTC | Streptomyces galbus |
| | | CAACGGAAGGCAGCAGTGGGGAATATTGCACAATGGCCGAAAGCCGAAGC | Streptomyces katrae |
| | | AACCGACGCCGCGTGAGGGATGACGGCCTTCGGGTTGTAAACCTCTTTCAGC | Streptomyces spinichromogenes |
| | | AGGGAAGAAGCGAAAGTGACGGTACCTGCAGAAGAAGCGCCGGCTAACTA | |
| | | CGTGCCAGCAGCCGCGGTAATACGTAGGGCGCAAGCGTTGTCCGGAATTAT | |
| | | TGGGCGTAAAGAGCTCGTAGGCGGCTTGTCACGTCGGATGTGAAAGCCCGA | |
| | | GGCTTAACCCCGGGTCTGCATTCGATACGGGCTAGCTAGAGTGTGGTAGGG | |
| | | GAGATCGGAATTCCTGGTGTAGCGGTGAAATGCGCAGATATCAGGAGGAAC | |
| | | ACCGGTGGCGAAGGCGGATCTCTGGGCCATTACTGACGCTGAGGAGCGAAA | |
| | | GCGTGGGGAGCGAACAGGATTAGATACCCTGGTAGTCCACGCCGTAAACGG | |
| | | TGGGAACTAGGTGTTGGCGACATTCCACGTCGTCGGTGCCGCAGCTAACGC | |
| | | ATTAAGTTCCCCGCCTGGGGAGTACGGCCGCAAGGCTAAAACTCAAAGGAA | |
| | | TTGACGGGGGCCCGCACAAGCAGCGGAGCATGTGGCTTAATTTCGACGCAA | |
| | | CGCGAAGAACCTTACCAAGGCTTGACATACACCGGAAACCACTAGAGACAA | |
| | | GCGCCCCCTTGTGGTCGGTATACAGGTGGTGCATGGCTGTCGTCAGCTCGTG | |
| | | GCCGGAAGAATGTTGGGTTAAGTCCCGCAACGAGCGCAACCCTTGTTCTGTG | |
| | | TTGCCAGCATGCCCTTCGGGGTGATGGGGACTCACAGGAGACCGCCGGGGT | |
| | | CAACTCGGAGGAAGGTGGGGACGACGTCAAGTCATCATGCCCCTTATGTCTT | |
| | | GGGCTGCACACGTGCTACAATGGCCGGTACAATGAGCTGCGATACCGTGAG | |
| | | GTGGAGCGAATCTCAAAAAGCCGGTCTCAGTTCGGATTGGGGTCTGCAACT | |
| | | CGACCCCATGAAGTCGGAGTTGCTAGTAATCGCAGATCAGCATGCTGCGGT | |
| | | AAATGCGAC | |

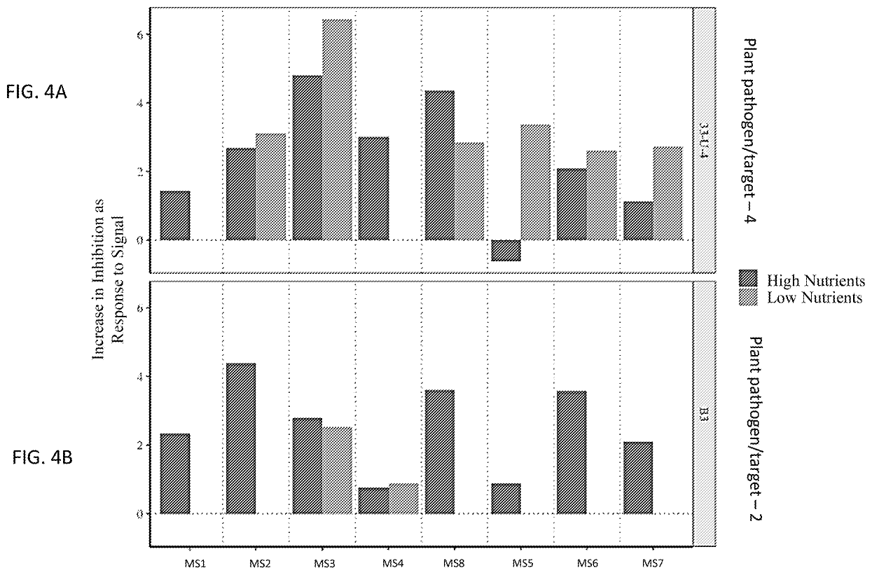

TABLE D
Exemplary Microbial Signalers and Examples of Enhanced Target Microbe Functions

| | | Taxa in commercial | |
|---|---|---|---|
| Name of microbial signaler isolate | Function | product | |
| JBS6220 JBS8493 JBS2943 JBS3418 JBS4783 JBS3946 | Pathogen suppression | Bacillus spp | FIG. 25 |
| JBS8473 JBS6762 JBS9261 JBS4549 | | | |
| JBS6899 JBS4020 JBS9225 JBS9264 JBS8135 | | | |
| JBS5867 JBS9311 JBS4046 JBS1726 | | | |
| JBS8612 JBS5020 JBS9780 JBS5523 JBS8753 | | | |
| JBS4761 JBS3673 JBS3880 JBS3368 | | | |
| JBS8054 JBS1452 JBS6900 JBS5011 JBS5180 | | | |
| JBS6069 JBS7630 JBS5615 | | | |
| JBS6220 JBS8493 JBS2943 JBS3418 JBS4783 | Pathogen suppression | Talaromyces spp | FIG. 23 |
| JBS3946 JBS8473 JBS6762 JBS9261 JBS4549 | | | |
| JBS6899 JBS4020 JBS9225 JBS9264 JBS8135 | | | |
| JBS5867 JBS9311 JBS4046 JBS1726 | | | |
| JBS8612 JBS5020 JBS9780 JBS5523 JBS8753 | | | |
| JBS4761 JBS3673 JBS3880 JBS3368 | | | |
| JBS8054 JBS1452 JBS6900 JBS5011 JBS5180 | | | |
| JBS6069 JBS7630 JBS5615 | | | |
| JBS6220 JBS8493 JBS2943 JBS3418 JBS4783 | Pathogen suppression | Streptomyces spp | FIG. 5-10 |
| JBS3946 JBS8473 JBS6762 JBS9261 JBS4549 | | | |
| JBS6899 JBS4020 JBS9225 JBS9264 JBS8135 | | | |
| JBS5867 JBS9311 JBS4046 JBS1726 | | | |
| JBS8612 JBS5020 JBS9780 JBS5523 JBS8753 | | | |
| JBS4761 JBS3673 JBS3880 JBS3368 | | | |
| JBS8054 JBS1452 JBS6900 JBS5011 JBS5180 | | | |
| JBS6069 JBS7630 JBS5615 | | | |
| JBS4783 JBS8135 JBS9311 JBS8753 JBS3880 | Pathogen suppression | Trichoderma spp | FIG. 13A, 13B, |
| JBS1726 JBS4761 JBS4549 | | | 14A, 14B, 15A, |
| | | | 15B, |
| JBS3418 JBS5867 JBS9311 JBS4046 | Pathogen suppression | Bacillus spp | FIG. 25 |
| JBS8753 JBS3880 JBS1452 JBS6900 | | | |
| JBS4783 JBS4046 JBS1452 JBS6900 | Pathogen suppression in | Streptomyces spp | FIG. 4A &4B |
| | reduced nutrient | | |
| | environment | | |
| JBS2943 JBS3418 JBS5867 JBS9311 JBS5020 JBS8753 JBS3880 | Phosphate solubilization | Streptomyces spp | FIG. 11C |
| JBS6220 JBS8493 JBS2943 JBS3418 JBS4783 | Phosphate solubilization | Bacillus spp | FIG. 11E |
| JBS3946 JBS8473 JBS6762 JBS9261 JBS4549 | | | |
| JBS6899 JBS4020 JBS9225 JBS9264 JBS8135 | | | |
| JBS5867 JBS9311 JBS4046 JBS1726 | | | |
| JBS8612 JBS5020 JBS9780 JBS5523 JBS8753 | | | |
| JBS4761 JBS3673 JBS3880 JBS3368 | | | |
| JBS8054 JBS1452 JBS6900 JBS5011 JBS5180 | | | |
| JBS6069 JBS7630 JBS5615 | | | |
| JBS6220 JBS8493 JBS2943 JBS3418 JBS4783 | Phosphate solubilization | Talaromyces spp | FIG. 11F |
| JBS3946 JBS8473 JBS6762 JBS9261 JBS4549 | | | |
| JBS6899 JBS4020 JBS9225 JBS9264 JBS8135 | | | |
| JBS5867 JBS9311 JBS4046 JBS1726 | | | |
| JBS8612 JBS5020 JBS9780 JBS5523 JBS8753 | | | |
| JBS4761 JBS3673 JBS3880 JBS3368 | | | |
| JBS8054 JBS1452 JBS6900 JBS5011 JBS5180 | | | |
| JBS6069 JBS7630 JBS5615 | | | |
| JBS6220 JBS8493 JBS2943 JBS3418 JBS4783 | Phosphate solubilization | Pseudomonas spp; | FIG. 11B, 22 |
| JBS3946 JBS8473 JBS6762 JBS9261 JBS4549 | | Comamonas spp; | |
| JBS6899 JBS4020 JBS9225 JBS9264 JBS8135 | | Citrobacter spp; | |
| JBS5867 JBS9311 JBS4046 JBS1726 | | Enterobacter spp | |
| JBS8612 JBS5020 JBS9780 JBS5523 JBS8753 | | | |
| JBS4761 JBS3673 JBS3880 JBS3368 | | | |
| JBS8054 JBS1452 JBS6900 JBS5011 JBS5180 | | | |
| JBS6069 JBS7630 JBS5615 | | | |
| JBS2943 JBS3418 JBS5867 JBS9311 JBS5020 | Phosphate solubilization | Streptomyces spp | FIG. 11A |
| JBS8753 JBS3880 | | | |
| JBS6220 JBS8493 JBS2943 JBS3418 JBS4783 | Phosphate solubilization | Trichoderma spp | FIG. 11D |
| JBS3946 JBS8473 JBS6762 JBS9261 JBS4549 | | | |
| JBS6899 JBS4020 JBS9225 JBS9264 JBS8135 | | | |
| JBS5867 JBS9311 JBS4046 JBS1726 | | | |
| JBS8612 JBS5020 JBS9780 JBS5523 JBS8753 | | | |
| JBS4761 JBS3673 JBS3880 JBS3368 | | | |
| JBS8054 JBS1452 JBS6900 JBS5011 JBS5180 | | | |
| JBS6069 JBS7630 JBS5615 | | | |
| JBS6220 JBS8493 JBS2943 JBS3418 JBS4783 | Phosphate solubilization | Talaromyces spp. | FIG. 24 |
| JBS3946 JBS8473 JBS6762 JBS9261 JBS4549 | | | |
| JBS6899 JBS4020 JBS9225 JBS9264 JBS8135 | | | |
| JBS5867 JBS9311 JBS4046 JBS1726 | | | |
| JBS8612 JBS5020 JBS9780 JBS5523 JBS8753 | | | |
| JBS4761 JBS3673 JBS3880 JBS3368 | | | |
| JBS8054 JBS1452 JBS6900 JBS5011 JBS5180 | | | |
| JBS6069 JBS7630 JBS5615 | | | |
| JBS5867 JBS3880 | Phosphate solubilization | Bacillus spp | FIG. 11E |
| JBS6220 JBS8493 JBS2943 JBS3418 JBS4783 | Zinc solubilization | Streptomyces spp | FIG. 12A, 12B |
| JBS3946 JBS8473 JBS6762 JBS9261 JBS4549 | | | |
| JBS6899 JBS4020 JBS9225 JBS9264 JBS8135 | | | |
| JBS5867 JBS9311 JBS4046 JBS1726 | | | |
| JBS8612 JBS5020 JBS9780 JBS5523 JBS8753 | | | |
| JBS4761 JBS3673 JBS3880 JBS3368 | | | |
| JBS8054 JBS1452 JBS6900 JBS5011 JBS5180 | | | |
| JBS6069 JBS7630 JBS5615 | | | |
| JBS6220 JBS8493 JBS2943 JBS3418 JBS4783 | Zinc solubilization | Talaromyces spp | FIG. 12D |
| JBS3946 JBS8473 JBS6762 JBS9261 JBS4549 | | | |
| JBS6899 JBS4020 JBS9225 JBS9264 JBS8135 | | | |
| JBS5867 JBS9311 JBS4046 JBS1726 | | | |
| JBS8612 JBS5020 JBS9780 JBS5523 JBS8753 | | | |
| JBS4761 JBS3673 JBS3880 JBS3368 | | | |
| JBS8054 JBS1452 JBS6900 JBS5011 JBS5180 | | | |
| JBS6069 JBS7630 JBS5615 | | | |
| JBS6220 JBS8493 JBS2943 JBS3418 JBS4783 | Zinc solubilization | Trichoderma spp | FIG. 12C |
| JBS3946 JBS8473 JBS6762 JBS9261 JBS4549 | | | |
| JBS6899 JBS4020 JBS9225 JBS9264 JBS8135 | | | |
| JBS5867 JBS9311 JBS4046 JBS1726 | | | |
| JBS8612 JBS5020 JBS9780 JBS5523 JBS8753 | | | |
| JBS4761 JBS3673 JBS3880 JBS3368 | | | |
| JBS8054 JBS1452 JBS6900 | | | |
| JBS5011 JBS5180 JBS6069 JBS7630 JBS5615 | | | |
| JBS4549 JBS4761 JBS6226 JBS3368 JBS1726 JBS7630 | Plant Growth Promotion | Bacillus spp | FIG. 20A, 20B, |
| | | | 20C, 21 |
| JBS4549 JBS4761 JBS6226 JBS3368 JBS1726 JBS7630 | Plant Growth Promotion | Azospirillum spp | FIG. 16A, 16B |
| JBS4549 JBS4761 JBS6226 JBS3368 JBS1726 JBS7630 | Nutrient acquisition, | Glomus spp. | FIG. 18 A, 18 B, 19 |
| | plant growth promotion | | |
| JBS4549 JBS4761 JBS6226 JBS3368 JBS1726 JBS7630 | Free-living nitrogen | Azospirillum spp. | FIG. 16A, 16B |
| | fixation | | |
| JBS9675 JBS9185 JBS7002 JBS8358 JBS6844 | Symbiotic nitrogen | Bradyrhizobium spp. | FIG. 17A, 17B |
| JBS9419 JBS6363 JBS9274 JBS6762 | fixation | | |
| JBS8671 JBS6287 JBS8568 JBS6717 JBS6713 | | | |
| JBS6647 JBS6603 JBS9634 JBS7170 | | | |
| JBS5086 JBS8807 JBS8750 JBS6226 JBS5867 | | | |
| JBS9311 JBS6938 JBS8326 JBS6604 | | | |
| JBS6195 JBS4046 JBS1726 JBS9143 JBS3286 | | | |
| JBS9172 JBS6880 JBS5011 | | | |

In some embodiments, the at least one microbial signaler is Streptomyces avidinii, Streptomyces colombiensis, Streptomyces lavendulae, Streptomyces roseochromogenus, Streptomyces spororaveus, Streptomyces sporoverrucosus, Streptomyces venezuelae, Streptomyces xanthophaeus, Streptomyces angustmyceticus, Streptomyces hygroscopicus, Streptomyces libani, Streptomyces lydicus, Streptomyces nigrescens, Streptomyces platensis, Streptomyces rimosus, Streptomyces tubercidicus, Streptomyces bungoensis, Streptomyces cyslabdanicus, Streptomyces galbus, Streptomyces kagawaensis, Streptomyces lasaliensis, Streptomyces lasalocidi, Streptomyces longwoodensis, Streptomyces spinichromogenes, Streptomyces cirratus, Streptomyces nojiriensis, Streptomyces verne, Streptomyces vinaceus, Streptomyces virginiae, Streptomyces catenulae, Streptomyces cinereus, Streptomyces griseocarneus, Streptomyces sioyaensis, Streptomyces subrutilus, Streptomyces atrolaccus, Streptomyces auratus, Streptomyces fagopyri, Streptomyces kaempferi, Streptomyces mirabilis, Streptomyces olivochromogenes, Streptomyces chattanoogensis, Streptomyces flaveus, Streptomyces goshikiensis, Streptomyces cinnamonensis, Streptomyces senoensis, Streptomyces echinatus, Streptomyces filipinensis, Streptomyces gulbargensis, Streptomyces myxogenes, Streptomyces novaecaesareae, Streptomyces spectabilis, Streptomyces tanashiensis, Streptomyces ginsengisoli, Streptomyces graminisoli, Streptomyces lucensis, Streptomyces yaanensis, Streptomyces canferus, Streptomyces decoyicus, Streptomyces glebosus, Streptomyces ossamyceticus, Streptomyces badius, Streptomyces cyaneofuscatus, Streptomyces flavogriseus, Streptomyces griseus, Streptomyces mediolani, Streptomyces praecox, Streptomyces pratensis, Streptomyces omiyaensis, Streptomyces aquilus, Streptomyces caeruleatus, Streptomyces griseochromogenes, Streptomyces pseudovenezuelae, Streptomyces viridochromogenes, Streptomyces argenteolus, Streptomyces chrestomyceticus, Streptomyces coelicolor, Streptomyces microsporus, Streptomyces aureus, Streptomyces lutosisoli, Streptomyces minoensis, Streptomyces rhizosphaerihabitans, Streptomyces griseoruber, Streptomyces scabiei, Streptomyces achromogenes, Streptomyces canarius, Streptomyces capoamus, Streptomyces cellostaticus, or Streptomyces katrae. In some embodiments, the at least one microbial signaler is Streptomyces echinatus, Streptomyces galbus, Streptomyces lavendulae, Streptomyces libani, Streptomyces lydicus, Streptomyces mirabilis, or Streptomyces venezuelae.

In some embodiments, the at least one microbial signaler belongs to the genus Streptomyces, Fusarium, or Bacillus. In some embodiments, the at least one microbial signaler belongs to the genus Streptomyces.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID Nos: 1-36. In some embodiments, the at least one microbial signaler comprises a 16S nucleic acid sequence having at least about 97% sequence identity to one or more of the following SEQ ID Nos: 1-36. In some embodiments, the at least one microbial signaler comprises a 16S nucleic acid sequence of any one of the following SEQ ID Nos: 1-36.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 1. In some embodiments, the at least one microbial signaler comprises a 16S nucleic acid sequence of any one of the following SEQ ID NO: 1. In some embodiments, the at least one microbial signaler comprises a 16S nucleic acid sequence having at least about 97% sequence identity to SEQ ID NO: 1. In some embodiments, the at least one microbial signaler is Streptomyces echinatus, Streptomyces filipinensis, Streptomyces gulbargensis, Streptomyces longwoodensis, Streptomyces myxogenes, Streptomyces novaecaesareae, Streptomyces spectabilis, or Streptomyces tanashiensis.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 2. In some embodiments, the at least one microbial signaler comprises a 16S nucleic acid sequence of any one of the following SEQ ID NO: 2. In some embodiments, the at least one microbial signaler comprises a 16S nucleic acid sequence having at least about 97% sequence identity to SEQ ID NO: 2. In some embodiments, the at least one microbial signaler is Streptomyces echinatus, Streptomyces ginsengisoli, Streptomyces graminisoli, Streptomyces gulbargensis, Streptomyces longwoodensis, Streptomyces lucensis, Streptomyces tanashiensis, or Streptomyces yaanensis.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 3. In some embodiments, the at least one microbial signaler comprises a 16S nucleic acid sequence of any one of the following SEQ ID NO: 3. In some embodiments, the at least one microbial signaler comprises a 16S nucleic acid sequence having at least about 97% sequence identity to SEQ ID NO: 3. In some embodiments, the at least one microbial signaler is Streptomyces bungoensis, Streptomyces cyslabdanicus, Streptomyces galbus, Streptomyces kagawaensis, Streptomyces lasaliensis, Streptomyces lasalocidi, Streptomyces longwoodensis, or Streptomyces spinichromogenes.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 4. In some embodiments, the at least one microbial signaler comprises a 16S nucleic acid sequence of any one of the following SEQ ID NO: 4. In some embodiments, the at least one microbial signaler comprises a 16S nucleic acid sequence having at least about 97% sequence identity to SEQ ID NO: 4. In some embodiments, the at least one microbial signaler is Streptomyces auratus, Streptomyces cyslabdanicus, Streptomyces fagopyri, Streptomyces galbus, Streptomyces kaempferi, Streptomyces mirabilis, or Streptomyces olivochromogenes.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 5. In some embodiments, the at least one microbial signaler comprises a 16S nucleic acid sequence of any one of the following SEQ ID NO: 5. In some embodiments, the at least one microbial signaler comprises a 16S nucleic acid sequence having at least about 97% sequence identity to SEQ ID NO: 5. In some embodiments, the at least one microbial signaler is Streptomyces achromogenes, Streptomyces bungoensis, Streptomyces canarius, Streptomyces capoamus, Streptomyces cellostaticus, Streptomyces galbus, Streptomyces katrae, or Streptomyces spinichromogenes.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 6. In some embodiments, the at least one microbial signaler comprises a 16S nucleic acid sequence of any one of the following SEQ ID NO: 6. In some embodiments, the at least one microbial signaler comprises a 16S nucleic acid sequence having at least about 97% sequence identity to SEQ ID NO: 6. In some embodiments, the at least one microbial signaler is Streptomyces avidinii, Streptomyces colombiensis, Streptomyces lavendulae, Streptomyces roseochromogenus, Streptomyces spororaveus, Streptomyces sporoverrucosus, Streptomyces venezuelae, or Streptomyces xanthophaeus.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 7. In some embodiments, the at least one microbial signaler comprises a 16S nucleic acid sequence of any one of the following SEQ ID NO: 7. In some embodiments, the at least one microbial signaler comprises a 16S nucleic acid sequence having at least about 97% sequence identity to SEQ ID NO: 7. In some embodiments, the at least one microbial signaler is Streptomyces avidinii, Streptomyces cirratus, Streptomyces lavendulae, Streptomyces nojiriensis, Streptomyces spororaveus, Streptomyces subrutilus, Streptomyces venezuelae, or Streptomyces xanthophaeus.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 8. In some embodiments, the at least one microbial signaler comprises a 16S nucleic acid sequence of any one of the following SEQ ID NO: 8. In some embodiments, the at least one microbial signaler comprises a 16S nucleic acid sequence having at least about 97% sequence identity to SEQ ID NO: 8. In some embodiments, the at least one microbial signaler is Streptomyces avidinii, Streptomyces colombiensis, Streptomyces flaveus, Streptomyces goshikiensis, Streptomyces lavendulae, Streptomyces spororaveus, Streptomyces subrutilus, or Streptomyces venezuelae.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 9. In some embodiments, the at least one microbial signaler comprises a 16S nucleic acid sequence of any one of the following SEQ ID NO: 9. In some embodiments, the at least one microbial signaler comprises a 16S nucleic acid sequence having at least about 97% sequence identity to SEQ ID NO: 9. In some embodiments, the at least one microbial signaler is Streptomyces cinnamonensis, Streptomyces cirratus, Streptomyces goshikiensis, Streptomyces lavendulae, Streptomyces senoensis, Streptomyces sporoverrucosus, Streptomyces vinaceus, or Streptomyces virginiae.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 10. In some embodiments, the at least one microbial signaler comprises a 16S nucleic acid sequence of any one of the following SEQ ID NO: 10. In some embodiments, the at least one microbial signaler comprises a 16S nucleic acid sequence having at least about 97% sequence identity to SEQ ID NO: 10. In some embodiments, the at least one microbial signaler is Streptomyces cinnamonensis, Streptomyces flaveus, Streptomyces lavendulae, Streptomyces nojiriensis, Streptomyces spororaveus, Streptomyces venezuelae, Streptomyces virginiae, or Streptomyces xanthophaeus.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 11. In some embodiments, the at least one microbial signaler comprises a 16S nucleic acid sequence of any one of the following SEQ ID NO: 11. In some embodiments, the at least one microbial signaler comprises a 16S nucleic acid sequence having at least about 97% sequence identity to SEQ ID NO: 11. In some embodiments, the at least one microbial signaler is Streptomyces auratus, Streptomyces cinnamonensis, Streptomyces lavendulae, Streptomyces sioyaensis, Streptomyces spororaveus, Streptomyces verne, Streptomyces virginiae, or Streptomyces xanthophaeus.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 12. In some embodiments, the at least one microbial signaler comprises a 16S nucleic acid sequence of any one of the following SEQ ID NO: 12. In some embodiments, the at least one microbial signaler comprises a 16S nucleic acid sequence having at least about 97% sequence identity to SEQ ID NO: 12. In some embodiments, the at least one microbial signaler is Streptomyces cinnamonensis, Streptomyces flaveus, Streptomyces lavendulae, Streptomyces nojiriensis, Streptomyces spororaveus, Streptomyces venezuelae, Streptomyces virginiae, or Streptomyces xanthophaeus.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 13. In some embodiments, the at least one microbial signaler comprises a 16S nucleic acid sequence of any one of the following SEQ ID NO: 13. In some embodiments, the at least one microbial signaler comprises a 16S nucleic acid sequence having at least about 97% sequence identity to SEQ ID NO: 13. In some embodiments, the at least one microbial signaler is Streptomyces badius, Streptomyces cyaneofuscatus, Streptomyces flavogriseus, Streptomyces griseus, Streptomyces lavendulae, Streptomyces mediolani, Streptomyces praecox, or Streptomyces pratensis.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 14. In some embodiments, the at least one microbial signaler comprises a 16S nucleic acid sequence of any one of the following SEQ ID NO: 14. In some embodiments, the at least one microbial signaler comprises a 16S nucleic acid sequence having at least about 97% sequence identity to SEQ ID NO: 14. In some embodiments, the at least one microbial signaler is Streptomyces avidinii, Streptomyces cirratus, Streptomyces lavendulae, Streptomyces nojiriensis, Streptomyces omiyaensis, Streptomyces spororaveus, Streptomyces subrutilus, or Streptomyces vinaceus.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 15. In some embodiments, the at least one microbial signaler comprises a 16S nucleic acid sequence of any one of the following SEQ ID NO: 15. In some embodiments, the at least one microbial signaler comprises a 16S nucleic acid sequence having at least about 97% sequence identity to SEQ ID NO: 15. In some embodiments, the at least one microbial signaler is Streptomyces cinnamonensis, Streptomyces flaveus, Streptomyces lavendulae, Streptomyces nojiriensis, Streptomyces sporoverrucosus, Streptomyces venezuelae, Streptomyces vinaceus, Streptomyces virginiae, or Streptomyces xanthophaeus.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 16. In some embodiments, the at least one microbial signaler comprises a 16S nucleic acid sequence of any one of the following SEQ ID NO: 16. In some embodiments, the at least one microbial signaler comprises a 16S nucleic acid sequence having at least about 97% sequence identity to SEQ ID NO: 16. In some embodiments, the at least one microbial signaler is Streptomyces lavendulae, Streptomyces nojiriensis, Streptomyces spororaveus, Streptomyces subrutilus, Streptomyces venezuelae, Streptomyces virginiae, or Streptomyces xanthophaeus.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 17. In some embodiments, the at least one microbial signaler comprises a 16S nucleic acid sequence of any one of the following SEQ ID NO: 17. In some embodiments, the at least one microbial signaler comprises a 16S nucleic acid sequence having at least about 97% sequence identity to SEQ ID NO: 17. In some embodiments, the at least one microbial signaler is Streptomyces cinnamonensis, Streptomyces flaveus, Streptomyces goshikiensis, Streptomyces lavendulae, Streptomyces nojiriensis, Streptomyces sporoverrucosus, Streptomyces venezuelae, Streptomyces virginiae, or Streptomyces xanthophaeus.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 18. In some embodiments, the at least one microbial signaler comprises a 16S nucleic acid sequence of any one of the following SEQ ID NO: 18. In some embodiments, the at least one microbial signaler comprises a 16S nucleic acid sequence having at least about 97% sequence identity to SEQ ID NO: 18. In some embodiments, the at least one microbial signaler is Streptomyces colombiensis, Streptomyces flaveus, Streptomyces lavendulae, Streptomyces senoensis, Streptomyces sporoverrucosus, Streptomyces venezuelae, Streptomyces vinaceus, Streptomyces virginiae, or Streptomyces xanthophaeus.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 19. In some embodiments, the at least one microbial signaler comprises a 16S nucleic acid sequence of any one of the following SEQ ID NO: 19. In some embodiments, the at least one microbial signaler comprises a 16S nucleic acid sequence having at least about 97% sequence identity to SEQ ID NO: 19. In some embodiments, the at least one microbial signaler is Streptomyces flaveus, Streptomyces lavendulae, Streptomyces sporoverrucosus, Streptomyces venezuelae, Streptomyces vinaceus, Streptomyces virginiae, or Streptomyces xanthophaeus.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 20. In some embodiments, the at least one microbial signaler comprises a 16S nucleic acid sequence of any one of the following SEQ ID NO: 20. In some embodiments, the at least one microbial signaler comprises a 16S nucleic acid sequence having at least about 97% sequence identity to SEQ ID NO: 20. In some embodiments, the at least one microbial signaler is Streptomyces angustmyceticus, Streptomyces hygroscopicus, Streptomyces libani, Streptomyces lydicus, Streptomyces nigrescens, Streptomyces platensis, Streptomyces rimosus, or Streptomyces tubercidicus.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 21. In some embodiments, the at least one microbial signaler comprises a 16S nucleic acid sequence of any one of the following SEQ ID NO: 21. In some embodiments, the at least one microbial signaler comprises a 16S nucleic acid sequence having at least about 97% sequence identity to SEQ ID NO: 21. In some embodiments, Streptomyces angustmyceticus, Streptomyces catenulae, Streptomyces cinereus, Streptomyces griseocarneus, Streptomyces hygroscopicus, Streptomyces libani, Streptomyces nigrescens, or Streptomyces sioyaensis.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 22. In some embodiments, the at least one microbial signaler comprises a 16S nucleic acid sequence of any one of the following SEQ ID NO: 22. In some embodiments, the at least one microbial signaler comprises a 16S nucleic acid sequence having at least about 97% sequence identity to SEQ ID NO: 22. In some embodiments, the at least one microbial signaler is Streptomyces atrolaccus, Streptomyces auratus, Streptomyces griseocarneus, Streptomyces hygroscopicus, Streptomyces libani, Streptomyces lydicus, Streptomyces sioyaensis, or Streptomyces tubercidicus.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 23. In some embodiments, the at least one microbial signaler comprises a 16S nucleic acid sequence of any one of the following SEQ ID NO: 23. In some embodiments, the at least one microbial signaler comprises a 16S nucleic acid sequence having at least about 97% sequence identity to SEQ ID NO: 23. In some embodiments, the at least one microbial signaler is Streptomyces atrolaccus, Streptomyces auratus, Streptomyces griseocarneus, Streptomyces hygroscopicus, Streptomyces libani, Streptomyces lydicus, Streptomyces sioyaensis, or Streptomyces tubercidicus.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 24. In some embodiments, the at least one microbial signaler comprises a 16S nucleic acid sequence of any one of the following SEQ ID NO: 24. In some embodiments, the at least one microbial signaler comprises a 16S nucleic acid sequence having at least about 97% sequence identity to SEQ ID NO: 24. In some embodiments, the at least one microbial signaler is Streptomyces angustmyceticus, Streptomyces atrolaccus, Streptomyces griseocarneus, Streptomyces hygroscopicus, Streptomyces libani, Streptomyces nigrescens, Streptomyces sioyaensis, or Streptomyces tubercidicus.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 25. In some embodiments, the at least one microbial signaler comprises a 16S nucleic acid sequence of any one of the following SEQ ID NO: 25. In some embodiments, the at least one microbial signaler comprises a 16S nucleic acid sequence having at least about 97% sequence identity to SEQ ID NO: 25. In some embodiments, the at least one microbial signaler is Streptomyces angustmyceticus, Streptomyces hygroscopicus, Streptomyces libani, Streptomyces lydicus, Streptomyces nigrescens, Streptomyces platensis, Streptomyces rimosus, or Streptomyces tubercidicus.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 26. In some embodiments, the at least one microbial signaler comprises a 16S nucleic acid sequence of any one of the following SEQ ID NO: 26. In some embodiments, the at least one microbial signaler comprises a 16S nucleic acid sequence having at least about 97% sequence identity to SEQ ID NO: 26. In some embodiments, the at least one microbial signaler is Streptomyces angustmyceticus, Streptomyces hygroscopicus, Streptomyces libani, Streptomyces lydicus, Streptomyces nigrescens, Streptomyces platensis, Streptomyces rimosus, or Streptomyces sioyaensis.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 27. In some embodiments, the at least one microbial signaler comprises a 16S nucleic acid sequence of any one of the following SEQ ID NO: 27. In some embodiments, the at least one microbial signaler comprises a 16S nucleic acid sequence having at least about 97% sequence identity to SEQ ID NO: 27. In some embodiments, the at least one microbial signaler is Streptomyces angustmyceticus, Streptomyces atrolaccus, Streptomyces chattanoogensis, Streptomyces libani, Streptomyces lydicus, Streptomyces nigrescens, Streptomyces sioyaensis, or Streptomyces tubercidicus.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 28. In some embodiments, the at least one microbial signaler comprises a 16S nucleic acid sequence of any one of the following SEQ ID NO: 28. In some embodiments, the at least one microbial signaler comprises a 16S nucleic acid sequence having at least about 97% sequence identity to SEQ ID NO: 28. In some embodiments, the at least one microbial signaler is Streptomyces angustmyceticus, Streptomyces hygroscopicus, Streptomyces libani, Streptomyces lydicus, Streptomyces nigrescens, Streptomyces platensis, Streptomyces rimosus, or Streptomyces tubercidicus.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 29. In some embodiments, the at least one microbial signaler comprises a 16S nucleic acid sequence of any one of the following SEQ ID NO: 29. In some embodiments, the at least one microbial signaler comprises a 16S nucleic acid sequence having at least about 97% sequence identity to SEQ ID NO: 29. In some embodiments, the at least one microbial signaler is Streptomyces angustmyceticus, Streptomyces atrolaccus, Streptomyces hygroscopicus, Streptomyces libani, Streptomyces lydicus, Streptomyces nigrescens, Streptomyces sioyaensis, or Streptomyces tubercidicus.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 30. In some embodiments, the at least one microbial signaler comprises a 16S nucleic acid sequence of any one of the following SEQ ID NO: 30. In some embodiments, the at least one microbial signaler comprises a 16S nucleic acid sequence having at least about 97% sequence identity to SEQ ID NO: 30. In some embodiments, the at least one microbial signaler is Streptomyces canferus, Streptomyces decoyicus, Streptomyces glebosus, Streptomyces hygroscopicus, Streptomyces libani, Streptomyces lydicus, Streptomyces ossamyceticus, or Streptomyces platensis.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 31. In some embodiments, the at least one microbial signaler comprises a 16S nucleic acid sequence of any one of the following SEQ ID NO: 31. In some embodiments, the at least one microbial signaler comprises a 16S nucleic acid sequence having at least about 97% sequence identity to SEQ ID NO: 31. In some embodiments, the at least one microbial signaler is Streptomyces angustmyceticus, Streptomyces catenulae, Streptomyces cinereus, Streptomyces libani, Streptomyces lydicus, Streptomyces nigrescens, Streptomyces platensis, or Streptomyces tubercidicus.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 32. In some embodiments, the at least one microbial signaler comprises a 16S nucleic acid sequence of any one of the following SEQ ID NO: 32. In some embodiments, the at least one microbial signaler comprises a 16S nucleic acid sequence having at least about 97% sequence identity to SEQ ID NO: 32. In some embodiments, the at least one microbial signaler is Streptomyces argenteolus, Streptomyces atrolaccus, Streptomyces chattanoogensis, Streptomyces chrestomyceticus, Streptomyces coelicolor, Streptomyces lydicus, Streptomyces microsporus, Streptomyces nigrescens, Streptomyces rimosus, or Streptomyces sioyaensis.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 33. In some embodiments, the at least one microbial signaler comprises a 16S nucleic acid sequence of any one of the following SEQ ID NO: 33. In some embodiments, the at least one microbial signaler comprises a 16S nucleic acid sequence having at least about 97% sequence identity to SEQ ID NO: 33. In some embodiments, the at least one microbial signaler is Streptomyces aquilus, Streptomyces caeruleatus, Streptomyces fagopyri, Streptomyces griseochromogenes, Streptomyces mirabilis, Streptomyces nojiriensis, Streptomyces pseudovenezuelae, Streptomyces viridochromogenes, or Streptomyces viridochromogenes.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 34. In some embodiments, the at least one microbial signaler comprises a 16S nucleic acid sequence of any one of the following SEQ ID NO: 34. In some embodiments, the at least one microbial signaler comprises a 16S nucleic acid sequence having at least about 97% sequence identity to SEQ ID NO: 34. In some embodiments, the at least one microbial signaler is Streptomyces aquilus, Streptomyces aureus, Streptomyces fagopyri, Streptomyces lutosisoli, Streptomyces minoensis, Streptomyces mirabilis, Streptomyces olivochromogenes, or Streptomyces rhizosphaerihabitans.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 35. In some embodiments, the at least one microbial signaler comprises a 16S nucleic acid sequence of any one of the following SEQ ID NO: 35. In some embodiments, the at least one microbial signaler comprises a 16S nucleic acid sequence having at least about 97% sequence identity to SEQ ID NO: 35. In some embodiments, the at least one microbial signaler is Streptomyces aquilus, Streptomyces fagopyri, Streptomyces griseoruber, Streptomyces lutosisoli, Streptomyces minoensis, Streptomyces mirabilis, Streptomyces olivochromogenes, or Streptomyces scabiei.

In some embodiments, the at least one microbial signaler comprises a 16S nucleic acid sequence having at least about 70% (for example, at least about 75%, at least about 80%, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity, including all subranges and values that lie therebetween) sequence identity to one or more of the following SEQ ID NO: 36. In some embodiments, the at least one microbial signaler comprises a 16S nucleic acid sequence of any one of the following SEQ ID NO: 36. In some embodiments, the at least one microbial signaler comprises a 16S nucleic acid sequence having at least about 97% sequence identity to SEQ ID NO: 36. In some embodiments, the at least one microbial signaler is Streptomyces cirratus, Streptomyces nojiriensis, Streptomyces sporoverrucosus, Streptomyces venezuelae, Streptomyces verne, Streptomyces vinaceus, Streptomyces virginiae, or Streptomyces xanthophaeus.

### Methods of Preparing Compositions Comprising Microbial Signalers Disclosed Herein

The disclosure provides methods of producing a composition, the method comprising: bringing at least one target microbe in the physical proximity of any one or more of the microbial signalers disclosed herein. The disclosure provides methods of producing a composition, the method comprising: bringing at least one target microbe in the physical proximity of at least one microbial signaler belonging to the genus Streptomyces, wherein the at least one microbial signaler is capable of enhancing a plant growth-promoting function of the at least one target microbe.

In some embodiments, the methods disclosed herein comprises bringing the target microbe in contact with the at least one microbial signaler. In some embodiments, the method comprises preparing a composition, comprising the target microbe and the at least one microbial signaler.

The isolation, identification, and culturing of the microbes of the present disclosure can be effected using standard microbiological techniques. Examples of such techniques may be found in Gerhardt, P. (ed.) Methods for General and Molecular Microbiology. American Society for Microbiology, Washington, D.C. (1994) and Lennette, E. H. (ed.) Manual of Clinical Microbiology, Third Edition. American Society for Microbiology, Washington, D.C. (1980), each of which is incorporated by reference.

Isolation can be effected by streaking the specimen on a solid medium (e.g., nutrient agar plates) to obtain a single colony, which is characterized by the phenotypic traits described herein (e.g., Gram positive/negative, capable of forming spores aerobically/anaerobically, cellular morphology, carbon source metabolism, acid/base production, enzyme secretion, metabolic secretions, etc.) and to reduce the likelihood of working with a culture which has become contaminated.

For example, for microbes of the disclosure, biologically pure isolates can be obtained through repeated subculture of biological samples, each subculture followed by streaking onto solid media to obtain individual colonies or colony forming units. Methods of preparing, thawing, and growing lyophilized bacteria are commonly known, for example, Gherna, R. L. and C. A. Reddy. 2007. Culture Preservation, p 1019-1033. In C. A. Reddy, T. J. Beveridge, J. A. Breznak, G. A. Marzluf, T. M. Schmidt, and L. R. Snyder, eds. American Society for Microbiology, Washington, D.C., 1033 pages; herein incorporated by reference. Thus freeze dried liquid formulations and cultures stored long term at −70° C. in solutions containing glycerol are contemplated for use in providing formulations of the present disclosure.

The microbes of the present disclosure can be propagated in a liquid or solid medium under aerobic conditions, or alternatively anaerobic conditions. Medium for growing the bacterial strains of the present disclosure may include a carbon source, a nitrogen source, and inorganic salts, as well as specially required substances such as vitamins, amino acids, nucleic acids and the like. In some embodiments, the media comprises water and agar. Examples of suitable carbon sources which can be used for growing the microbes include, but are not limited to, starch, peptone, yeast extract, amino acids, sugars such as glucose, arabinose, mannose, glucosamine, maltose, and the like; salts of organic acids such as acetic acid, fumaric acid, adipic acid, propionic acid, citric acid, gluconic acid, malic acid, pyruvic acid, malonic acid and the like; alcohols such as ethanol and glycerol and the like; oil or fat such as soybean oil, rice bran oil, olive oil, corn oil, sesame oil. The amount of the carbon source added varies according to the kind of carbon source and is typically between 1 to 100 gram(s) per liter of medium. Preferably, glucose, starch, and/or peptone is contained in the medium as a major carbon source, at a concentration of 0.1-5% (W/V). Examples of suitable nitrogen sources which can be used for growing the bacterial strains of the present disclosure include, but are not limited to, amino acids, yeast extract, tryptone, beef extract, peptone, potassium nitrate, ammonium nitrate, ammonium chloride, ammonium sulfate, ammonium phosphate, ammonia or combinations thereof. The amount of nitrogen source varies according to the type of nitrogen source, typically between 0.1 to 30 gram(s) per liter of medium. The inorganic salts, potassium dihydrogen phosphate, dipotassium hydrogen phosphate, disodium hydrogen phosphate, magnesium sulfate, magnesium chloride, ferric sulfate, ferrous sulfate, ferric chloride, ferrous chloride, manganous sulfate, manganous chloride, zinc sulfate, zinc chloride, cupric sulfate, calcium chloride, sodium chloride, calcium carbonate, sodium carbonate can be used alone or in combination. The amount of inorganic acid varies according to the kind of the inorganic salt, typically between 0.001 to 10 gram(s) per liter of medium. Examples of specially required substances include, but are not limited to, vitamins, nucleic acids, yeast extract, peptone, meat extract, malt extract, dried yeast and combinations thereof. Cultivation can be effected at a temperature, which allows the growth of the microbial strains, essentially, between 20° C. and 46° C. In some embodiments, a temperature range is 30° C.-39° C. For optimal growth, in some embodiments, the medium can be adjusted to pH 6.0-7.4. It will be appreciated that commercially available media may also be used to culture the microbial strains, such as Nutrient Broth or Nutrient Agar available from Difco, Detroit, MI. It will be appreciated that cultivation time may differ depending on the type of culture medium used and the concentration of sugar as a major carbon source.

In some embodiments, cultivation lasts between about 24 to about 96 hours. In some embodiments, cultivation lasts longer than 96 hours, such as, for example, about 4 days, about 5 days, about 1 week, about 2 weeks, about 3 weeks, about 4 weeks, about 6 weeks, or about 2 months. Microbial cells thus obtained are isolated using methods, which are well known in the art. Examples include, but are not limited to, membrane filtration and centrifugal separation. The pH may be adjusted using sodium hydroxide and the like and the culture may be dried using a freeze dryer, until the water content becomes equal to 4% or less. Microbial co-cultures may be obtained by propagating each strain as described hereinabove. In some embodiments, microbial multi-strain cultures may be obtained by propagating two or more of the strains described hereinabove. It will be appreciated that the microbial strains may be cultured together when compatible culture conditions can be employed.

The disclosure provides compositions produced using any one of the methods of producing compositions disclosed herein.

### Methods of Enhancing Plant Growth Promoting Function of Target Microbes

The disclosure provides methods of enhancing a plant growth-promoting function of a target microbe, the method comprising: bringing the target microbe in the physical proximity of any one or more of the microbial signalers disclosed herein. The disclosure also provides methods of enhancing a plant growth-promoting function of a target microbe, the method comprising: bringing the target microbe in the physical proximity of at least one microbial signaler belonging to the genus Streptomyces disclosed herein.

In some embodiments, the methods comprise increasing the plant growth-promoting function of the target microbe by at least about 1% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween). In some embodiments, the plant growth-promoting function comprises: (a) plant pathogen-inhibiting function, (b) zinc solubilizing function, (c) phosphate solubilizing function, (d) production of an antibiotic, (e) nitrogen fixing function, (f) a function of improving a plant's nutrient acquisition, (f) production of plant growth hormones, or (g) any combination thereof.

In some embodiments, the methods disclosed herein enhance the plant pathogen-inhibiting function of the target microbe by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween). In some embodiments, the method enhances the plant pathogen-inhibiting function of the target microbe by at least about 5%.

In some embodiments, the method disclosed herein enhance the zinc solubilizing function of the target microbe by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween). In some embodiments, the method enhances the zinc solubilizing function of the target microbe by at least about 5%.

In some embodiments, the method disclosed herein enhance the phosphate solubilizing function of the target microbe by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween). In some embodiments, the method enhances the phosphate solubilizing function of the target microbe by at least about 5%.

In some embodiments, the method disclosed herein enhance the plant-growth promoting function of the target microbe under low nutrient conditions.

### Microbial Compositions

The disclosure provides microbial compositions comprising any one or more of the microbial signalers disclosed herein and/or compositions disclosed herein. In some embodiments, the microbial compositions may further comprise suitable carrier and other additives. In some embodiments, the microbial compositions of the present disclosure are solid. Where solid compositions are used, it may be desired to include one or more carrier materials including, but not limited to: mineral earths such as silicas, talc, kaolin, limestone, chalk, clay, dolomite, diatomaceous earth; calcium sulfate; magnesium sulfate; magnesium oxide; zeolites, calcium carbonate; magnesium carbonate; trehalose; chitosan; shellac; and starch.

In some embodiments, the microbial compositions of the present disclosure are liquid. In further embodiments, the liquid comprises a solvent that may include water or an alcohol or a saline or carbohydrate solution, and other plant-safe solvents. In some embodiments, the microbial compositions of the present disclosure include binders such as plant-safe polymers, carboxymethylcellulose, starch, polyvinyl alcohol, and the like.

In some embodiments, the microbial compositions of the present disclosure comprise thickening agents such as silica, clay, natural extracts of seeds or seaweed, synthetic derivatives of cellulose, guar gum, locust bean gum, alginates, and methylcelluloses. In some embodiments, the microbial compositions comprise anti-settling agents such as modified starches, polyvinyl alcohol, xanthan gum, and the like.

In some embodiments, the microbial compositions of the present disclosure comprise colorants including organic chromophores classified as nitroso; nitro; azo, including monoazo, bisazo and polyazo; acridine, anthraquinone, azine, diphenylmethane, indamine, indophenol, methine, oxazine, phthalocyanine, thiazine, thiazole, triarylmethane, xanthene. In some embodiments, the microbial compositions of the present disclosure comprise trace nutrients such as salts of iron, manganese, boron, copper, cobalt, molybdenum and zinc. In some embodiments, the microbial compositions comprise dyes, both natural and artificial.

In some embodiments, the microbial compositions of the present disclosure may include combinations of fungal spores and bacterial spores, fungal spores and bacterial vegetative cells, fungal vegetative cells and bacterial spores, fungal vegetative cells and bacterial vegetative cells. In some embodiments, compositions of the present disclosure comprise bacteria only in the form of spores. In some embodiments, compositions of the present disclosure comprise bacteria only in the form of vegetative cells. In some embodiments, compositions of the present disclosure comprise bacteria in the absence of fungi. In some embodiments, compositions of the present disclosure comprise fungi in the absence of bacteria. In some embodiments, compositions of the present disclosure comprise viable but non-culturable (VBNC) bacteria and/or fungi. In some embodiments, compositions of the present disclosure comprise bacteria and/or fungi in a quiescent state. In some embodiments, compositions of the present disclosure include dormant bacteria and/or fungi. Bacterial spores may include endospores and akinetes. Fungal spores may include statismospores, ballistospores, autospores, aplanospores, zoospores, mitospores, megaspores, microspores, meiospores, chlamydospores, urediniospores, teliospores, oospores, carpospores, tetraspores, sporangiospores, zygospores, ascospores, basidiospores, ascospores, and asciospores.

In some embodiments, the microbial compositions of the present disclosure comprise a plant-safe virucide, parasiticide, bacteriocide, fungicide, biopesticide, or nematicide. In some embodiments, microbial compositions of the present disclosure comprise one or more oxygen scavengers, denitrifies, nitrifiers, heavy metal chelators, and/or dechlorinators; and combinations thereof.

In some embodiments, microbial compositions of the present disclosure comprise one or more preservatives. The preservatives may be in liquid or gas formulations. The preservatives may be selected from one or more of monosaccharide, disaccharide, trisaccharide, polysaccharide, acetic acid, ascorbic acid, calcium ascorbate, erythorbic acid, iso-ascorbic acid, erythrobic acid, potassium nitrate, sodium ascorbate, sodium erythorbate, sodium iso-ascorbate, sodium nitrate, sodium nitrite, nitrogen, benzoic acid, calcium sorbate, ethyl lauroyl arginate, methyl-p-hydroxy benzoate, methyl paraben, potassium acetate, potassium benzoiate, potassium bisulphite, potassium diacetate, potassium lactate, potassium metabisulphite, potassium sorbate, propyl-p-hydroxy benzoate, propyl paraben, sodium acetate, sodium benzoate, sodium bisulphite, sodium nitrite, sodium diacetate, sodium lactate, sodium metabisulphite, sodium salt of methyl-p-hydroxy benzoic acid, sodium salt of propyl-p-hydroxy benzoic acid, sodium sulphate, sodium sulfite, sodium dithionite, sulphurous acid, calcium propionate, dimethyl dicarbonate, natamycin, potassium sorbate, potassium bisulfite, potassium metabisulfite, propionic acid, sodium diacetate, sodium propionate, sodium sorbate, sorbic acid, ascorbic acid, ascorbyl palmitate, ascorbyl stearate, butylated hydro-xyanisole, butylated hydroxytoluene (BHT), butylated hydroxyl anisole (BHA), citric acid, citric acid esters of mono- and/or diglycerides, L-cysteine, L-cysteine hydrochloride, gum guaiacum, gum guaiac, lecithin, lecithin citrate, monoglyceride citrate, monoisopropyl citrate, propyl gallate, sodium metabisulphite, tartaric acid, tertiary butyl hydroquinone, stannous chloride, thiodipropionic acid, dilauryl thiodipropionate, distearyl thiodipropionate, ethoxyquin, sulfur dioxide, formic acid, or tocopherol(s).

In some embodiments, the microbial compositions are shelf stable in a refrigerator (35-40° F.) for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial compositions are shelf stable in a refrigerator (35-40° F.) for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks. In some embodiments, the microbial compositions are shelf stable in a refrigerator (35-40° F.) for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 years.

In some embodiments, the microbial compositions are shelf stable at room temperature (68-72° F.) or between 50-77° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial compositions are shelf stable at room temperature (68-72° F.) or between 50-77° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks. In some embodiments, the microbial compositions are shelf stable at room temperature (68-72° F.) or between 50-77° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 years.

In some embodiments, the microbial compositions are shelf stable at −23-35° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial compositions are shelf stable at −23-35° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks. In some embodiments, the microbial compositions are shelf stable at −23-35° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 years.

In some embodiments, the microbial compositions are shelf stable at 77-100T for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial compositions are shelf stable at 77-100T for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks. In some embodiments, the microbial compositions are shelf stable at 77-100T for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 years.

In some embodiments, the microbial compositions are shelf stable at 101-213T for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial compositions are shelf stable at 101-213° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks. In some embodiments, the microbial compositions are shelf stable at 101-213° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 years.

In some embodiments, the microbial compositions of the present disclosure are shelf stable at refrigeration temperatures (35-40° F.), at room temperature (68-72° F.), between 50-77° F., between −23-35° F., between 70-100T, or between 101-213° F. for a period of about 1 to 100, about 1 to 95, about 1 to 90, about 1 to 85, about 1 to 80, about 1 to 75, about 1 to 70, about 1 to 65, about 1 to 60, about 1 to 55, about 1 to 50, about 1 to 45, about 1 to 40, about 1 to 35, about 1 to 30, about 1 to 25, about 1 to 20, about 1 to 15, about 1 to 10, about 1 to 5, about 5 to 100, about 5 to 95, about 5 to 90, about 5 to 85, about 5 to 80, about 5 to 75, about 5 to 70, about 5 to 65, about 5 to 60, about 5 to 55, about 5 to 50, about 5 to 45, about 5 to 40, about 5 to 35, about 5 to 30, about 5 to 25, about 5 to 20, about 5 to 15, about 5 to 10, about 10 to 100, about 10 to 95, about 10 to 90, about 10 to 85, about 10 to 80, about 10 to 75, about 10 to 70, about 10 to 65, about 10 to 60, about 10 to 55, about 10 to 50, about 10 to 45, about 10 to 40, about 10 to 35, about 10 to 30, about 10 to 25, about 10 to 20, about 10 to 15, about 15 to 100, about 15 to 95, about 15 to 90, about 15 to 85, about 15 to 80, about 15 to 75, about 15 to 70, about 15 to 65, about 15 to 60, about 15 to 55, about 15 to 50, about 15 to 45, about 15 to 40, about 15 to 35, about 15 to 30, about 15 to 25, about 15 to 20, about 20 to 100, about 20 to 95, about 20 to 90, about 20 to 85, about 20 to 80, about 20 to 75, about 20 to 70, about 20 to 65, about 20 to 60, about 20 to 55, about 20 to 50, about 20 to 45, about 20 to 40, about 20 to 35, about 20 to 30, about 20 to 25, about 25 to 100, about 25 to 95, about 25 to 90, about 25 to 85, about 25 to 80, about 25 to 75, about 25 to 70, about 25 to 65, about 25 to 60, about 25 to 55, about 25 to 50, about 25 to 45, about 25 to 40, about 25 to 35, about 25 to 30, about 30 to 100, about 30 to 95, about 30 to 90, about 30 to 85, about 30 to 80, about 30 to 75, about 30 to 70, about 30 to 65, about 30 to 60, about 30 to 55, about 30 to 50, about 30 to 45, about 30 to 40, about 30 to 35, about 35 to 100, about 35 to 95, about 35 to 90, about 35 to 85, about 35 to 80, about 35 to 75, about 35 to 70, about 35 to 65, about 35 to 60, about 35 to 55, about 35 to 50, about 35 to 45, about 35 to 40, about 40 to 100, about 40 to 95, about 40 to 90, about 40 to 85, about 40 to 80, about 40 to 75, about 40 to 70, about 40 to 65, about 40 to 60, about 40 to 55, about 40 to 50, about 40 to 45, about 45 to 100, about 45 to 95, about 45 to 90, about 45 to 85, about 45 to 80, about 45 to 75, about 45 to 70, about 45 to 65, about 45 to 60, about 45 to 55, about 45 to 50, about 50 to 100, about 50 to 95, about 50 to 90, about 50 to 85, about 50 to 80, about 50 to 75, about 50 to 70, about 50 to 65, about 50 to 60, about 50 to 55, about 55 to 100, about 55 to 95, about 55 to 90, about 55 to 85, about 55 to 80, about 55 to 75, about 55 to 70, about 55 to 65, about 55 to 60, about 60 to 100, about 60 to 95, about 60 to 90, about 60 to 85, about 60 to 80, about 60 to 75, about 60 to 70, about 60 to 65, about 65 to 100, about 65 to 95, about 65 to 90, about 65 to 85, about 65 to 80, about 65 to 75, about 65 to 70, about 70 to 100, about 70 to 95, about 70 to 90, about 70 to 85, about 70 to 80, about 70 to 75, about 75 to 100, about 75 to 95, about 75 to 90, about 75 to 85, about 75 to 80, about 80 to 100, about 80 to 95, about 80 to 90, about 80 to 85, about 85 to 100, about 85 to 95, about 85 to 90, about 90 to 100, about 90 to 95, or 95 to 100 weeks.

In some embodiments, the microbial compositions of the present disclosure are shelf stable at refrigeration temperatures (35-40° F.), at room temperature (68-72° F.), between 50-77° F., between −23-35° F., between 70-100T, or between 101-213° F. for a period of 1 to 100, 1 to 95, 1 to 90, 1 to 85, 1 to 80, 1 to 75, 1 to 70, 1 to 65, 1 to 60, 1 to 55, 1 to 50, 1 to 45, 1 to 40, 1 to 35, 1 to 30, 1 to 25, 1 to 20, 1 to 5, 1 to 10, 1 to 5, 5 to 00, 5 to 95, 5 to 90, 5 to 85, 5 to 80, 5 to 75, 5 to 70, 5 to 65, 5 to 60, 5 to 55, 5 to 50, 5 to 45, 5 to 40, 5 to 35, 5 to 30, 5 to 25, 5 to 20, 5 to 15, 5 to 10, 10 to 100, 10 to 95, 10 to 90, 10 to 85, 10 to 80, 10 to 75, 10 to 70, 10 to 65, 10 to 60, 10 to 55, 10 to 50, 10 to 45, 10 to 40, 10 to 35, 10 to 30, 10 to 25, 10 to 20, 10 to 15, 15 to 100, 15 to 95, 15 to 90, 15 to 85, 15 to 80, 15 to 75, 15 to 70, 15 to 65, 15 to 60, 15 to 55, 15 to 50, 15 to 45, 15 to 40, 15 to 35, 15 to 30, 15 to 25, 15 to 20, 20 to 100, 20 to 95, 20 to 90, 20 to 85, 20 to 80, 20 to 75, 20 to 70, 20 to 65, 20 to 60, 20 to 55, 20 to 50, 20 to 45, 20 to 40, 20 to 35, 20 to 30, 20 to 25, 25 to 100, 25 to 95, 25 to 90, 25 to 85, 25 to 80, 25 to 75, 25 to 70, 25 to 65, 25 to 60, 25 to 55, 25 to 50, 25 to 45, 25 to 40, 25 to 35, 25 to 30, 30 to 100, 30 to 95, 30 to 90, 30 to 85, 30 to 80, 30 to 75, 30 to 70, 30 to 65, 30 to 60, 30 to 55, 30 to 50, 30 to 45, 30 to 40, 30 to 35, 35 to 100, 35 to 95, 35 to 90, 35 to 85, 35 to 80, 35 to 75, 35 to 70, 35 to 65, 35 to 60, 35 to 55, 35 to 50, 35 to 45, 35 to 40, 40 to 100, 40 to 95, 40 to 90, 40 to 85, 40 to 80, 40 to 75, 40 to 70, 40 to 65, 40 to 60, 40 to 55, 40 to 50, 40 to 45, 45 to 100, 45 to 95, 45 to 90, 45 to 85, 45 to 80, 45 to 75, 45 to 70, 45 to 65, 45 to 60, 45 to 55, 45 to 50, 50 to 100, 50 to 95, 50 to 90, 50 to 85, 50 to 80, 50 to 75, 50 to 70, 50 to 65, 50 to 60, 50 to 55, 55 to 100, 55 to 95, 55 to 90, 55 to 85, 55 to 80, 55 to 75, 55 to 70, 55 to 65, 55 to 60, 60 to 100, 60 to 95, 60 to 90, 60 to 85, 60 to 80, 60 to 75, 60 to 70, 60 to 65, 65 to 100, 65 to 95, 65 to 90, 65 to 85, 65 to 80, 65 to 75, 65 to 70, 70 to 100, 70 to 95, 70 to 90, 70 to 85, 70 to 80, 70 to 75, 75 to 100, 75 to 95, 75 to 90, 75 to 85, 75 to 80, 80 to 100, 80 to 95, 80 to 90, 80 to 85, 85 to 100, 85 to 95, 85 to 90, 90 to 100, 90 to 95, or 95 to 100 weeks.

In some embodiments, the microbial compositions of the present disclosure are shelf stable at refrigeration temperatures (35-40° F.), at room temperature (68-72° F.), between 50-77° F., between −23-35° F., between 70-100T, or between 101-213° F. for a period of about 1 to 36, about 1 to 34, about 1 to 32, about 1 to 30, about 1 to 28, about 1 to 26, about 1 to 24, about 1 to 22, about 1 to 20, about 1 to 18, about 1 to 16, about 1 to 14, about 1 to 12, about 1 to 10, about 1 to 8, about 1 to 6, about 1 one 4, about 1 to 2, about 4 to 36, about 4 to 34, about 4 to 32, about 4 to 30, about 4 to 28, about 4 to 26, about 4 to 24, about 4 to 22, about 4 to 20, about 4 to 18, about 4 to 16, about 4 to 14, about 4 to 12, about 4 to 10, about 4 to 8, about 4 to 6, about 6 to 36, about 6 to 34, about 6 to 32, about 6 to 30, about 6 to 28, about 6 to 26, about 6 to 24, about 6 to 22, about 6 to 20, about 6 to 18, about 6 to 16, about 6 to 14, about 6 to 12, about 6 to 10, about 6 to 8, about 8 to 36, about 8 to 34, about 8 to 32, about 8 to 30, about 8 to 28, about 8 to 26, about 8 to 24, about 8 to 22, about 8 to 20, about 8 to 18, about 8 to 16, about 8 to 14, about 8 to 12, about 8 to 10, about 10 to 36, about 10 to 34, about 10 to 32, about 10 to 30, about 10 to 28, about 10 to 26, about 10 to 24, about 10 to 22, about 10 to 20, about 10 to 18, about 10 to 16, about 10 to 14, about 10 to 12, about 12 to 36, about 12 to 34, about 12 to 32, about 12 to 30, about 12 to 28, about 12 to 26, about 12 to 24, about 12 to 22, about 12 to 20, about 12 to 18, about 12 to 16, about 12 to 14, about 14 to 36, about 14 to 34, about 14 to 32, about 14 to 30, about 14 to 28, about 14 to 26, about 14 to 24, about 14 to 22, about 14 to 20, about 14 to 18, about 14 to 16, about 16 to 36, about 16 to 34, about 16 to 32, about 16 to 30, about 16 to 28, about 16 to 26, about 16 to 24, about 16 to 22, about 16 to 20, about 16 to 18, about 18 to 36, about 18 to 34, about 18 to 32, about 18 to 30, about 18 to 28, about 18 to 26, about 18 to 24, about 18 to 22, about 18 to 20, about 20 to 36, about 20 to 34, about 20 to 32, about 20 to 30, about 20 to 28, about 20 to 26, about 20 to 24, about 20 to 22, about 22 to 36, about 22 to 34, about 22 to 32, about 22 to 30, about 22 to 28, about 22 to 26, about 22 to 24, about 24 to 36, about 24 to 34, about 24 to 32, about 24 to 30, about 24 to 28, about 24 to 26, about 26 to 36, about 26 to 34, about 26 to 32, about 26 to 30, about 26 to 28, about 28 to 36, about 28 to 34, about 28 to 32, about 28 to 30, about 30 to 36, about 30 to 34, about 30 to 32, about 32 to 36, about 32 to 34, or about 34 to 36 months.

In some embodiments, the microbial compositions of the present disclosure are shelf stable at refrigeration temperatures (35-40° F.), at room temperature (68-72° F.), between 50-77° F., between −23-35° F., between 70-100T, or between 101-213° F. for a period of 1 to 36, 1 to 34, 1 to 32, 1 to 30, 1 to 28, 1 to 26, 1 to 24, 1 to 22, 1 to 20, 1 to 18, 1 to 16, 1 to 14, 1 to 12, 1 to 10, 1 to 8, 1 to 6, 1 to 4, 1 to 2, 4 to 36, 4 to 34, 4 to 32, 4 to 30, 4 to 28, 4 to 26, 4 to 24, 4 to 22, 4 to 20, 4 to 18, 4 to 16, 4 to 14, 4 to 12, 4 to 10, 4 to 8, 4 to 6, 6 to 36, 6 to 34, 6 to 32, 6 to 30, 6 to 28, 6 to 26, 6 to 24, 6 to 22, 6 to 20, 6 to 18, 6 to 16, 6 to 14, 6 to 12, 6 to 10, 6 to 8, 8 to 36, 8 to 34, 8 to 32, 8 to 30, 8 to 28, 8 to 26, 8 to 24, 8 to 22, 8 to 20, 8 to 18, 8 to 16, 8 to 14, 8 to 12, 8 to 10, 10 to 36, 10 to 34, 10 to 32, 10 to 30, 10 to 28, 10 to 26, 10 to 24, 10 to 22, 10 to 20, 10 to 18, 10 to 16, 10 to 14, 10 to 12, 12 to 36, 12 to 34, 12 to 32, 12 to 30, 12 to 28, 12 to 26, 12 to 24, 12 to 22, 12 to 20, 12 to 18, 12 to 16, 12 to 14, 14 to 36, 14 to 34, 14 to 32, 14 to 30, 14 to 28, 14 to 26, 14 to 24, 14 to 22, 14 to 20, 14 to 18, 14 to 16, 16 to 36, 16 to 34, 16 to 32, 16 to 30, 16 to 28, 16 to 26, 16 to 24, 16 to 22, 16 to 20, 16 to 18, 18 to 36, 18 to 34, 18 to 32, 18 to 30, 18 to 28, 18 to 26, 18 to 24, 18 to 22, 18 to 20, 20 to 36, 20 to 34, 20 to 32, 20 to 30, 20 to 28, 20 to 26, 20 to 24, 20 to 22, 22 to 36, 22 to 34, 22 to 32, 22 to 30, 22 to 28, 22 to 26, 22 to 24, 24 to 36, 24 to 34, 24 to 32, 24 to 30, 24 to 28, 24 to 26, 26 to 36, 26 to 34, 26 to 32, 26 to 30, 26 to 28, 28 to 36, 28 to 34, 28 to 32, 28 to 30, 30 to 36, 30 to 34, 30 to 32, 32 to 36, 32 to 34, or 34 to 36 months.

In some embodiments, the microbial compositions of the present disclosure are shelf stable at refrigeration temperatures (35-40° F.), at room temperature (68-72° F.), between 50-77° F., between −23-35° F., between 70-100T, or between 101-213° F. for a period of about 1 to 36, about 1 to 34, about 1 to 32, about 1 to 30, about 1 to 28, about 1 to 26, about 1 to 24, about 1 to 22, about 1 to 20, about 1 to 18, about 1 to 16, about 1 to 14, about 1 to 12, about 1 to 10, about 1 to 8, about 1 to 6, about 1 one 4, about 1 to 2, about 4 to 36, about 4 to 34, about 4 to 32, about 4 to 30, about 4 to 28, about 4 to 26, about 4 to 24, about 4 to 22, about 4 to 20, about 4 to 18, about 4 to 16, about 4 to 14, about 4 to 12, about 4 to 10, about 4 to 8, about 4 to 6, about 6 to 36, about 6 to 34, about 6 to 32, about 6 to 30, about 6 to 28, about 6 to 26, about 6 to 24, about 6 to 22, about 6 to 20, about 6 to 18, about 6 to 16, about 6 to 14, about 6 to 12, about 6 to 10, about 6 to 8, about 8 to 36, about 8 to 34, about 8 to 32, about 8 to 30, about 8 to 28, about 8 to 26, about 8 to 24, about 8 to 22, about 8 to 20, about 8 to 18, about 8 to 16, about 8 to 14, about 8 to 12, about 8 to 10, about 10 to 36, about 10 to 34, about 10 to 32, about 10 to 30, about 10 to 28, about 10 to 26, about 10 to 24, about 10 to 22, about 10 to 20, about 10 to 18, about 10 to 16, about 10 to 14, about 10 to 12, about 12 to 36, about 12 to 34, about 12 to 32, about 12 to 30, about 12 to 28, about 12 to 26, about 12 to 24, about 12 to 22, about 12 to 20, about 12 to 18, about 12 to 16, about 12 to 14, about 14 to 36, about 14 to 34, about 14 to 32, about 14 to 30, about 14 to 28, about 14 to 26, about 14 to 24, about 14 to 22, about 14 to 20, about 14 to 18, about 14 to 16, about 16 to 36, about 16 to 34, about 16 to 32, about 16 to 30, about 16 to 28, about 16 to 26, about 16 to 24, about 16 to 22, about 16 to 20, about 16 to 18, about 18 to 36, about 18 to 34, about 18 to 32, about 18 to 30, about 18 to 28, about 18 to 26, about 18 to 24, about 18 to 22, about 18 to 20, about 20 to 36, about 20 to 34, about 20 to 32, about 20 to 30, about 20 to 28, about 20 to 26, about 20 to 24, about 20 to 22, about 22 to 36, about 22 to 34, about 22 to 32, about 22 to 30, about 22 to 28, about 22 to 26, about 22 to 24, about 24 to 36, about 24 to 34, about 24 to 32, about 24 to 30, about 24 to 28, about 24 to 26, about 26 to 36, about 26 to 34, about 26 to 32, about 26 to 30, about 26 to 28, about 28 to 36, about 28 to 34, about 28 to 32, about 28 to 30, about 30 to 36, about 30 to 34, about 30 to 32, about 32 to 36, about 32 to 34, or about 34 to 36 years.

In some embodiments, the microbial compositions of the present disclosure are shelf stable at refrigeration temperatures (35-40° F.), at room temperature (68-72° F.), between 50-77° F., between −23-35° F., between 70-100T, or between 101-213° F. for a period of 1 to 36, 1 to 34, 1 to 32, 1 to 30, 1 to 28, 1 to 26, 1 to 24, 1 to 22, 1 to 20, 1 to 18, 1 to 16, 1 to 14, 1 to 12, 1 to 10, 1 to 8, 1 to 6, 1 to 4, 1 to 2, 4 to 36, 4 to 34, 4 to 32, 4 to 30, 4 to 28, 4 to 26, 4 to 24, 4 to 22, 4 to 20, 4 to 18, 4 to 16, 4 to 14, 4 to 12, 4 to 10, 4 to 8, 4 to 6, 6 to 36, 6 to 34, 6 to 32, 6 to 30, 6 to 28, 6 to 26, 6 to 24, 6 to 22, 6 to 20, 6 to 18, 6 to 16, 6 to 14, 6 to 12, 6 to 10, 6 to 8, 8 to 36, 8 to 34, 8 to 32, 8 to 30, 8 to 28, 8 to 26, 8 to 24, 8 to 22, 8 to 20, 8 to 18, 8 to 16, 8 to 14, 8 to 12, 8 to 10, 10 to 36, 10 to 34, 10 to 32, 10 to 30, 10 to 28, 10 to 26, 10 to 24, 10 to 22, 10 to 20, 10 to 18, 10 to 16, 10 to 14, 10 to 12, 12 to 36, 12 to 34, 12 to 32, 12 to 30, 12 to 28, 12 to 26, 12 to 24, 12 to 22, 12 to 20, 12 to 18, 12 to 16, 12 to 14, 14 to 36, 14 to 34, 14 to 32, 14 to 30, 14 to 28, 14 to 26, 14 to 24, 14 to 22, 14 to 20, 14 to 18, 14 to 16, 16 to 36, 16 to 34, 16 to 32, 16 to 30, 16 to 28, 16 to 26, 16 to 24, 16 to 22, 16 to 20, 16 to 18, 18 to 36, 18 to 34, 18 to 32, 18 to 30, 18 to 28, 18 to 26, 18 to 24, 18 to 22, 18 to 20, 20 to 36, 20 to 34, 20 to 32, 20 to 30, 20 to 28, 20 to 26, 20 to 24, 20 to 22, 22 to 36, 22 to 34, 22 to 32, 22 to 30, 22 to 28, 22 to 26, 22 to 24, 24 to 36, 24 to 34, 24 to 32, 24 to 30, 24 to 28, 24 to 26, 26 to 36, 26 to 34, 26 to 32, 26 to 30, 26 to 28, 28 to 36, 28 to 34, 28 to 32, 28 to 30, 30 to 36, 30 to 34, 30 to 32, 32 to 36, 32 to 34, or 34 to 36 years.

In some embodiments, the microbial compositions of the present disclosure are shelf stable at any of the disclosed temperatures and/or temperature ranges and spans of time at a relative humidity of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, or 98%.

In some embodiments, the microbial composition of the present disclosure possesses a water activity (a_{w}) of less than 0.750, 0.700, 0.650, 0.600, 0.550, 0.500, 0.475, 0.450, 0.425, 0.400, 0.375, 0.350, 0.325, 0.300, 0.275, 0.250, 0.225, 0.200, 0.190, 0.180, 0.170, 0.160, 0.150, 0.140, 0.130, 0.120, 0.110, 0.100, 0.095, 0.090, 0.085, 0.080, 0.075, 0.070, 0.065, 0.060, 0.055, 0.050, 0.045, 0.040, 0.035, 0.030, 0.025, 0.020, 0.015, 0.010, or 0.005.

In some embodiments, the microbial composition of the present disclosure possesses a water activity (a_{w}) of less than about 0.750, about 0.700, about 0.650, about 0.600, about 0.550, about 0.500, about 0.475, about 0.450, about 0.425, about 0.400, about 0.375, about 0.350, about 0.325, about 0.300, about 0.275, about 0.250, about 0.225, about 0.200, about 0.190, about 0.180, about 0.170, about 0.160, about 0.150, about 0.140, about 0.130, about 0.120, about 0.110, about 0.100, about 0.095, about 0.090, about 0.085, about 0.080, about 0.075, about 0.070, about 0.065, about 0.060, about 0.055, about 0.050, about 0.045, about 0.040, about 0.035, about 0.030, about 0.025, about 0.020, about 0.015, about 0.010, or about 0.005.

The water activity values are determined by the method of Saturated Aqueous Solutions (Multon, “Techniques d'Analyse E De Controle Dans Les Industries Agroalimentaires” APRIA (1981)) or by direct measurement using a viable Robotronic BT hygrometer or other hygrometer or hygroscope.

In some embodiments, the microbial composition comprises at least two different microbes, and wherein the at least two microbes are present in the composition at a ratio of 1:2, 1:3, 1:3, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:40, 1:50, 1:60, 1:100, 1:125, 1:150, 1:175, or 1:200 or the inverse thereof. In some embodiments, the microbial composition comprises at least three different microbes, and wherein the three microbes are present in the composition at a ratio of 1:2:1, 1:1:2, 2:2:1, 1:3:1, 1:1:3, 3:1:1, 3:3:1, 1:5:1, 1:1:5, 5:1:1, 5:5:1, or 1:5:5.

### Encapsulating Compositions

In some embodiments, any one of the microbial signalers, microbial compositions of the disclosure are encapsulated in an encapsulating composition. An encapsulating composition protects the microbes from external stressors. In some embodiments, external stressors include thermal and physical stressors. In some embodiments, external stressors include chemicals present in the compositions. Encapsulating compositions further create an environment that may be beneficial to the microbes, such as minimizing the oxidative stresses of an aerobic environment on anaerobic microbes. See Kalsta et al. (U.S. Pat. No. 5,104,662A), Ford (U.S. Pat. No. 5,733,568A), and Mosbach and Nilsson (U.S. Pat. No. 4,647,536A) for encapsulation compositions of microbes, and methods of encapsulating microbes.

In one embodiment, any one of the microbes, or microbial compositions of the present disclosure exhibits a thermal tolerance, which is used interchangeably with heat tolerance and heat resistance. In one embodiment, thermal tolerant compositions of the present disclosure are resistant to heat-killing and denaturation of the cell wall components and the intracellular environment.

In one embodiment, any one of the microbes, or microbial compositions of the present disclosure exhibits a pH tolerance, which is used interchangeably with acid tolerance and base tolerance. In one embodiment, pH tolerant compositions of the present disclosure are tolerant of the rapid swings in pH (high to low, low to high, high to neutral, low to neutral, neutral to high, and neutral to low) associated with one or more steps of preparing the composition.

In one embodiment, the encapsulation is a reservoir-type encapsulation. In one embodiment, the encapsulation is a matrix-type encapsulation. In one embodiment, the encapsulation is a coated matrix-type encapsulation. Burgain et al. (2011. J. Food Eng. 104:467-483) discloses numerous encapsulation embodiments and techniques.

In some embodiments, the microbes, microbial compositions of the present disclosure are encapsulated in one or more of the following: gellan gum, xanthan gum, K-Carrageenan, cellulose acetate phthalate, chitosan, starch, milk fat, whey protein, Ca-alginate, raftilose, raftiline, pectin, saccharide, glucose, maltodextrin, gum arabic, guar, seed flour, alginate, dextrins, dextrans, celluloase, gelatin, gelatin, albumin, casein, gluten, acacia gum, tragacanth, wax, paraffin, stearic acid, monodiglycerides, and diglycerides. In some embodiments, the compositions of the present disclosure are encapsulated by one or more of a polymer, carbohydrate, sugar, plastic, glass, polysaccharide, lipid, wax, oil, fatty acid, or glyceride. In one embodiment, the microbial composition is encapsulated by glucose. In one embodiment, the microbial composition is encapsulated by a glucose-containing composition. In one embodiment, formulations of the microbial composition comprise a glucose encapsulant. In one embodiment, formulations of the microbial composition comprise a glucose-encapsulated composition.

In some embodiments, the encapsulation of the microbes, or microbial compositions of the present disclosure is carried out by an extrusion, emulsification, coating, agglomeration, lyophilization, vitrification, foam drying, preservation by vaporization, vacuum-drying, or spray-drying.

In some embodiments, the encapsulated compositions of the present disclosure are vitrified. In some embodiments, encapsulation involves a process of drying a composition of the present disclosure in the presence of a substance which forms a glassy, amorphous solid state, a process known as vitrification, and in doing so encapsulates the composition. In some embodiments, the vitrified composition is protected from degradative conditions that would typically destroy or degrade microbes. Many common substances have the property of vitrification; that is, they will form a glassy solid state under certain conditions. Among these substances are several sugars, including sucrose and maltose, and other more complex compounds, such as polyvinylpyrrolidone (PVP). As any solution dries down, the molecules in the solution can either crystalize, or they can vitrify. A solute which has an extensive asymmetry may be a superior vitrifier, because of the hindrances to nucleation of crystals during drying. A substance that inhibits the crystallization of another substance may result in the combined substances forming a superior vitrification, such as raffinose in the presence of sucrose. See U.S. Pat. Nos. 5,290,765 and 9,469,835.

In some embodiments, a microbial composition is produced that is encapsulated in a vitrified substance. The vitrified composition may be created by selecting a mixture including cells; combining said mixture with sufficient quantity of one or more vitrifying solutes to protect said mixture during drying and to inhibit destructive reactions; and drying said combination by exposing said combination to a desiccant, or desiccating conditions, at a temperature above that which said combination will freeze and below that at which said vitrifying solutes achieve the vitrified state, at approximately normal atmospheric pressure, until said combination is substantially dry.

In one embodiment, the encapsulating composition comprises microcapsules having a multiplicity of liquid cores encapsulated in a solid shell material. For purposes of the disclosure, a “multiplicity” of cores is defined as two or more.

One category of fusible materials useful as encapsulating shell materials is that of waxes. Representative waxes contemplated for use herein are as follows: animal waxes, such as beeswax, lanolin, shell wax, and Chinese insect wax; vegetable waxes, such as carnauba, candelilla, bayberry, and sugar cane; mineral waxes, such as paraffin, microcrystalline petroleum, ozocerite, ceresin, and montan; synthetic waxes, such as low molecular weight polyolefin (e.g., CARBOWAX), and polyol ether-esters (e.g., sorbitol); Fischer-Tropsch process synthetic waxes; and mixtures thereof. Water-soluble waxes, such as CARBOWAX and sorbitol, are not contemplated herein if the core is aqueous. Still other fusible compounds useful herein are fusible natural resins, such as rosin, balsam, shellac, and mixtures thereof.

In some embodiments, the microbes, or microbial compositions of the present disclosure is embedded in a wax, such as the waxes described in the present disclosure. In some embodiments, the microbes or microbial composition is embedded in wax balls. In some embodiments, the microbes or microbial composition is already encapsulated prior to being embedded in wax balls. In some embodiments, the wax balls are 10 microns, 20 microns, 30 microns, 40 microns, 50 microns, 60 microns, 70 microns, 80 microns, 90 microns, 100 microns, 150 microns, 200 microns, 250 microns, 300 microns, 350 microns, 400 microns, 450 microns, 500 microns, 550 microns, 600 microns, 650 microns, 700 microns, 750 microns, 800 microns, 850 microns, 900 microns, 950 microns, or 1,000 microns in diameter.

In some embodiments, the wax balls are about 10 microbes, about 20 microns, about 30 microns, about 40 microns, about 50 microns, about 60 microns, about 70 microns, about 80 microns, about 90 microns, about 100 microns, about 150 microns, about 200 microns, about 250 microns, about 300 microns, about 350 microns, about 400 microns, about 450 microns, about 500 microns, about 550 microns, about 600 microns, about 650 microns, about 700 microns, about 750 microns, about 800 microns, about 850 microns, about 900 microns, about 950 microns, or about 1,000 microns in diameter.

In some embodiments, the wax balls are between 10-20 microns, 10-30 microns, 10-40 microns, 10-50 microns, 10-60 microns, 10-70 microns, 10-80 microns, 10-90 microns, 10-100 microns, 10-250 microns, 10-500 microns, 10-750 microns, 10-1,000 microns, 20-30 microns, 20-40 microns, 20-50 microns, 20-60 microns, 20-70 microns, 20-80 microns, 20-90 microns, 20-100 microns, 20-250 microns, 20-500 microns, 20-750 microns, 20-1,000 microns, 30-40 microns, 30-50 microns, 30-60 microns, 30-70 microns, 30-80 microns, 30-90 microns, 30-100 microns, 30-250 microns, 30-500 microns, 30-750 microns, 30-1,000 microns, 40-50 microns, 40-60 microns, 40-70 microns, 40-80 microns, 40-90 microns, 40-100 microns, 40-250 microns, 40-500 microns, 40-750 microns, 40-1,000 microns, 50-60 microns, 50-70 microns, 50-80 microns, 50-90 microns, 50-100 microns, 50-250 microns, 50-500 microns, 50-750 microns, 50-1,000 microns, 60-70 microns, 60-80 microns, 60-90 microns, 60-100 microns, 60-250 microns, 60-500 microns, 60-750 microns, 60-1,000 microns, 70-80 microns 70-90 microns, 70-90 microns, 70-100 microns, 70-250 microns, 70-500 microns, 70-750 microns, 70-1,000 microns, 80-90 microns, 80-100 microns, 80-250 microns, 80-500 microns, 80-500 microns, 80-750 microns, 80-1,000 microns, 90-100 microns, 90-250 microns, 90-500 microns, 90-750 microns, 90-1,000 microns, 100-250 microns, 100-500 microns, 100-750 microns, 100-1,000 microns, 250-500 microns, 250-750 microns, 250-1,000 microns, 500-750 microns, 500-1,000 microns, or 750-1,000 microns in diameter.

In some embodiments, the wax balls are between about 10-20 microns, about 10-30 microns, about 10-40 microns, about 10-50 microns, about 10-60 microns, about 10-70 microns, about 10-80 microns, about 10-90 microns, about 10-100 microns, about 10-250 microns, about 10-500 microns, about 10-750 microns, about 10-1,000 microns, about 20-30 microns, about 20-40 microns, about 20-50 microns, about 20-60 microns, about 20-70 microns, about 20-80 microns, about 20-90 microns, about 20-100 microns, about 20-250 microns, about 20-500 microns, about 20-750 microns, about 20-1,000 microns, about 30-40 microns, about 30-50 microns, about 30-60 microns, about 30-70 microns, about 30-80 microns, about 30-90 microns, about 30-100 microns, about 30-250 microns, about 30-500 microns, about 30-750 microns, about 30-1,000 microns, about 40-50 microns, about 40-60 microns, about 40-70 microns, about 40-80 microns, about 40-90 microns, about 40-100 microns, about 40-250 microns, about 40-500 microns, about 40-750 microns, about 40-1,000 microns, about 50-60 microns, about 50-70 microns, about 50-80 microns, about 50-90 microns, about 50-100 microns, about 50-250 microns, about 50-500 microns, about 50-750 microns, about 50-1,000 microns, about 60-70 microns, about 60-80 microns, about 60-90 microns, about 60-100 microns, about 60-250 microns, about 60-500 microns, about 60-750 microns, about 60-1,000 microns, about 70-80 microns about 70-90 microns, about 70-90 microns, about 70-100 microns, about 70-250 microns, about 70-500 microns, about 70-750 microns, about 70-1,000 microns, about 80-90 microns, about 80-100 microns, about 80-250 microns, about 80-500 microns, about 80-500 microns, about 80-750 microns, about 80-1,000 microns, about 90-100 microns, about 90-250 microns, about 90-500 microns, about 90-750 microns, about 90-1,000 microns, about 100-250 microns, about 100-500 microns, about 100-750 microns, about 100-1,000 microns, about 250-500 microns, about 250-750 microns, about 250-1,000 microns, about 500-750 microns, about 500-1,000 microns, or about 750-1,000 microns in diameter.

Various adjunct materials are contemplated for incorporation in fusible materials according to the present disclosure. For example, antioxidants, light stabilizers, dyes and lakes, flavors, essential oils, anti-caking agents, fillers, pH stabilizers, sugars (monosaccharides, disaccharides, trisaccharides, and polysaccharides) and the like can be incorporated in the fusible material in amounts which do not diminish its utility for the present disclosure.

The core material contemplated herein constitutes from about 0.1% to about 50%, about 1% to about 35%, or about 5% to about 30% by weight of the microcapsules. In some embodiments, the core material contemplated herein constitutes no more than about 30% by weight of the microcapsules. In some embodiments, the core material contemplated herein constitutes about 5% by weight of the microcapsules. The core material is contemplated as either a liquid or solid at contemplated storage temperatures of the microcapsules.

The cores may include other additives well-known in the agricultural art, including other potentially useful supplemental core materials will be apparent to those of ordinary skill in the art. Emulsifying agents may be employed to assist in the formation of stable emulsions. Representative emulsifying agents include glyceryl monostearate, polysorbate esters, ethoxylated mono- and diglycerides, and mixtures thereof.

For ease of processing, and particularly to enable the successful formation of a reasonably stable emulsion, the viscosities of the core material and the shell material should be similar at the temperature at which the emulsion is formed. In particular, the ratio of the viscosity of the shell to the viscosity of the core, expressed in centipoise or comparable units, and both measured at the temperature of the emulsion, should be from about 22:1 to about 1:1, desirably from about 8:1 to about 1:1, and preferably from about 3:1 to about 1:1. A ratio of 1:1 would be ideal, but a viscosity ratio within the recited ranges is useful.

Encapsulating compositions are not limited to microcapsule compositions as disclosed above. In some embodiments encapsulating compositions encapsulate the microbial compositions in an adhesive polymer that can be natural or synthetic without toxic effect. In some embodiments, the encapsulating composition may be a matrix selected from sugar matrix, gelatin matrix, polymer matrix, silica matrix, starch matrix, foam matrix, glass/glassy matrix etc. See Pirzio et al. (U.S. Pat. No. 7,488,503). In some embodiments, the encapsulating composition may be selected from polyvinyl acetates; polyvinyl acetate copolymers; ethylene vinyl acetate (EVA) copolymers; polyvinyl alcohols; polyvinyl alcohol copolymers; celluloses, including ethylcelluloses, methylcelluloses, hydroxymethylcelluloses, hydroxypropylcelluloses and carboxymethylcellulose; polyvinylpyrolidones; polysaccharides, including starch, modified starch, dextrins, maltodextrins, alginate and chitosans; monosaccharides; fats; fatty acids, including oils; proteins, including gelatin and zeins; gum arabics; shellacs; vinylidene chloride and vinylidene chloride copolymers; calcium lignosulfonates; acrylic copolymers; polyvinylacrylates; polyethylene oxide; acrylamide polymers and copolymers; polyhydroxyethyl acrylate, methylacrylamide monomers; and polychloroprene.

In some embodiments, the encapsulating compositions comprise at least one layer of encapsulation. In some embodiments, the encapsulating compositions comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 layers of encapsulation/encapsulants.

In some embodiments, the encapsulating compositions comprise at least two layers of encapsulation. In some embodiments, each layer of encapsulation confers a different characteristic to the composition. In some embodiments, no two consecutive layers confer the same characteristic. In some embodiments, at least one layer of the at least two layers of encapsulation confers thermostability, shelf stability, ultraviolet resistance, moisture resistance, hydrophobicity, hydrophilicity, lipophobicity, lipophilicity, pH stability, acid resistance, and base resistance.

In some embodiments, the encapsulating compositions comprise two layers of encapsulation; the first layer confers thermostability and/or shelf stability, and the second layer provides pH resistance. In some embodiments, the encapsulating layers confer a timed release of the microbial composition held in the center of the encapsulating layers. In some embodiments, the greater the number of layers confers a greater amount of time before the microbial composition is exposed, post administration.

In some embodiments, the encapsulating shell of the present disclosure can be up to 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 510 μm, 520 μm, 530 μm, 540 μm, 550 μm, 560 μm, 570 μm, 580 μm, 590 μm, 600 μm, 610 μm, 620 μm, 630 μm, 640 μm, 650 μm, 660 μm, 670 μm, 680 μm, 690 μm, 700 μm, 710 μm, 720 μm, 730 μm, 740 μm, 750 μm, 760 μm, 770 μm, 780 μm, 790 μm, 800 μm, 810 μm, 820 μm, 830 μm, 840 μm, 850 μm, 860 μm, 870 μm, 880 μm, 890 μm, 900 μm, 910 μm, 920 μm, 930 μm, 940 μm, 950 μm, 960 μm, 970 μm, 980 μm, 990 μm, 1000 μm, 1010 μm, 1020 μm, 1030 μm, 1040 μm, 1050 μm, 1060 μm, 1070 μm, 1080 μm, 1090 μm, 1100 μm, 1110 μm, 1120 μm, 1130 μm, 1140 μm, 1150 μm, 1160 μm, 1170 μm, 1180 μm, 1190 μm, 1200 μm, 1210 μm, 1220 μm, 1230 μm, 1240 μm, 1250 μm, 1260 μm, 1270 μm, 1280 μm, 1290 μm, 1300 μm, 1310 μm, 1320 μm, 1330 μm, 1340 μm, 1350 μm, 1360 μm, 1370 μm, 1380 μm, 1390 μm, 1400 μm, 1410 μm, 1420 μm, 1430 μm, 1440 μm, 1450 μm, 1460 μm, 1470 μm, 1480 μm, 1490 μm, 1500 μm, 1510 μm, 1520 μm, 1530 μm, 1540 μm, 1550 μm, 1560 μm, 1570 μm, 1580 μm, 1590 μm, 1600 μm, 1610 μm, 1620 μm, 1630 μm, 1640 μm, 1650 μm, 1660 μm, 1670 μm, 1680 μm, 1690 μm, 1700 μm, 1710 μm, 1720 μm, 1730 μm, 1740 μm, 1750 μm, 1760 μm, 1770 μm, 1780 μm, 1790 μm, 1800 μm, 1810 μm, 1820 μm, 1830 μm, 1840 μm, 1850 μm, 1860 μm, 1870 μm, 1880 μm, 1890 μm, 1900 μm, 1910 μm, 1920 μm, 1930 μm, 1940 μm, 1950 μm, 1960 μm, 1970 μm, 1980 μm, 1990 μm, 2000 μm, 2010 μm, 2020 μm, 2030 μm, 2040 μm, 2050 μm, 2060 μm, 2070 μm, 2080 μm, 2090 μm, 2100 μm, 2110 μm, 2120 μm, 2130 μm, 2140 μm, 2150 μm, 2160 μm, 2170 μm, 2180 μm, 2190 μm, 2200 μm, 2210 μm, 2220 μm, 2230 μm, 2240 μm, 2250 μm, 2260 μm, 2270 μm, 2280 μm, 2290 μm, 2300 μm, 2310 μm, 2320 μm, 2330 μm, 2340 μm, 2350 μm, 2360 μm, 2370 μm, 2380 μm, 2390 μm, 2400 μm, 2410 μm, 2420 μm, 2430 μm, 2440 μm, 2450 μm, 2460 μm, 2470 μm, 2480 μm, 2490 μm, 2500 μm, 2510 μm, 2520 μm, 2530 μm, 2540 μm, 2550 μm, 2560 μm, 2570 μm, 2580 μm, 2590 μm, 2600 μm, 2610 μm, 2620 μm, 2630 μm, 2640 μm, 2650 μm, 2660 μm, 2670 μm, 2680 μm, 2690 μm, 2700 μm, 2710 μm, 2720 μm, 2730 μm, 2740 μm, 2750 μm, 2760 μm, 2770 μm, 2780 μm, 2790 μm, 2800 μm, 2810 μm, 2820 μm, 2830 μm, 2840 μm, 2850 μm, 2860 μm, 2870 μm, 2880 μm, 2890 μm, 2900 μm, 2910 μm, 2920 μm, 2930 μm, 2940 μm, 2950 μm, 2960 μm, 2970 μm, 2980 μm, 2990 μm, or 3000 μm thick.

In some embodiments, the encapsulation composition of the present disclosure possesses a water activity (a_{w}) of less than 0.750, 0.700, 0.650, 0.600, 0.550, 0.500, 0.475, 0.450, 0.425, 0.400, 0.375, 0.350, 0.325, 0.300, 0.275, 0.250, 0.225, 0.200, 0.190, 0.180, 0.170, 0.160, 0.150, 0.140, 0.130, 0.120, 0.110, 0.100, 0.095, 0.090, 0.085, 0.080, 0.075, 0.070, 0.065, 0.060, 0.055, 0.050, 0.045, 0.040, 0.035, 0.030, 0.025, 0.020, 0.015, 0.010, or 0.005.

In some embodiments, the encapsulation composition of the present disclosure possesses a water activity (a_{w}) of less than about 0.750, about 0.700, about 0.650, about 0.600, about 0.550, about 0.500, about 0.475, about 0.450, about 0.425, about 0.400, about 0.375, about 0.350, about 0.325, about 0.300, about 0.275, about 0.250, about 0.225, about 0.200, about 0.190, about 0.180, about 0.170, about 0.160, about 0.150, about 0.140, about 0.130, about 0.120, about 0.110, about 0.100, about 0.095, about 0.090, about 0.085, about 0.080, about 0.075, about 0.070, about 0.065, about 0.060, about 0.055, about 0.050, about 0.045, about 0.040, about 0.035, about 0.030, about 0.025, about 0.020, about 0.015, about 0.010, or about 0.005.

In one embodiment, the microbe(s) are first dried by spray dry, lyophilization, or foam drying along with excipients that may include one or more sugars, sugar alcohols, disaccharides, trisaccharides, polysaccharides, salts, amino acids, amino acid salts, or polymers.

In some embodiments, the microbes or compositions comprising the microbes are milled to a size of 10 microns, 20 microns, 30 microns, 40 microns, 50 microns, 60 microns, 70 microns, 80 microns, 90 microns, 100 microns, 150 microns, 200 microns, 250 microns, 300 microns, 350 microns, 400 microns, 450 microns, 500 microns, 550 microns, 600 microns, 650 microns, 700 microns, 750 microns, 800 microns, 850 microns, 900 microns, 950 microns, or 1,000 microns in diameter.

In some embodiments, the microbes or compositions comprising the microbes are milled to a size of about 10 microns, about 20 microns, about 30 microns, about 40 microns, about 50 microns, about 60 microns, about 70 microns, about 80 microns, about 90 microns, about 100 microns, about 150 microns, about 200 microns, about 250 microns, about 300 microns, about 350 microns, about 400 microns, about 450 microns, about 500 microns, about 550 microns, about 600 microns, about 650 microns, about 700 microns, about 750 microns, about 800 microns, about 850 microns, about 900 microns, about 950 microns, or about 1,000 microns in diameter.

In some embodiments, the microbes or compositions comprising the microbes are milled to a size of between 10-20 microns, 10-30 microns, 10-40 microns, 10-50 microns, 10-60 microns, 10-70 microns, 10-80 microns, 10-90 microns, 10-100 microns, 10-250 microns, 10-500 microns, 10-750 microns, 10-1,000 microns, 20-30 microns, 20-40 microns, 20-50 microns, 20-60 microns, 20-70 microns, 20-80 microns, 20-90 microns, 20-100 microns, 20-250 microns, 20-500 microns, 20-750 microns, 20-1,000 microns, 30-40 microns, 30-50 microns, 30-60 microns, 30-70 microns, 30-80 microns, 30-90 microns, 30-100 microns, 30-250 microns, 30-500 microns, 30-750 microns, 30-1,000 microns, 40-50 microns, 40-60 microns, 40-70 microns, 40-80 microns, 40-90 microns, 40-100 microns, 40-250 microns, 40-500 microns, 40-750 microns, 40-1,000 microns, 50-60 microns, 50-70 microns, 50-80 microns, 50-90 microns, 50-100 microns, 50-250 microns, 50-500 microns, 50-750 microns, 50-1,000 microns, 60-70 microns, 60-80 microns, 60-90 microns, 60-100 microns, 60-250 microns, 60-500 microns, 60-750 microns, 60-1,000 microns, 70-80 microns 70-90 microns, 70-90 microns, 70-100 microns, 70-250 microns, 70-500 microns, 70-750 microns, 70-1,000 microns, 80-90 microns, 80-100 microns, 80-250 microns, 80-500 microns, 80-500 microns, 80-750 microns, 80-1,000 microns, 90-100 microns, 90-250 microns, 90-500 microns, 90-750 microns, 90-1,000 microns, 100-250 microns, 100-500 microns, 100-750 microns, 100-1,000 microns, 250-500 microns, 250-750 microns, 250-1,000 microns, 500-750 microns, 500-1,000 microns, or 750-1,000 microns in diameter.

In some embodiments, the microbes or compositions comprising the microbes are milled to a size of between about 10-20 microns, about 10-30 microns, about 10-40 microns, about 10-50 microns, about 10-60 microns, about 10-70 microns, about 10-80 microns, about 10-90 microns, about 10-100 microns, about 10-250 microns, about 10-500 microns, about 10-750 microns, about 10-1,000 microns, about 20-30 microns, about 20-40 microns, about 20-50 microns, about 20-60 microns, about 20-70 microns, about 20-80 microns, about 20-90 microns, about 20-100 microns, about 20-250 microns, about 20-500 microns, about 20-750 microns, about 20-1,000 microns, about 30-40 microns, about 30-50 microns, about 30-60 microns, about 30-70 microns, about 30-80 microns, about 30-90 microns, about 30-100 microns, about 30-250 microns, about 30-500 microns, about 30-750 microns, about 30-1,000 microns, about 40-50 microns, about 40-60 microns, about 40-70 microns, about 40-80 microns, about 40-90 microns, about 40-100 microns, about 40-250 microns, about 40-500 microns, about 40-750 microns, about 40-1,000 microns, about 50-60 microns, about 50-70 microns, about 50-80 microns, about 50-90 microns, about 50-100 microns, about 50-250 microns, about 50-500 microns, about 50-750 microns, about 50-1,000 microns, about 60-70 microns, about 60-80 microns, about 60-90 microns, about 60-100 microns, about 60-250 microns, about 60-500 microns, about 60-750 microns, about 60-1,000 microns, about 70-80 microns about 70-90 microns, about 70-90 microns, about 70-100 microns, about 70-250 microns, about 70-500 microns, about 70-750 microns, about 70-1,000 microns, about 80-90 microns, about 80-100 microns, about 80-250 microns, about 80-500 microns, about 80-500 microns, about 80-750 microns, about 80-1,000 microns, about 90-100 microns, about 90-250 microns, about 90-500 microns, about 90-750 microns, about 90-1,000 microns, about 100-250 microns, about 100-500 microns, about 100-750 microns, about 100-1,000 microns, about 250-500 microns, about 250-750 microns, about 250-1,000 microns, about 500-750 microns, about 500-1,000 microns, or about 750-1,000 microns in diameter.

In some embodiments, the microbes or compositions comprising the microbes are combined with a wax, fat, oil, fatty acid, or fatty alcohol, and spray congealed into beads of about 10 microns, about 20 microns, about 30 microns, about 40 microns, about 50 microns, about 60 microns, about 70 microns, about 80 microns, about 90 microns, about 100 microns, about 150 microns, about 200 microns, about 250 microns, about 300 microns, about 350 microns, about 400 microns, about 450 microns, about 500 microns, about 550 microns, about 600 microns, about 650 microns, about 700 microns, about 750 microns, about 800 microns, about 850 microns, about 900 microns, about 950 microns, or about 1,000 microns in diameter.

In some embodiments, the microbes or compositions comprising the microbes are combined with a wax, fat, oil, fatty acid, or fatty alcohol, and spray congealed into beads of between 10-20 microns, 10-30 microns, 10-40 microns, 10-50 microns, 10-60 microns, 10-70 microns, 10-80 microns, 10-90 microns, 10-100 microns, 10-250 microns, 10-500 microns, 10-750 microns, 10-1,000 microns, 20-30 microns, 20-40 microns, 20-50 microns, 20-60 microns, 20-70 microns, 20-80 microns, 20-90 microns, 20-100 microns, 20-250 microns, 20-500 microns, 20-750 microns, 20-1,000 microns, 30-40 microns, 30-50 microns, 30-60 microns, 30-70 microns, 30-80 microns, 30-90 microns, 30-100 microns, 30-250 microns, 30-500 microns, 30-750 microns, 30-1,000 microns, 40-50 microns, 40-60 microns, 40-70 microns, 40-80 microns, 40-90 microns, 40-100 microns, 40-250 microns, 40-500 microns, 40-750 microns, 40-1,000 microns, 50-60 microns, 50-70 microns, 50-80 microns, 50-90 microns, 50-100 microns, 50-250 microns, 50-500 microns, 50-750 microns, 50-1,000 microns, 60-70 microns, 60-80 microns, 60-90 microns, 60-100 microns, 60-250 microns, 60-500 microns, 60-750 microns, 60-1,000 microns, 70-80 microns 70-90 microns, 70-90 microns, 70-100 microns, 70-250 microns, 70-500 microns, 70-750 microns, 70-1,000 microns, 80-90 microns, 80-100 microns, 80-250 microns, 80-500 microns, 80-500 microns, 80-750 microns, 80-1,000 microns, 90-100 microns, 90-250 microns, 90-500 microns, 90-750 microns, 90-1,000 microns, 100-250 microns, 100-500 microns, 100-750 microns, 100-1,000 microns, 250-500 microns, 250-750 microns, 250-1,000 microns, 500-750 microns, 500-1,000 microns, or 750-1,000 microns in diameter.

In some embodiments, the microbes or compositions comprising the microbes are combined with a wax, fat, oil, fatty acid, or fatty alcohol, and spray congealed into beads of between about 10-20 microns, about 10-30 microns, about 10-40 microns, about 10-50 microns, about 10-60 microns, about 10-70 microns, about 10-80 microns, about 10-90 microns, about 10-100 microns, about 10-250 microns, about 10-500 microns, about 10-750 microns, about 10-1,000 microns, about 20-30 microns, about 20-40 microns, about 20-50 microns, about 20-60 microns, about 20-70 microns, about 20-80 microns, about 20-90 microns, about 20-100 microns, about 20-250 microns, about 20-500 microns, about 20-750 microns, about 20-1,000 microns, about 30-40 microns, about 30-50 microns, about 30-60 microns, about 30-70 microns, about 30-80 microns, about 30-90 microns, about 30-100 microns, about 30-250 microns, about 30-500 microns, about 30-750 microns, about 30-1,000 microns, about 40-50 microns, about 40-60 microns, about 40-70 microns, about 40-80 microns, about 40-90 microns, about 40-100 microns, about 40-250 microns, about 40-500 microns, about 40-750 microns, about 40-1,000 microns, about 50-60 microns, about 50-70 microns, about 50-80 microns, about 50-90 microns, about 50-100 microns, about 50-250 microns, about 50-500 microns, about 50-750 microns, about 50-1,000 microns, about 60-70 microns, about 60-80 microns, about 60-90 microns, about 60-100 microns, about 60-250 microns, about 60-500 microns, about 60-750 microns, about 60-1,000 microns, about 70-80 microns about 70-90 microns, about 70-90 microns, about 70-100 microns, about 70-250 microns, about 70-500 microns, about 70-750 microns, about 70-1,000 microns, about 80-90 microns, about 80-100 microns, about 80-250 microns, about 80-500 microns, about 80-500 microns, about 80-750 microns, about 80-1,000 microns, about 90-100 microns, about 90-250 microns, about 90-500 microns, about 90-750 microns, about 90-1,000 microns, about 100-250 microns, about 100-500 microns, about 100-750 microns, about 100-1,000 microns, about 250-500 microns, about 250-750 microns, about 250-1,000 microns, about 500-750 microns, about 500-1,000 microns, or about 750-1,000 microns in diameter.

In some embodiments, the microbes or compositions comprising the microbes are combined with a wax, fat, oil, fatty acid, or fatty alcohol as well as a water-soluble polymer, salt, polysaccharide, sugar, polypeptide, protein, or sugar alcohol and spray congealed into beads, the size of which are described herein. In some embodiments, the water-soluble polymer, salt, polysaccharide, sugar, or sugar alcohol serves as a disintegrant. In some embodiments, the disintegrant forms pores once the beads are dispersed in the soil.

In some embodiments, the composition of the water-soluble polymer, salt, polysaccharide, sugar, polypeptide, protein, or sugar alcohol is modified such that the disintegrant dissolves within 1, 5, 10, 15, 20, 25, 30, 35, 40, 45, 50, 55, 60 minutes of being administered. In some embodiments, the composition of the water-soluble polymer, salt, polysaccharide, sugar, polypeptide, protein, or sugar alcohol is modified such that the disintegrant dissolves within about 1, about 5, about 10, about 15, about 20, about 25, about 30, about 35, about 40, about 45, about 50, about 55, or about 60 minutes of being administered.

In some embodiments, the composition of the water-soluble polymer, salt, polysaccharide, sugar, polypeptide, protein, or sugar alcohol is modified such that the disintegrant dissolves within 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 hours of being administered. In some embodiments, the composition of the water-soluble polymer, salt, polysaccharide, sugar, polypeptide, protein, or sugar alcohol is modified such that the disintegrant dissolves within about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, or about 12 hours of being administered.

In some embodiments, the composition of the water-soluble polymer, salt, polysaccharide, sugar, polypeptide, protein, or sugar alcohol is modified such that the disintegrant dissolves at a temperature of at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50° C. In some embodiments, the composition of the water-soluble polymer, salt, polysaccharide, sugar, polypeptide, protein, or sugar alcohol is modified such that the disintegrant dissolves at a temperature of at least about 10, least about 11, least about 12, least about 13, least about 14, least about 15, least about 16, least about 17, least about 18, least about 19, least about 20, least about 21, least about 22, least about 23, least about 24, least about 25, least about 26, least about 27, least about 28, least about 29, least about 30, least about 31, least about 32, least about 33, least about 34, about 35, about 36, about 37, about 38, about 39, about 40, about 41, about 42, about 43, about 44, least about 45, least about 46, least about 47, least about 48, least about 49, or least about 50° C.

In some embodiments, the composition of the water-soluble polymer, salt, polysaccharide, sugar, polypeptide, protein, or sugar alcohol is modified such that the disintegrant dissolves at a pH of at least 3.8, 3.9, 4. 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 or 10.0. In some embodiments, the composition of the water-soluble polymer, salt, polysaccharide, sugar, polypeptide, protein, or sugar alcohol is modified such that the disintegrant dissolves at a pH of at least about 3.8, least about 3.9, least about 4. least about 4.1, least about 4.2, least about 4.3, least about 4.4, least about 4.5, least about 4.6, least about 4.7, least about 4.8, least about 4.9, least about 5.0, least about 5.1, least about 5.2, least about 5.3, least about 5.4, least about 5.5, least about 5.6, least about 5.7, least about 5.8, least about 5.9, least about 6.0, least about 6.2, least about 6.3, least about 6.4, least about 6.5, least about 6.6, least about 6.7, least about 6.8, least about 6.9, least about 7.0, least about 7.1, least about 7.2, least about 7.3, least about 7.4, least about 7.5, least about 7.6, least about 7.7, least about 7.8, least about 7.9, least about 8.0, least about 8.1, least about 8.2, least about 8.3, least about 8.4, least about 8.5, least about 8.6, least about 8.7, least about 8.8, least about 8.9, least about 9.0, least about 9.1, least about 9.2, least about 9.3, least about 9.4, least about 9.5, least about 9.6, least about 9.7, least about 9.8, least about 9.9, or least about 10.0.

In some embodiments, the microbes or compositions comprising the microbes are coated with a polymer, a polysaccharide, sugar, sugar alcohol, gel, wax, fat, fatty alcohol, or fatty acid

In some embodiments, the microbes or compositions comprising the microbes are coated with a polymer, a polysaccharide, sugar, sugar alcohol, gel, wax, fat, fatty alcohol, or fatty acid.

In some embodiments, the coating of the microbes or compositions comprising the microbes is modified such that the coating dissolves within 1, 5, 10, 15, 20, 25, 30, 35, 40, 45, 50, 55, 60 minutes of being administered. In some embodiments, the coating of the microbes or compositions comprising the microbes is modified such that the coating dissolves within about 1, about 5, about 10, about 15, about 20, about 25, about 30, about 35, about 40, about 45, about 50, about 55, or about 60 minutes of being administered.

In some embodiments, the coating of the microbes or compositions comprising the microbes is modified such that the coating dissolves within 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 hours of being administered. In some embodiments, the coating of the microbes or compositions comprising the microbes is modified such that the coating dissolves within about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, or about 12 hours of being administered.

In some embodiments, the coating of the microbes or compositions comprising the microbes is modified such that the coating dissolves at a temperature of at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50° C. In some embodiments, the coating of the microbes or compositions comprising the microbes is modified such that the coating dissolves at a temperature of at least about 10, least about 11, least about 12, least about 13, least about 14, least about 15, least about 16, least about 17, least about 18, least about 19, least about 20, least about 21, least about 22, least about 23, least about 24, least about 25, least about 26, least about 27, least about 28, least about 29, least about 30, least about 31, least about 32, least about 33, least about 34, about 35, about 36, about 37, about 38, about 39, about 40, about 41, about 42, about 43, about 44, least about 45, least about 46, least about 47, least about 48, least about 49, or least about 50° C.

In some embodiments, the coating of the microbes or compositions comprising the microbes is modified such that the coating dissolves at a pH of at least 3.8, 3.9, 4. 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 or 10.0. In some embodiments, the coating of the microbes or compositions comprising the microbes is modified such that the coating dissolves at a pH of at least about 3.8, least about 3.9, least about 4. least about 4.1, least about 4.2, least about 4.3, least about 4.4, least about 4.5, least about 4.6, least about 4.7, least about 4.8, least about 4.9, least about 5.0, least about 5.1, least about 5.2, least about 5.3, least about 5.4, least about 5.5, least about 5.6, least about 5.7, least about 5.8, least about 5.9, least about 6.0, least about 6.2, least about 6.3, least about 6.4, least about 6.5, least about 6.6, least about 6.7, least about 6.8, least about 6.9, least about 7.0, least about 7.1, least about 7.2, least about 7.3, least about 7.4, least about 7.5, least about 7.6, least about 7.7, least about 7.8, least about 7.9, least about 8.0, least about 8.1, least about 8.2, least about 8.3, least about 8.4, least about 8.5, least about 8.6, least about 8.7, least about 8.8, least about 8.9, least about 9.0, least about 9.1, least about 9.2, least about 9.3, least about 9.4, least about 9.5, least about 9.6, least about 9.7, least about 9.8, least about 9.9, or least about 10.0.

### Agricultural Applications of Microbial Compositions

The microbial compositions disclosed herein may be in the form of a dry powder, a slurry of powder and water, a granular material, or a flowable seed treatment. The compositions comprising microbe populations disclosed herein may be coated on a surface of a seed, and may be in liquid form.

The composition can be fabricated in bioreactors such as continuous stirred tank reactors, batch reactors, and on the farm. In some examples, compositions can be stored in a container, such as a jug or in mini bulk. In some examples, compositions may be stored within an object selected from the group consisting of a bottle, jar, ampule, package, vessel, bag, box, bin, envelope, carton, container, silo, shipping container, truck bed, and/or case.

In some examples, one or more compositions may be coated onto a seed. In some examples, one or more compositions may be coated onto a seedling. In some examples, one or more compositions may be coated onto a surface of a seed. In some examples, one or more compositions may be coated as a layer above a surface of a seed. In some examples, a composition that is coated onto a seed may be in liquid form, in dry product form, in foam form, in a form of a slurry of powder and water, or in a flowable seed treatment. In some examples, one or more compositions may be applied to a seed and/or seedling by spraying, immersing, coating, encapsulating, and/or dusting the seed and/or seedling with the one or more compositions. In some examples, multiple bacteria or bacterial populations can be coated onto a seed and/or a seedling of the plant. In some examples, at least two, at least three, at least four, at least five, at least six, at least seven, at least eight, at least nine, at least ten, or more than ten bacteria of a bacterial combination can be selected from any one of the microbes disclosed herein.

Examples of compositions may include seed coatings for commercially important agricultural crops, for example, sorghum, canola, tomato, strawberry, barley, rice, maize, and wheat. Examples of compositions can also include seed coatings for corn, soybean, canola, sorghum, potato, rice, vegetables, cereals, and oilseeds. Seeds as provided herein can be genetically modified organisms (GMO), non-GMO, organic, or conventional. In some examples, compositions may be sprayed on the plant aerial parts, or applied to the roots by inserting into furrows in which the plant seeds are planted, watering to the soil, or dipping the roots in a suspension of the composition. In some examples, compositions may be dehydrated in a suitable manner that maintains cell viability and the ability to artificially inoculate and colonize host plants.

The bacterial species may be present in compositions at a concentration of between 10₈ to 10₁₀ CFU/ml. In some examples, compositions may be supplemented with trace metal ions, such as molybdenum ions, iron ions, manganese ions, or combinations of these ions. The concentration of ions in examples of compositions as described herein may between about 0.1 mM and about 50 mM. Some examples of compositions may also be formulated with a carrier, such as beta-glucan, carboxylmethyl cellulose (CMC), bacterial extracellular polymeric substance (EPS), sugar, animal milk, or other suitable carriers. In some examples, peat or planting materials can be used as a carrier, or biopolymers in which a composition is entrapped in the biopolymer can be used as a carrier. The compositions comprising the bacterial populations described herein can improve plant traits, such as promoting plant growth, maintaining high chlorophyll content in leaves, increasing fruit or seed numbers, and increasing fruit or seed unit weight.

The compositions comprising the bacterial populations described herein may be coated on to the surface of a seed. As such, compositions comprising a seed coated with one or more bacteria described herein are also contemplated. The seed coating can be formed by mixing the bacterial population with a porous, chemically inert granular carrier. Alternatively, the compositions may be inserted directly into the furrows into which the seed is planted or sprayed onto the plant leaves or applied by dipping the roots into a suspension of the composition. An effective amount of the composition can be used to populate the sub-soil region adjacent to the roots of the plant with viable bacterial growth, or populate the leaves of the plant with viable bacterial growth. In general, an effective amount is an amount sufficient to result in plants with improved traits (e.g. a desired level of nitrogen fixation).

In some embodiments, the microbes, or microbial compositions of the present disclosure may be formulated using an agriculturally acceptable carrier. The formulation useful for these embodiments may include at least one member selected from the group consisting of a tackifier, a microbial stabilizer, a fungicide, a biopesticide, an antibacterial agent, a preservative, a stabilizer, a surfactant, an anti-complex agent, an herbicide, a nematicide, an insecticide, a plant growth regulator, a fertilizer, a rodenticide, a dessicant, a bactericide, a nutrient, a hormone, or any combination thereof. In some examples, compositions may be shelf-stable. For example, any of the compositions described herein can include an agriculturally acceptable carrier (e.g., one or more of a fertilizer such as a nonnaturally occurring fertilizer, an adhesion agent such as a non-naturally occurring adhesion agent, and a pesticide such as a non-naturally occurring pesticide). A non-naturally occurring adhesion agent can be, for example, a polymer, copolymer, or synthetic wax. For example, any of the coated seeds, seedlings, or plants described herein can contain such an agriculturally acceptable carrier in the seed coating. In any of the compositions or methods described herein, an agriculturally acceptable carrier can be or can include a non-naturally occurring compound (e.g., a non-naturally occurring fertilizer, a non-naturally occurring adhesion agent such as a polymer, copolymer, or synthetic wax, or a non-naturally occurring pesticide). Non-limiting examples of agriculturally acceptable carriers are described below. Additional examples of agriculturally acceptable carriers are known in the art.

In some cases, the microbes, or microbial compositions of the present disclosure may be mixed with an agriculturally acceptable carrier. The carrier can be a solid carrier or liquid carrier, and in various forms including microspheres, powders, emulsions and the like. The carrier may be any one or more of a number of carriers that confer a variety of properties, such as increased stability, wettability, or dispersability. Wetting agents such as natural or synthetic surfactants, which can be nonionic or ionic surfactants, or a combination thereof can be included in the composition. Water-in-oil emulsions can also be used to formulate a composition that includes the isolated bacteria (see, for example, U.S. Pat. No. 7,485,451). Suitable formulations that may be prepared include wettable powders, granules, gels, agar strips or pellets, thickeners, and the like, microencapsulated particles, and the like, liquids such as aqueous flowables, aqueous suspensions, water-in-oil emulsions, etc. The formulation may include grain or legume products, for example, ground grain or beans, broth or flour derived from grain or beans, starch, sugar, or oil.

In some embodiments, the agricultural carrier may be soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the bacteria, such as barley, rice, or other biological materials such as seed, plant parts, sugar cane bagasse, hulls or stalks from grain processing, ground plant material or wood from building site refuse, sawdust or small fibers from recycling of paper, fabric, or wood.

For example, a fertilizer can be used to help promote the growth or provide nutrients to a seed, seedling, or plant. Non-limiting examples of fertilizers include nitrogen, phosphorous, potassium, calcium, sulfur, magnesium, boron, chloride, manganese, iron, zinc, copper, molybdenum, and selenium (or a salt thereof). Additional examples of fertilizers include one or more amino acids, salts, carbohydrates, vitamins, glucose, NaCl, yeast extract, NH₄H₂PO₄, (NH₄)₂SO₄, glycerol, valine, L-leucine, lactic acid, propionic acid, succinic acid, malic acid, citric acid, KH tartrate, xylose, lyxose, and lecithin. In one embodiment, the formulation can include a tackifier or adherent (referred to as an adhesive agent) to help bind other active agents to a substance (e.g., a surface of a seed). Such agents are useful for combining bacteria with carriers that can contain other compounds (e.g., control agents that are not biologic), to yield a coating composition. Such compositions help create coatings around the plant or seed to maintain contact between the microbe and other agents with the plant or plant part. In one embodiment, adhesives are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers.

In some embodiments, the adhesives can be, e.g. a wax such as carnauba wax, beeswax, Chinese wax, shellac wax, spermaceti wax, candelilla wax, castor wax, ouricury wax, and rice bran wax, a polysaccharide (e.g., starch, dextrins, maltodextrins, alginate, and chitosans), a fat, oil, a protein (e.g., gelatin and zeins), gum arables, and shellacs. Adhesive agents can be nonnaturally occurring compounds, e.g., polymers, copolymers, and waxes. For example, nonlimiting examples of polymers that can be used as an adhesive agent include: polyvinyl acetates, polyvinyl acetate copolymers, ethylene vinyl acetate (EVA) copolymers, polyvinyl alcohols, polyvinyl alcohol copolymers, celluloses (e.g., ethylcelluloses, methylcelluloses, hydroxymethylcelluloses, hydroxypropylcelluloses, and carboxymethylcelluloses), polyvinylpyrolidones, vinyl chloride, vinylidene chloride copolymers, calcium lignosulfonates, acrylic copolymers, polyvinylacrylates, polyethylene oxide, acylamide polymers and copolymers, polyhydroxyethyl acrylate, methylacrylamide monomers, and polychloroprene.

In some examples, one or more of the adhesion agents, anti-fungal agents, growth regulation agents, and pesticides (e.g., insecticide) are non-naturally occurring compounds (e.g., in any combination). Additional examples of agriculturally acceptable carriers include dispersants (e.g., polyvinylpyrrolidone/vinyl acetate PVPIVA S-630), surfactants, binders, and filler agents. The formulation can also contain a surfactant. Non-limiting examples of surfactants include nitrogen-surfactant blends such as Prefer 28 (Cenex), Surf-N (US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

In certain cases, the formulation includes a microbial stabilizer. Such an agent can include a desiccant, which can include any compound or mixture of compounds that can be classified as a desiccant regardless of whether the compound or compounds are used in such concentrations that they in fact have a desiccating effect on a liquid inoculant. Such desiccants are ideally compatible with the bacterial population used, and should promote the ability of the microbial population to survive application on the seeds and to survive desiccation. Examples of suitable desiccants include one or more of trehalose, sucrose, glycerol, and Methylene glycol. Other suitable desiccants include, but are not limited to, non-reducing sugars and sugar alcohols (e.g., mannitol or sorbitol). The amount of desiccant introduced into the formulation can range from about 5% to about 50% by weight/volume, for example, between about 10% to about 40%, between about 15% to about 35%, or between about 20% to about 30%. In some cases, it is advantageous for the formulation to contain agents such as a fungicide, a biopesticide, an antibacterial agent, an herbicide, a nematicide, an insecticide, a plant growth regulator, a rodenticide, bactericide, or a nutrient. In some examples, agents may include protectants that provide protection against seed surface-borne pathogens. In some examples, protectants may provide some level of control of soil-borne pathogens. In some examples, protectants may be effective predominantly on a seed surface.

### Methods of Improving Soil and Promoting Plant Growth

The disclosure provides methods of producing an improved soil for plant growth, comprising: applying any one of the compositions disclosed herein to soil, thereby producing the improved soil for plant growth. In some embodiments, the method comprises allowing a plant to grow in the improved soil.

In some embodiments, the growth of the plant is more enhanced in the improved soil, as compared to the growth of the plant in a negative control soil, wherein the composition is not applied to the negative control soil. In some embodiments, the method inhibits a plant pathogen in the improved soil.

In some embodiments, the growth of the plant is more enhanced in the improved soil, as compared to the growth of the plant in a negative control soil, wherein the composition is not applied to the negative control soil. In some embodiments, the method inhibits a plant pathogen in the improved soil. In some embodiments, the inhibition of a plant pathogen in the improved soil is higher than in a negative control soil, wherein the composition is not applied to the negative control soil. In some embodiments, the inhibition of a plant pathogen in the improved soil is at least about 1% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%, including all values and subranges that lie therebetween) higher than in a negative control soil, wherein the composition is not applied to the negative control soil.

In some embodiments, the number, density and/or function of a plant pathogen in the improved soil is lower than in a negative control soil, wherein the composition is not applied to the negative control soil. In some embodiments, the number, density and/or function of a plant pathogen in the improved soil is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%, including all values and subranges that lie therebetween) lower than in a negative control soil, wherein the composition is not applied to the negative control soil. In some embodiments, the number, density and/or function of a plant pathogen in the improved soil is at least about 5% lower than in a negative control soil, wherein the composition is not applied to the negative control soil.

In some embodiments, the method increases the amount and/or concentration of soluble zinc in the improved soil. In some embodiments, the amount and/or concentration of soluble zinc in the improved soil is higher than in a negative control soil, wherein the composition is not applied to the negative control soil. In some embodiments, the amount and/or concentration of soluble zinc in the improved soil is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween) higher than in a negative control soil, wherein the composition is not applied to the negative control soil. In some embodiments, the amount and/or concentration of soluble zinc in the improved soil is higher than in a negative control soil, wherein the composition is not applied to the negative control soil. In some embodiments, the amount and/or concentration of soluble zinc in the improved soil is at least about 5% higher than in a negative control soil, wherein the composition is not applied to the negative control soil.

In some embodiments, the method increases the amount and/or concentration of soluble phosphate in the soil. In some embodiments, the amount and/or concentration of soluble phosphate in the improved soil is higher than in a negative control soil, wherein the composition is not applied to the negative control soil. In some embodiments, the amount and/or concentration of soluble phosphate in the improved soil is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween) higher than in a negative control soil, wherein the composition is not applied to the negative control soil. In some embodiments, the amount and/or concentration of soluble phosphate in the improved soil is at least about 5% higher than in a negative control soil, wherein the composition is not applied to the negative control soil.

In some embodiments, the growth of the plant is more enhanced in the improved soil, as compared to the growth of the plant in a comparator control soil, wherein the at least one target microbe is applied to comparator control soil and the at least one microbial signaler is not applied to the comparator control soil. In some embodiments, the growth of the plant is at least about 1% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween) higher in the improved soil, as compared to the growth of the plant in a comparator control soil, wherein the at least one target microbe is applied to comparator control soil and the at least one microbial signaler is not applied to the comparator control soil.

The parameter used to measure plant growth is not limited. For instance, plant growth may be measured using the following exemplary parameters: fresh aboveground biomass, dry aboveground biomass, the number or frequency of plants that reach a particular vegetative growth stage in a given time period (e.g. the number or frequency of plants that reach vegetative growth stage 4 in corn), internodal length, root length, fresh belowground biomass, dry belowground biomass, increase in average growth stage (e.g. among soybean plants), improved plant vigor, reduction in frequency of purple coloration (e.g. associated with nutrient stress), increase in frequency of healthy unifolate leaves (e.g. in soybean), plant height, and reduction in frequency of chlorotic leaves.

In some embodiments, the inhibition of a plant pathogen in the improved soil is higher than in a comparator control soil, wherein the at least one target microbe is applied to comparator control soil and the at least one microbial signaler is not applied to the comparator control soil. In some embodiments, the inhibition of a plant pathogen in the improved soil is at least about 1% higher than in a comparator control soil, wherein the at least one target microbe is applied to comparator control soil and the at least one microbial signaler is not applied to the comparator control soil.

In some embodiments, the method increases the amount and/or concentration of soluble zinc in the improved soil. In some embodiments, the amount and/or concentration of soluble zinc in the improved soil is higher than in a comparator control soil, wherein the at least one target microbe is applied to comparator control soil and the at least one microbial signaler is not applied to the comparator control soil. In some embodiments, the amount and/or concentration of soluble zinc in the improved soil is at least about 1% higher than in a comparator control soil, wherein the at least one target microbe is applied to comparator control soil and the at least one microbial signaler is not applied to the comparator control soil.

In some embodiments, the method increases the amount and/or concentration of soluble phosphate in the soil. In some embodiments, the amount and/or concentration of soluble phosphate in the improved soil is higher than in a comparator control soil, wherein the at least one target microbe is applied to comparator control soil and the at least one microbial signaler is not applied to the comparator control soil. In some embodiments, the amount and/or concentration of soluble phosphate in the improved soil is at least about 1% higher than in a comparator control soil, wherein the at least one target microbe is applied to comparator control soil and the at least one microbial signaler is not applied to the comparator control soil.

In some embodiments, the method improves the suppression of a disease associated with, promoted by, or caused by a pathogen in the plant. In some embodiments, the suppression of the disease associated with, promoted by, or caused by the pathogen in the plant is higher than in a comparator control soil, wherein the at least one target microbe is applied to comparator control soil and the at least one microbial signaler is not applied to the comparator control soil. In some embodiments, the suppression of the disease associated with, promoted by, or caused by the pathogen in the plant is at least about 1% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween) higher than in a comparator control soil, wherein the at least one target microbe is applied to comparator control soil and the at least one microbial signaler is not applied to the comparator control soil.

In some embodiments, the method increases the above-ground biomass of the plant. In some embodiments, the above-ground biomass of the plant is higher than in a comparator control soil, wherein the at least one target microbe is applied to comparator control soil and the at least one microbial signaler is not applied to the comparator control soil. In some embodiments, the above-ground biomass of the plant is at least about 1% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween) higher than in a comparator control soil, wherein the at least one target microbe is applied to comparator control soil and the at least one microbial signaler is not applied to the comparator control soil.

In some embodiments, the method increases the below-ground biomass of the plant. In some embodiments, the below-ground biomass of the plant is higher than in a comparator control soil, wherein the at least one target microbe is applied to comparator control soil and the at least one microbial signaler is not applied to the comparator control soil. In some embodiments, the below-ground biomass of the plant is at least about 1% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween) higher than in a comparator control soil, wherein the at least one target microbe is applied to comparator control soil and the at least one microbial signaler is not applied to the comparator control soil.

In some embodiments, the composition is applied before planting, after plant germination, as a seed treatment, as a spray, and/or as a soil drench.

In some embodiments, the plant pathogen belongs to one of the following genera: Pseudomonas, Erwinia, Raltsonia, Rhizomonas, Agrobacterium, Streptomyces, Bacillus, Sclerotium, Rhizoctonia, Fusarium, Pythium, Phytophthora, Synchytrium, Rhizopus, Alternaria, Macrophomina, Drechslera, Bipolaris, Curvularia, Phomopsis, Caloscypha fulgens, usarium circinatum, Fusarium oxysporum, Fusarium moniliforme var. moniliforme, Lasiodiplodia theobromae, Sirococcus conigenus, Diplodia pinea, Ustilago nuda, Pyrenophora graminea, Pyrenophora teres, Xanthomonas translucens, Pseudomonas syringae, Fusarium graminearum, Bipolaris sorokiniana, Xanthomonas campestris, Aciculosporium, Mycosphaerella, Ceratobasidium, Albugo, Alternaria, Myrothecium, Cochliobolus, Hyaloperonospora, Alveopora, Neonectria, Colletotrichum, Peronospora, Cadophora, Oculimacula, Curvularia, Phytophthora, Calyptella Omphalotus, Cylindrocladiella, Plasmopara, Chrysomyxa, Peyronellaea, Fusarium, Pythiogeton, Cladophialophora, Phaeoacremonium, Heterobasidion, Pythium, Coleosporium, Pseudocercospora, Magnaporthe, Colletotrichum, Puccinia, Microdochium, Corynespora, Pucciniastrum, Olpidium, Craterocolla, Pseudotetraploa, Phoma, Cronartium, Septoria, Plectosphaerella Didymella, Sphacelotheca, Pyrenochaeta, Drechslera, Spongipellis, Rhizoctonia, Endocronartium, Stenocarpella, Setophoma, Entyloma, Sydowia, Spongospora, Fomitopsis, Taphrina, Thielaviopsis, Fusarium, Tritirachium, Typhula, Ganoderma, Urocystis, Verticillium, Hypohelion, Ustilago, Waitea, Itersonilia, Venturia, Leptosphaerulina, Verticillium, and Monilinia.

In some embodiments, the plant pathogen: (a) belongs to the genus Colletotrichum, Fusarium, Verticillium, Phytophthora, Cercospora, Rhizoctonia, Septoria, Pythium, Aphanomyces, Bremia, Monosporascus, Sclerotinia, or Stagnospora; or (b) is a member of Plasmodiophoromyces, Zygomycetes, Oomycetes, Ascomycetes, and Basidiomycetes; or (c) is Rusarium Rhizoctonia, Plasmodiophora brassicae, Spongospora subterranean, Macrophomina phaseolina, Monosporascus cannonballus, Pythium aphanidermatum, or Sclerotium rolfsii.

In some embodiments, the plant pathogen is a species of a genus selected from the group consisting of Erwinia, Rhizomonas, Streptomyces scabies, Pseudomonas, and Xanthomonas.

In some embodiments, the microbial composition is administered in a dose volume comprising a total of, or at least 0.5 ml, 1 ml, 2 ml, 3 ml, 4 ml, 5 ml, 6 ml, 7 ml, 8 ml, 9 ml, 10 ml, 11 ml, 12 ml, 13 ml, 14 ml, 15 ml, 16 ml, 17 ml, 18 ml, 19 ml, 20 ml, 21 ml, 22 ml, 23 ml, 24 ml, 25 ml, 26 ml, 27 ml, 28 ml, 29 ml, 30 ml, 31 ml, 32 ml, 33 ml, 34 ml, 35 ml, 36 ml, 37 ml, 38 ml, 39 ml, 40 ml, 41m, 42 ml, 43 ml, 44 ml, 45 ml, 46 ml, 47 ml, 48 ml, 49 ml, 50 ml, 60 ml, 70 ml, 80 ml, 90 ml, 100 ml, 200 ml, 300 ml, 400 ml, 500 ml, 600 ml, 700 ml, 800 ml, 900 ml, or 1,000 ml.

In some embodiments, the microbial composition is administered in a dose comprising a total of, or at least, 10¹⁸, 10¹⁷, 10¹⁶, 10¹⁵, 10¹⁴, 10¹³, 10¹², 10¹¹, 10¹⁰, 10⁹, 10⁸, 10⁷, 10⁶, 10⁵, 10⁴, 10³, or 10² microbial cells. In some embodiments, these microbial cells are quantified by colony forming units (CFUs).

In some embodiments, the dose of the microbial composition is administered such that there exists 10² to 10¹², 10³ to 10¹², 10⁴ to 10¹², 10⁵ to 10¹², 10⁶ to 10¹², 10⁷ to 10¹², 10⁸ to 10¹², 10⁹ to 10¹², 10¹⁰ to 10¹², 10¹¹ to 10¹², 10² to 10¹¹, 10³ to 10¹¹, 10⁴ to 10¹¹, 10⁵ to 10¹¹, 10⁶ to 10¹¹, 10⁷ to 10¹¹, 10⁸ to 10¹¹, 10⁹ to 10¹¹, 10¹⁰ to 10¹¹, 10² to 10¹⁰, 10³ to 10¹⁰, 10⁴ to 10¹⁰, 10⁵ to 10¹⁰, 10⁶ to 10¹⁰, 10⁷ to 10¹⁰, 10⁸ to 10¹⁰, 10⁹ to 10¹⁰, 10² to 10⁹, 10³ to 10⁹, 10⁴ to 10⁹, 10⁵ to 10⁹, 10⁶ to 10⁹, 10⁷ to 10⁹, 10⁸ to 10⁹, 10² to 10⁸, 10³ to 10⁸, 10⁴ to 10⁸, 10⁵ to 10⁸, 10⁶ to 10⁸, 10⁷ to 10⁸, 10² to 10⁷, 10³ to 10⁷, 10⁴ to 10⁷, 10⁵ to 10⁷, 10⁶ to 10⁷, 10² to 10⁶, 10³ to 10⁶, 10⁴ to 10⁶, 10⁵ to 10⁶, 10² to 10⁵, 10³ to 10⁵, 10⁴ to 10⁵, 10² to 10⁴, 10³ to 10⁴, 10² to 10³, 10¹², 10¹¹, 10¹⁰, 10⁹, 10⁸, 10⁷, 10⁶, 10⁵, 10⁴, 10³, or 10² total microbial cells per gram or milliliter of the composition.

In some embodiments, the administered dose of the microbial composition comprises 10² to 10¹⁸, 10³ to 10¹⁸, 10⁴ to 10¹⁸, 10⁵ to 10¹⁸, 10⁶ to 10¹⁸, 10⁷ to 10¹⁸, 10⁸ to 10¹⁸, 10⁹ to 10¹⁸, 10¹⁰ to 10¹⁸, 10¹¹ to 10¹⁸, 10¹² to 10¹⁸, 10¹³ to 10¹⁸, 10¹⁴ to 10¹⁸, 10¹⁵ to 10¹⁸, 10¹⁶ to 10¹⁸, 10¹⁷ to 10¹⁸, 10² to 10¹², 10³ to 10¹², 10⁴ to 10¹², 10⁵ to 10¹², 10⁶ to 10¹², 10⁷ to 10¹², 10⁸ to 10¹², 10⁹ to 10¹², 10¹⁰ to 10¹², 10¹¹ to 10¹², 10² to 10¹¹, 10³ to 10¹¹, 10⁴ to 10¹¹, 10⁵ to 10¹¹, 10⁶ to 10¹¹, 10⁷ to 10¹¹, 10⁸ to 10¹¹, 10⁹ to 10¹¹, 10¹⁰ to 10¹¹, 10² to 10¹⁰, 10³ to 10¹⁰, 10⁴ to 10¹⁰, 10⁵ to 10¹⁰, 10⁶ to 10¹⁰, 10⁷ to 10¹⁰, 10⁸ to 10¹⁰, 10⁹ to 10¹⁰, 10² to 10⁹, 10³ to 10⁹, 10⁴ to 10⁹, 10⁵ to 10⁹, 10⁶ to 10⁹, 10⁷ to 10⁹, 10⁸ to 10⁹, 10² to 10⁸, 10³ to 10⁸, 10⁴ to 10⁸, 10⁵ to 10⁸, 10⁶ to 10⁸, 10⁷ to 10⁸, 10² to 10⁷, 10³ to 10⁷, 10⁴ to 10⁷, 10⁵ to 10⁷, 10⁶ to 10⁷, 10² to 10⁶, 10³ to 10⁶, 10⁴ to 10⁶, 10⁵ to 10⁶, 10² to 10⁵, 10³ to 10⁵, 10⁴ to 10⁵, 102 to 10⁴, 10³ to 10⁴, 10² to 10³, 10¹⁸, 10¹⁷, 10¹⁶, 10¹⁵, 10¹⁴, 10¹³, 10¹², 10¹¹, 10¹⁰, 10⁹, 10⁸, 10⁷, 10⁶, 10⁵, 10⁴, 10³, or 10² total microbial cells.

In some embodiments, the composition is administered 1 or more times per month. In some embodiments, the composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 times per week.

In some embodiments, the microbial composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 times per month.

In some embodiments, the microbial composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 times per year.

In some embodiments, the microbial cells can be coated freely onto any number of compositions or they can be formulated in a liquid or solid composition before being coated onto a composition. For example, a solid composition comprising the microorganisms can be prepared by mixing a solid carrier with a suspension of the spores until the solid carriers are impregnated with the spore or cell suspension. This mixture can then be dried to obtain the desired particles.

In some embodiments, it is contemplated that the solid or liquid microbial compositions of the present disclosure further contain functional agents e.g., activated carbon, minerals, vitamins, and other agents capable of improving the quality of the products or a combination thereof.

In some embodiments, the microbes or microbial compositions of the present disclosure exhibit a synergistic effect, on one or more of the traits described herein, in the presence of one or more of the microbes or microbial compositions coming into contact with one another.

The synergistic effect obtained by the taught methods can be quantified, for example, according to Colby's formula (i.e., (E)=X+Y−(X*Y/100)). See Colby, R. S., “Calculating Synergistic and Antagonistic Responses of Herbicide Combinations,” 1967. Weeds. Vol. 15, pp. 20-22, incorporated herein by reference in its entirety. Thus, “synergistic” is intended to reflect an outcome/parameter/effect that has been increased by more than an additive amount.

In some embodiments, the microbes or microbial compositions are administered in a time-release fashion between 1 to 5, 1 to 10, 1 to 15, 1 to 20, 1 to 24, 1 to 25, 1 to 30, 1 to 35, 1 to 40, 1 to 45, 1 to 50, 1 to 55, 1 to 60, 1 to 65, 1 to 70, 1 to 75, 1 to 80, 1 to 85, 1 to 90, 1 to 95, or 1 to 100 hours.

In some embodiments, the microbes or microbial compositions are administered in a time-release fashion between 1 to 2, 1 to 3, 1 to 4, 1 to 5, 1 to 6, 1 to 7, 1 to 8, 1 to 9, 1 to 10, 1 to 11, 1 to 12, 1 to 13, 1 to 14, 1 to 15, 1 to 16, 1 to 17, 1 to 18, 1 to 19, 1 to 20, 1 to 21, 1 to 22, 1 to 23, 1 to 24, 1 to 25, 1 to 26, 1 to 27, 1 to 28, 1 to 29, or 1 to 30 days.

As used herein, the term “amendment” refers broadly to any material added to soil to improve its physical or chemical properties. As used herein, the terms “carbon-based soil amendment” or “carbon amendment” encompass any carbon-based material that, when added to the soil, yields an amended soil having improved physical or chemical properties. Non-limiting examples of carbon-based soil amendments include simple nutrients such as sugars, e.g. fructose, glucose, sucrose, lactose, galactose, dextrose, maltose, raffinose, ribose, ribulose, xylulose, xylose, amylase, arabinose, etc.; and sugar alcohols, e.g. adonitol, sorbitol, mannitol, maltitol, ribitol, galactitol, glucitol, etc., as well as complex substrates, including cellulose and lignin. In some embodiments, the carbon amendment comprises a combination of one or more simple nutrients, sugar alcohols or complex substrates disclosed herein.

It is to be understood that the description above as well as the examples that follow are intended to illustrate, and not limit, the scope of the invention. Other aspects, advantages and modifications within the scope of the invention will be apparent to those skilled in the art to which the invention pertains.

### EXAMPLES

### Example 1: Microbial Signalers Enhance Pathogen Inhibition by Target Microbes in Commercial Products

Microbical signalers disclosed herein—MS1, MS2, MS3, MS4, MS5, MS6, and MS7 (Table 3)—were evaluated for their ability to influence the pathogen inhibiting activity of the target microbe, Streptomyces spp. in two different commercial products. The following study was performed to test whether the activity of Streptomyces spp. to inhibit the growth of plant pathogens such as, Pythium, Verticillium, and Phytophthora was influenced by the physical proximity of the microbial signalers disclosed herein.

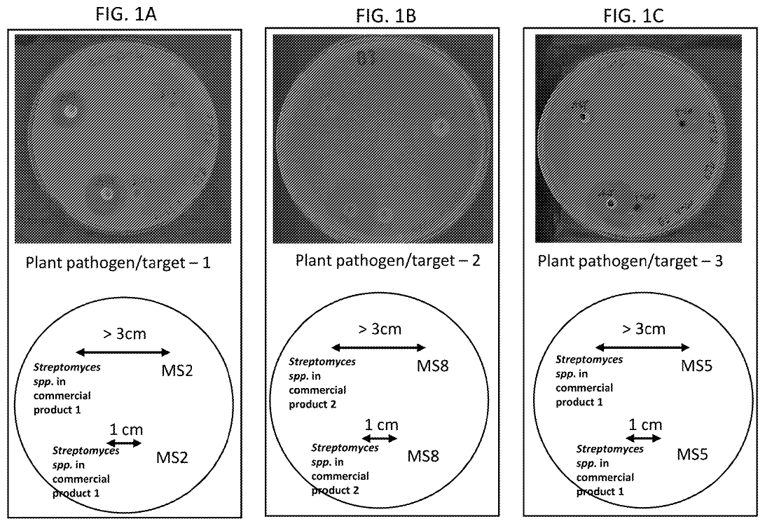

Cultures of Streptomyces spp. were inoculated on nutrient medium, along with each of the microbial signalers, MS1, MS2, MS3, MS4, MS5, MS6, and MS7, either >3 cm apart (referred to herein as inoculated “alone”) or 1 cm apart (referred to herein as inoculated “adjacent” to each other). After 3 days growth, the culture plates were overlaid with a second layer of growth medium, onto which a indicator microbe was plated. After 24 hours, the effectiveness of the microbial signaler in increasing indicator microbe inhibition by the commercial product was determined by comparing the size of the inhibition zones induced by Streptomyces spp. when inoculated >3 cm away from the microbial signaler with the size of the inhibition zone induced by Streptomyces spp. when 1 cm away from the microbial signaler. As shown in FIG. 1A, when Streptomyces spp. in commercial product 1 is inoculated 1 cm away from (or adjacent to) MS2, the inhibition zone (marked by the clear region around the colony) around Streptomyces spp., which indicates suppression of indicator microbe 22-D-2, is larger than when Streptomyces spp. is inoculated alone.

FIG. 1C similarly shows a remarkably enhanced indicator microbe-inhibiting activity of Streptomyces spp. in commercial product 1 only when inoculated adjacent to the microbial signaler MS5. These data indicate that the indicator microbe suppressing function of Streptomyces spp. is enhanced by the proximity of microbial signalers MS2 and MS5. The results from experiments evaluating the change in the inhibition zone size of Streptomyces spp. in commercial product 1 when inoculated adjacent to a microbial signaler disclosed herein relative to the inhibition zone size of Streptomyces spp. in commercial product 1 when inoculated alone for different indicator microbes are summarized in Table 2.

TABLE 2
Enhancement of Inhibitory Activity of Streptomyces spp.
in commercial product 1 by Microbial Signalers (MS)

| | Microbial Signaler | | | | | | |
|---|---|---|---|---|---|---|---|
| Indicator Microbe^{#} | MS1 | MS2 | MS3 | MS4 | MS5 | MS6 | MS7 |
| Bacillus spp. 22-D-2 | XX | X | XX | X | XX | XXX | X |
| Bacillus spp. 33-U-4 | X | X | X | X | — | XXX | X |
| Bacillus spp. 52-U-1 | XX | XXX | X | — | — | X | X |
| Bacillus spp. 62-U-2 | X | X | X | XX | — | X | X |
| Bacillus spp. B3 | XXX | XX | X | — | X | X | X |

X: 2 to 5-fold increase in inhibition compared to Streptomyces spp. in commercial product 1 alone
XX: 5 to 10-fold increase compared to Streptomyces spp. in commercial product 1 alone
XXX: more than 10-fold increase compared to Streptomyces spp. in commercial product 1 alone
^{#}As used herein “indicator microbes” are microbes that exhibit sensitivity to plant pathogen-inhibitory antibiotics. Thus, the suppression of indicator microbes may be used as a read out for suppression of pathogens.

TABLE 3

| Name of microbial | Corresponding name of microbial |
|---|---|
| isolate in FIGS. 1-4 | isolate in Table 1 |
| MS1 | JBS3418 |
| MS2 | JBS5867 |
| MS3 | JBS9311 |
| MS4 | JBS4046 |
| MS5 | JBS3880 |
| MS6 | JBS1452 |
| MS7 | JBS6900 |

Similar results were obtained with Streptomyces spp. in commercial product 2. As shown in FIG. 11B, when Streptomyces spp. in commercial product 2 is inoculated 1 cm away from (or adjacent to) the microbial signaler MS8, the inhibition zone around Streptomyces spp., which indicates suppression of indicator microbe B3, is larger than when Streptomyces spp. in commercial product 2 is inoculated alone. This indicates that the pathogen suppressing function of Streptomyces spp. in commercial product 2 is enhanced by the proximity of microbial signaler MS8.

Overall, these data demonstrate that the microbial signalers disclosed herein can increase the pathogen suppression function of commercial biopesticides, comprising Streptomyces spp. Without being bound by a theory, it is thought that the microbial signalers may be capable of enhancing and/or inducing the antibiotic production of the active microbes in the commercial biopesticides. In fact, in some cases, the microbial signalers disclosed herein were able to induce pathogen suppression activity that was not seen when the commercial biopesticide was inoculated alone.

### Example 2: Microbial Signalers are Capable of Reducing Constraints on Antibiotic Production Under Low Nutrient Conditions

Microbical signalers disclosed herein—MS1, MS2, MS3, MS4, MS5, MS6, MS7 and MS8—were evaluated for their ability to influence the pathogen inhibiting activity of Streptomyces spp. in commercial product 1 under high nutrient conditions and low nutrient conditions. Cultures of the active microbe in Streptomyces spp. in commercial product 1 was inoculated on low or high nutrient medium, along with each of the microbial signalers, MS1, MS2, MS3, MS4, MS5, MS6, MS7, and MS8 either >3 cm apart (referred to herein as inoculated “alone”) or 1 cm apart (referred to herein as inoculated “adjacent” to each other). While the high nutrient medium comprised standard nutrient concentration, the low nutrient medium comprised 1/10^{th} the standard nutrient concentration. After 3 days of growth, the culture plates were overlaid with a second layer of growth medium, onto which a indicator microbe was plated. After 24 hours, the effectiveness of the microbial signaler in increasing indicator microbe inhibition by Streptomyces spp. in commercial product 1 was evaluated on the high nutrient medium and the low nutrient medium.

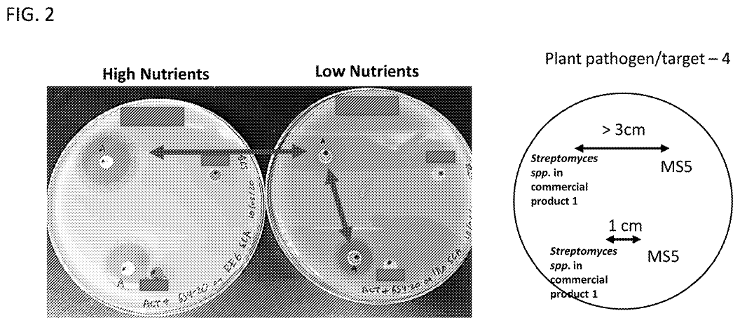

As shown in FIG. 2, under high nutrient conditions, the inhibition zone around Streptomyces spp. in commercial product 1 is clearly visible in the presence of the indicator microbe. In sharp contrast, Streptomyces spp. in commercial product 1 is unable to suppress the growth of indicator microbe under low nutrient conditions, indicating that nutrient status has a significant impact on inhibition of pathogens and on antibiotic production by Streptomyces spp. in commercial product 1. Surprisingly, when Streptomyces spp. is inoculated adjacent to the microbial signaler MS5, it shows indicator microbe inhibition activity even under low nutrient conditions, as marked by the clean inhibition zone around Streptomyces spp. in commercial product 1.

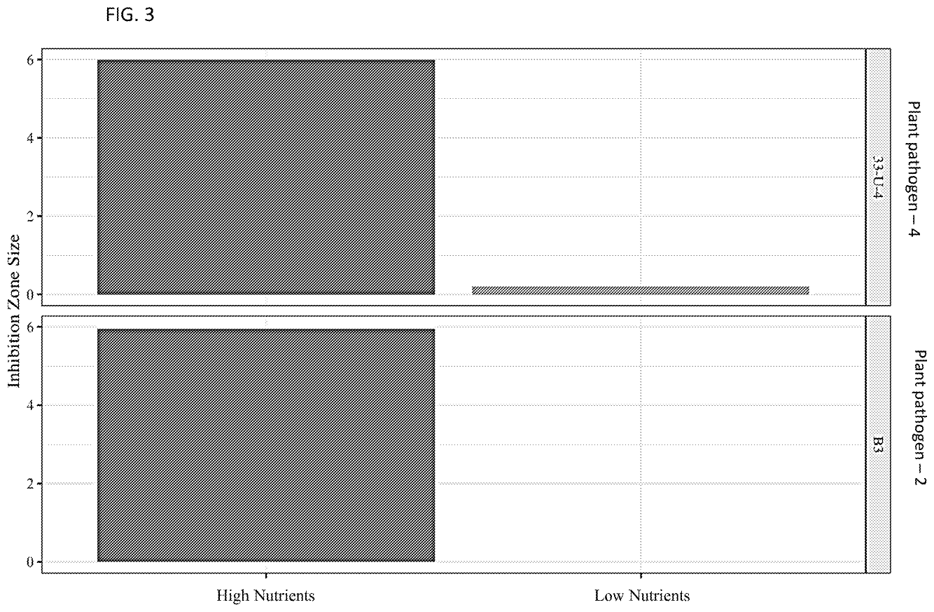

FIG. 3 shows the change in the size of the inhibition zone of Streptomyces spp. in commercial product 1 in the presence of indicator microbes—33-U-4 or B3—under low or high nutrient conditions. FIGS. 4A and 4B shows the increase in inhibition zone size seen in the presence of indicator microbes 33-U-4 or B3 when Streptomyces spp. in commercial product 1 is inoculated in the presence of any one of the microbial signalers MS1 through MS8 in a low or high nutrient medium. These results show that in a low nutrient environment, the microbial signalers disclosed herein are capable of boosting the pathogen suppression function of Streptomyces spp. in commercial product 1 and other commercial biopesticides. For instance, the suppression of indicator microbe 33-U-4 by Streptomyces spp. in commercial product 1 under low nutrient media is increased by physical proximity to the microbial signaler MS2, MS3, MS5, MS6 and MS7. Further, the suppression of indicator microbe B3 by Streptomyces spp. in commercial product 1 under low nutrient media is increased by physical proximity to the microbial signaler MS3 and MS4. Therefore, the microbial signalers can potentially extend the range of habitats under which pathogen antagonism can occur, and can enhance the consistency of pathogen suppression across habitats.

### Example 3: Microbial Signalers Enhance the Plant Pathogen-Inhibiting Function of Target Microbes

The capability of microbial signalers disclosed herein to enhance the plant pathogen-inhibiting function of target microbes was assessed. Signaling between the microbial signaler and the target microbe in a commercial product was determined by characterizing the magnitude of inhibition of a specific pathogen by the target microbe in a commercial product alone, or by the target microbe in a commercial product in the presence of each of the microbial signalers in vitro. The target microbe was cultivated individually, or in close proximity to each of the microbial signalers, in the presence of a plant pathogen. Changes in inhibition of the pathogen by the target microbe in the presence vs. absence of a microbial signaler reflect the capacity of the microbial signaler to alter the target function (inhibition of the target pathogen).

FIGS. 5A-5G depict the percentage increase in the inhibition of a Bacillus spp. indicator microbe or a Streptomyces scabies plant pathogen in the presence of a combination of: (a) the microbial signaler listed on the X axis, and (b) Streptomyces spp. in commercial product 1, as compared to the inhibition of the indicator microbe in the presence of Streptomyces spp. in commercial product 1 alone. Similarly, FIGS. 6A-6B, 7A-7B, 8A-8F, 9, and 10A-10E depict the percentage increase in the inhibition of the indicated plant pathogen (e.g., Colletotrichum graminicola, Pectobacterium caratovorum, Rhizoctonia solani, Sclerotinia sclerotiorum, Bacillus spp., Pythium irregulare, Streptomyces scabies, Fusarium culmorum, Fusarium graminearum, Fusarium oxysporum) in the presence of a combination of: (a) the microbial signaler listed on the X axis, and (b) a commercial product comprising one of the following target microbes: Bacillus spp., Streptomyces spp., or Talaromyces spp., respectively, as compared to the inhibition of the plant pathogen in the presence of the respective target microbe alone.

Furthermore, to further test the capability of the microbial signalers disclosed herein to enhance the pathogen suppression activity of a target microbe in a commercial product, the following two assays were performed. Assay 1 measures pathogen inhibition communicated via signals that can diffuse through a solid medium, while assay 2 measures pathogen inhibition communicated via signals that diffuse through the air.

Assay 1: A microbial signaler disclosed herein was spotted onto an agar plate at specific distance from a spotted suspension of a target microbe (e.g. a Trichoderma spp. strain in a commercial product) with known antimicrobial activity (e.g. against Fusarium culmorum). Both the microbial signaler and the target microbe were also spotted alone onto plates. Plates were incubated at 28° C. followed by chemical deactivation to kill the microbes. A fresh medium specific to pathogen nutrient preferences was overlaid on the plate and the pathogen of interest (e.g. Fusarium culmorum) was spread-plated onto the medium. Following incubation at pathogen-specific temperature and time, the zone of pathogen inhibition was measured and recorded for each target microbe alone, and in association with the microbial signaler. The area of inhibition induced by the product in the presence of the microbial signaler is compared to that of the target microbe alone.

Assay 2: A 5 mm plug of a fully-grown microbial signaler is plug-spotted at specific distance from a commercial product (5 mm plug) on an agar plate. This is denoted as Plate A. The pathogen of interest is plug-spotted on a second agar plate (Plate B). Both plates are opened under the biosafety cabinet and Plate B (Top) is sandwiched unto Plate A (Bottom) with parafilm. The sandwiched plates are subsequently incubated under pathogen-specific conditions. The zone of pathogen growth inhibition is measured and recorded accordingly. The area of pathogen growth inhibition in the presence of microbial signalers paired with the target microbe is compared to that of the target microbe alone.

FIGS. 13A-13B and FIGS. 14A-14B show results from performing assays 1 and 2 to measure the capability of the microbial signalers disclosed herein to enhance the pathogen suppression activity (e.g. suppression of Fusarium culmorum or Phytophthora sojae) of a target microbe in a commercial product (e.g. a Trichoderma spp. strain in a commercial product).

As shown in FIGS. 13A-13B, the inhibition of Fusarium culmorum in the presence of a combination of: (a) the microbial signaler listed on the X axis, and (b) a Trichoderma spp. microbe in a commercial product, is remarkably higher as compared to the inhibition of the plant pathogen in the presence of the Trichoderma spp. microbe in the commercial product alone. Additionally, this effect extends to other plant pathogens, since FIG. 14A shows an increase in the inhibition of a plant pathogen (Phytophthora sojae) as measured by Assay 1 described above in the presence of a combination of: (a) each of the microbial signalers listed on the X axis, and (b) a Trichoderma spp. microbe in a commercial product, as compared to the inhibition of the plant pathogen (Phytophthora sojae) in the presence of the Trichoderma spp. microbe in the commercial product on its own. FIG. 14B also shows a similar the percentage increase in the inhibition of a plant pathogen (Phytophthora sojae) as measured by Assay 2 described above in the presence of a combination of: (a) each of the microbial signalers listed on the X axis, and (b) a Trichoderma spp. microbe in a commercial product, as compared to the inhibition of the plant pathogen (Phytophthora sojae) in the presence of the Trichoderma spp. microbe in the commercial product on its own.

Furthermore, FIG. 25 depicts the percentage increase in the inhibition of Fusarium culmorum in the presence of a combination of: (a) JBS6226, and (b) a Bacillus spp. microbe in a commercial product, as compared to the inhibition of the plant pathogen in the presence of the Bacillus spp. microbe in the commercial product alone. These results show that pathogen suppression is remarkably enhanced in the presence of the microbial signalers disclosed herein in combination with the Bacillus spp. commercial product.

Increase in plant pathogen inhibition was also seen for any one or more of the microbial signaler isolates disclosed herein (e.g. JBS6220, JBS3946, JBS9264, JBS5867, JBS9311, JBS8612, JBS9780, JBS8753, JBS4761, JBS3673, JBS8054, JBS1452, JBS6900, JBS6069, JBS3418, JBS8493, JBS4783, JBS8473, JBS6762, JBS9261, JBS4549, JBS6899, JBS9225, JBS8135, JBS5020, JBS5523, or JBS3880) in combination with Pseudomonas spp. in a commercial product, as compared to the inhibition of the plant pathogen in the presence of Pseudomonas spp. alone.

These data demonstrate that the microbial signalers disclosed herein are capable of enhancing the plant pathogen-inhibiting function of the target microbes disclosed herein (e.g. target microbes in the commercial products disclosed herein). Therefore, the microbial signalers disclosed herein, when used in combination with the commercial products (and/or the target microbes present in the commercial products), have unexpectedly superior effects on plant growth, at least in part, through the enhanced suppression of plant disease.

### Example 4: Microbial Signalers Enhance Plant Disease Suppression In Vivo

To test the capability of the microbial signalers disclosed herein to enhance the plant disease suppression function (e.g. a disease caused by pathogenic Phytophthora sojae) of a target microbe in a commercial product (e.g. a Trichoderma spp. strain in a commercial product), the following experiment was performed.

Soybean seeds were sown into containers (24 cm×7 cm) filled with commercial greenhouse soil mix inoculated with pathogenic Phytophthora sojae (thoroughly mixed into soil). Plants were inoculated at planting with either the target microbe (e.g. a Trichoderma spp. strain in a commercial product) alone, a microbial signaler and the target microbe, or sterile water (water control). Plants were watered alternate days and maintained under soybean-specific temperature and light/dark cycle. After 14-21 days, soybean plants were harvested (n=20 plants per treatment), and disease severity (1-5 index), and above- and below-ground fresh and dry weights were determined for every plant.

FIG. 15A depicts the percentage increase in Phytophthora sojae disease suppression (wherein a reduction in disease incidence is measured based on a reduction in the proportion of infected plants) on soybean plants inoculated with: (a) each of the microbial signalers listed on the X axis, and (b) a Trichoderma spp. microbe in a commercial product, as compared to the Trichoderma spp. microbe in the commercial product on its own.

FIG. 15B depicts the percentage increase Phytophthora sojae disease suppression (wherein a reduction in disease severity is assessed on a scale of 1 through 5) on soybean plants inoculated with: (a) each of the microbial signalers listed on the X axis, and (b) a Trichoderma spp. microbe in a commercial product, as compared to the Trichoderma spp. microbe in the commercial product on its own.

Furthermore, FIG. 23 depicts the percentage reduction in Pythium disease severity in plants inoculated with: (a) a combination of microbial signalers JBS4783, JBS8135, JBS3880, and (b) a Talaromyces spp. target microbe in a commercial product, as compared to the Talaromyces spp. target microbe in a commercial product on its own. These results show that the microbial signalers disclosed herein enhanced the suppression of Pythium disease by Talaromyces spp. target microbe in a commercial product by more than 5-fold.

Taken together, the results demonstrate that the presence of the microbial signalers disclosed herein in combination with the target microbes disclosed herein can enhance pathogen suppression not only in vitro but in vivo. Furthermore, the microbial signalers disclosed herein improved the suppression of disease incidence as well as disease severity by the target microbes.

### Example 5: Microbial Signalers Enhance the Phosphate Solubilization Function of Target Microbes

The capability of microbial signalers disclosed herein to enhance phosphate solubilizing function of target microbes was assessed. Signaling between the microbial signaler and the target commercial product was determined by characterizing the magnitude of phosphate solubilization by the commercial product alone, or by the commercial product in the presence of each of the microbial signalers in vitro. The commercial product was cultivated individually, or in close proximity to each of the microbial signalers, in the presence of insoluble phosphate. Changes in the solubilization of the phosphate by the commercial product in the presence vs. absence of a microbial signaler reflect the capacity of the microbial signaler to alter the target function (solubilization of phosphate).

FIG. 11A depicts the percentage increase in the phosphate solubilization in the presence of a combination of: (a) the microbial signaler listed on the X axis, and (b) Streptomyces spp. in commercial product 1, as compared to the phosphate solubilization in the presence of Streptomyces spp. in commercial product 1 alone. Similarly, FIGS. 11B-11F depict the percentage increase in the phosphate solubilization in the presence of a combination of: (a) the microbial signaler listed on the X axis, and (b) a commercial product comprising one or more of the following target microbes: Pseudomonas spp., Comamonas spp., Citrobacter spp., Enterobacter spp., Streptomyces spp., Trichoderma spp., Bacillus spp., and Talaromyces spp., respectively, as compared to the phosphate solubilization in the presence of the respective target microbe alone.

As another example, FIG. 24 depicts the percentage increase in the phosphate solubilization in the presence of a combination of: (a) each of the microbial signalers listed on the X axis, and (b)) a Talaromyces spp. target microbe in a commercial product, as compared to the phosphate solubilization in the presence of) a Talaromyces spp. target microbe in a commercial product alone.

Moreover, the data described below show that the microbial signalers disclosed herein enhance phosphate solubilization functions of target microbes even at dramatically reduced target microbe densities. For instance, the following experiment was performed in this regard.

A pre-defined concentration of a microbial signaler was spotted at a specified distance from a target microbe (Pseudomonas spp.; Comamonas spp.; Citrobacter spp.; and Enterobacter spp. in a commercial product) in triplicate on a single plate. In addition, the microbial isolates and the target microbes were spotted alone on plates of the same medium. The total experiment was duplicated for multiple target microbe concentrations (as indicated in FIG. 22). Plates were incubated for 14 days at 27° C. The area of phosphate solubilization in the presence of the microbial signalers disclosed herein paired with the target microbe was compared to that of the target microbe alone.

FIG. 22 depicts the percentage increase in in vitro phosphate solubilization by Pseudomonas spp.; Comamonas spp.; Citrobacter spp.; and Enterobacter spp. in a commercial product at three different inoculum densities (1× undiluted, 10× diluted, 100× diluted) in the presence of JBS9225.

These data demonstrate that the microbial signalers disclosed herein are capable of enhancing the phosphate solubilizing function of the target microbes disclosed herein. Therefore, the microbial signalers disclosed herein, when used in combination with the commercial products (and/or the target microbes present in the commercial products), potentially have unexpectedly superior effects on plant growth, at least in part, through the enhanced phosphate solubilization.

### Example 6: Microbial Signalers Enhance the Zinc Solubilization Function of Target Microbes

The capability of microbial signalers disclosed herein to enhance zinc solubilizing function of target microbes was assessed. Signaling between the microbial signaler and the target commercial product was determined by characterizing the magnitude of zinc solubilization by the commercial product alone, or by the commercial product in the presence of each of the microbial signalers in vitro. The commercial product was cultivated individually, or in close proximity to each of the microbial signalers, in the presence of insoluble zinc. Changes in the solubilization of the zinc by the commercial product in the presence vs. absence of a microbial signaler reflect the capacity of the microbial signaler to alter the target function (solubilization of zinc).

FIG. 12A depicts the percentage increase in the zinc solubilization in the presence of a combination of: (a) the microbial signaler listed on the X axis, and (b) Streptomyces spp. in commercial product 1, as compared to the zinc solubilization in the presence of Streptomyces spp. in commercial product 1 alone. Similarly, FIGS. 12B-12D depict the percentage increase in the zinc solubilization in the presence of a combination of: (a) the microbial signaler listed on the X axis, and (b) a commercial product comprising one or more of the following target microbes: Streptomyces spp., Trichoderma spp., and Talaromyces spp., respectively, as compared to the zinc solubilization in the presence of the respective target microbe alone.

These data demonstrate that the microbial signalers disclosed herein are capable of enhancing the zinc solubilizing function of the target microbes disclosed herein. Therefore, the microbial signalers disclosed herein, when used in combination with the commercial products (and/or the target microbes present in the commercial products), potentially have unexpectedly superior effects on plant growth, at least in part, through the enhanced zinc solubilization.

### Example 7: Microbial Signalers Enhance the Plant Growth Promoting Function of Target Bacteria

To test the capability of the microbial signalers disclosed herein to enhance the plant growth promoting function of a target bacteria in a commercial product (e.g. a Bacillus spp. strain in a commercial product), the following experiment was performed.

Corn or soybean seeds were sown into containers (24 cm×7 cm) and inoculated either with the target microbe alone, the microbial signaler and the target microbe in a commercial product, or sterile water (water control). Plants were watered every two days. After germination, plants were watered every 7 days with a low-nitrogen solution. The full-nutrient controls were inoculated with sterile water and grown under the same conditions, however the plants received Hoagland with normal levels of nitrogen. Plants (n=15 per treatment) were arranged in a randomized complete block design and grown at corn or soybean-specific temperature and light/dark cycle. Growth parameters were measured weekly. After 31 days plants were harvested, and fresh and dry weights were determined for every plant.

As shown in FIG. 20A, the percentage of fresh belowground biomass of corn plants inoculated with the Bacillus spp. in a commercial product is higher than corn plants treated with water. Notably, the presence of the microbial signalers disclosed herein further enhances plant growth, since the percentage in fresh belowground biomass of corn plants inoculated with: (a) each of the microbial signalers listed on the X axis, and (b) a Bacillus spp. in a commercial product, is significantly higher as compared to the Bacillus spp. in a commercial product on its own.

Similarly, FIG. 20B shows that the percentage of dry aboveground biomass of soybean plants inoculated with: (a) each of the microbial signalers listed on the X axis, and (b) a Bacillus spp. in a commercial product, is remarkably higher as compared to the Bacillus spp. in a commercial product on its own. Finally, FIG. 20C shows that the frequency of healthy unifoliate leaves of soybean inoculated with: (a) each of the microbial signalers listed on the X axis, and (b) a Bacillus spp. in a commercial product, is much higher as compared to the Bacillus spp. in a commercial product on its own, further highlighting the plant growth enhancing properties of the microbial signalers and microbial corsortia disclosed herein.

Additionally, FIG. 21 shows that soybean plants inoculated with: (a) the indicated microbial signaler, and (b) a Bacillus spp. in a commercial product, have noticeably increased vigor and reduced frequency of chlorotic leaves, as compared to the Bacillus spp. in a commercial product on its own or just water.

Finally, FIG. 16B shows that a remarkably increased percentage of corn plants reach vegetative growth stage 4 (V4) when the plants are inoculated with: (a) the indicated microbial signaler, and (b) a Azospirillum spp. in a commercial product, as compared to the Azospirillum spp. target microbe on its own.

Take together, these data underline the ability of the microbial signalers and compositions disclosed herein to enhance the plant growth promoting functions of target microbes in commercial products.

### Example 8: Microbial Signalers Enhance the Plant Growth Promoting Function of Target Fungi

To test the capability of the microbial signalers disclosed herein to enhance the plant growth promoting function of a target fungus in a commercial product (e.g. a Glomus spp. strain in a commercial product), the following experiment was performed.

Corn or soybean seeds were sown into containers (24 cm×7 cm) and inoculated either with a target microbe alone, a microbial signaler and a target microbe in a commercial product, or sterile water (water control). Plants were watered every two days. After germination, plants were watered every 7 days with a low-nitrogen solution. The full-nutrient controls were inoculated with sterile water and grown under the same conditions, however the plants received Hoagland with normal levels of nitrogen. Plants (n=15 per treatment) were arranged in a randomized complete block design and grown at corn or soybean-specific temperature and light/dark cycle. Growth parameters were measured weekly. After 31 days plants were harvested, and fresh and dry weights were determined for every plant.

As shown in FIG. 18A, the percentage of dry belowground biomass of corn plants inoculated with the Glomus spp. in a commercial product is higher than in corn plants treated with water. Notably, the presence of the microbial signalers disclosed herein further enhances plant growth, since the percentage in dry belowground biomass of corn plants inoculated with: (a) each of the microbial signalers listed on the X axis, and (b) a Glomus spp. in a commercial product, is significantly higher as compared to the Glomus spp. in a commercial product on its own.

Furthermore, FIG. 18B shows that the growth of soybean plants inoculated with: (a) the microbial signaler listed on the X axis, and (b) a Glomus spp. in a mycorrhizal commercial product, is much higher than the growth of soybean plants inoculated with just the Glomus spp. in a mycorrhizal commercial product.

Finally, FIG. 19 shows that corn plants inoculated with: (a) the indicated microbial signaling isolate, and (b) a Glomus spp. in a mycorrhizal commercial product, have noticeably increased vigor and reduced purple coloration associated with nutrient stress, as compared to the corn plants inoculated with Glomus spp. in a mycorrhizal commercial product on its own.

Taken together, these data underline the ability of the microbial signalers and compositions disclosed herein to enhance the plant growth promoting functions of target fungi in commercial mycorrhizal products. Without being bound by a theory, it is thought that the enhancement of plant growth promoting function of the target microbe by the microbial signalers disclosed herein may be associated with, result from, or be caused by an increase in nutrient acquisition and/or uptake by the plants in the presence of the microbial signalers.

### Example 7: Microbial Signalers Enhance the Plant Growth Promoting Function of Free-Living and Symbiotic Nitrogen Fixing Target Microbes

To test the capability of the microbial signalers disclosed herein to enhance the plant growth promoting function of free-living nitrogen fixing bacteria in a commercial product (e.g. a Azospirillum spp. strain in a commercial product), the following experiment was performed.

Corn seeds were sown into containers (24 cm×7 cm) and inoculated with either a target microbe alone, a microbial signaler and a target microbe, or sterile water (water control). Plants were watered alternate day. In addition, after germination plants were watered every 7 days with modified Hoagland solution with low nitrogen concentration. The full-nutrient control group were inoculated with sterile water, however the plants received Hoagland with standard nitrogen concentration. Plants (n=15 per treatment) were arranged in a randomized complete block design and grown at corn-specific temperature and light/dark cycle. Growth parameters were measured weekly. After 31 days plants were harvested, roots were washed and both fresh and dry weights were determined for all plants.

As shown in FIG. 16A, the percentage of dry aboveground biomass of corn plants inoculated with the Azospirillum spp. in a commercial product is higher than in corn plants treated with water. Notably, the presence of the microbial signalers disclosed herein further enhances plant growth, since the percentage in dry aboveground biomass of corn plants inoculated with: (a) each of the microbial signalers listed on the X axis, and (b) a Azospirillum spp. in a commercial product, is significantly higher as compared to the Azospirillum spp. in a commercial product on its own.

Furthermore, FIG. 16B shows that a remarkably increased percentage of corn plants reach growth stage 4 when the plants are inoculated with: (a) the indicated microbial signaler, and (b) a Azospirillum spp. in a commercial product, as compared to the Azospirillum spp. target microbe on its own.

To further test the capability of the microbial signalers disclosed herein to enhance the plant growth promoting function of symbiotic nitrogen fixing bacteria in a commercial product (e.g. a Bradyrhizobium spp. strain in a commercial product), the following experiment was performed.

Soybean seeds were sown into containers (24 cm×7 cm) filled with commercial greenhouse soil mix. Plants were inoculated with either a target microbe (e.g., Bradyrhizobium spp.) alone, a microbial signaler and a Bradyrhizobium nitrogen-fixing symbiont at planting, or sterile water (water control). Plants were watered alternate days and maintained under soybean-specific temperature and light/dark cycle. After 5 weeks, soybean plants were harvested (n=25 plants per treatment), and above- and belowground dry weights were determined for every plant.

As shown in FIG. 17A, the percentage of aboveground biomass of soybean inoculated with: (a) each of the microbial signalers listed on the X axis, and (b) a Bradyrhizobium spp. microbe in a commercial product, is significantly increased as compared to the Bradyrhizobium spp. microbe in the commercial product on its own. Similarly, FIG. 17B depicts the percentage increase in belowground biomass of soybean inoculated with: (a) each of the microbial signalers listed on the X axis, and (b) a Bradyrhizobium spp. microbe in a commercial product, as compared to the Bradyrhizobium spp. microbe in the commercial product on its own.

Taken together, these data underline the ability of the microbial signalers and compositions disclosed herein to enhance the plant growth promoting functions of target fungi in commercial products. Without being bound by a theory, it is thought that the enhancement of plant growth promoting function of the target microbe by the microbial signalers disclosed herein may be associated with, result from, or be caused by an increase in nitrogen fixation function by the target microbes in the presence of the microbial signalers.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### NUMBERED EMBODIMENTS

The following list of embodiments is included herein for illustration purposes only and is not intended to be comprehensive or limiting. The subject matter to be claimed is expressly not limited to the following embodiments.

Embodiment 1. A composition, comprising at least one microbial signaler, wherein the at least one microbial signaler is capable of enhancing a plant growth-promoting function of at least one target microbe.

Embodiment 2. The composition of embodiment 1, comprising: the at least one target microbe.

Embodiment 3. The composition of embodiment 1 or embodiment 2, wherein the at least one microbial signaler belongs to the genus Streptomyces, Fusarium, or Bacillus.

Embodiment 4. The composition of any one of embodiments 1-3, wherein the at least one microbial signaler belongs to the genus Streptomyces.

Embodiment 5. The composition of any one of embodiments 1-4, wherein the plant growth-promoting function comprises: (a) plant pathogen-inhibiting function, (b) zinc solubilizing function, (c) phosphate solubilizing function, (d) production of an antibiotic, (e) nitrogen fixing function, (f) a function of improving a plant's nutrient acquisition, (f) production of plant growth hormones, or (g) any combination thereof.

Embodiment 6. The composition of of embodiment 5, wherein the at least one microbial signaler is capable of enhancing the plant pathogen-inhibiting function of the target microbe by at least about 5%.

Embodiment 7. The composition of of embodiment 5 or embodiment 6, wherein the at least one microbial signaler is capable of enhancing the zinc solubilizing function of the target microbe by at least about 5%.

Embodiment 8. The composition of of any one of embodiments 5-7, wherein the at least one microbial signaler is capable of enhancing the phosphate solubilizing function of the target microbe by at least about 5%.

Embodiment 9. The composition of any one of embodiments 5-8, wherein the at least one microbial signaler is capable of enhancing the plant growth-promoting function of the at least one target microbe under low nutrient conditions.

Embodiment 10. The composition of any one of embodiments 1-9, wherein the at least one microbial signaler is Streptomyces avidinii, Streptomyces colombiensis, Streptomyces lavendulae, Streptomyces roseochromogenus, Streptomyces spororaveus, Streptomyces sporoverrucosus, Streptomyces venezuelae, Streptomyces xanthophaeus, Streptomyces angustmyceticus, Streptomyces hygroscopicus, Streptomyces libani, Streptomyces lydicus, Streptomyces nigrescens, Streptomyces platensis, Streptomyces rimosus, Streptomyces tubercidicus, Streptomyces bungoensis, Streptomyces cyslabdanicus, Streptomyces galbus, Streptomyces kagawaensis, Streptomyces lasaliensis, Streptomyces lasalocidi, Streptomyces longwoodensis, Streptomyces spinichromogenes, Streptomyces cirratus, Streptomyces nojiriensis, Streptomyces verne, Streptomyces vinaceus, Streptomyces virginiae, Streptomyces catenulae, Streptomyces cinereus, Streptomyces griseocarneus, Streptomyces sioyaensis, Streptomyces subrutilus, Streptomyces atrolaccus, Streptomyces auratus, Streptomyces fagopyri, Streptomyces kaempferi, Streptomyces mirabilis, Streptomyces olivochromogenes, Streptomyces chattanoogensis, Streptomyces flaveus, Streptomyces goshikiensis, Streptomyces cinnamonensis, Streptomyces senoensis, Streptomyces echinatus, Streptomyces filipinensis, Streptomyces gulbargensis, Streptomyces myxogenes, Streptomyces novaecaesareae, Streptomyces spectabilis, Streptomyces tanashiensis, Streptomyces ginsengisoli, Streptomyces graminisoli, Streptomyces lucensis, Streptomyces yaanensis, Streptomyces caniferus, Streptomyces decoyicus, Streptomyces glebosus, Streptomyces ossamyceticus, Streptomyces badius, Streptomyces cyaneofuscatus, Streptomyces flavogriseus, Streptomyces griseus, Streptomyces mediolani, Streptomyces praecox, Streptomyces pratensis, Streptomyces omiyaensis, Streptomyces aquilus, Streptomyces caeruleatus, Streptomyces griseochromogenes, Streptomyces pseudovenezuelae, Streptomyces viridochromogenes, Streptomyces argenteolus, Streptomyces chrestomyceticus, Streptomyces coelicolor, Streptomyces microsporus, Streptomyces aureus, Streptomyces lutosisoli, Streptomyces minoensis, Streptomyces rhizosphaerihabitans, Streptomyces griseoruber, Streptomyces scabiei, Streptomyces achromogenes, Streptomyces canarius, Streptomyces capoamus, Streptomyces cellostaticus, or Streptomyces katrae.

Embodiment 11. The composition of any one of embodiments 1-10, wherein the at least one microbial signaler comprises a 16S nucleic acid sequence having at least about 97% sequence identity to one or more of the following SEQ ID Nos: 1-36.

Embodiment 12. The composition of of any one of embodiments 1-11, wherein the at least one microbial signaler comprises a 16S nucleic acid sequence of any one of the following SEQ ID Nos: 1-36.

Embodiment 13. The composition of any one of embodiments 1-11, wherein the at least one microbial signaler comprises a 16S nucleic acid sequence having at least about 97% sequence identity to SEQ ID NO: 1.

Embodiment 14. The composition of embodiment 13, wherein the at least one microbial signaler is Streptomyces echinatus, Streptomyces filipinensis, Streptomyces gulbargensis, Streptomyces longwoodensis, Streptomyces myxogenes, Streptomyces novaecaesareae, Streptomyces spectabilis, or Streptomyces tanashiensis.

Embodiment 15. The composition of any one of embodiments 1-11, wherein the at least one microbial signaler comprises a 16S nucleic acid sequence having at least about 97% sequence identity to SEQ ID NO: 2.

Embodiment 16. The composition of embodiment 15, wherein the at least one microbial signaler is Streptomyces echinatus, Streptomyces ginsengisoli, Streptomyces graminisoli, Streptomyces gulbargensis, Streptomyces longwoodensis, Streptomyces lucensis, Streptomyces tanashiensis, or Streptomyces yaanensis.

Embodiment 17. The composition of any one of embodiments 1-11, wherein the at least one microbial signaler comprises a 16S nucleic acid sequence having at least about 97% sequence identity to SEQ ID NO: 3.

Embodiment 18. The composition of embodiment 17, wherein the at least one microbial signaler is Streptomyces bungoensis, Streptomyces cyslabdanicus, Streptomyces galbus, Streptomyces kagawaensis, Streptomyces lasaliensis, Streptomyces lasalocidi, Streptomyces longwoodensis, or Streptomyces spinichromogenes.

Embodiment 19. The composition of any one of embodiments 1-11, wherein the at least one microbial signaler comprises a 16S nucleic acid sequence having at least about 97% sequence identity to SEQ ID NO: 4.

Embodiment 20. The composition of embodiment 19, wherein the at least one microbial signaler is Streptomyces auratus, Streptomyces cyslabdanicus, Streptomyces fagopyri, Streptomyces galbus, Streptomyces kaempferi, Streptomyces mirabilis, or Streptomyces olivochromogenes.

Embodiment 21. The composition of any one of embodiments 1-11, wherein the at least one microbial signaler comprises a 16S nucleic acid sequence having at least about 97% sequence identity to SEQ ID NO: 5.

Embodiment 22. The composition of embodiment 21, wherein the at least one microbial signaler is Streptomyces achromogenes, Streptomyces bungoensis, Streptomyces canarius, Streptomyces capoamus, Streptomyces cellostaticus, Streptomyces galbus, Streptomyces katrae, or Streptomyces spinichromogenes.

Embodiment 23. The composition of any one of embodiments 1-11, wherein the at least one microbial signaler comprises a 16S nucleic acid sequence having at least about 97% sequence identity to SEQ ID NO: 6.

Embodiment 24. The composition of of embodiment 23, wherein the at least one microbial signaler is Streptomyces avidinii, Streptomyces colombiensis, Streptomyces lavendulae, Streptomyces roseochromogenus, Streptomyces spororaveus, Streptomyces sporoverrucosus, Streptomyces venezuelae, or Streptomyces xanthophaeus.

Embodiment 25. The composition of any one of embodiments 1-11, wherein the at least one microbial signaler comprises a 16S nucleic acid sequence having at least about 97% sequence identity to SEQ ID NO: 7.

Embodiment 26. The composition of of embodiment 25, wherein the at least one microbial signaler is Streptomyces avidinii, Streptomyces cirratus, Streptomyces lavendulae, Streptomyces nojiriensis, Streptomyces spororaveus, Streptomyces subrutilus, Streptomyces venezuelae, or Streptomyces xanthophaeus.

Embodiment 27. The composition of any one of embodiments 1-11, wherein the at least one microbial signaler comprises a 16S nucleic acid sequence having at least about 97% sequence identity to SEQ ID NO: 8.

Embodiment 28. The composition of embodiment 27, wherein the at least one microbial signaler is Streptomyces avidinii, Streptomyces colombiensis, Streptomyces flaveus, Streptomyces goshikiensis, Streptomyces lavendulae, Streptomyces spororaveus, Streptomyces subrutilus, or Streptomyces venezuelae.

Embodiment 29. The composition of any one of embodiments 1-11, wherein the at least one microbial signaler comprises a 16S nucleic acid sequence having at least about 97% sequence identity to SEQ ID NO: 9.

Embodiment 30. The composition of embodiment 29, wherein the at least one microbial signaler is Streptomyces cinnamonensis, Streptomyces cirratus, Streptomyces goshikiensis, Streptomyces lavendulae, Streptomyces senoensis, Streptomyces sporoverrucosus, Streptomyces vinaceus, or Streptomyces virginiae.

Embodiment 31. The composition of any one of embodiments 1-11, wherein the at least one microbial signaler comprises a 16S nucleic acid sequence having at least about 97% sequence identity to SEQ ID NO: 10.

Embodiment 32. The composition of embodiment 31, wherein the at least one microbial signaler is Streptomyces cinnamonensis, Streptomyces flaveus, Streptomyces lavendulae, Streptomyces nojiriensis, Streptomyces spororaveus, Streptomyces venezuelae, Streptomyces virginiae, or Streptomyces xanthophaeus.

Embodiment 33. The composition of any one of embodiments 1-11, wherein the at least one microbial signaler comprises a 16S nucleic acid sequence having at least about 97% sequence identity to SEQ ID NO: 11.

Embodiment 34. The composition of of embodiment 33, wherein the at least one microbial signaler is Streptomyces auratus, Streptomyces cinnamonensis, Streptomyces lavendulae, Streptomyces sioyaensis, Streptomyces spororaveus, Streptomyces verne, Streptomyces virginiae, or Streptomyces xanthophaeus.

Embodiment 35. The composition of any one of embodiments 1-11, wherein the at least one microbial signaler comprises a 16S nucleic acid sequence having at least about 97% sequence identity to SEQ ID NO: 12.

Embodiment 36. The composition of embodiment 35, wherein the at least one microbial signaler is Streptomyces cinnamonensis, Streptomyces flaveus, Streptomyces lavendulae, Streptomyces nojiriensis, Streptomyces spororaveus, Streptomyces venezuelae, Streptomyces virginiae, or Streptomyces xanthophaeus.

Embodiment 37. The composition of any one of embodiments 1-11, wherein the at least one microbial signaler comprises a 16S nucleic acid sequence having at least about 97% sequence identity to SEQ ID NO: 13.

Embodiment 38. The composition of embodiment 37, wherein the at least one microbial signaler is Streptomyces badius, Streptomyces cyaneofuscatus, Streptomyces flavogriseus, Streptomyces griseus, Streptomyces lavendulae, Streptomyces mediolani, Streptomyces praecox, or Streptomyces pratensis.

Embodiment 39. The composition of any one of embodiments 1-11, wherein the at least one microbial signaler comprises a 16S nucleic acid sequence having at least about 97% sequence identity to SEQ ID NO: 14.

Embodiment 40. The composition of embodiment 39, wherein the at least one microbial signaler is Streptomyces avidinii, Streptomyces cirratus, Streptomyces lavendulae, Streptomyces nojiriensis, Streptomyces omiyaensis, Streptomyces spororaveus, Streptomyces subrutilus, or Streptomyces vinaceus.

Embodiment 41. The composition of any one of embodiments 1-11, wherein the at least one microbial signaler comprises a 16S nucleic acid sequence having at least about 97% sequence identity to SEQ ID NO: 15.

Embodiment 42. The composition of embodiment 41, wherein the at least one microbial signaler is Streptomyces cinnamonensis, Streptomyces flaveus, Streptomyces lavendulae, Streptomyces nojiriensis, Streptomyces sporoverrucosus, Streptomyces venezuelae, Streptomyces vinaceus, Streptomyces virginiae, or Streptomyces xanthophaeus.

Embodiment 43. The composition of any one of embodiments 1-11, wherein the at least one microbial signaler comprises a 16S nucleic acid sequence having at least about 97% sequence identity to SEQ ID NO: 16.

Embodiment 44. The composition of embodiment 43, wherein the at least one microbial signaler is Streptomyces lavendulae, Streptomyces nojiriensis, Streptomyces spororaveus, Streptomyces subrutilus, Streptomyces venezuelae, Streptomyces virginiae, or Streptomyces xanthophaeus.

Embodiment 45. The composition of any one of embodiments 1-11, wherein the at least one microbial signaler comprises a 16S nucleic acid sequence having at least about 97% sequence identity to SEQ ID NO: 17.

Embodiment 46. The composition of embodiment 45, wherein the at least one microbial signaler is Streptomyces cinnamonensis, Streptomyces flaveus, Streptomyces goshikiensis, Streptomyces lavendulae, Streptomyces nojiriensis, Streptomyces sporoverrucosus, Streptomyces venezuelae, Streptomyces virginiae, or Streptomyces xanthophaeus.

Embodiment 47. The composition of any one of embodiments 1-11, wherein the at least one microbial signaler comprises a 16S nucleic acid sequence having at least about 97% sequence identity to SEQ ID NO: 18.

Embodiment 48. The composition of embodiment 47, wherein the at least one microbial signaler is Streptomyces colombiensis, Streptomyces flaveus, Streptomyces lavendulae, Streptomyces senoensis, Streptomyces sporoverrucosus, Streptomyces venezuelae, Streptomyces vinaceus, Streptomyces virginiae, or Streptomyces xanthophaeus.

Embodiment 49. The composition of any one of embodiments 1-11, wherein the at least one microbial signaler comprises a 16S nucleic acid sequence having at least about 97% sequence identity to SEQ ID NO: 19.

Embodiment 50. The composition of embodiment 49, wherein the at least one microbial signaler is Streptomyces flaveus, Streptomyces lavendulae, Streptomyces sporoverrucosus, Streptomyces venezuelae, Streptomyces vinaceus, Streptomyces virginiae, or Streptomyces xanthophaeus.

Embodiment 51. The composition of any one of embodiments 1-11, wherein the at least one microbial signaler comprises a 16S nucleic acid sequence having at least about 97% sequence identity to SEQ ID NO: 20.

Embodiment 52. The composition of embodiment 51, wherein the at least one microbial signaler is Streptomyces angustmyceticus, Streptomyces hygroscopicus, Streptomyces libani, Streptomyces lydicus, Streptomyces nigrescens, Streptomyces platensis, Streptomyces rimosus, or Streptomyces tubercidicus.

Embodiment 53. The composition of any one of embodiments 1-11, wherein the at least one microbial signaler comprises a 16S nucleic acid sequence having at least about 97% sequence identity to SEQ ID NO: 21.

Embodiment 54. The composition of embodiment 53, wherein the at least one microbial signaler is Streptomyces angustmyceticus, Streptomyces catenulae, Streptomyces cinereus, Streptomyces griseocarneus, Streptomyces hygroscopicus, Streptomyces libani, Streptomyces nigrescens, or Streptomyces sioyaensis.

Embodiment 55. The composition of any one of embodiments 1-11, wherein the at least one microbial signaler comprises a 16S nucleic acid sequence having at least about 97% sequence identity to SEQ ID NO: 22.

Embodiment 56. The composition of embodiment 55, wherein the at least one microbial signaler is Streptomyces atrolaccus, Streptomyces auratus, Streptomyces griseocarneus, Streptomyces hygroscopicus, Streptomyces libani, Streptomyces lydicus, Streptomyces sioyaensis, or Streptomyces tubercidicus.

Embodiment 57. The composition any one of embodiments 1-11, wherein the at least one microbial signaler comprises a 16S nucleic acid sequence having at least about 97% sequence identity to SEQ ID NO: 23.

Embodiment 58. The composition of embodiment 57, wherein the at least one microbial signaler is Streptomyces atrolaccus, Streptomyces auratus, Streptomyces griseocarneus, Streptomyces hygroscopicus, Streptomyces libani, Streptomyces lydicus, Streptomyces sioyaensis, or Streptomyces tubercidicus.

Embodiment 59. The composition of any one of embodiments 1-11, wherein the at least one microbial signaler comprises a 16S nucleic acid sequence having at least about 97% sequence identity to SEQ ID NO: 24.

Embodiment 60. The composition of embodiment 59, wherein the at least one microbial signaler is Streptomyces angustmyceticus, Streptomyces atrolaccus, Streptomyces griseocarneus, Streptomyces hygroscopicus, Streptomyces libani, Streptomyces nigrescens, Streptomyces sioyaensis, or Streptomyces tubercidicus.

Embodiment 61. The composition of any one of embodiments 1-11, wherein the at least one microbial signaler comprises a 16S nucleic acid sequence having at least about 97% sequence identity to SEQ ID NO: 25.

Embodiment 62. The composition of embodiment 61, wherein the at least one microbial signaler is Streptomyces angustmyceticus, Streptomyces hygroscopicus, Streptomyces libani, Streptomyces lydicus, Streptomyces nigrescens, Streptomyces platensis, Streptomyces rimosus, or Streptomyces tubercidicus.

Embodiment 63. The composition of any one of embodiments 1-11, wherein the at least one microbial signaler comprises a 16S nucleic acid sequence having at least about 97% sequence identity to SEQ ID NO: 26.

Embodiment 64. The composition of embodiment 63, wherein the at least one microbial signaler is Streptomyces angustmyceticus, Streptomyces hygroscopicus, Streptomyces libani, Streptomyces lydicus, Streptomyces nigrescens, Streptomyces platensis, Streptomyces rimosus, or Streptomyces sioyaensis.

Embodiment 65. 65. The composition of any one of embodiments 1-11, wherein the at least one microbial signaler comprises a 16S nucleic acid sequence having at least about 97% sequence identity to SEQ ID NO: 27.

Embodiment 66. The composition of embodiment 65, wherein the at least one microbial signaler is Streptomyces angustmyceticus, Streptomyces atrolaccus, Streptomyces chattanoogensis, Streptomyces libani, Streptomyces lydicus, Streptomyces nigrescens, Streptomyces sioyaensis, or Streptomyces tubercidicus.

Embodiment 67. The composition of any one of embodiments 1-11, wherein the at least one microbial signaler comprises a 16S nucleic acid sequence having at least about 97% sequence identity to SEQ ID NO: 28.

Embodiment 68. The composition of embodiment 67, wherein the at least one microbial signaler is Streptomyces angustmyceticus, Streptomyces hygroscopicus, Streptomyces libani, Streptomyces lydicus, Streptomyces nigrescens, Streptomyces platensis, Streptomyces rimosus, or Streptomyces tubercidicus.

Embodiment 69. The composition of any one of embodiments 1-11, wherein the at least one microbial signaler comprises a 16S nucleic acid sequence having at least about 97% sequence identity to SEQ ID NO: 29.

Embodiment 70. The composition of embodiment 69, wherein the at least one microbial signaler is Streptomyces angustmyceticus, Streptomyces atrolaccus, Streptomyces hygroscopicus, Streptomyces libani, Streptomyces lydicus, Streptomyces nigrescens, Streptomyces sioyaensis, or Streptomyces tubercidicus.

Embodiment 71. The composition any one of embodiments 1-11, wherein the at least one microbial signaler comprises a 16S nucleic acid sequence having at least about 97% sequence identity to SEQ ID NO: 30.

Embodiment 72. The composition of embodiment 71, wherein the at least one microbial signaler is Streptomyces caniferus, Streptomyces decoyicus, Streptomyces glebosus, Streptomyces hygroscopicus, Streptomyces libani, Streptomyces lydicus, Streptomyces ossamyceticus, or Streptomyces platensis.

Embodiment 73. The composition of any one of embodiments 1-11, wherein the at least one microbial signaler comprises a 16S nucleic acid sequence having at least about 97% sequence identity to SEQ ID NO: 31.

Embodiment 74. The composition of embodiment 73, wherein the at least one microbial signaler is Streptomyces angustmyceticus, Streptomyces catenulae, Streptomyces cinereus, Streptomyces libani, Streptomyces lydicus, Streptomyces nigrescens, Streptomyces platensis, or Streptomyces tubercidicus.

Embodiment 75. The composition of any one of embodiments 1-11, wherein the at least one microbial signaler comprises a 16S nucleic acid sequence having at least about 97% sequence identity to SEQ ID NO: 32.

Embodiment 76. The composition of embodiment 75, wherein the at least one microbial signaler is Streptomyces argenteolus, Streptomyces atrolaccus, Streptomyces chattanoogensis, Streptomyces chrestomyceticus, Streptomyces coelicolor, Streptomyces lydicus, Streptomyces microsporus, Streptomyces nigrescens, Streptomyces rimosus, or Streptomyces sioyaensis.

Embodiment 77. The composition of any one of embodiments 1-11, wherein the at least one microbial signaler comprises a 16S nucleic acid sequence having at least about 97% sequence identity to SEQ ID NO: 33.

Embodiment 78. The composition of embodiment 77, wherein the at least one microbial signaler is Streptomyces aquilus, Streptomyces caeruleatus, Streptomyces fagopyri, Streptomyces griseochromogenes, Streptomyces mirabilis, Streptomyces nojiriensis, Streptomyces pseudovenezuelae, Streptomyces viridochromogenes, or Streptomyces viridochromogenes.

Embodiment 79. The composition of any one of embodiments 1-11, wherein the at least one microbial signaler comprises a 16S nucleic acid sequence having at least about 97% sequence identity to SEQ ID NO: 34.

Embodiment 80. The composition of embodiment 79, wherein the at least one microbial signaler is Streptomyces aquilus, Streptomyces aureus, Streptomyces fagopyri, Streptomyces lutosisoli, Streptomyces minoensis, Streptomyces mirabilis, Streptomyces olivochromogenes, or Streptomyces rhizosphaerihabitans.

Embodiment 81. The composition of any one of embodiments 1-11, wherein the at least one microbial signaler comprises a 16S nucleic acid sequence having at least about 97% sequence identity to SEQ ID NO: 35.

Embodiment 82. The composition of embodiment 81, wherein the at least one microbial signaler is Streptomyces aquilus, Streptomyces fagopyri, Streptomyces griseoruber, Streptomyces lutosisoli, Streptomyces minoensis, Streptomyces mirabilis, Streptomyces olivochromogenes, or Streptomyces scabiei.

Embodiment 83. The composition of any one of embodiments 1-11, wherein the at least one microbial signaler comprises a 16S nucleic acid sequence having at least about 97% sequence identity to SEQ ID NO: 36.

Embodiment 84. The composition of embodiment 83, wherein the at least one microbial signaler is Streptomyces cirratus, Streptomyces nojiriensis, Streptomyces sporoverrucosus, Streptomyces venezuelae, Streptomyces verne, Streptomyces vinaceus, Streptomyces virginiae, or Streptomyces xanthophaeus.

Embodiment 85. The composition of any one of embodiments 1-84, wherein the at least one target microbe belongs to any one of the following genera: Talaromyces, Trichoderma, Bacillus, Streptomyces, Azospirillum, Pseudomonas, Comamonas, Citrobacter, Enterobacter, Bradyrhizobium, Rhizobium, Rhizophagus, and Glomus.

Embodiment 86. The composition of any one of embodiments 1-85, wherein the at least one target microbe belongs to any one of the following genera: Talaromyces, Streptomyces, Bacillus, Trichoderma, Pseudomonas, Comamonas, or Enterobacter.

Embodiment 87. The composition of any one of embodiments 1-85, wherein the at least one target microbe is Talaromyces flavus, Trichoderma harzianum, Bacillus amyloliquefaciens, Streptomyces sp., Bacillus subtilis, Bacillus amyloliquefaciens, Streptomyces lydicus, Pseudomonas chlororaphis, Bacillus subtilis, Azospirillum brasilense, Trichoderma asperellum, Trichoderma gamsii, Pseudomonas putida, Comamonas testosterone, Citrobacter freundii, Enterobacter cloacae, Streptomyces spp., Trichoderma viride, Bacillus megaterium, Azospirillum spp., Bradyrhizobium japonicum, Rhizobium leguminosarum biovar viciae, Bradyrhizobium spp., Rhizobium leguminosarum, Azospirillum amazonense, Azospirillum lipoferum, Glomus intraradices, Rhizophagus intraradices, Glomus mosseaem, or any combination thereof.

Embodiment 88. The composition of embodiment 87, wherein the at least one target microbe is Talaromyces flavus SAY-Y-94-01.

Embodiment 89. The composition of embodiment 1 or embodiment 87, wherein the at least one target microbe is Streptomyces lydicus WYEC 108.

Embodiment 90. A method of producing the composition of any one of embodiments 2-89, the method comprising: bringing the at least one target microbe in the physical proximity of the at least one microbial signaler.

Embodiment 91. A method of enhancing a plant growth-promoting function of a target microbe, the method comprising: bringing the target microbe in the physical proximity of the composition of any one of embodiments 1-89.

Embodiment 92. The method of embodiment 91, wherein the method comprises increasing the plant growth-promoting function of the target microbe by at least about 1%.

Embodiment 93. The method of any one of embodiments 91-92, wherein the plant growth-promoting function is a plant pathogen-inhibiting function, and wherein the method comprises increasing the plant pathogen-inhibiting function of the target microbe by at least about 1%.

Embodiment 94. The method of any one of embodiments 91-93, wherein the plant growth-promoting function is a zinc solubilizing function, and wherein the method comprises increasing the zinc solubilizing function of the target microbe by at least about 1%.

Embodiment 95. The method of any one of embodiments 91-94, wherein the plant growth-promoting function is a phosphate solubilizing function, and wherein the method comprises enhancing the phosphate solubilizing function of the target microbe by at least about 1%.

Embodiment 96. The method of any one of embodiments 91-95, wherein the method comprises enhancing the plant-growth promoting function of the target microbe under low nutrient conditions.

Embodiment 97. The method of any one of embodiments 90-96, wherein the method comprises bringing the target microbe in contact with the at least one microbial signaler.

Embodiment 98. The method of any one of embodiments 90-97, wherein the method comprises preparing a composition, comprising the target microbe and the at least one microbial signaler.

Embodiment 99. A method of producing an improved soil for growth of a plant, comprising: applying the composition of any one of embodiments 1-89 to soil, thereby producing the improved soil for plant growth.

Embodiment 100. A method of producing an improved soil for growth of a plant, comprising: applying the composition of any one of embodiments 2-89 to soil, thereby producing the improved soil for plant growth.

Embodiment 101. The method of embodiment 99 or embodiment 100, comprising allowing a plant to grow in the improved soil.

Embodiment 102. The method of any one of embodiments 99-101, wherein the growth of the plant is more enhanced in the improved soil, as compared to the growth of the plant in a negative control soil, wherein the composition is not applied to the negative control soil.

Embodiment 103. The method of any one of embodiments 99-102, wherein the method inhibits a plant pathogen in the improved soil.

Embodiment 104. The method of embodiment 103, wherein the inhibition of a plant pathogen in the improved soil is higher than in a negative control soil, wherein the composition is not applied to the negative control soil.

Embodiment 105. The method of embodiment 104, wherein the inhibition of a plant pathogen in the improved soil is at least about 1% higher than in a negative control soil, wherein the composition is not applied to the negative control soil.

Embodiment 106. The method of any one of embodiments 99-105, wherein the method increases the amount and/or concentration of soluble zinc in the improved soil.

Embodiment 107. The method of embodiment 106, wherein the amount and/or concentration of soluble zinc in the improved soil is higher than in a negative control soil, wherein the composition is not applied to the negative control soil.

Embodiment 108. The method of embodiment 107, wherein the amount and/or concentration of soluble zinc in the improved soil is at least about 1% higher than in a negative control soil, wherein the composition is not applied to the negative control soil.

Embodiment 109. The method of any one of embodiments 99-111, wherein the method increases the amount and/or concentration of soluble phosphate in the soil.

Embodiment 110. The method of embodiment 109, wherein the amount and/or concentration of soluble phosphate in the improved soil is higher than in a negative control soil, wherein the composition is not applied to the negative control soil.

Embodiment 111. The method of embodiment 110, wherein the amount and/or concentration of soluble phosphate in the improved soil is at least about 1% higher than in a control soil, wherein the composition is not applied to the negative control soil.

Embodiment 112. The method of embodiment 100, wherein the growth of the plant is more enhanced in the improved soil, as compared to the growth of the plant in a comparator control soil, wherein the at least one target microbe is applied to comparator control soil and the at least one microbial signaler is not applied to the comparator control soil.

Embodiment 113. The method of embodiment 112, wherein the growth of the plant is at least about 1% higher in the improved soil, as compared to the growth of the plant in a comparator control soil, wherein the at least one target microbe is applied to comparator control soil and the at least one microbial signaler is not applied to the comparator control soil.

Embodiment 114. The method of any one of embodiments 100, 112 and 113, wherein the inhibition of a plant pathogen in the improved soil is higher than in a comparator control soil, wherein the at least one target microbe is applied to comparator control soil and the at least one microbial signaler is not applied to the comparator control soil.

Embodiment 115. The method of any one of embodiments 100 and 112-114, wherein the inhibition of a plant pathogen in the improved soil is at least about 1% higher than in a comparator control soil, wherein the at least one target microbe is applied to comparator control soil and the at least one microbial signaler is not applied to the comparator control soil.

Embodiment 116. The method of any one of embodiments 100 and 112-115, wherein the method increases the amount and/or concentration of soluble zinc in the improved soil.

Embodiment 117. The method of embodiment 116, wherein the amount and/or concentration of soluble zinc in the improved soil is higher than in a comparator control soil, wherein the at least one target microbe is applied to comparator control soil and the at least one microbial signaler is not applied to the comparator control soil.

Embodiment 118. The method of embodiment 116 or embodiment 117, wherein the amount and/or concentration of soluble zinc in the improved soil is at least about 1% higher than in a comparator control soil, wherein the at least one target microbe is applied to comparator control soil and the at least one microbial signaler is not applied to the comparator control soil.

Embodiment 119. The method of any one of embodiments 100 and 112-118, wherein the method increases the amount and/or concentration of soluble phosphate in the soil.

Embodiment 120. The method of embodiment 119, wherein the amount and/or concentration of soluble phosphate in the improved soil is higher than in a comparator control soil, wherein the at least one target microbe is applied to comparator control soil and the at least one microbial signaler is not applied to the comparator control soil.

Embodiment 121. The method of embodiment 119 or 120, wherein the amount and/or concentration of soluble phosphate in the improved soil is at least about 1% higher than in a comparator control soil, wherein the at least one target microbe is applied to comparator control soil and the at least one microbial signaler is not applied to the comparator control soil.

Embodiment 122. The method of any one of embodiments 100 and 112-121, wherein the method improves the suppression of a disease associated with, promoted by, or caused by a pathogen in the plant.

Embodiment 123. The method of embodiment 122, wherein the suppression of the disease associated with, promoted by, or caused by the pathogen in the plant is higher than in a comparator control soil, wherein the at least one target microbe is applied to comparator control soil and the at least one microbial signaler is not applied to the comparator control soil.

Embodiment 124. The method of embodiment 123, wherein the suppression of the disease associated with, promoted by, or caused by the pathogen in the plant is at least about 1% higher than in a comparator control soil, wherein the at least one target microbe is applied to comparator control soil and the at least one microbial signaler is not applied to the comparator control soil.

Embodiment 125. The method of any one of embodiments 100 and 112-124, wherein the method increases the above-ground biomass of the plant.

Embodiment 126. The method of embodiment 125, wherein the above-ground biomass of the plant is higher than in a comparator control soil, wherein the at least one target microbe is applied to comparator control soil and the at least one microbial signaler is not applied to the comparator control soil.

Embodiment 127. The method of embodiment 126, wherein the above-ground biomass of the plant is at least about 1% higher than in a comparator control soil, wherein the at least one target microbe is applied to comparator control soil and the at least one microbial signaler is not applied to the comparator control soil.

Embodiment 128. The method of any one of embodiments 100 and 112-124, wherein the method increases the below-ground biomass of the plant.

Embodiment 129. The method of embodiment 128, wherein the below-ground biomass of the plant is higher than in a comparator control soil, wherein the at least one target microbe is applied to comparator control soil and the at least one microbial signaler is not applied to the comparator control soil.

Embodiment 130. The method of embodiment 129, wherein the below-ground biomass of the plant is at least about 1% higher than in a comparator control soil, wherein the at least one target microbe is applied to comparator control soil and the at least one microbial signaler is not applied to the comparator control soil.

Embodiment 131. The method of any one of embodiments 99-130, wherein the composition is applied before planting, after plant germination, as a seed treatment, as a spray, and/or as a soil drench.

Embodiment 132. The method of any one of embodiments 103-105, 114-115 and 122-124, or the composition of any one of embodiments 5-6, wherein the plant pathogen belongs to one of the following genera: Pseudomonas, Erwinia, Raltsonia, Rhizomonas, Agrobacterium, Streptomyces, Bacillus, Sclerotium, Rhizoctonia, Fusarium, Pythium, Phytophthora, Synchytrium, Rhizopus, Alternaria, Macrophomina, Drechslera, Bipolaris, Curvularia, Phomopsis, Caloscypha fulgens, usarium circinatum, Fusarium oxysporum, Fusarium moniliforme var. moniliforme, Lasiodiplodia theobromae, Sirococcus conigenus, Diplodia pinea, Ustilago nuda, Pyrenophora graminea, Pyrenophora teres, Xanthomonas translucens, Pseudomonas syringae, Fusarium graminearum, Bipolaris sorokiniana, Xanthomonas campestris, Aciculosporium, Mycosphaerella, Ceratobasidium, Albugo, Alternaria, Myrothecium, Cochliobolus, Hyaloperonospora, Alveopora, Neonectria, Colletotrichum, Peronospora, Cadophora, Oculimacula, Curvularia, Phytophthora, Calyptella Omphalotus, Cylindrocladiella, Plasmopara, Chrysomyxa, Peyronellaea, Fusarium, Pythiogeton, Cladophialophora, Phaeoacremonium, Heterobasidion, Pythium, Coleosporium, Pseudocercospora, Magnaporthe, Colletotrichum, Puccinia, Microdochium, Corynespora, Pucciniastrum, Olpidium, Craterocolla, Pseudotetraploa, Phoma, Cronartium, Septoria, Plectosphaerella Didymella, Sphacelotheca, Pyrenochaeta, Drechslera, Spongipellis, Rhizoctonia, Endocronartium, Stenocarpella, Setophoma, Entyloma, Sydowia, Spongospora, Fomitopsis, Taphrina, Thielaviopsis, Fusarium, Tritirachium, Typhula, Ganoderma, Urocystis, Verticillium, Hypohelion, Ustilago, Waitea, Itersonilia, Venturia, Leptosphaerulina, Verticillium, and Monilinia.

What is claimed is:

1. A composition, comprising at least one microbial signaler, wherein the at least one microbial signaler is capable of enhancing a plant growth-promoting function of at least one target microbe.

2. The composition of claim 1, comprising: the at least one target microbe.

3. The composition of claim 1, wherein the at least one microbial signaler belongs to the genus Streptomyces, Fusarium, or Bacillus.

4. The composition of claim 1, wherein the at least one microbial signaler belongs to the genus Streptomyces.

5. The composition of claim 1, wherein the plant growth-promoting function comprises: (a) plant pathogen-inhibiting function, (b) zinc solubilizing function, (c) phosphate solubilizing function, (d) production of an antibiotic, (e) nitrogen fixing function, (f) a function of improving a plant's nutrient acquisition, (f) production of plant growth hormones, or (g) any combination thereof.

6. The composition of of claim 5, wherein the at least one microbial signaler is capable of enhancing the plant pathogen-inhibiting function of the target microbe by at least about 5%.

7. The composition of of claim 5, wherein the at least one microbial signaler is capable of enhancing the zinc solubilizing function of the target microbe by at least about 5%.

8. The composition of claim 5, wherein the at least one microbial signaler is capable of enhancing the phosphate solubilizing function of the target microbe by at least about 5%.

9. The composition of claim 5, wherein the at least one microbial signaler is capable of enhancing the plant growth-promoting function of the at least one target microbe under low nutrient conditions.

10. The composition of claim 1, wherein the at least one microbial signaler is Streptomyces avidinii, Streptomyces colombiensis, Streptomyces lavendulae, Streptomyces roseochromogenus, Streptomyces spororaveus, Streptomyces sporoverrucosus, Streptomyces venezuelae, Streptomyces xanthophaeus, Streptomyces angustmyceticus, Streptomyces hygroscopicus, Streptomyces libani, Streptomyces lydicus, Streptomyces nigrescens, Streptomyces platensis, Streptomyces rimosus, Streptomyces tubercidicus, Streptomyces bungoensis, Streptomyces cyslabdanicus, Streptomyces galbus, Streptomyces kagawaensis, Streptomyces lasaliensis, Streptomyces lasalocidi, Streptomyces longwoodensis, Streptomyces spinichromogenes, Streptomyces cirratus, Streptomyces nojiriensis, Streptomyces verne, Streptomyces vinaceus, Streptomyces virginiae, Streptomyces catenulae, Streptomyces cinereus, Streptomyces griseocarneus, Streptomyces sioyaensis, Streptomyces subrutilus, Streptomyces atrolaccus, Streptomyces auratus, Streptomyces fagopyri, Streptomyces kaempferi, Streptomyces mirabilis, Streptomyces olivochromogenes, Streptomyces chattanoogensis, Streptomyces flaveus, Streptomyces goshikiensis, Streptomyces cinnamonensis, Streptomyces senoensis, Streptomyces echinatus, Streptomyces filipinensis, Streptomyces gulbargensis, Streptomyces myxogenes, Streptomyces novaecaesareae, Streptomyces spectabilis, Streptomyces tanashiensis, Streptomyces ginsengisoli, Streptomyces graminisoli, Streptomyces lucensis, Streptomyces yaanensis, Streptomyces canferus, Streptomyces decoyicus, Streptomyces glebosus, Streptomyces ossamyceticus, Streptomyces badius, Streptomyces cyaneofuscatus, Streptomyces flavogriseus, Streptomyces griseus, Streptomyces mediolani, Streptomyces praecox, Streptomyces pratensis, Streptomyces omiyaensis, Streptomyces aquilus, Streptomyces caeruleatus, Streptomyces griseochromogenes, Streptomyces pseudovenezuelae, Streptomyces viridochromogenes, Streptomyces argenteolus, Streptomyces chrestomyceticus, Streptomyces coelicolor, Streptomyces microsporus, Streptomyces aureus, Streptomyces lutosisoli, Streptomyces minoensis, Streptomyces rhizosphaerihabitans, Streptomyces griseoruber, Streptomyces scabiei, Streptomyces achromogenes, Streptomyces canarius, Streptomyces capoamus, Streptomyces cellostaticus, or Streptomyces katrae.

11. The composition of claim 1, wherein the at least one microbial signaler comprises a 16S nucleic acid sequence having at least about 97% sequence identity to one or more of the following SEQ ID Nos: 1-36.

12. The composition of claim 1, wherein the at least one microbial signaler comprises a 16S nucleic acid sequence of any one of the following SEQ ID Nos: 1-36.

13. The composition of claim 1, wherein the at least one microbial signaler comprises a 16S nucleic acid sequence having at least about 97% sequence identity to SEQ ID NO: 1.

14. The composition of claim 13, wherein the at least one microbial signaler is Streptomyces echinatus, Streptomyces filipinensis, Streptomyces gulbargensis, Streptomyces longwoodensis, Streptomyces myxogenes, Streptomyces novaecaesareae, Streptomyces spectabilis, or Streptomyces tanashiensis.

15. The composition of claim 1, wherein the at least one microbial signaler comprises a 16S nucleic acid sequence having at least about 97% sequence identity to SEQ ID NO: 2.

16. The composition of claim 15, wherein the at least one microbial signaler is Streptomyces echinatus, Streptomyces ginsengisoli, Streptomyces graminisoli, Streptomyces gulbargensis, Streptomyces longwoodensis, Streptomyces lucensis, Streptomyces tanashiensis, or Streptomyces yaanensis.

17. The composition of claim 1, wherein the at least one microbial signaler comprises a 16S nucleic acid sequence having at least about 97% sequence identity to SEQ ID NO: 3.

18. The composition of claim 17, wherein the at least one microbial signaler is Streptomyces bungoensis, Streptomyces cyslabdanicus, Streptomyces galbus, Streptomyces kagawaensis, Streptomyces lasaliensis, Streptomyces lasalocidi, Streptomyces longwoodensis, or Streptomyces spinichromogenes.

19. The composition of claim 1, wherein the at least one microbial signaler comprises a 16S nucleic acid sequence having at least about 97% sequence identity to SEQ ID NO: 4.

20. The composition of claim 19, wherein the at least one microbial signaler is Streptomyces auratus, Streptomyces cyslabdanicus, Streptomyces fagopyri, Streptomyces galbus, Streptomyces kaempferi, Streptomyces mirabilis, or Streptomyces olivochromogenes.

21. The composition of claim 1, wherein the at least one microbial signaler comprises a 16S nucleic acid sequence having at least about 97% sequence identity to SEQ ID NO: 5.

22. The composition of claim 21, wherein the at least one microbial signaler is Streptomyces achromogenes, Streptomyces bungoensis, Streptomyces canarius, Streptomyces capoamus, Streptomyces cellostaticus, Streptomyces galbus, Streptomyces katrae, or Streptomyces spinichromogenes.

23. The composition of claim 1, wherein the at least one microbial signaler comprises a 16S nucleic acid sequence having at least about 97% sequence identity to SEQ ID NO: 6.

24. The composition of of claim 23, wherein the at least one microbial signaler is Streptomyces avidinii, Streptomyces colombiensis, Streptomyces lavendulae, Streptomyces roseochromogenus, Streptomyces spororaveus, Streptomyces sporoverrucosus, Streptomyces venezuelae, or Streptomyces xanthophaeus.

25. The composition of claim 1, wherein the at least one microbial signaler comprises a 16S nucleic acid sequence having at least about 97% sequence identity to SEQ ID NO: 7.

26. The composition of of claim 25, wherein the at least one microbial signaler is Streptomyces avidinii, Streptomyces cirratus, Streptomyces lavendulae, Streptomyces nojiriensis, Streptomyces spororaveus, Streptomyces subrutilus, Streptomyces venezuelae, or Streptomyces xanthophaeus.

27. The composition of claim 1, wherein the at least one microbial signaler comprises a 16S nucleic acid sequence having at least about 97% sequence identity to SEQ ID NO: 8.

28. The composition of claim 27, wherein the at least one microbial signaler is Streptomyces avidinii, Streptomyces colombiensis, Streptomyces flaveus, Streptomyces goshikiensis, Streptomyces lavendulae, Streptomyces spororaveus, Streptomyces subrutilus, or Streptomyces venezuelae.

29. The composition of claim 1, wherein the at least one microbial signaler comprises a 16S nucleic acid sequence having at least about 97% sequence identity to SEQ ID NO: 9.

30. The composition of claim 29, wherein the at least one microbial signaler is Streptomyces cinnamonensis, Streptomyces cirratus, Streptomyces goshikiensis, Streptomyces lavendulae, Streptomyces senoensis, Streptomyces sporoverrucosus, Streptomyces vinaceus, or Streptomyces virginiae.

31. The composition of claim 1, wherein the at least one microbial signaler comprises a 16S nucleic acid sequence having at least about 97% sequence identity to SEQ ID NO: 10.

32. The composition of claim 31, wherein the at least one microbial signaler is Streptomyces cinnamonensis, Streptomyces flaveus, Streptomyces lavendulae, Streptomyces nojiriensis, Streptomyces spororaveus, Streptomyces venezuelae, Streptomyces virginiae, or Streptomyces xanthophaeus.

33. The composition of claim 1, wherein the at least one microbial signaler comprises a 16S nucleic acid sequence having at least about 97% sequence identity to SEQ ID NO: 11.

34. The composition of of claim 33, wherein the at least one microbial signaler is Streptomyces auratus, Streptomyces cinnamonensis, Streptomyces lavendulae, Streptomyces sioyaensis, Streptomyces spororaveus, Streptomyces verne, Streptomyces virginiae, or Streptomyces xanthophaeus.

35. The composition of claim 1, wherein the at least one microbial signaler comprises a 16S nucleic acid sequence having at least about 97% sequence identity to SEQ ID NO: 12.

36. The composition of claim 35, wherein the at least one microbial signaler is Streptomyces cinnamonensis, Streptomyces flaveus, Streptomyces lavendulae, Streptomyces nojiriensis, Streptomyces spororaveus, Streptomyces venezuelae, Streptomyces virginiae, or Streptomyces xanthophaeus.

37. The composition of claim 1, wherein the at least one microbial signaler comprises a 16S nucleic acid sequence having at least about 97% sequence identity to SEQ ID NO: 13.

38. The composition of claim 37, wherein the at least one microbial signaler is Streptomyces badius, Streptomyces cyaneofuscatus, Streptomyces flavogriseus, Streptomyces griseus, Streptomyces lavendulae, Streptomyces mediolani, Streptomyces praecox, or Streptomyces pratensis.

39. The composition of claim 1, wherein the at least one microbial signaler comprises a 16S nucleic acid sequence having at least about 97% sequence identity to SEQ ID NO: 14.

40. The composition of claim 39, wherein the at least one microbial signaler is Streptomyces avidinii, Streptomyces cirratus, Streptomyces lavendulae, Streptomyces nojiriensis, Streptomyces omiyaensis, Streptomyces spororaveus, Streptomyces subrutilus, or Streptomyces vinaceus.

41. The composition of claim 1, wherein the at least one microbial signaler comprises a 16S nucleic acid sequence having at least about 97% sequence identity to SEQ ID NO: 15.

42. The composition of claim 41, wherein the at least one microbial signaler is Streptomyces cinnamonensis, Streptomyces flaveus, Streptomyces lavendulae, Streptomyces nojiriensis, Streptomyces sporoverrucosus, Streptomyces venezuelae, Streptomyces vinaceus, Streptomyces virginiae, or Streptomyces xanthophaeus.

43. The composition of claim 1, wherein the at least one microbial signaler comprises a 16S nucleic acid sequence having at least about 97% sequence identity to SEQ ID NO: 16.

44. The composition of claim 43, wherein the at least one microbial signaler is Streptomyces lavendulae, Streptomyces nojiriensis, Streptomyces spororaveus, Streptomyces subrutilus, Streptomyces venezuelae, Streptomyces virginiae, or Streptomyces xanthophaeus.

45. The composition of claim 1, wherein the at least one microbial signaler comprises a 16S nucleic acid sequence having at least about 97% sequence identity to SEQ ID NO: 17.

46. The composition of claim 45, wherein the at least one microbial signaler is Streptomyces cinnamonensis, Streptomyces flaveus, Streptomyces goshikiensis, Streptomyces lavendulae, Streptomyces nojiriensis, Streptomyces sporoverrucosus, Streptomyces venezuelae, Streptomyces virginiae, or Streptomyces xanthophaeus.

47. The composition of claim 1, wherein the at least one microbial signaler comprises a 16S nucleic acid sequence having at least about 97% sequence identity to SEQ ID NO: 18.

48. The composition of claim 47, wherein the at least one microbial signaler is Streptomyces colombiensis, Streptomyces flaveus, Streptomyces lavendulae, Streptomyces senoensis, Streptomyces sporoverrucosus, Streptomyces venezuelae, Streptomyces vinaceus, Streptomyces virginiae, or Streptomyces xanthophaeus.

49. The composition of claim 1, wherein the at least one microbial signaler comprises a 16S nucleic acid sequence having at least about 97% sequence identity to SEQ ID NO: 19.

50. The composition of claim 49, wherein the at least one microbial signaler is Streptomyces flaveus, Streptomyces lavendulae, Streptomyces sporoverrucosus, Streptomyces venezuelae, Streptomyces vinaceus, Streptomyces virginiae, or Streptomyces xanthophaeus.

51. The composition of claim 1, wherein the at least one microbial signaler comprises a 16S nucleic acid sequence having at least about 97% sequence identity to SEQ ID NO: 20.

52. The composition of claim 51, wherein the at least one microbial signaler is Streptomyces angustmyceticus, Streptomyces hygroscopicus, Streptomyces libani, Streptomyces lydicus, Streptomyces nigrescens, Streptomyces platensis, Streptomyces rimosus, or Streptomyces tubercidicus.

53. The composition of claim 1, wherein the at least one microbial signaler comprises a 16S nucleic acid sequence having at least about 97% sequence identity to SEQ ID NO: 21.

54. The composition of claim 53, wherein the at least one microbial signaler is Streptomyces angustmyceticus, Streptomyces catenulae, Streptomyces cinereus, Streptomyces griseocarneus, Streptomyces hygroscopicus, Streptomyces libani, Streptomyces nigrescens, or Streptomyces sioyaensis.

55. The composition of claim 1, wherein the at least one microbial signaler comprises a 16S nucleic acid sequence having at least about 97% sequence identity to SEQ ID NO: 22.

56. The composition of claim 55, wherein the at least one microbial signaler is Streptomyces atrolaccus, Streptomyces auratus, Streptomyces griseocarneus, Streptomyces hygroscopicus, Streptomyces libani, Streptomyces lydicus, Streptomyces sioyaensis, or Streptomyces tubercidicus.

57. The composition claim 1, wherein the at least one microbial signaler comprises a 16S nucleic acid sequence having at least about 97% sequence identity to SEQ ID NO: 23.

58. The composition of claim 57, wherein the at least one microbial signaler is Streptomyces atrolaccus, Streptomyces auratus, Streptomyces griseocarneus, Streptomyces hygroscopicus, Streptomyces libani, Streptomyces lydicus, Streptomyces sioyaensis, or Streptomyces tubercidicus.

59. The composition of claim 1, wherein the at least one microbial signaler comprises a 16S nucleic acid sequence having at least about 97% sequence identity to SEQ ID NO: 24.

60. The composition of claim 59, wherein the at least one microbial signaler is Streptomyces angustmyceticus, Streptomyces atrolaccus, Streptomyces griseocarneus, Streptomyces hygroscopicus, Streptomyces libani, Streptomyces nigrescens, Streptomyces sioyaensis, or Streptomyces tubercidicus.

61. The composition of claim 1, wherein the at least one microbial signaler comprises a 16S nucleic acid sequence having at least about 97% sequence identity to SEQ ID NO: 25.

62. The composition of claim 61, wherein the at least one microbial signaler is Streptomyces angustmyceticus, Streptomyces hygroscopicus, Streptomyces libani, Streptomyces lydicus, Streptomyces nigrescens, Streptomyces platensis, Streptomyces rimosus, or Streptomyces tubercidicus.

63. The composition of claim 1, wherein the at least one microbial signaler comprises a 16S nucleic acid sequence having at least about 97% sequence identity to SEQ ID NO: 26.

64. The composition of claim 63, wherein the at least one microbial signaler is Streptomyces angustmyceticus, Streptomyces hygroscopicus, Streptomyces libani, Streptomyces lydicus, Streptomyces nigrescens, Streptomyces platensis, Streptomyces rimosus, or Streptomyces sioyaensis.

65. The composition of claim 1, wherein the at least one microbial signaler comprises a 16S nucleic acid sequence having at least about 97% sequence identity to SEQ ID NO: 27.

66. The composition of claim 65, wherein the at least one microbial signaler is Streptomyces angustmyceticus, Streptomyces atrolaccus, Streptomyces chattanoogensis, Streptomyces libani, Streptomyces lydicus, Streptomyces nigrescens, Streptomyces sioyaensis, or Streptomyces tubercidicus.

67. The composition of claim 1, wherein the at least one microbial signaler comprises a 16S nucleic acid sequence having at least about 97% sequence identity to SEQ ID NO: 28.

68. The composition of claim 67, wherein the at least one microbial signaler is Streptomyces angustmyceticus, Streptomyces hygroscopicus, Streptomyces libani, Streptomyces lydicus, Streptomyces nigrescens, Streptomyces platensis, Streptomyces rimosus, or Streptomyces tubercidicus.

69. The composition of claim 1, wherein the at least one microbial signaler comprises a 16S nucleic acid sequence having at least about 97% sequence identity to SEQ ID NO: 29.

70. The composition of claim 69, wherein the at least one microbial signaler is Streptomyces angustmyceticus, Streptomyces atrolaccus, Streptomyces hygroscopicus, Streptomyces libani, Streptomyces lydicus, Streptomyces nigrescens, Streptomyces sioyaensis, or Streptomyces tubercidicus.

71. The composition of claim 1, wherein the at least one microbial signaler comprises a 16S nucleic acid sequence having at least about 97% sequence identity to SEQ ID NO: 30.

72. The composition of claim 71, wherein the at least one microbial signaler is Streptomyces canferus, Streptomyces decoyicus, Streptomyces glebosus, Streptomyces hygroscopicus, Streptomyces libani, Streptomyces lydicus, Streptomyces ossamyceticus, or Streptomyces platensis.

73. The composition of claim 1, wherein the at least one microbial signaler comprises a 16S nucleic acid sequence having at least about 97% sequence identity to SEQ ID NO: 31.

74. The composition of claim 73, wherein the at least one microbial signaler is Streptomyces angustmyceticus, Streptomyces catenulae, Streptomyces cinereus, Streptomyces libani, Streptomyces lydicus, Streptomyces nigrescens, Streptomyces platensis, or Streptomyces tubercidicus.

75. The composition of claim 1, wherein the at least one microbial signaler comprises a 16S nucleic acid sequence having at least about 97% sequence identity to SEQ ID NO: 32.

76. The composition of claim 75, wherein the at least one microbial signaler is Streptomyces argenteolus, Streptomyces atrolaccus, Streptomyces chattanoogensis, Streptomyces chrestomyceticus, Streptomyces coelicolor, Streptomyces lydicus, Streptomyces microsporus, Streptomyces nigrescens, Streptomyces rimosus, or Streptomyces sioyaensis.

77. The composition of claim 1, wherein the at least one microbial signaler comprises a 16S nucleic acid sequence having at least about 97% sequence identity to SEQ ID NO: 33.

78. The composition of claim 77, wherein the at least one microbial signaler is Streptomyces aquilus, Streptomyces caeruleatus, Streptomyces fagopyri, Streptomyces griseochromogenes, Streptomyces mirabilis, Streptomyces nojiriensis, Streptomyces pseudovenezuelae, Streptomyces viridochromogenes, or Streptomyces viridochromogenes.

79. The composition of claim 1, wherein the at least one microbial signaler comprises a 16S nucleic acid sequence having at least about 97% sequence identity to SEQ ID NO: 34.

80. The composition of claim 79, wherein the at least one microbial signaler is Streptomyces aquilus, Streptomyces aureus, Streptomyces fagopyri, Streptomyces lutosisoli, Streptomyces minoensis, Streptomyces mirabilis, Streptomyces olivochromogenes, or Streptomyces rhizosphaerihabitans.

81. The composition of claim 1, wherein the at least one microbial signaler comprises a 16S nucleic acid sequence having at least about 97% sequence identity to SEQ ID NO: 35.

82. The composition of claim 81, wherein the at least one microbial signaler is Streptomyces aquilus, Streptomyces fagopyri, Streptomyces griseoruber, Streptomyces lutosisoli, Streptomyces minoensis, Streptomyces mirabilis, Streptomyces olivochromogenes, or Streptomyces scabiei.

83. The composition of claim 1, wherein the at least one microbial signaler comprises a 16S nucleic acid sequence having at least about 97% sequence identity to SEQ ID NO: 36.

84. The composition of claim 83, wherein the at least one microbial signaler is Streptomyces cirratus, Streptomyces nojiriensis, Streptomyces sporoverrucosus, Streptomyces venezuelae, Streptomyces verne, Streptomyces vinaceus, Streptomyces virginiae, or Streptomyces xanthophaeus.

85. The composition of claim 1, wherein the at least one target microbe belongs to any one of the following genera: Talaromyces, Trichoderma, Bacillus, Streptomyces, Azospirillum, Pseudomonas, Comamonas, Citrobacter, Enterobacter, Bradyrhizobium, Rhizobium, Rhizophagus, and Glomus.

86. The composition of claim 1, wherein the at least one target microbe belongs to any one of the following genera: Talaromyces, Streptomyces, Bacillus, Trichoderma, Pseudomonas, Comamonas, or Enterobacter.

87. The composition of claim 2, wherein the at least one target microbe is Talaromyces flavus, Trichoderma harzianum, Bacillus amyloliquefaciens, Streptomyces sp., Bacillus subtilis, Bacillus amyloliquefaciens, Streptomyces lydicus, Pseudomonas chlororaphis, Bacillus subtilis, Azospirillum brasilense, Trichoderma asperellum, Trichoderma gamsii, Pseudomonas putida, Comamonas testosterone, Citrobacter freundii, Enterobacter cloacae, Streptomyces spp., Trichoderma viride, Bacillus megaterium, Azospirillum spp., Bradyrhizobium japonicum, Rhizobium leguminosarum biovar viciae, Bradyrhizobium spp., Rhizobium leguminosarum, Azospirillum amazonense, Azospirillum lipoferum, Glomus intraradices, Rhizophagus intraradices, Glomus mosseaem, or any combination thereof.

88. The composition of claim 87, wherein the at least one target microbe is Talaromyces flavus SAY-Y-94-01.

89. The composition of claim 87, wherein the at least one target microbe is Streptomyces lydicus WYEC 108.

90. A method of producing the composition of claim 2, the method comprising: bringing the at least one target microbe in the physical proximity of the at least one microbial signaler.

91. A method of enhancing a plant growth-promoting function of a target microbe, the method comprising: bringing the target microbe in the physical proximity of the composition of claim 1.

92. The method of claim 91, wherein the method comprises increasing the plant growth-promoting function of the target microbe by at least about 1%.

93. The method of claim 91, wherein the plant growth-promoting function is a plant pathogen-inhibiting function, and wherein the method comprises increasing the plant pathogen-inhibiting function of the target microbe by at least about 1%.

94. The method of claim 91, wherein the plant growth-promoting function is a zinc solubilizing function, and wherein the method comprises increasing the zinc solubilizing function of the target microbe by at least about 1%.

95. The method of claim 91, wherein the plant growth-promoting function is a phosphate solubilizing function, and wherein the method comprises enhancing the phosphate solubilizing function of the target microbe by at least about 1%.

96. The method of claim 91, wherein the method comprises enhancing the plant-growth promoting function of the target microbe under low nutrient conditions.

97. The method of claim 90, wherein the method comprises bringing the target microbe in contact with the at least one microbial signaler.

98. The method of claim 90, wherein the method comprises preparing a composition, comprising the target microbe and the at least one microbial signaler.

99. A method of producing an improved soil for growth of a plant, comprising: applying the composition of claim 1 to soil, thereby producing the improved soil for plant growth.

100. A method of producing an improved soil for growth of a plant, comprising: applying the composition of claim 2 to soil, thereby producing the improved soil for plant growth.

101. The method of claim 99, comprising allowing a plant to grow in the improved soil.

102. The method of claim 99, wherein the growth of the plant is more enhanced in the improved soil, as compared to the growth of the plant in a negative control soil, wherein the composition is not applied to the negative control soil.

103. The method of claim 99, wherein the method inhibits a plant pathogen in the improved soil.

104. The method of claim 103, wherein the inhibition of a plant pathogen in the improved soil is higher than in a negative control soil, wherein the composition is not applied to the negative control soil.

105. The method of claim 104, wherein the inhibition of a plant pathogen in the improved soil is at least about 1% higher than in a negative control soil, wherein the composition is not applied to the negative control soil.

106. The method of claim 99, wherein the method increases the amount and/or concentration of soluble zinc in the improved soil.

107. The method of claim 106, wherein the amount and/or concentration of soluble zinc in the improved soil is higher than in a negative control soil, wherein the composition is not applied to the negative control soil.

108. The method of claim 107, wherein the amount and/or concentration of soluble zinc in the improved soil is at least about 1% higher than in a negative control soil, wherein the composition is not applied to the negative control soil.

109. The method of claim 99, wherein the method increases the amount and/or concentration of soluble phosphate in the soil.

110. The method of claim 109, wherein the amount and/or concentration of soluble phosphate in the improved soil is higher than in a negative control soil, wherein the composition is not applied to the negative control soil.

111. The method of claim 110, wherein the amount and/or concentration of soluble phosphate in the improved soil is at least about 1% higher than in a control soil, wherein the composition is not applied to the negative control soil.

112. The method of claim 100, wherein the growth of the plant is more enhanced in the improved soil, as compared to the growth of the plant in a comparator control soil, wherein the at least one target microbe is applied to comparator control soil and the at least one microbial signaler is not applied to the comparator control soil.

113. The method of claim 112, wherein the growth of the plant is at least about 1% higher in the improved soil, as compared to the growth of the plant in a comparator control soil, wherein the at least one target microbe is applied to comparator control soil and the at least one microbial signaler is not applied to the comparator control soil.

114. The method of claim 100, wherein the inhibition of a plant pathogen in the improved soil is higher than in a comparator control soil, wherein the at least one target microbe is applied to comparator control soil and the at least one microbial signaler is not applied to the comparator control soil.

115. The method of claim 114, wherein the inhibition of a plant pathogen in the improved soil is at least about 1% higher than in a comparator control soil, wherein the at least one target microbe is applied to comparator control soil and the at least one microbial signaler is not applied to the comparator control soil.

116. The method of claim 100, wherein the method increases the amount and/or concentration of soluble zinc in the improved soil.

117. The method of claim 116, wherein the amount and/or concentration of soluble zinc in the improved soil is higher than in a comparator control soil, wherein the at least one target microbe is applied to comparator control soil and the at least one microbial signaler is not applied to the comparator control soil.

118. The method of claim 116, wherein the amount and/or concentration of soluble zinc in the improved soil is at least about 1% higher than in a comparator control soil, wherein the at least one target microbe is applied to comparator control soil and the at least one microbial signaler is not applied to the comparator control soil.

119. The method of claim 100, wherein the method increases the amount and/or concentration of soluble phosphate in the soil.

120. The method of claim 119, wherein the amount and/or concentration of soluble phosphate in the improved soil is higher than in a comparator control soil, wherein the at least one target microbe is applied to comparator control soil and the at least one microbial signaler is not applied to the comparator control soil.

121. The method of claim 119, wherein the amount and/or concentration of soluble phosphate in the improved soil is at least about 1% higher than in a comparator control soil, wherein the at least one target microbe is applied to comparator control soil and the at least one microbial signaler is not applied to the comparator control soil.

122. The method of claim 100, wherein the method improves the suppression of a disease associated with, promoted by, or caused by a pathogen in the plant.

123. The method of claim 122, wherein the suppression of the disease associated with, promoted by, or caused by the pathogen in the plant is higher than in a comparator control soil, wherein the at least one target microbe is applied to comparator control soil and the at least one microbial signaler is not applied to the comparator control soil.

124. The method of claim 123, wherein the suppression of the disease associated with, promoted by, or caused by the pathogen in the plant is at least about 1% higher than in a comparator control soil, wherein the at least one target microbe is applied to comparator control soil and the at least one microbial signaler is not applied to the comparator control soil.

125. The method of claim 100, wherein the method increases the above-ground biomass of the plant.

126. The method of claim 125, wherein the above-ground biomass of the plant is higher than in a comparator control soil, wherein the at least one target microbe is applied to comparator control soil and the at least one microbial signaler is not applied to the comparator control soil.

127. The method of claim 126, wherein the above-ground biomass of the plant is at least about 1% higher than in a comparator control soil, wherein the at least one target microbe is applied to comparator control soil and the at least one microbial signaler is not applied to the comparator control soil.

128. The method of claim 100, wherein the method increases the below-ground biomass of the plant.

129. The method of claim 128, wherein the below-ground biomass of the plant is higher than in a comparator control soil, wherein the at least one target microbe is applied to comparator control soil and the at least one microbial signaler is not applied to the comparator control soil.

130. The method of claim 129, wherein the below-ground biomass of the plant is at least about 1% higher than in a comparator control soil, wherein the at least one target microbe is applied to comparator control soil and the at least one microbial signaler is not applied to the comparator control soil.

131. The method of claim 99, wherein the composition is applied before planting, after plant germination, as a seed treatment, as a spray, and/or as a soil drench.

132. The method of claim 103, wherein the plant pathogen belongs to one of the following genera: Pseudomonas, Erwinia, Raltsonia, Rhizomonas, Agrobacterium, Streptomyces, Bacillus, Sclerotium, Rhizoctonia, Fusarium, Pythium, Phytophthora, Synchytrium, Rhizopus, Alternaria, Macrophomina, Drechslera, Bipolaris, Curvularia, Phomopsis, Caloscypha fulgens, usarium circinatum, Fusarium oxysporum, Fusarium moniliforme var. moniliforme, Lasiodiplodia theobromae, Sirococcus conigenus, Diplodia pinea, Ustilago nuda, Pyrenophora graminea, Pyrenophora teres, Xanthomonas translucens, Pseudomonas syringae, Fusarium graminearum, Bipolaris sorokiniana, Xanthomonas campestris, Aciculosporium, Mycosphaerella, Ceratobasidium, Albugo, Alternaria, Myrothecium, Cochliobolus, Hyaloperonospora, Alveopora, Neonectria, Colletotrichum, Peronospora, Cadophora, Oculimacula, Curvularia, Phytophthora, Calyptella Omphalotus, Cylindrocladiella, Plasmopara, Chrysomyxa, Peyronellaea, Fusarium, Pythiogeton, Cladophialophora, Phaeoacremonium, Heterobasidion, Pythium, Coleosporium, Pseudocercospora, Magnaporthe, Colletotrichum, Puccinia, Microdochium, Corynespora, Pucciniastrum, Olpidium, Craterocolla, Pseudotetraploa, Phoma, Cronartium, Septoria, Plectosphaerella, Didymella, Sphacelotheca, Pyrenochaeta, Drechslera, Spongipellis, Rhizoctonia, Endocronartium, Stenocarpella, Setophoma, Entyloma, Sydowia, Spongospora, Fomitopsis, Taphrina, Thielaviopsis, Fusarium, Tritirachium, Typhula, Ganoderma, Urocystis, Verticillium, Hypohelion, Ustilago, Waitea, Itersonilia, Venturia, Leptosphaerulina, Verticillium, and Monilinia.

133. The method of claim 114, wherein the plant pathogen belongs to one of the following genera: Pseudomonas, Erwinia, Raltsonia, Rhizomonas, Agrobacterium, Streptomyces, Bacillus, Sclerotium, Rhizoctonia, Fusarium, Pythium, Phytophthora, Synchytrium, Rhizopus, Alternaria, Macrophomina, Drechslera, Bipolaris, Curvularia, Phomopsis, Caloscypha fulgens, usarium circinatum, Fusarium oxysporum, Fusarium moniliforme var. moniliforme, Lasiodiplodia theobromae, Sirococcus conigenus, Diplodia pinea, Ustilago nuda, Pyrenophora graminea, Pyrenophora teres, Xanthomonas translucens, Pseudomonas syringae, Fusarium graminearum, Bipolaris sorokiniana, Xanthomonas campestris, Aciculosporium, Mycosphaerella, Ceratobasidium, Albugo, Alternaria, Myrothecium, Cochliobolus, Hyaloperonospora, Alveopora, Neonectria, Colletotrichum, Peronospora, Cadophora, Oculimacula, Curvularia, Phytophthora, Calyptella Omphalotus, Cylindrocladiella, Plasmopara, Chrysomyxa, Peyronellaea, Fusarium, Pythiogeton, Cladophialophora, Phaeoacremonium, Heterobasidion, Pythium, Coleosporium, Pseudocercospora, Magnaporthe, Colletotrichum, Puccinia, Microdochium, Corynespora, Pucciniastrum, Olpidium, Craterocolla, Pseudotetraploa, Phoma, Cronartium, Septoria, Plectosphaerella, Didymella, Sphacelotheca, Pyrenochaeta, Drechslera, Spongipellis, Rhizoctonia, Endocronartium, Stenocarpella, Setophoma, Entyloma, Sydowia, Spongospora, Fomitopsis, Taphrina, Thielaviopsis, Fusarium, Tritirachium, Typhula, Ganoderma, Urocystis, Verticillium, Hypohelion, Ustilago, Waitea, Itersonilia, Venturia, Leptosphaerulina, Verticillium, and Monilinia.

134. The method of claim 122, wherein the plant pathogen belongs to one of the following genera: Pseudomonas, Erwinia, Raltsonia, Rhizomonas, Agrobacterium, Streptomyces, Bacillus, Sclerotium, Rhizoctonia, Fusarium, Pythium, Phytophthora, Synchytrium, Rhizopus, Alternaria, Macrophomina, Drechslera, Bipolaris, Curvularia, Phomopsis, Caloscypha fulgens, usarium circinatum, Fusarium oxysporum, Fusarium moniliforme var. moniliforme, Lasiodiplodia theobromae, Sirococcus conigenus, Diplodia pinea, Ustilago nuda, Pyrenophora graminea, Pyrenophora teres, Xanthomonas translucens, Pseudomonas syringae, Fusarium graminearum, Bipolaris sorokiniana, Xanthomonas campestris, Aciculosporium, Mycosphaerella, Ceratobasidium, Albugo, Alternaria, Myrothecium, Cochliobolus, Hyaloperonospora, Alveopora, Neonectria, Colletotrichum, Peronospora, Cadophora, Oculimacula, Curvularia, Phytophthora, Calyptella Omphalotus, Cylindrocladiella, Plasmopara, Chrysomyxa, Peyronellaea, Fusarium, Pythiogeton, Cladophialophora, Phaeoacremonium, Heterobasidion, Pythium, Coleosporium, Pseudocercospora, Magnaporthe, Colletotrichum, Puccinia, Microdochium, Corynespora, Pucciniastrum, Olpidium, Craterocolla, Pseudotetraploa, Phoma, Cronartium, Septoria, Plectosphaerella, Didymella, Sphacelotheca, Pyrenochaeta, Drechslera, Spongipellis, Rhizoctonia, Endocronartium, Stenocarpella, Setophoma, Entyloma, Sydowia, Spongospora, Fomitopsis, Taphrina, Thielaviopsis, Fusarium, Tritirachium, Typhula, Ganoderma, Urocystis, Verticillium, Hypohelion, Ustilago, Waitea, Itersonilia, Venturia, Leptosphaerulina, Verticillium, and Monilinia.

135. The composition of claim 5, wherein the plant pathogen belongs to one of the following genera: Pseudomonas, Erwinia, Raltsonia, Rhizomonas, Agrobacterium, Streptomyces, Bacillus, Sclerotium, Rhizoctonia, Fusarium, Pythium, Phytophthora, Synchytrium, Rhizopus, Alternaria, Macrophomina, Drechslera, Bipolaris, Curvularia, Phomopsis, Caloscypha fulgens, usarium circinatum, Fusarium oxysporum, Fusarium moniliforme var. moniliforme, Lasiodiplodia theobromae, Sirococcus conigenus, Diplodia pinea, Ustilago nuda, Pyrenophora graminea, Pyrenophora teres, Xanthomonas translucens, Pseudomonas syringae, Fusarium graminearum, Bipolaris sorokiniana, Xanthomonas campestris, Aciculosporium, Mycosphaerella, Ceratobasidium, Albugo, Alternaria, Myrothecium, Cochliobolus, Hyaloperonospora, Alveopora, Neonectria, Colletotrichum, Peronospora, Cadophora, Oculimacula, Curvularia, Phytophthora, Calyptella Omphalotus, Cylindrocladiella, Plasmopara, Chrysomyxa, Peyronellaea, Fusarium, Pythiogeton, Cladophialophora, Phaeoacremonium, Heterobasidion, Pythium, Coleosporium, Pseudocercospora, Magnaporthe, Colletotrichum, Puccinia, Microdochium, Corynespora, Pucciniastrum, Olpidium, Craterocolla, Pseudotetraploa, Phoma, Cronartium, Septoria, Plectosphaerella, Didymella, Sphacelotheca, Pyrenochaeta, Drechslera, Spongipellis, Rhizoctonia, Endocronartium, Stenocarpella, Setophoma, Entyloma, Sydowia, Spongospora, Fomitopsis, Taphrina, Thielaviopsis, Fusarium, Tritirachium, Typhula, Ganoderma, Urocystis, Verticillium, Hypohelion, Ustilago, Waitea, Itersonilia, Venturia, Leptosphaerulina, Verticillium, and Monilinia.


SEQUENCE LISTING (36 sequences)

SEQ ID NO: 1 - DNA, 1327 bp, unidentified
  source 1..1327: mol_type = genomic DNA; note = multiple possible Streptomyces sp.
aatcatggcgccgggctaacacatgcagtccaacgaagaagcccggcggggtggattagaggcgaaacggtgagtaacacgtgagcaacctgcaccgcactctgggacaagccctggaaaaggggtccaagaccggatataagactgagaggcagcatccccgggtgtaaagctccgccggtgcaagatgagccccccgcctatcagctagctggtgagagaaaggctcaccaaggcgacgacgggtagcccgcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgagagtgacggtacctgcagaagaagcgccggctaactacgtgccagcagccgcggtaatacgtagggcgcaagcgttgtccggaattattgggcgtaaagagctcgtaggcggcttgtcacgtcgattgtgaaagcccgaggcttaacctcgggtctgcagtcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatacaccggaaagcattagagatagtgccccccttgtggtcggtgtaacaggtggtgcatggctgtcgtcagctcgtgtcgggagatgttgggttaagtcccgcaacgagcgcaacccttgttctgtgttgccagcatgcccttcgggggaatgggaacccacagaaaacccccgggcaaaactcgaagaaagggacggacgacctcaagtcatcatgcccctcatatctcgggctgcaaacgtgctacaatggccggtacaatgaactgcaaaaccgagaggtagagcgaatctcaaaaagccggtctcagttcggattggggtctgcaactcgaccccatgaagtcggagtcgctagtaatcgcagatcagcatgctgcgtatgcgac

SEQ ID NO: 2 - DNA, 1324 bp, unidentified
  source 1..1324: mol_type = genomic DNA; note = multiple possible Streptomyces sp.
agcgccgcgtgcgtacacaaacagtccaacgacgaacaccatcgggaagcatcagtggagaacgggtgaggaaaacgtgagcaaccggcactgcaccatgggacaagccctggaaacgcggacgaatacagaagatgaacctgggaggcatctcccggcgggtaaaactccggcggtgcagcagaagacccccgcctatcaacctgtgggggaggtaatggctcaccaaggcgacgacgggtagccggccgaaaagggccaacggccacactgggactgagacacggcccagactcctacgggaggcagcagtggggaatattgcacaatgggcgaaaacctgatgcagcgacgccgcgtgagggatgacggccttcgggttgtaaacctctttcagcagggaagaagcgagagtgacggtacctgcagaagaagcgccggctaactacgtgccagcagccgcggtaatacgtagggcgcaagcgttgtccggaattattgggcgtaaagagctcgtaggcggcttgtcacgtcgattgtgaaagcccgaggcttaacctcgggtctgcagtcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatacaccggaaagcattagagatagtgccccccttgtggtcggtgtacaggtggtgcatggctgtcgtcagctcgtgtcgtgagatgttgggttaagtcccgcaacgagcgcaacccttgtccggggttgccagcatgccctccggggtgatggggactcaaaagaaaccgccggggtcaactcggagaaaggtgggaacaacgtcaagtcatcatgccccttatgtcttgggccggcaacacgtgctacaatggccggtacaatgagctgcgataccgtgaggtggagcgaatctcaaaaagccggtctcagttcggattggggtctgcaactcgaccccatgaagtcggagtcgctagtaatcgcagatcagcatgctgcggtaatgcgc

SEQ ID NO: 3 - DNA, 1324 bp, unidentified
  source 1..1324: mol_type = genomic DNA; note = multiple possible Streptomyces sp.
cggggcgcgtgcttacacatgcactaaaacaatcaaacaagtcgaaacgaaataccagccaacgggagaataacacggggacaacctgccattgcacactcggaaaagccagcgaaacggggtctaataccggataccaatccagcagcaacatcccggagtcgaaagctccgccggagaaggatgaaccagcccccgataagcagctgggtgaggtagtggctcaccaaggcgacgacgggtagccgcccagagagagcgacaccccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccgggaattattgggcgtaaagagctcgtaggcggcttgtcacgtcgggtgtgaaagcccggggcttaaccccgggtctgcattcgatacgggcaggctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaaccttacaaaggcttgacatacaccggaaacgtctggagacaggcgcccccttgtggtcggtgtacaaggggggcaaggctgtcgtcagctcgtgtcgtgagatgttgggttaagtcccgcaacgagcgcaaccctggccctgtgttgccagcatgcccttcggggtgatgggaacccacaggaaaccgccggggccaactcggaggaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagtcggagttgctagtaatcgcagatcagcatgctgcgtaatgctc

SEQ ID NO: 4 - DNA, 1330 bp, unidentified
  source 1..1330: mol_type = genomic DNA; note = multiple possible Streptomyces sp.
aggggcggggtgcttacacatgcaagtcgaacgatgaaccccggcgggctggaacactggcaaacgaacgaggaacacctcgacaatatgccaccgtcacaccgggacaagccctgcaaacacagcctaaaaccggataacacatccacgcgaagccaggtcggtgaaaagctccgccggcgaaagatgagcccgcggcccatcagctgggtggtgaggtagtgacgcaacaaggcgacgacgggtagccggcccgaaaaggccaaccgccacactgggactgagacacggcccagaaccctacgggaagcagcagtggggaatatgcaaaaatgggccaaaacctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccgggaattattgggcgtaaagagctcgtaggcggcttgtcacgtcggatgtgaaagcccggggcttaaccccgggtctgcattcgatacgggcaagctagagtgcggtaggggagatcggaattcctggtgtagcggtgaaatgcgcagatatcaggaggaacaccggtggcgaaggcggatctctgggccaatactgacgctgaggagcgaaagcgtggggagcgaacaggattagataccctggtagtccacgccgtaaacggtgggaactaggtgtgggcgacattccacgtcgtccgtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaaacccccagagacaggcgcccccctgggggccgtgtacaggtggtgcatggctgtcgtcagctcgtgtcgtgagatgttgggttaagtcccgcaacgagcgcaacccttgttctgtgttgccagcatgcccttccgggggaaggggactcacaggaaaacggccggggtcaaactcggaagaaggtggggacgacgtcaagtcatcatgccccctatatcttcggggcctgcacacgtgctaaaaaagccagtaaaatgaactgcgaaaccccgagagcgaaacaacctcaaaaagccggtctcagttcggattggggtctgcaactcgaccccatgaagtcggagttgctagtaatcgcagatcagcatgctgcgtatggg

SEQ ID NO: 5 - DNA, 1336 bp, unidentified
  source 1..1336: mol_type = genomic DNA; note = multiple possible Streptomyces sp.
tcgtggcgcgcgcgtgctaacacatgaaagtcgaacaaggaacctccttcgcgagaagactagtggcaaacgggtgagtaacaccggagaaatctgccattcactacgggacaagccctggaaaacgggtctaataccggataccaatacacgaggcagcacctggaggtggaaagctccggcggtgaagaagaagccccccgcctatcagctgggtggtgaggtaacgccccaacaaagcgacgacgggtagcagccccgaaaaggccaacggcaacactgggactgagacacggcccagactccaacggaaggcagcagtggggaatattgcacaatggccgaaagccgaagcaaccgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccgaggcttaaccccgggtctgcattcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaatttcgacgcaacgcgaagaaccttaccaaggcttgacatacaccggaaaccactagagacaagcgcccccttgtggtcggtatacaggtggtgcatggctgtcgtcagctcgtggccggaagaatgttgggttaagtcccgcaacgagcgcaacccttgttctgtgttgccagcatgcccttcggggtgatggggactcacaggagaccgccggggtcaactcggaggaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagtcggagttgctagtaatcgcagatcagcatgctgcggtaaatgcgac

SEQ ID NO: 6 - DNA, 1320 bp, unidentified
  source 1..1320: mol_type = genomic DNA; note = multiple possible Streptomyces sp.
aggcgcgtcataacacatgcagccgaacgatcaaacactgcaaggtgaagaagaccccaacggaggagtaacaaacgggcaacctgacattcactcagggacaagccctgcaaaacgagtctaatacacgataccactcctccacgcatggccaggggttgaaaaagcccgcggtgaaggatgagcccccgccccatcagcttgttggggggggaatgacccaacaaggcgacgacgggtagccggcctgagagggccaaccgccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccgggaattattgggcgtaaagagctcgtaggcggcttgtcacgtcggatgtgaaagcccgaggcttaaccccgggtctgcattcgatacgggcaagctagagtgcggtaggggagatcggaattcctggtgtagcggtgaaatgcgcagatatcaggaggaacaccggtggcgaaggcggatctctgggccaatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaaaccatgaagacagggccccccttgtggtcggtatacaggtggtgcatggctgtcgtcagctcgtgtcgtgagatgttgggttaagtcccgcaacgagcgcaacccttgtcctgtgttgccagcatgcccttcggggtgatggggactcacaggaaaccgccggggtcaactcggagaaaggggacgacgacagcaagtcatcatgccccaaaggtcctggccgccacacgtgctacaagagccggtacaatgaccgcagataccgcgacgtgaagcgaagatcaaaaacccgatctcagtacgaattgaggactgcaactcaacaccagaaacgcgaaacggccagtaatcgcagatcagcatgctgcgtattccgg

SEQ ID NO: 7 - DNA, 1322 bp, unidentified
  source 1..1322: mol_type = genomic DNA; note = multiple possible Streptomyces sp.
aggggaggtgcttacacacgcactcgaaccatgaagacatcaggggtgaaagagtagccaacggggaaataacacacgggcaaactgcactgcactcggagacaaaccctggaaaccgggtcaaacaacagacaccacgcatgcctgcatgggcgggggtggaaagcaacgccgcggaaagatgagcccgcgccatacaaccgtgtgggtggggtaatgccccacaaaagcgacgacggcgagacggccgcagagagcgacacgcaaaacgggaacgaaaacacggcccagactcctacgggaggcagcagtggggaatattgcacaatgggcgaaaacctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccgaggcttaaccccgggtctgcattcgatacgggcaagctagagtgcggtaggggagatcggaattcctggtgtagcggtgaaatgcgcagatatcaggaggaacaccggtggcgaaggcggatctctgggccaatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaaacactagagacagggccccccttggggtcggtatacaggtggtgcatggctgtcgtcagctcgtgtcgtgagatgttgggttaagtcccgcaacgagcgcaacccttgtcctgtgtgccaacaagcccttccgggggaagggacacacacaaaagacggcacgcgaaaacgcggaggaaggtggggacgacggcaagtcatcatgccccttatgtctggcgctgcacacgtgctacaagcgcacatacaatgaactgcgataccggcgaggtggagacaatctcaaaaagccggtctcagttcggattggggtctgcaactcgaccccatgaagtcggagttgctagtaatcgcagatcagcatgctgcgtaatgccct

SEQ ID NO: 8 - DNA, 1332 bp, unidentified
  source 1..1332: mol_type = genomic DNA; note = multiple possible Streptomyces sp.
ccagccggtgcttaaacacacagtacaagtcgacaaaagataaagcccttcggaggggattagtggcaaacgggtgagtaacacgtgagcaacctgccattcaccctcagacaaacaagcggaaacggagtctaatacacgataccactcctccacgcatggtccgggggtggaaagctccggcggtgaagaaggagcccgccgcctatcagctggtggggggggtaagagcccaccaaggcgacgaccgggaacccgcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccgaggcttaaccccgggtctgcattcgatacgggcaagctagagtgcggtaggggagatcggaattcctggtgtagcggtgaaatgcgcagatatcaggaggaacaccggtggcgaaggcggatctctgggccaatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaaacactagagacagggccccccttgtggtcggtatacaggtggtgcatggctgtcgtcagctcgtgtcgtgagatgttgggttaagtccccaaacgagcgcaacccttgtcctgtgttgccagcatgcccttcggggtgatgggaacccacaagaaaccgccggggtcaactcgaagaaagggggggaacaactcaagtcatcatgcccctaatctcttgggctgcacacgtgctacaatggccggtacaatgagctgcgataccgcgaggtggagcgaatctcaaaaagccggtctcagttcggattggggtctgcaactcgaccccatgaagtcggagttgctagtaatcgcagatcagcatgctgcggtaatgctc

SEQ ID NO: 9 - DNA, 1323 bp, unidentified
  source 1..1323: mol_type = genomic DNA; note = multiple possible Streptomyces sp.
aaggcgcgcgaacacaaatgcaagtccaaaaaggaaacccttcgaggtggaataggcgagaacgggtgagtaacacgggggcaaccggcactgcactcacggacaagcccaagaaaaacggtctaatacacgataatactcctgccgcaatgggccggggttgaaagctccggcgcggaaagatgagcccgcggcctatcagctggttggtggggtaatggcccacaaaggcgacgacgggtagccagccgcagagagcgacacgccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccgggctaactacgtgccagcagccgcggtaatacgtagggcgcaagcgttgtccggaattattgggcgtaaagagctcgtaggcggcttgtcacgtcggatgtgaaagcccgaggcttaacctcgggtctgcattcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacgttgggaactaggtgttggcgacattccacgtcgtcggtgccgcagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatataccggaaagcattagagatagtgccccccttgtggtcggtatacaggtggtgcatggctgtcgtcagctcgtgtcgtgagatgttgggttaagtcccgcaacgagcgcaaccctggcccggtgttgccagcatgcccttcgggggaagggacaccaaaagaaaaccgccgcggtcaactcgaagaaaggtggggacgacgtcaagtcatcaagccccttatggctcgggctgcacacgtgctacaatgcccggtacaatgaactgccataccgagaagtgaagcgaatctcaaaaagccggccccacatccgattgggggcggcaactcgaccccatgaagtcggagtcgctagtaatcgcagatcagcatgctgcggttatgcta

SEQ ID NO: 10 - DNA, 1323 bp, unidentified
  source 1..1323: mol_type = genomic DNA; note = multiple possible Streptomyces sp.
agggcggcgtgctgacacataaaactcaaacgatgaagcaagtccgaatcaatgaatgccgaaagggaagaggaacacgtgagcaaccggccaatcactccgcgacaagccctggaaacggggtctaatacacgataccaatcctgcctgcatgaccggggggtgaaagctaaggcggcgaaaaaagaacccgcggccaatcagcttggcggtgggggaaaagcccaccaaggcgacgacgggtagccgccctgaaaaggccaacggccacactgggactgagacacggcccagactcctacgggaggcagcagtggggaatattgcacaatgggcgaaaacctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccgaggcttaacctcgggtctgcattcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacgttgggaactaggtgttggcgacattccacgtcgtcggtgccgcagctaacgcattaagttccccgcctggggagtacggccgcaaggctaaaactcaaaggaattgacgggggcccgcacaagcggcggagcatgtggcttaattcgacgcaacgcgaagaacctaaccaaggcttgacatataccggaaagcattagagatagtgcccccctgggggtcgcgaaaaaagtggtgcatggctgtcgtcagctcgtgtcgtgagatgttgggtaaagcccccaaaagagcacaacccttgtcctctgtggccagcatgcccttcgggggaaggaggactcacaggaaacccccggggtcaactcggagaaaggtggggacgacctcaagtcatcatgccccagacgtcttgggctgcacacgtgctacaatggccggtacaatgagctgcgataccgtgaggtggagcgaatctcaaaaagccggtctcagttcggattggggtctgcaactcgaccccatgaagtcggagtcgctagtaatcgcagatcagcatgctgcgtaatggcc

SEQ ID NO: 11 - DNA, 1327 bp, unidentified
  source 1..1327: mol_type = genomic DNA; note = multiple possible Streptomyces sp.
agggggggcgagcatacacacgaagtccaaccacgaacatccttcgcgaaacgatgagcgccgaaaagatgagcaacacgtgggcaagataacctgcacactgccacaagcacagcaaaaacggactaataccggataacaaatcaaaccgcatgcacggcggggggaaagctccggcggtgaaagatgagcccgcggcctatcagctgggtggggaggccacggcctaccaaggcgaaaacgacgacccgaccgcagagagcgacacgccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccgggaattattgggcgtaaagagctcgtaggcggcttgtcacgtcggatgtgaaagcccgaggcttaaccccgggtctgcattcgatacgggcaagctagagtgcggtaggggagatcggaattcctggtgtagcggtgaaatgcgcagatatcaggaggaacaccggtggcgaaggcggatctctgggccaatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaagaaatgaacgggggcccgcacaagcagcggagcatgtggcttaattcgacgcaaacccaaaaaacctaacaaagcttgacatacaccggaaaacactaaaaacagggccccccttgtggtcggtatacaggggggcaaggcctgtcgtcagctcgtgtcgtgagatgttgggttaagtcccgcaacgagcgcaacccttgtcctgtgttgccagcatgcccttcggggtgatggggactcacaggagaccgccggggtcaactcggaggaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagtcggagtcgctagtaatcgcagatcagcatgctgcggtaatgggc

SEQ ID NO: 12 - DNA, 1323 bp, unidentified
  source 1..1323: mol_type = genomic DNA; note = multiple possible Streptomyces sp.
acgcggcggtgcttaaacacgcaagcgaacgatgaagcccttcggggtggattagagaagaacgggtgagtaacacacgggcaacctgccattcactccggcacaaaccctggaaacagggtctaataaccgataccactacaacctgcagcagccggggttgaaagctcccgcggtgaagaaagaacccgcggcctatcagctggttggtgggggaaaggcccaacaaagcgacgacgggtagccgcccagagaaggccaacggcaaaacgggaacggagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccgaggcttaacctcgggtctgcattcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacgttgggaactaggtgttggcgacattccacgtcgtcggtgccgcagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatataccggaaagcattagagatagtgccccccttgtggtcggtatacaggtggtgcatggctgtcgtcagctcgtgtcgtgaaaggtggggtaaagtcccgcaacaagcgcaacccttgtcctgtgttgccagcatgcccttcggggtgatggggactcacaggagaccgccggggtcaactcggaggaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagtcggagtcgctagtaatcgcagatcagcatgctgcggtaatgcgtc

SEQ ID NO: 13 - DNA, 1330 bp, unidentified
  source 1..1330: mol_type = genomic DNA; note = multiple possible Streptomyces sp.
cgggcgggtgcttacacatgcagtcgaacaatgaaccacatcggggtagattagtaaccaacggatgagatatgaggaacacgtgagcaaactgccattcacactgggacaagccctggaaacggaaccgaatacacgataacactctgtcccgcatcgcacgaccggtaaaagctccggcggtgaaagatgagaccccgccctatcagcttgtgggtggggtaatggcctacaaaagcgacgacgggtagcccgcccgaagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccggggcttaaccccgggtctgcattcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacgttgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatataccggaaagcatcagagatggtgccccccttgtggtcggtatacaggtggtgcatggctgtcgtcagctcgtgtcgtgagatgttgggttaagtcccgcaacgagcgcaacccttgttctgtgttgccagcatgccctccgggggaagggacaccaaaagaagactgccggggtcaaccccgaaaaaaatggggacgacgtcaagtcatcatgccccttatgtcttgggctgcacacgtgctacaatggccggtacaatgagctgcgatgccgcgaggcggagcgaatctcaaaaagccggtctcagttcggattggggtctgcaactcgaccccatgaagtcggagttgctagtaatcgcagatcagcatgctgcgtaatgcctc

SEQ ID NO: 14 - DNA, 1319 bp, unidentified
  source 1..1319: mol_type = genomic DNA; note = multiple possible Streptomyces sp.
cgggggcacggtctaaacttgtaactccaaaaatgaaccccaatcgagagaggatcagtggaataatgaaacggtgaataaacgggcatatgttcttgctcattggacgacaagctaaaaacggtctaatacaagaaaaccacttgccgcatgtgcagggcggggaaaaaactccgcggaaaaaaaaaacccccgcccccatcagctgtggtggggtaaaggcccaccaaggcaaaacaacaagaagcccgctgagagggacacccccacactgggacagagacacgccccacactctaacggaggcagcagggggaaaaatgcacaatgggcgaaagcccaagcaaccaccccccgtgaagaaggacggccttcgggttgtaaacctccttcagcagggaagaagcgaaagtgacggtactgcagaagaagcgccggctaactacgtgccagcagccgcggtaatacgtagggcgcaagcgttgtccggaattattgggcgtaaagagctcgtaggcggcttgtcacgtcggatgtgaaagcccgaggcttaacctcgggtctgcattcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacgttgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatataccggaaagcattagagatagtgccccccttgtggtcggtatacaggtggtgcatggctgtcgtcagctcgtgtcgtgagatgttgggttaagtcccgcaacgagcgcaacccttgtcctgtgttgccagcatgcccttcggggtgatggggactcacaggagaccgccggggtcaactcggaggaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagttggagttgctagtaatcgcagatcagcatgctgcggaatccggc

SEQ ID NO: 15 - DNA, 1323 bp, unidentified
  source 1..1323: mol_type = genomic DNA; note = multiple possible Streptomyces sp.
cgggcgcgtgcttacacatgcagtcgaacgacaaaaaccgtcggcgtcgattactgcaaaacacgtgagcaacacggggacaagccaacctgcaacctgggaaaacccctggaaaccggaacgaacaaccgatacaactcctgcatgcatgggcgggggttgaaagcgaagcccgcgaaagatgagcccgcggcatatcagcagctgggtggggtaatgccacaccaaggcgacgacgggtagccgccctcagagagcgacacgccacactgggactgagacacggcccagacccctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccgggaattattgggcgtaaagagctcgtaggcggcttgtcacgtcggatgtgaaagcccgaggcttaacctcgggtctgcattcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacgttgggaactaggtgttggcgacattccacgtcgtcggtgccgcagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatataccggaaagcattagagatagtgccccccttggggtcggtatacagggggtgcatggctgtcgtcagctcgtgtcgtgagaaggtggggtaaagcccccaaacaacccaacccttgtcctgtgttgccagcatgcccttcggggtgatggggactcacaggaaaccgccggggcaaacccgaaagaaggggaccaccacatcaactcatcaggccccaaatgtcttgggctgcacacgtgctacaatggccggtacaatgagctgcgataccgtgaggtggagcgaatctcaaaaagccggtctcagttcggattggggtctgcaactcgaccccatgaagtcggagtcgctagtaatcgcagatcagcatgctgcgtaatggcgtc

SEQ ID NO: 16 - DNA, 1326 bp, unidentified
  source 1..1326: mol_type = genomic DNA; note = multiple possible Streptomyces sp.
cggggggcgtgcttacacatgcaagtcgaacgataaaacactccgcaatggaatacagacgaacccgtcagtaacacgtggaaaatcgcaacgtcactaacggacaagccctgcaaacggacaagaagaccggtttataccaacaccgcctgcatggccgggggttgaaagctccggcgcggaaagatgagcccgcgccctatcagctggttggtgggggaaaagcccaccaaggcgacgacgggtagccgcccgcagagagcgacaccccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccgggaattattgggcgtaaagagctcgtaggcggcttgtcacgtcggatgtgaaagcccgaggcttaacctcgggtctgcattcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacgttgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatataccggaaagcattagagatagtgccccccttgtggtcggtatacaggggggccatggctgtcgtcagctcgtgtcgtgagatgttgggttaagtcccgcaacgagcgcaacccttgtcctgtgttgccagcatgcccttcggggtgatggggactcacaggagaccgccggggtcaactcggaggaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagtcggagtcgctagtaatcgcagatcagcatgctgcgtatgctaa

SEQ ID NO: 17 - DNA, 1339 bp, unidentified
  source 1..1339: mol_type = genomic DNA; note = multiple possible Streptomyces sp.
gagtcagcaccgcgtcgcgcgggggctaaaaaatgcaagtcgaacaaagaagcccttcggggtggattagtggcgaacggggaaataacaacggggaaaactgcccatcactctgggacaagccctgcaaacacagccgaacacccgataccacgcaagcctgcatgcacggcgggtgaaagctccggcgcggaaagatgagcccgcggcctaatcagcttgttggtggggtaaggccccaacaaggcgacgacgggtagccggccgcagagagcgacacgccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccgggaattattgggcgtaaagagctcgtaggcggcttgtcacgtcggatgtgaaagcccgaggcttaacctcgggtctgcattcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacgttgggaactaggtgttggcgacattccacgtcgtcggtgccgcagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatataccggaaagcattagagatagtgccccccttgtggtcggtatacaggtggtgcatggctgtcgtcagctcgtgtcgtgagatgttgggttaagtcccgcaacgagcgcaacccttgtcctgtgttgccagcatgccctccggggtgatggggacccaaaagagacacccccgggcaacaccgacgaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagtcggagtcgctagtaatcgcagatcagcatgctgcgtaatgcgtg

SEQ ID NO: 18 - DNA, 1325 bp, unidentified
  source 1..1325: mol_type = genomic DNA; note = multiple possible Streptomyces sp.
cgggtgcgtgcttacacatgcagtcgaacgatgaacccactcgcagtggattagtggcaaacgagagagtaacacgtgaacaagcgcaccttcacgctaggacaagccatggaaacgggacagaagaccggacaacacgacagcatccagcggcagggcaggaaagaaccgccgcggaaagatgagcccgcggcctatcagcttggtggtggagtaaaggcccaccaaggcgaccaacgacagcagccctgagagggcgaccgccaacactgggactgagacacggcccagactcctacgggaggcagcagtggggaatattgcacaatgggcgaaagccgaatgcagcgacgccgcgtgagggatgaacgcccttcgggttgtaaacctctttcagcagggaagaagcgaaagtgacggtacctgcagaagaagcgccggctaactacgtgccagcagccgcggtaatacgtagggcgcaagcgttgtccgggaattattgggcgtaaagagctcgtaggcggcttgtcacgtcggatgtgaaagcccgaggcttaacctcgggtctgcattcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacgttgggaactaggtgttggcgacattccacgtcgtcggtgccgcagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatataccggaaagcattagagatagtgccccccttgtggtcggtatacaggtggtgcatggctgtcgtcagctcgtgtcgtgagatgttgggttaagtcccgcaacgagcgcaacccttgtcctgtgttgccagcatgcccttcggggtgatggggactcacaggagaccgccggggtcaactcggagaaaggggacgacgacgtcaagtcatcatgcccctaaggtcttgggctgcacacgtgctacaaggcccacaacaacgaccaaccataacgggagatctcaagcgaatcaaaaaaagccgatcgcaactcgaacaggagtcggaaactcgaccccatgaagtccgagtcactagcaatcacagatcagcatgctgcgtaaagcgtg

SEQ ID NO: 19 - DNA, 1337 bp, unidentified
  source 1..1337: mol_type = genomic DNA; note = multiple possible Streptomyces sp.
cgggcgggtgcttacacatgcagtcgaacgatgaagcccttcggggtggattaagaacaaacgggcgagtctaacaccaaggcaagctgccctacactcgaagacaagcacgggaaaacgggccaaagaccgcataacaagcctcacgcctccatgcgagcgaggcgaaacatcccgcggtgaaggatcagcgggagatgacccgcggcctatcagctggttggtgggggaaacgcccaccaaggcgacgacgggaacccggcccgaaaaggccaacgccaacactgggactgagacacggcccagactcctacgggaggcagcagtggggaatattgcacaatggccgaaaacctgatgcagcgacgccgcgtgagggatgacggccttccgggtgtaaacctctttcagcagggaagaagcgaaagtgacggtaactgcagaagaagcgccggctaactacgtgccagcagccgcggtaatacgtagggcgcaagcgttgtccgggaattattgggcgtaaagagctcgtaggcggcttgtcacgtcggatgtgaaagcccgaggcttaacctcgggtctgcattcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacgttgggaactaggtgttggcgacattccacgtcgtcggtgccgcagctaacgcattaagttccccgcctggggaagaacgccccaaagccaaaaaccaaaggaattgacgggggcccgcacaagcggcggagcatgtggcttaattcgacgcaacgcgaagaaccttaccaaggcttgacatataccggaaagcattagagatagtgccccccttgtggtcggtaaaaaaggtggtgcatggctgtcgtcagctcgtgtcgtgagatgttgggttaagtcccgcaacgagcgcaacccttgtcctgtgttgccagcatgcccttcggggtgatggggactcacaggagaccgccggggtcaactcggaggaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagtcggagtcgctagtaatcgcagatcagcatgctgcgtatgcggg

SEQ ID NO: 20 - DNA, 1323 bp, unidentified
  source 1..1323: mol_type = genomic DNA; note = multiple possible Streptomyces sp.
agggcgggtgcttaccaagcaactcgaaacatgaaactcccaagggagaacagtaaaggcgaaaggggaaataacacgtaggaaatcggcaattcaccatcggacaagccctggaaaccgggtccaatacacgatacgacctccaacaccatagcatcggggggaaaagctccgccggagaaggatgagcacccgccctatcagctgggtgaaggagtgatggcccacaaaagcgacgacgggtagcagccccgaaaaggccaacggccacactgggactgagacacggcccagactcctacgggaggcagcagtggggaatatgcaaaaaggggccaaaacctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccggggcttaaccccgggtctgcattcgatacgggcaagctagagtgcggtaggggagatcggaattcctggtgtagcggtgaaatgcgcagatatcaggaggaacaccggtggcgaaggcggatctctgggccaatactgacgctgaggagcgaaagcgtggggagcgaacaggattagataccctggtagtccacgccgtaaacggtgggaactaggtgtgggcgacattccacgtcgtccgtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaaaccccagagaacagcgcccccttgtggtcggtgtacaggtggtgcatggctgtcgtcagctcgtgtcgtgagatgttgggttaagtcccgcaacgagcgcaacccttgttctggtgttgccagcatgcccttcggggtgatggggactcaaaagaaaacgcccgggtcaactcggagaaaggggacgacgaagtcaacacatcatgccccttatgtcttgggctgcaaacggccaaaaagggcacgtacaatgaccggcaaacccgagaggcggaacgaatctcaaaaagccagtcgaagggcgcactgcactctgcaacgcaacccaatgaaatcggaggagctagtaatcgcagatcagcatgctgcgtatgcag

SEQ ID NO: 21 - DNA, 1329 bp, unidentified
  source 1..1329: mol_type = genomic DNA; note = multiple possible Streptomyces sp.
cggggcgactcatgacaaatgcaaaaagaaagatgaacccccttcgcgaggggattagtggcgaaacggtgagtaacacgtgcacaacctgcacttcactctgggacaaaacccggaaaccgagtctaataccggatacgaacacacaccgcagcatcgcggggtggaaagctccggcggtgaagaagaagcccgcggcctataacagcggtggtggggtaatggcccaccaaggcggacgacgggtagccggcccgaaaaggccaacggccacactgggactgagaaaacgcccaaaaccctacgggaggcagcagtggggaatattgcacaatgggcgaaaacctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccgaggcttaaccccgggtctgcattcgatacgggcaagctagagtgcggtaggggagatcggaattcctggtgtagcggtgaaatgcgcagatatcaggaggaacaccggtggcgaaggcggatctctgggccaatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaagccttgacatacaccggaaaacactagagacagggccccccttgtgggccgtatacaggtggtgcatggctgtcgtcagctcgtgtcgtgagaatgttgggttaagtcccgcaacgagcgcaacccttgtcctgtgttgccagcatgcccttcggggtgatggggactcacaggagaccgccggggcaaactccgaagaaggtggggacaacgtcaagtcatcatgccccttaagtcctgggctgcacaaggccaaaaagcgccacaacaatgacatgcgataccgcgaggtggagcgaatctcaaaaagccgctctcagtccgaatggaagacagcaactcgaccccatgaagtcggagtcgctagtaatcgcagatcagcatgctgcgttaatgctc

SEQ ID NO: 22 - DNA, 1326 bp, unidentified
  source 1..1326: mol_type = genomic DNA; note = multiple possible Streptomyces sp.
tcgggcccctaaacacacaggcaagtccaacgatgaagcctttcgggaccgatgagccgcgaaagggagaggaaaacgtgagcaacctgcccatcactctggcacaagaccagcaaacgacggctaaaacacgatacgacaaacgaccgcatggtctgggtgtggaaagctccggcggtgaaagaagaccccgcgacctatcagcttggtggtggggtgatggcataccaaagcgacgacggggaacccgcctgagagggcgaccgccaacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaaaccctggagacagggcccccctgggggtcggtgtacaggtggggcaaggcctgtcgtcagctcgtgtcgtgagatgttgggttaagtcccgcaacgagcgcaacccttgttctgtgttgccagcaagcacttccgggggagagggactcacaggagactgccggggtcaactcggaggaaggtggggacgacgtcaagtcatcatgccccttatgtcttgggctgcacacgtgctacaatggccggtacaatgagctgcgataccgcgaggtggagcgaatctcaaaaagccggtctcagttcggattggggtctgcaactcgaccccatgaagtcggagttgctagtaatcgcagatcagcatgctgcggtaatgcttc

SEQ ID NO: 23 - DNA, 1326 bp, unidentified
  source 1..1326: mol_type = genomic DNA; note = multiple possible Streptomyces sp.
cggggcggcctgcctaaacacgcaagtcgaacgatgaagcccttcacggaggactagcgccgaacgaatgaggaaaacgtgagcaacctgcactgcactccgggacaagacctggaaacggggtctaaaaccggataagacaaaccaacgcaacgcatgtgcgtggaaagcgaagccgcggaaggatgagcccgcggcccaatcagcttgttggtggggtgatgacccaacaaggcgacgacgggtagccggcctgagagggcgaacggccacactgggactgagacacggcccagactcctacgggaggcagcagtggggaatattgcacaatgggcgaaaacctgatgcagcgacgccgcgtgagggatgacggccttccgggtgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcagcggagcatgtggcttaattcgacgcaacccaaagaaccttaccaaggcttgacatacaccggaaaaaccctggagacaaggtcccccttgtggtcggtgtacaggtggtgcatggctgtcgtcagctcgtgtcgtgagatgttgggttaagtcccgcaacgagcgcaacccttgttctgtgttgccagcatgcccttcggggtgatggggactcacaggagactgccggggtcaactcggaggaaggtggggacgacgtcaagtcatcatgccccttatgtcttgggctgcacacgtgctacaatggccggtacaatgagctgcgataccgcgaggtggagcgaatctcaaaaagccggtctcagttcggattggggtctgcaactcgaccccatgaagtcggagttgctagtaatcgcagatcagcatgctgcgtaatgcta

SEQ ID NO: 24 - DNA, 1328 bp, unidentified
  source 1..1328: mol_type = genomic DNA; note = multiple possible Streptomyces sp.
cgggaggtgcctacaaaaccactcgaaagacaaacctacgactccgcagaggaataggggcgaacgggtgagtaacaccggggaaaactgcactgcactcagggacaagccctggaaacgggctctaaaaccagaaacgacatacgaccgcaacgcatggtcgtggaggaagctccgccggtgaaggaagagcccccgccctatcagctggttggtggggtgatgacccaacaaggcgacgacgggtagccggccgaaaagggcgaccggccacactgggactgagaaaacgcccaaaaccctacgggaggcagcagtggggaatatgcaaaaatgggccaaaacctgatgcagcgacgccgcgtgagggatgacggcctccgggttggaaaaccccttcagcagggaagaagcgagagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaaaccctggagacagggtcccccttgtggtcggtgtacaggtggtgcatggctgtcgtcagctcgtgtcgtgagatgttgggtaaagtcccgcaacgagcgcaacccttgttctgtgtgccaagcatgcccttcggggtgatggacactcacaggagactgccggggcaaacgagaaggaaggacgggacgaacgcaagtcatcatgcccctaatctcgtggcctgcacaccggctacaatgacccgtacaatgaactgcgaaaaccccaagtggagcaaatctcaaaaagccagacccaattcggactgcactcggcaactcgaacccatgaacccggaattgctaataaacacacaacacaatgctgcgtaatgccgtc

SEQ ID NO: 25 - DNA, 1328 bp, unidentified
  source 1..1328: mol_type = genomic DNA; note = multiple possible Streptomyces sp.
cgggcgggtgcttaaacatgcacgcaaacgatgaacctccctcgtcggaggggaatagtggcaaacgggtgagtaacacgtggacaacctgccattcactacgcgacaagccctggaaacggacaccaaaaccggataagaccaccgacccgaagcatctcgtggtggaaagctccgccggtgaaagatgagcccgccgccaatcagcttgtggggggggtgatggccgacaaaggcgacgaacgggaacccgcctgagagggcgaccggccacactgggactgagacacggcccagactcctacgggaggcagcagtggggaatattgcacaatgggcgaaagcctgatgcagcgacgccgcgtgagggatgaccgcccttcgggttgtaaacctctttcagcagggaagaagcgagagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaaaccctggagacagggtcccccttgtggtcggtgtacaggtggtgcatggctgtcgtcagctcgtgtcgtgaagatgttgggttaagtcccgcaacgagcgcaaccctggtccggggtggccagcatgcccttcggggtgatggggactcacagaaaacggccggggtcaactcggaggaaggtggggacgacgtcaagtcatcatgccccttatgtcttgggctgcacacgtgctacaatggccggtacaatgagctgcgataccgcgaggtggagcgaatctcaaaaagccggtctcagttcggattggggtctgcaactcgaccccatgaagtcggagttgctagtaatcgcagatcagcatgctgcgtatgcggc

SEQ ID NO: 26 - DNA, 1339 bp, unidentified
  source 1..1339: mol_type = genomic DNA; note = multiple possible Streptomyces sp.
tgattcgaaaggccgcggctaacacatgcaggcgaacgatgaacctccttcgggaggggattagtggcgaacgggtgagtaacacctcgacaatatgccaatcacgcaggaacaagccatggaaacagggacaaataaacggtatatcaagaccacccaacgcatggtctgctgggggaaagctcccgcggcgaaagatgagcccccgccctatcagctgggtggggggatgatggcctacaaaagcgacgacgggtagccgaccgcagagagcgacacgccacactgggactgagacacggcccagactcctacgggaggcagcagtggggaatatgcaaaaaggggccaaaacctgatgcagcgacgccgcgtgagggatgacggccttcgggttgtaaacctctttcagcagggaagaagcgagagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaaaccctggagaacagggtcccccttgtggtcggtgtacaggtggtgcatggctgtcgtcagctcgtgtcgtgaaatgttgggataaagccccgcaacgagcgcaaacccttgttctgtgttgccagcatgcccctccgggggaaggggactcacagaaaactgccggggccaactcgaagaaaggggacgacgaagtcaagtcatcaggcccctaaggtcttgggctgcaaacgtgccaaaatggccggtacaatgagctgcgataccgcgaggtggagcgaatctcaaaaagccggtctcagttcggattggggtctgcaactcgaccccatgaagtcggagttgctagtaatcgcagatcagcatgctgcgtaatgcgc

SEQ ID NO: 27 - DNA, 1323 bp, unidentified
  source 1..1323: mol_type = genomic DNA; note = multiple possible Streptomyces sp.
cgggcgggtgcttacacatgcagtcgaacgatgaacctccttcgggaggggattagtggcaaacgcgtgagtaacacgtgaacaacatgcacgtaactcgagaacaagacctggaaacgcaggctaatgacagcagacgacaccggatcgaacgaccaccacgtcgaaaactccggccgcgaaggataagcccgcggcctatcagctggttggtggggtgagggacgacaaaagcgacgacgggtagcagccctgagagggcgacacgccacactgggactgagacacgccacagacacccaacggaggcagcagtggggaaaatgcaaaaatgggcgaaagcctgatgcagcgacgccgcgtgagggatgacggccgcccgggtgtaaacctctttcagcagggaagaagcgagagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaaaccctggagacagggtcccccttgtggtcggtgtacaggtggtgcatggctgtcgtcacctcgtgtcgtgagatgttgggttaagtcccgcaacaaccgcaacccttgttccggggtgccagcatgcccttcggggtgatgggcactcacaagaaacggcccgggtcaaccccgaggaaggtggggacgacgtcaagtcatcatgccccttatgtcttgggctgcacacgtgctacaatggccggtacaatgagctgcgataccgcgaggtggagcgaatctcaaaaagccggtctcagttcggattggggtctgcaactcgaccccatgaagtcggagttgctagtaatcgcagatcagcatgctgcgtatgcac

SEQ ID NO: 28 - DNA, 1325 bp, unidentified
  source 1..1325: mol_type = genomic DNA; note = multiple possible Streptomyces sp.
cggtgccgacgaacacaggcaaggcgaaagaacaaccaactgcgagaagggataagccgcgaacgaataaggaaaacgtgagcaacctgcccatcactccagcacaaaccctggaaacggggtctaatacacgatacgacctccgaccgcaaggtctgggggtggaaagctccgccggagaaggaagaacccgcggcctatcagctgggtgggggggtgatgccctacaaaggcgacgacgggtagccggcctgagagggcgaccacccaaaatgggactgagacacggcccagactcctacgggaggcagcagtggggaatatggcaaaaagggccaaaacctgatgcagcgacgccgcgtgagggatgacggccttcgggttggaaacctctttcagcagggaagaagcgagagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaaaccctggagacagggtcccccttgtggtcggtgtacaggtggtgcatggctgtcgtcagctcgtgtcgtgagatgttgggttaagtcccgcaacgagcgcaacccttgttctgtgttgccagcatgcccttcggggtgatggggacccaaaagagactgccggggccaactcggagaaaggggaccacgacagcaagtcatcatgcccctaatgtctggcgccaaacacgtgctacaagggaccggacaatgaactgccataccgcgaaggtggaacgaatctcaaaaagccggtctcagttcggattggggtctgcaactcgaccccatgaagtcggagttgctagtaatcgcagatcagcatgctgcggtattgctc

SEQ ID NO: 29 - DNA, 1325 bp, unidentified
  source 1..1325: mol_type = genomic DNA; note = multiple possible Streptomyces sp.
cgggcgcgtgcttacacatgcaagtcgaaccaaaaaccaccgtcaagaggcgataagtggcgaacgggtgacaaacacggaggcaatatgccccacactcaggaacaagccatggaaaacgggacaaataacgggatacaacatacgaccgcaacgcatggtcgtggaaaactccggccgcgaaagatgagcccgcgccatatcagcttgcagctggggtgagggcataccaaagcgacgacgggtagccggccgcagagagcgacaccccacactgggactgagacacggcccagactcctacgggaggcagcagtggggaatattgcacaatgggcgaaagcctgatgcagcgacgccgcgtgagggatgacggccttccgggtggaaacctctttcagcagggaagaagcgagagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaaaccctggagacagggtcccccttgtggtcggtgtacaggtggtgcatggctgtcgtcagctcgtgtcgtgagaaggtgggttaagtcccgcaacgagcgcaacccttgttctgtgttgcaaccaagcccttcggggtgatggggactcacaggagactgccggggtcaacccggaagaaagtggggacgacgtcaagtcatcatgccccttatgtcttgggctgcacacgtgctacaatggccggtacaatgagctgcgataccgcgaggtggagcgaatctcaaaaagccggtctcagttcggattggggtctgcaactcgaccccatgaagtcggagttgctagtaatcgcagatcagcatgctgcgtaatgcac

SEQ ID NO: 30 - DNA, 1329 bp, unidentified
  source 1..1329: mol_type = genomic DNA; note = multiple possible Streptomyces sp.
cgggcgcgcgaaaaaacatgcaagtcgaacaatgaacccccttcggaaggagattagtggcgaacgggtgaggaaaacgtgggcaatctgcccttcactaacggacaagccccggaaaccggaccgaataaccgatacgaccacacaacgcatggtctggtgggggaaagctccgccggtgaaagatgagcccgcgccctatcagctggtgggtgaggtgatggcctaccaaagcgacgacgggtagcagcccgcagagagcgacacgccacactgggactgagacacggcccagacccctacgggaggcagcagtggggaatattgcacaatgggcgaaagcctgatgcagcgacgccgcgtgagggatgacggccttcgggttgtaaacctctttcagcagggaagaagcgagagtgacggtacctgcagaagaagcgccggctaactacgtgccagcagccgcggtaatacgtagggcgcaagcgttgtccgggaattattgggcgtaaagagctcgtaggcggcttgtcacgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaacgtctggagacaggcgcccccttgtggtcggtgtacaggtggtgcatggctgtcgtcagctcgtgtcgtgagatgttgggttaagtcccgcaacgagcgcaacccttgttctgtgttgccagcatgcccttcgggggaaggggaacccacaggagactgccgggcacaactcggagaaagggggggaacaacgcaagtcatcatgccccttatgtctggcgcccaacacgtgctacaatggccggtacaaggaactgcaacacagcgaaatggagccaatctcaaaaagccggtctcaattcgaattggaggcaccaactcgaccccaagaagtcggagtggctaataatccaatcacagatcagcacgctgcgtatgcgc

SEQ ID NO: 31 - DNA, 1324 bp, unidentified
  source 1..1324: mol_type = genomic DNA; note = multiple possible Streptomyces sp.
ggggcgcgtgcttacacatgcagtcgaacgatgaacctccttagggagaagagtaaggccgaacgagtgagaaacacacgggcaaactgccattcactcagggacaagccctggaaacgacagctaaaaccggacaagaccacacaccccacggcatggcggaggaaaactccggcggtgaaagatgagcccgcgccctatcagcagctggggggggtaatggcccaccaaggcgacgacgggaacccgccctgaaaaggccaacggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccgggctaactacgtgccagcagccgcggtaatacgtagggcgcaagcgttgtccggaattattgggcgtaaagagctcgtaggcggcttgtcacgtcggatgtgaaagcccgaggcttaaccccgggtctgcattcgatacgggcaagctagagtgcggtaggggagatcggaattcctggtgtagcggtgaaatgcgcagatatcaggaggaacaccggtggcgaaggcggatctctgggccaatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaaacactagagacagggccccccttgtggtccgtatacaggtggtgcatggctgtcgtcagctcgtgtcgtgagatgttgggttaagtcccgcaacgagcgcaacccttgtcctgtgttgccagcatgcccttcggggtgatggggactcacaggagaccgccggggtcaactcggagaaaggggaggaccaacgcaagtcatcatgccccttatgtcctgggctgcacacgtgctacaatgcccggaaaaaaaagctgccaaaccgcgaggtggagcgaatctcaaaaacccgggcccagttcggattgagagatgcaactccaacccaggaactcggagtcgctagaaatcccagataaacacgctgcggcatgcgc

SEQ ID NO: 32 - DNA, 1328 bp, unidentified
  source 1..1328: mol_type = genomic DNA; note = multiple possible Streptomyces sp.
cgcgggcgctgcctaaacacgcaaggcgaacgatgaacatcatcccgcagaggaagagtggcgaacggcaaagtaacaactggccaatccgcccttcactctgggacaagccctggaaaaccggtctaatacccgatacaacacggggtcgcatgacccccgtgtggaaagccccggcggtgaaagatgagcccgcgccatatcagcttgtgggtggggtgatggccgaccaaagcgacgacgggtagccggcccgaaaaagcgacacgccacactgggactgagaaaacgcccaaaaccctacgggaggcagcagtggggaatattgcacaatgggcgaaagcctgatgcagcgacgccgcgtgagggatgacggccttcgggttgtaaacctctttcagcagggaagaagcgagagtgacggtacctgcagaagaagcgccggctaactacgtgccagcagccgcggtaatacgtagggcgcaagcgttgtccgggaattattgggcgtaaagagctcgtaggcggcttgtcacgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaaaccctggagacagggtcccccttgtggtcggtgtacaggtggtgcatggctgtcgtcagctcgtgtcgtgagatgttgggttaagtcccccaaacaacccaacccttgttctgtgttgccagcatgcccttccaggtgatggggactcacaggaaaacgcccggggtcaaccccgaggaaggggacgacgacgtcaagtcatcatgccccttatgtcctgggctgcacacgggctacaagcgcacataaaagcagctgcgaaaccgccaaggggaaccaatctcaaaaagccagtctcagtccgaattggggtctgcaacccgaacacaagaactcagaggcgctaggaaccccagatcagcatgctgcttaatgcgac

SEQ ID NO: 33 - DNA, 1335 bp, unidentified
  source 1..1335: mol_type = genomic DNA; note = multiple possible Streptomyces sp.
caggggggtgcttacacatgcagtcgaacgatcaacaactgcgcgctggattaacagcgaaagggtgagtaaaacgtgggcaatatgaccttcactcagagacaagaacgggaaaaccgggccaataccgcataacacctgcaacggcatccggatacgataaaagctccggcatgcaggtgaagctccgcgaaagatgagcccgcgccataacagctggttggtgagggaggggatcacaaaggagacgacgacgagcagccctgagagggcgaccgccaacactgggactgagacacggcccagactccaacgggaggcagcagtggggaatatgcaacaaagggccaaaacctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccgggaattattgggcgtaaagagctcgtaggcggcttgtcacgtcggatgtgaaagcccgaggcttaaccccgggtctgcattcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaaccaccagagatagtccccccctgggggtcggtatacaggtggtgcaaggctgtcgtcagctcgtgtcgtgagatgttgggtaaagcccccaaacaaccccaacccttgttccggtgttgccagcatgcccttccgggtgatggggactcacaggagaccgccggggtcaactcggaggaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagtcggagtcgctagtaatcgcagatcagcatgctgcgtatccg

SEQ ID NO: 34 - DNA, 1325 bp, unidentified
  source 1..1325: mol_type = genomic DNA; note = multiple possible Streptomyces sp.
ctggcgcgtccaaacacaatgcaagtccaacgaagaagcccttcggggtggattagtggccaacgagtgagtaaccaacggggacaaactgccaatcactcacggacaagacaagcaaaacgggtctaataccagaaaatacacctgccagcatcggcgggggtggaaaaagccggcgggcaagaagaagacccccgcctatcagctggtgggggaggtaatggcccaccaaagcgacgacgggtagcccgccgaaaagggcgaccgccaacactgggactgaaaaaacggcccagactcctacgggaggcagcagtggggaatattgcacaatgggcgaaagcctgatgcagcgacgccgcgtgagggatgacggcctccgggttggaaaaccccttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccgaggcttaaccccgggtctgcattcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaaccaccagagatagtccccccctgggggccggagaaaaggtggtgcatggctgtcgtcagctcgtgtcgtgagatgttgggttaagtcccgcaacgagcgcaacccttgttctgtgttgccagcatgcccttcggggtgatggggactcacaggagactgccggggtcaactcggaggaaggtggggacgacgtcaagtcatcatgccccttatgtcttgggctgcacacgtgctacaatggcaggtacaatgagctgcgaagccgtgaggcggagcgaatctcaaaaagcctgtctcagttcggattggggtctgcaactcgaccccatgaagtcggagttgctagtaatcgcagatcagcatgctgcgtatgccta

SEQ ID NO: 35 - DNA, 1326 bp, unidentified
  source 1..1326: mol_type = genomic DNA; note = multiple possible Streptomyces sp.
cggggggcgtgaatacacatgcaatgtcgaaccataaaacccttcggggaggattagtggcgaacgggtaaggaaaacgtgagcaacctgcacgccactcggggacaagccctggaaacggaccagaataacggataacaccagacaccgcaaccagctgagagaaaagctccggcggtgaaggatgagcccgcggcctatcagctggtgggtgaagtagtggctcaccaaggcaacgacgacgagcagccccgagagagccaacgccaacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcgggtgtgaaagcccggggcttaaccccgggtctgcattcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacggtgggaactaggtgttggcgacattccacgtcgtcggtgccgcagctaacgcattaagttccccgcctggggagtacggccgcaaggctaaaactcaaagaaatgaacgggggcccgcacaagcagcggagcatgtggcttaattccaaccaacgcgaagaaccttaccaaggcttgacatacaccggaaacggccagagatggtcgccccctggggggccgtgtacaggtggtgcatggctgtcgtcagctcgtgtcggaagatgttgggttaagtcccgcaacgagcgcaacccttgttctgtgttgccagcatgcccttcggggtgatggggactcacaggagactgccggggtcaactcggaggaaggtggggacgacgtcaagtcatcatgccccttatgtcttgggctgcacacgtgctacaatggcaggtacaatgagctgcgaagccgtgaggcggagcgaatctcaaaaagcctgtctcagttcggattggggtctgcaactcgaccccatgaagtcggagttgctagtaatcgcagatcagcatgctgcgtaattgccgtg

SEQ ID NO: 36 - DNA, 1335 bp, unidentified
  source 1..1335: mol_type = genomic DNA; note = multiple possible Streptomyces sp.
aagggcggcgagatcacacacgaaagtcgaacgacgaaacactgcggggtggattagtggcgaacgggacagtaacaccagggcaatatgccaacgtataacacgggcattcgcacaaccacagcaaaaacggtctaataccagataataataccgcctgcatggccggggggtgaaagccccggcggtgaaagatgagcccgcggcctatcagcttgttggggggggaaaggcccaccaaggcgacgacgggtagcccgcctgaaaaggccaacggccacactgggactgagacacggcccagactcctacgggaggcagcagtggggaatattgcacaatgggcgaaaacctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccgaggcttaacctcgggtctgcattcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacgttgggaactaggtgttggcgacattccacgtcgtcggtgccgcagctaacgcattaagttccccgcctggggagtacggccgcaaggctaaaaccaaaagaaatgaacgggggcccgcacaagcggcggagcatgtggcttaattcgacgcaacgcgaagaaccttaccaaggcttgacatataccggaaagcattagagatagggccccccttgtggtcggtatacagggggtgcatggctgtcgtcagctcgtgtccggagagatgttgggttaagtcccgcaacgagcgcaacccctggcctgtgttgccagcatgcccttcggggggatggggactcacaggaaaccgccggggcaaacccggaggaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagtcggagtcgctagtaatcgcagatcagcatgctgcggtatccgc